(19) United States
(12) Patent Application Publication
(10) Pub. No.: US 2022/0227830 A1
(43) Pub. Date: Jul. 21, 2022

(54) QD DOSING OF GIP RECEPTOR AGONIST PEPTIDE COMPOUNDS AND USES THEREOF

(71) Applicant: Takeda Pharmaceutical Company Limited, Osaka (JP)

(72) Inventors: Antoine Charles Olivier Henninot, San Diego, CA (US); Derek Cecil Cole, San Diego, CA (US); Nicholas Scorah, San Diego, CA (US)

(21) Appl. No.: 17/554,539

(22) Filed: Dec. 17, 2021

Related U.S. Application Data
(63) Continuation of application No. PCT/JP2021/014423, filed on Mar. 25, 2021.
(60) Provisional application No. 62/994,716, filed on Mar. 25, 2020.

(51) Int. Cl. C07K 14/605; A61K 9/20; A61K 9/08; A61K 9/00; A61P 1/08

(52) U.S. Cl. CPC C07K 14/605; A61K 9/2018; A61K 9/2059; A61K 38/00; A61K 9/0019; A61P 1/08; A61K 9/08

(57) ABSTRACT

The present disclosure provides GIP receptor agonist peptide compounds suitable for once per day dosing (QD), said peptide compounds having an activating action on GIP receptors and use of the GIP receptor agonist peptide as a medicament for the treatment and/or prevention of emesis, or a symptom or condition associated with emesis. Specifically, a GIP receptor agonist peptide containing a sequence represented by any of the formulas (I)-(V) or a salt thereof, and a medicament comprising the same are provided.

### CROSS-REFERENCE TO RELATED APPLICATIONS

This application is a by-pass continuation application under 35 U.S.C. § 111(a) of PCT Application No. PCT/JP2021/014423, filed on Mar. 25, 2021, which claims priority to U.S. Provisional Application Ser. No. 62/994,716, filed on Mar. 25, 2020, the entire contents of which are incorporated by reference herein.

### TECHNICAL FIELD

The present disclosure relates to a novel peptide compound having an activating action on GIP receptors and use of the peptide compound as a medicament which may be dosed in a once daily dosing regimen.

### BACKGROUND

The statements in this section merely provide background information related to the present disclosure and may not constitute prior art.

Both glucagon-like peptide-1 (GLP-1) and glucose-dependent insulinotropic polypeptide (GIP) are peptides called incretin. GLP-1 and GIP are secreted from small intestinal L cells and K cells, respectively.

GLP-1 acts via GLP-1 receptors and is known to have a glucose-dependent insulinotropic action and a feeding suppressive action. On the other hand, GIP is known to have a glucose-dependent insulinotropic action via the GIP receptors (GIPr), though an influence of GIP only on feeding is not clear.

Attempts have been made to search for peptides having GLP-1 receptor/GIP receptor coagonist or glucagon receptor/GLP-1 receptor/GIP receptor triagonist activity and modifications thereof and develop these peptides as anti-obesity drugs, therapeutic drugs for diabetes, or therapeutic drugs for neurodegenerative disorders based on the structure of natural glucagon, GIP, or GLP-1. However, the peptide compound and the compound having a selective activating action on GIP receptors of the present disclosure for the use in treating emesis and similar symptoms associated with nausea and vomiting have not been disclosed.

Patients who experience nausea and vomiting are often unwilling or unable to take their medication regularly; several studies have shown that a less frequent dosing results in higher degree of compliance and thus eventually better treatment of the patients. Therefore, there is an unmet need for long acting preparations of antiemetic medicine. In particular there is a need for long acting preparations of antiemetic GIP receptor agonist peptides that represent an alternative to twice per day (BID) dosing formulations in order to make a change in dosing regimen, frequency of medication or type of medication, more flexible. Extending the duration of action will also provide benefit in diseases where the duration of emetic episodes is longer.

All publications, patents, and patent applications cited herein are incorporated herein by reference in their entirety.

### SUMMARY

It is an object of the present invention to provide a GIP receptor agonist peptide compound which has a GIP receptor activation action and is useful as a preventive/therapeutic agent for diabetes, obesity, and/or an antiemetic agent to prevent/treat diseases accompanied by vomiting or nausea.

The present disclosure provides GIPr agonist peptide compounds comprising a sequence represented by formulae (I)-(V) that are useful as therapeutic agents for the prevention or treatment of emesis as described herein. Surprisingly, the compounds of formulae (I)-(V) exhibit excellent GIP receptor activation action, a longer ½ life of elimination and improved solubility. Unexpectedly, in some instances, the peptides of formulae (I)-(V) relative to other known GIPr agonist peptides in the art possess improved properties in one or more of: (1) stability in serum, (2) half-life of elimination and (3) solubility. In certain embodiments of this disclosure, the peptides of formulae (I)-(V) relative to other known GIPr agonist peptides that are dosed once per day to treat emesis, or which may be useful as preventative agents of nausea and/or vomiting and other symptoms of emesis, possess improved properties in one or more of: (1) stability in serum, (2) half-life of elimination and (3) solubility.

More specifically, the present disclosure includes the following embodiments:

Embodiment (1). A GIP receptor agonist peptide represented by formula (I): P¹-Tyr-A2-Glu-Gly-Thr-Phe-Ile-Ser-A9-Tyr-Ser-Ile-A13-A14-Asp-A16-A17-A18-Gln-A20-A21-Phe-Val-A24-Trp-A26-Leu-A28-Gln-A30-A31-A32-A33-A34-A35-A36-A37-A38-A39-A40-P², or a pharmaceutically acceptable salt thereof;

wherein
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Gly, or Pro;
A9: represents Asp or Leu;
A13: represents Aib, or Ala;
A14: represents Leu, Aib, Lys;
A16: represents Arg, Ser, or Lys;
A17: represents Aib, Gln, or Ile;
A18: represents Ala, His, or Lys;
A19: represents Gln, or Ala;
A20: represents Aib, Gln, Lys, or Ala;
A21: represents Asp, Asn, or Lys;
A24: represents Asn, or Glu;
A26: represents Leu or Lys;
A28: represents Ala, Lys, or Aib;
A29: represents Gln, Lys, Gly, or Aib;
A30: represents Arg, Gly, Ser, or Lys;
A31: represents Gly, Pro, or a deletion;
A32: represents Ser, Gly, or a deletion;
A33: represents Ser, Gly, or a deletion;
A34: represents Gly, Lys, Asn, or a deletion;
A35: represents Ala, Asp, Ser, Lys, or a deletion;
A36: represents Pro, Trp, Lys, or a deletion;
A37: represents Pro, Lys, Gly, or a deletion;
A38: represents Pro, His, Lys, or a deletion;
A39: represents Ser, Asn, Gly, Lys, or a deletion; and
A40: represents Ile, Lys or a deletion.

Embodiment (2). A GIP receptor agonist peptide, represented by formula (II): P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-A26-Leu-Ala-A29-A30-A31-A32-A33-A34-A35-A36-A37-A38-A39-A40-P², or a pharmaceutically acceptable salt thereof, wherein:

P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, Ser, Ala, D-Ala, or Gly;
A13: represents Aib, Tyr, or Ala;
A14: represents Leu, or Lys(R);
A16: represents Arg, Ser, or Lys;
A17: represents Aib, Ile, Gln, or Lys(R);
A18: represents Ala, His, or Lys(R);
A19: represents Gln or Ala;
A20: represents Aib, Gln, or Lys(R);
A21: represents Asn, Glu, Asp, or Lys(R);
A24: represents Asn, or Glu;
A26: represents Leu or Lys(R);
A28: represents Ala, Aib, or Lys(R);
A29: represents Gln, Aib, or Lys(R)
A30: represents Arg, Gly, Lys, Ser, or Lys(R);
A31: represents Gly, Pro, or a deletion;
A32: represents Ser, Lys, Pro, Gly, or a deletion;
A33: represents Ser, Lys, Gly, or a deletion;
A34: represents Gly, Lys, Asn, or a deletion;
A35: represents Ala, Asp, Ser, Lys, or a deletion;
A36: represents Pro, Trp, Lys, or a deletion;
A37: represents Pro, Lys, Gly, or a deletion;
A38: represents Pro, His, Lys, or a deletion;
A39: represents Ser, Asn, Lys, Gly, or a deletion;
A40: represents Ile, Lys(R), or a deletion;
wherein in the residue Lys(R), the (R) portion represents X-L-, wherein L represents a linker, and is selected from the following group consisting of gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE; and X represents a lipid.

Embodiment (3). A GIP receptor agonist peptide represented by formula (IV): P¹-Tyr-A2-Glu-Gly-Thr-A6-A7-Ser-Asp-Tyr-Ser-Ile-A13-A14-Asp-A16-A17-A18-Gln-A20-A21-Phe-Val-Asn-Trp-Leu-Leu-A28-A29-A30-A31-A32-A33-A34-A35-A36-A37-A38-A39-P², or a pharmaceutically acceptable salt thereof;

### Wherein

P¹ represents H, C₁₋₆ alkyl, or absent;
P² represents —NH₂ or —OH;
A2 represents Aib, Gly, or Ser;
A6 represents Phe or Leu;
A7 represents Ile or Thr;
A13 represents Ala, Aib, or Tyr;
A14 represents Leu, Lys, or Lys(R);
A16 represents Lys, Arg, or Ser;
A17 represents Aib, Ile, Lys, or Lys(R);
A18 represents Ala, His, Lys, or Lys(R);
A20 represents Gln, Lys, Lys(R), or Aib;
A21 represents Asp, Lys, Lys(R), or Asn;
A28 represents Ala, Aib, or, Lys, Lys(R);
A29 represents Gln, Lys, Lys(R), or Aib;
A30 represents Lys, Ser, Arg, Lys(R), or Lys(Ac);
A31 represents Pro, Gly, or a deletion;
A32 represents Ser, Gly, or a deletion;
A33 represents Ser, Gly, or a deletion;
A34 represents Gly, Lys, or a deletion;
A35 represents Ala, Ser, Lys, or a deletion;
A36 represents Pro, Lys, or a deletion;
A37 represents Pro, Lys, Gly, or a deletion;
A38 represents Pro, Lys, or a deletion; and
A39 represents Ser, Gly, Lys, or a deletion,
wherein in the residue Lys(R), the (R) portion represents X-L-, wherein L represents a linker and is selected from the group consisting of 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE, and OEGgEgEgE; and X represents C₁₄-C₁₈ monoacid or C₁₄-C₁₈ diacid.

Embodiment (4). The GIP receptor agonist peptide according to embodiment (3) or the pharmaceutically acceptable salt thereof, wherein

A14 represents Leu or Lys(R);
A17 represents Aib, Ile, or Lys(R);
A18 represents Ala, His, or Lys(R);
A20 represents Gln, Lys(R), or Aib;
A21 represents Asp, Lys(R), or Asn;
A28 represents Ala, Aib, or Lys(R);
A29 represents Gln, Lys(R), or Aib, and
A30 represents Lys, Ser, Arg, Lys(R), or Lys(Ac).

Embodiment (5). The GIP receptor agonist peptide or the pharmaceutically acceptable salt thereof according to embodiment (4) has a solubility of at least 15 mg/mL in phosphate buffer at pH 7.4.

Embodiment (6). The GIP receptor agonist peptide according to embodiment (3) or the pharmaceutically acceptable salt thereof, wherein

A2 represents Aib;
A17 represents Aib, Lys, or Lys(R);
A20 represents Aib; and
A28 represents Ala or Aib,
wherein L is selected from the group consisting of 2OEG, 2OEGgE, 2OEGgEgE, G2E3, G4gE, G4gEgE, G5, G5E, G5gE, G5gEgE, gEgEgE, GGEEE, GGPAPAP, OEGgEgE, and OEGgEgEgE.

Embodiment (7). The GIP receptor agonist peptide according to embodiment (6) or the pharmaceutically acceptable salt thereof, wherein

A14 represents Leu or Lys(R);
A17 represents Aib or Lys(R).
A18 represents Ala, His, or Lys(R);
A21 represents Asp, Lys(R), or Asn;
A29 represents Gln, Lys(R), or Aib; and
A30 represents Lys, Ser, Arg, Lys(R), or Lys(Ac).

Embodiment (8). The GIP receptor agonist peptide or the pharmaceutically acceptable salt thereof according to embodiment (7) has a solubility of at least 30 mg/mL in phosphate buffer at pH 7.4.

Embodiment (9). The GIP receptor agonist peptide according to any one of embodiments (1)-(8) or the pharmaceutically acceptable salt thereof, wherein A31 is Gly, and A32-A39 are deletion; or A32 is Gly and 33-A39 are deletion.

Embodiment (10). The GIP receptor agonist peptide according to any one of embodiments (1)-(9) or the pharmaceutically acceptable salt thereof, wherein P² is —OH.

Embodiment (11). The GIP receptor agonist peptide according to any one of embodiments (2)-(10) or the pharmaceutically acceptable salt thereof, wherein Lys(R) is a Lys residue, and wherein the side chain of said Lys residue is substituted with (R).

Embodiment (12). The GIP receptor agonist peptide according to embodiment (11) or the pharmaceutically acceptable salt thereof, wherein Lys(R) is a Lys residue substituted with (R), and (R) is represented by X-L-, wherein L is selected from the group consisting of 1OEGgE, 2OEG, 2OEGgE, 2OEGgEgE, 3OEGgE, G2E3, G3gEgE, G4E2, G4gE, G4gEgE, GGGGG, G5E, G5gE, G5gEgE, gEgEgE, GGEEE, GGPAPAP, OEGgEgE, and OEGgEgEgE.

Embodiment (13). The GIP receptor agonist peptide according to embodiment (12) or the pharmaceutically acceptable salt thereof, wherein L is selected from 2OEGgEgE, OEGgEgE, 2OEGgE, GGGGG, G5gEgE, 2OEG and G5gEgE; and X is a C₁₄-C₁₆ monoacid or diacid group or X is a C₁₅-C₁₈ diacid.

Embodiment (14). The GIP receptor agonist peptide according to embodiment (13) or the pharmaceutically acceptable salt thereof, wherein L is 2OEGgEgE or GGGGG.

Embodiment (15). The GIP receptor agonist peptide according to embodiment (13) or the pharmaceutically acceptable salt thereof, wherein X is C₁₅ diacid or C₁₆ diacid.

Embodiment (16). The GIP receptor agonist peptide according to embodiment (15) or the pharmaceutically acceptable salt thereof, wherein X is C₁₅ diacid.

Embodiment (17). The GIP receptor agonist peptide according to embodiment (13) or the pharmaceutically acceptable salt thereof, wherein the linker (L) is 2OEGgEgE or GGGGG, and (R) is 2OEGgEgE-C₁₅ diacid or (R) is 2OEGgEgE-C₁₆ diacid.

Embodiment (18). The GIPR agonist peptide according to any one of embodiments (2)-(15) or the pharmaceutically acceptable salt thereof, represented by formula (V): P¹-Tyr-Aib-Glu-Gly-The-Phe-Ile-Ser-Asp-Tyr-Ser-Ile-A13-Leu-Asp-Arg-Aib-A18-Gln-Aib-A21-Phe-Val-Asn-Trp-Leu-Leu-Ala-Gln-A30-A31-A32-P², wherein

P¹ is methyl;
P² is OH or NH₂;
A13 represents Ala or Aib;
A18 represents Ala, Lys, or Lys(R);
A21 represents Lys, Lys(R), or Asp;
A30 represents Lys or Ser;
A31 represents Gly or Pro; and
A32 represents Gly or deletion;
wherein (R) represents X-L-, L represents 2OEGgEgE or GGGGG; and X represents a C₁₅ diacid or C₁₆ diacid.

Embodiment (19). The GIPR agonist peptide of embodiment (18) or the pharmaceutically acceptable salt thereof, wherein

A18 represents Ala or Lys(R); and
A21 represents Lys(R) or Asp.

Embodiment (20). The GIP receptor agonist peptide according to any one of embodiments (2)-(5), or the pharmaceutically acceptable salt thereof, wherein the amino acid sequence comprises: P¹-Y-Aib-E-G-T-F-I-S-D-Y-S-I-A-L-D-R-Aib-A-Q-Aib-Km-F-V-N-W-L-L-A-Q-R-P²; wherein Km is Lys-2OEGgEgE-C₁₅ diacid.

Embodiment (21). The GIP receptor agonist peptide according to embodiment (20), or the pharmaceutically acceptable salt thereof, wherein the amino acid sequence comprises: Me-Tyr-Aib-Glu-Gly-Thr-Phe-Ile-Ser-Asp-Tyr-Ser-Ile-Ala-Leu-Asp-Arg-Aib-Ala-Gln-Aib-Lys(R)-Phe-Val-Asn-Trp-Leu-Leu-Ala-Gln-Arg-NH2; wherein Lys(R) is Lys-2OEGgEgE-C₁₅ diacid.

Embodiment (22). The GIP receptor agonist peptide according to any one of embodiments (2)-(5), or the pharmaceutically acceptable salt thereof, wherein the amino acid sequence comprises: P¹-Y-Aib-E-G-T-F-I-S-D-Y-S-I-A-L-D-R-Aib-Km-Q-Aib-N-F-V-N-W-L-L-A-Q-S-P-S-S-G-A-P-P-P-S-P²; wherein Km is Lys-2OEGgEgE-C₁₅ diacid.

Embodiment (23). The GIP receptor agonist peptide according to any one of embodiments (2)-(19), or the pharmaceutically acceptable salt thereof, wherein the amino acid sequence comprises: P¹-Y-Aib-E-G-T-F-I-S-D-Y-S-I-A-L-D-R-Aib-A-Q-Aib-Km-F-V-N-W-L-L-A-Q-K-G-P²;

wherein Km is Lys-2OEGgEgE-C₁₅ diacid.

Embodiment (24). The GIPR agonist peptide of embodiment (23) or the pharmaceutically acceptable salt thereof, represented by the formula: Me-Tyr-Aib-Glu-Gly-Thr-Phe-Ile-Ser-Asp-Tyr-Ser-Ile-Ala-Leu-Asp-Arg-Aib-Ala-Gln-Aib-Lys(R)-Phe-Val-Asn-Trp-Leu-Leu-Ala-Gln-Lys-Gly-OH; wherein Lys(R) is Lys-2OEGgEgE-C₁₅ diacid.

Embodiment (25). The GIP receptor agonist peptide according to any one of embodiments (2)-(5), or the pharmaceutically acceptable salt thereof, wherein the amino acid sequence comprises: P¹-Y-Aib-E-G-T-F-I-S-D-Y-S-I-Aib-Km-D-R-Aib-A-Q-Aib-D-F-V-N-W-L-L-A-Q-R-G-P²; wherein Km is Lys-GGGGG-C₁₅ diacid.

Embodiment (26). The GIP receptor agonist peptide according to any one of embodiments (2)-(5), or the pharmaceutically acceptable salt thereof, wherein the amino acid sequence comprises: P¹-Y-Aib-E-G-T-F-I-S-D-Y-S-I-Aib-L-D-R-Aib-A-Q-Aib-N-F-V-N-W-L-L-A-Q-Km-P-S-S-G-A-P-P-P-S-P²; wherein Km is Lys-2OEGgEgE-C₁₅ diacid.

Embodiment (27). The GIP receptor agonist peptide according to any one of embodiments (2)-(5), or the pharmaceutically acceptable salt thereof, wherein the amino acid sequence comprises: P¹-Y-Aib-E-G-T-F-I-S-D-Y-S-I-A-Km-D-R-Aib-A-Q-Aib-N-F-V-N-W-L-L-A-Q-S-P-S-S-G-A-P-P-P-S-P²; wherein Km is Lys-GGGGG-C₁₅ diacid.

Embodiment (28). The GIP receptor agonist peptide according to any one of embodiments (2)-(5), or the pharmaceutically acceptable salt thereof, wherein the amino acid sequence comprises: P¹-Y-Aib-E-G-T-F-I-S-D-Y-S-I-Aib-L-D-R-Km-A-Q-Aib-N-F-V-N-W-L-L-A-Q-R-P-S-S-G-A-P-P-P-S-P²; wherein Km is Lys-2OEGgEgE-C₁₅ diacid.

Embodiment (29). The GIP receptor agonist peptide according to any one of embodiments (2)-(19) or the pharmaceutically acceptable salt thereof, wherein the amino acid sequence comprises: P¹-Y-Aib-E-G-T-F-I-S-D-Y-S-I-A-L-D-R-Aib-A-Q-Aib-Km-F-V-N-W-L-L-A-Q-K-G-P²; wherein Km is Lys-2OEGgEgE-C₁₆ diacid.

Embodiment (30). The GIPR agonist peptide of embodiment (29), or the pharmaceutically acceptable salt thereof, represented by the formula: Me-Tyr-Aib-Glu-Gly-Thr-Phe-Ile-Ser-Asp-Tyr-Ser-Ile-Ala-Leu-Asp-Arg-Aib-Ala-Gln-Aib-Lys(R)-Phe-Val-Asn-Trp-Leu-Leu-Ala-Gln-Lys-Gly-OH; wherein Lys(R) is Lys-2OEGgEgE-C₁₆ diacid.

Embodiment (31). The GIP receptor agonist peptide according to any one of embodiments (2)-(19), or the pharmaceutically acceptable salt thereof, wherein the amino acid sequence comprises: P¹-Y-Aib-E-G-T-F-I-S-D-Y-S-I-Aib-L-D-R-Aib-Km-Q-Aib-D-F-V-N-W-L-L-A-Q-S-P-G-P²; wherein Km is Lys-2OEGgEgE-C₁₆ diacid.

Embodiment (32). The GIPR agonist peptide of embodiment (31) or the pharmaceutically acceptable salt thereof, represented by the formula: Me-Tyr-Aib-Glu-Gly-Thr-Phe-Ile-Ser-Asp-Tyr-Ser-Ile-Aib-Leu-Asp-Arg-Aib-Lys(R)-Gln-Aib-Asp-Phe-Val-Asn-Trp-Leu-Leu-Ala-Gln-Ser-Pro-Gly-OH; wherein Lys(R) is Lys-2OEGgEgE-C₁₆ diacid.

Embodiment (33). The GIP receptor agonist peptide according to any one of embodiments (2)-(8) or the pharmaceutically acceptable salt thereof, wherein the amino acid sequence comprises: Me-Tyr-Aib-Glu-Gly-Thr-Phe-Ile-Ser-Asp-Tyr-Ser-Ile-Aib-Lys(R)-Asp-Arg-Aib-Ala-Gln-Aib-Asn-Phe-Val-Asn-Trp-Leu-Leu-Ala-Gln-Ser-Pro-Ser-Ser-Gly-Ala-Pro-Pro-Pro-Ser-OH; wherein Lys(R) is Lys-GGGGG-C₁₅ diacid.

Embodiment (34). The GIP receptor agonist peptide according to any one of embodiments (1)-(19), wherein P¹ is Methyl- (Me) and P² is —OH, or NH₂.

Embodiment (35). The GIP receptor agonist peptide according to any one of embodiments (1)-(34), wherein the GIP receptor agonist peptide has a selectivity ratio, expressed as a ratio of (GLP1R EC₅₀/GIPR EC₅₀) of greater than 10, or greater than 100, or greater than 1,000, or greater than 100,000.

Embodiment (36). A medicament comprising the GIP receptor agonist peptide according to any one of embodiments 1-35, or a pharmaceutically acceptable salt thereof.

Embodiment (37). A pharmaceutical composition comprising the GIP receptor agonist peptide according to any one of embodiments 1-35, or a pharmaceutically acceptable salt thereof.

Embodiment (38). The GIP receptor agonist peptide or the salt thereof according to any one of embodiments (1)-(35), or the medicament according to embodiment (36), or the pharmaceutical composition according to embodiment (37), which is administered once per day (QD), or once every 24 hrs to alleviate or treat emesis as a monotherapy or as an adjunct therapy.

Embodiment (39). Use of the GIP receptor agonist peptide according to any one of embodiments (1)-(35) or a salt thereof, or the medicament according to embodiment (36), or the pharmaceutical composition according to embodiment (37), for the manufacture of a suppressant for vomiting or nausea.

Embodiment (40). The peptide of according to any one of embodiments (1)-(35), or a salt thereof, or the medicament according to embodiment (36), or the pharmaceutical composition according to embodiment (37), for use in suppressing vomiting or nausea.

Embodiment (41). A method for preventing or treating emesis in a subject, comprising administering an effective amount of the peptide according to any one of embodiments (1)-(35), or a salt thereof, or the medicament according to embodiment (36), or the pharmaceutical composition according to embodiment (37), to the subject.

Embodiment (42). The medicament according to embodiment (36), the use according to embodiment (39), the peptide or a salt thereof, the medicament, or the pharmaceutical composition according to embodiment (40), the method according to embodiment (41), wherein the emesis, vomiting or the nausea is caused by one or more conditions or causes selected from the following (1) to (10):

(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome (CVS); chemotherapy induced nausea and vomiting (CINV), post-operative nausea and vomiting (PONV), chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea cexaused by a vestibular disorder such as motion sickness or dizziness
(7) Opioid withdrawal;
(8) Vomiting and nausea caused by post-operative nausea and vomiting;
(9) A vestibular disorder such as motion sickness or dizziness; and
(10) A physical injury causing local, systemic, acute or chronic pain.

Embodiment (43). The method according to embodiment (41), wherein emesis is treated in a subject not taking a medicament to control a metabolic syndrome disorder.

Embodiment (44). A GIP receptor agonist peptide of any one of embodiments (1)-(35) or a salt thereof, wherein the peptide selectively activates the GIP receptor and demonstrates an antiemetic action in vivo, and wherein the antiemetic action is achieved by dosing the peptide to a subject in need thereof, once per day, or once per 24 hours.

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Other features and advantages of the disclosure will be apparent from the following Detailed Description, the drawings, and the claims.

### BRIEF DESCRIPTION OF THE FIGURES

FIGS. 1A-1H. Exemplary GIP receptor agonist peptides of the present disclosure which are represented by any one of formulas (I)-(V).

FIG. 2. Effect of Compound 14 on PYY (T-481, 10 μg/kg, s.c.) induced vomiting in dogs.

FIG. 3. Effect of Compound 25, Compound 48, Compound 58, and Compound 260 on PYY (T-481, 10 μg/kg, s.c.) induced vomiting in dogs.

FIGS. 4A-4C. Effect of Compound 25 on morphine (0.6 mg/kg, s.c.)-induced emesis in ferrets.

FIGS. 5A-5C. Effect of Compound 14 on morphine (0.6 mg/kg, s.c.)-induced emesis in ferrets.

### DETAILED DESCRIPTION OF THE EMBODIMENTS

The definition of each substituent used in the present specification is described in detail in the following. Unless otherwise specified, each substituent has the following definition.

In the present specification, examples of the “halogen atom” include fluorine, chlorine, bromine and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “optionally halogenated C₁₋₆ alkyl group” include a C₁₋₆ alkyl group optionally having 1 to 7, or 1 to 5, halogen atoms. Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

In the present specification, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, or 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy group” include a C₁₋₆ alkoxy group optionally having 1 to 7, or 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, or 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, or 1 to 5, halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl.

In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, or 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent(s) selected from the following substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl)amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group and
(62) a C₆₋₁₄ aryl group.

The number of the above-mentioned substituents in the “optionally substituted hydrocarbon group” is, for example, 1 to 5, or 1 to 3. When the number of the substituents is two or more, the respective substituents may be the same or different.

In the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 hetero atoms selected from a nitrogen atom, a sulfur atom and an oxygen atom.

In the present specification, examples of the “aromatic heterocyclic group” (including “5- to 14-membered aromatic heterocyclic group”) include a 5- to 14-membered (or 5- to 10-membered) aromatic heterocyclic group containing, as a ring-constituting atom besides carbon atom, 1 to 4 hetero atoms selected from a nitrogen atom, a sulfur atom and an oxygen atom.

Examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (e.g., bi or tricyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, β-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (or 4- to 10-membered) non-aromatic heterocyclic group containing, as a ring-constituting atom besides carbon atom, 1 to 4 hetero atoms selected from a nitrogen atom, a sulfur atom and an oxygen atom.

Examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (e.g., bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the aforementioned substituent group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” (also referred to as “Ac”) also include a hydrocarbon-sulfonyl group, a heterocyclylsulfonyl group, a hydrocarbon-sulfinyl group and a heterocyclylsulfinyl group.

In some embodiments, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from substituent group A”.

Examples of the optionally substituted amino group include an amino group, a mono- or di-(optionally halogenated C₁₋₆ alkyl)amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl)amino group (e.g., methylcarbamoylamino), a (mono- or di-C₇₋₁₆ aralkyl-carbamoyl)amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl)(C₁₋₆ alkyl-carbonyl)amino group (e.g., N-acetyl-N-methylamino) and a (C₁₋₆ alkyl)(C₆₋₁₄ aryl-carbonyl)amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from substituent group A”.

Examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from substituent group A” and a halogenated sulfanyl group.

Examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from substituent group A”.

Examples of the optionally substituted silyl group include a tri-C₁₋₆ alkylsilyl group (e.g., trimethylsilyl, tert-butyl(dimethyl)silyl).

For descriptions of amino acid residues, the following conventions may be exemplified: Asp=D=Aspartic Acid; Ala=A=Alanine; Arg=R=Arginine; Asn=N=Asparagine; Cys=C=Cysteine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; Phe=F=Phenylalanine; Pro=P=Proline; Ser=S=Serine; Thr=T=Threonine; Trp=W=Tryptophan; Tyr=Y=Tyrosine; and Val=V=Valine.

Also for convenience, and readily known to one skilled in the art, the following abbreviations or symbols are used to represent the moieties, reagents and the like used in present disclosure:

Aib is alpha-aminoisobutyric acid;

mono-halo Phe—mono-halo phenylalanine;

bis-halo Phe—bis-halo phenylalanine;

mono-halo Tyr—mono-halo tyrosine;

bis-halo Tyr—bis-halo Tyrosine;

(D)-Tyr—D-tyrosine;

(D)-Ala—D-Alanine

DesNH₂-Tyr—desaminotyrosine;

(D)-Phe—D-phenylalanine;

DesNH₂-Phe—desaminophenylalanine;

(D)-Trp—D-tryptophan;

(D)₃Pya—D-3-pyridylalanine;

2-Cl-(D)Phe—D-2-chlorophenylalanine;

3-Cl-(D)Phe—D-3-chlorophenylalanine;

4-Cl-(D)Phe—D-4-chlorophenylalanine;

2-F-(D)Phe—D-2-fluorophenylalanine;

3-F(D)Phe—D-3-fluorophenylalanine;

3,5-DiF-(D)Phe—D-3,5-difluorophenylalanine;

3,4,5-TriF-(D)Phe—D-3,4,5-trifluorophenylalanine;

D-Iva—D-Isovaline

SSA—succinimidyl succinamide;

PEG—polyethylene glycol;

PEGₘ—(methoxy)polyethylene glycol;

PEGₘ(12,000)—(methoxy)polyethylene glycol having a molecular weight of about 12 kD;

PEGₘ(20,000)—(methoxy)polyethylene glycol having a molecular weight of about 20 kD;

PEGₘ(30,000)—(methoxy)polyethylene glycol having a molecular weight of about 30 kD;

Fmoc—9-fluorenylmethyloxycarbonyl;

DMF—dimethylformamide;

DIPEA—N,N-diisopropylethylamine;

TFA—trifluoroacetic acid;

HOBT—N-hydroxybenzotriazole;

BOP—benzotriazol-1-yloxy-tris-(dimethylamino)phosphoniumhexafluorophosphate;

HBTU—2-(1H-benzotriazole-1-yl)-1,1,3,3-tetramethyluroniumhexafluorophosphate;

NMP—N-methyl-pyrrolidone;

FAB-MS fast atom bombardment mass spectrometry;

ES-MS—electro spray mass spectrometry.

Abu: α-aminobutyric acid;

Acc: 1-amino-1-cyclo(C₃-C₉)alkyl carboxylic acid;

A3c: 1-amino-1-cyclopropane carboxylic acid;

A4c: 1-amino-1-cyclobutanecarboxylic acid;

A5c: 1-amino-1-cyclopentanecarboxylic acid;

A6c: 1-amino-1-cyclohexanecarboxylic acid;

Act: 4-amino-4-carboxytetrahydropyran;

Ado: 12-aminododecanoic acid;

Aib: alpha-aminoisobutyric acid;

Aic: 2-aminoindan-2-carboxylic acid;

β-Ala: beta-alanine;

Amp: 4-amino-phenylalanine;

Apc: 4-amino-4-carboxypiperidine;

hArg: homoarginine;

Aun: 11-aminoundecanoic acid;

Ava: 5-aminovaleric acid;

Cha: β-cyclohexylalanine;

Dhp: 3,4-dehydroproline;

Dmt: 5,5-dimethylthiazolidine-4-carboxylic acid;

Gaba: γ-aminobutyric acid;

4Hppa: 3-(4-hydroxyphenyl)propionic acid;

Hyp:—hydroxyproline

3Hyp: 3-hydroxyproline;

4Hyp: 4-hydroxyproline;

hPro: homoproline;

4Ktp: 4-ketoproline;

Nle: norleucine;

NMe-Tyr: N-methyl-tyrosine;

1Nal or 1-Nal: β-(1-naphthyl)alanine;

2Nal or 2-Nal: β-(2-naphthyl)alanine;

Nva: norvaline;

Orn: ornithine;

2Pal or 2-Pal: β-(2-pyridinyl)alanine;

3Pal or 3-Pal: β-(3-pyridinyl)alanine;

4Pal or 4-Pal: β-(4-pyridinyl)alanine;

Pen: penicillamine;

(3,4,5F)Phe: 3,4,5-trifluorophenylalanine;

(2,3,4,5,6)Phe: 2,3,4,5,6-pentafluorophenylalanine;

Psu: N-propylsuccinimide;

Iva: Isovaline;

Sar: Sarcosine;

Taz: β-(4-thiazolyl)alanine;

3Thi: β-(3-thienyl)alanine;

Thz: thioproline;

Tic: tetrahydroisoquinoline-3-carboxylic acid;

Tle: tert-leucine;

Act: acetonitrile;

Boc: tert-butyloxycarbonyl;

BSA: bovine serum albumin;

DCM: dichloromethane;

DTT: dithiothrieitol;

ESI: electrospray ionization;

Fmoc: 9-fluorenylmethyloxycarbonyl;

HBTU: 2-(1H-benzotriazole-1-yl)-1,1,3,3-tetramethyluronium hexafluorophosphate;

HPLC: high performance liquid chromatography;

IBMX: isobutylmethylxanthine;

LC-MS: liquid chromatography-mass spectrometry;

Mtt: methyltrityl;

NMP: N-methylpyrrolidone;

5K PEG: polyethylene glycol, which may include other functional groups or moieties such as a linker, and which is either linear or branched as defined herein below, with a weight average molecular weight of about 5,000 Daltons.

10K PEG: polyethylene glycol, which may include other functional groups or moieties such as a linker, and which is either linear or branched as defined herein below, with a weight average molecular weight of about 10,000 Daltons.

20K PEG: polyethylene glycol, which may include other functional groups or moieties such as a linker, and which is either linear or branched as defined herein below, with a weight average molecular weight of about 20,000 Daltons.

30K PEG: polyethylene glycol, which may include other functional groups or moieties such as a linker, and which is either linear or branched as defined herein below, with a weight average molecular weight of about 30,000 Daltons.

40K PEG: polyethylene glycol, which may include other functional groups or moieties such as a linker, and which is either linear or branched as defined herein below, with a weight average molecular weight of about 40,000 Daltons.

50K PEG: polyethylene glycol, which may include other functional groups or moieties such as a linker, and which is either linear or branched as defined herein below, with a weight average molecular weight of about 50,000 Daltons.

60K PEG: polyethylene glycol, which may include other functional groups or moieties such as a linker, and which is either linear or branched as defined herein below, with a weight average molecular weight of about 60,000 Daltons.

PEG is available in a variety of molecular weights based on the number of repeating subunits of ethylene oxide (i.e. —OCH₂CH₂—) within the molecule. mPEG formulations are usually followed by a number that corresponds to their average molecular weight. For example, PEG-200 has a weight average molecular weight of 200 Daltons and may have a molecular weight range of 190-210 Daltons. Molecular weight in the context of a water-soluble polymer, such as PEG, can be expressed as either a number average molecular weight or a weight average molecular weight. Unless otherwise indicated, all references to molecular weight of mPEG herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight.

tBu: tert-butyl

TIS: triisopropylsilane

Trt: trityl

Z: benzyloxycarbonyl

As used herein, “PEG moiety” refers to polyethylene glycol (PEG) or a derivative thereof, for example (methoxy)polyethylene glycol (PEGₘ).

As used herein, “PEGylated peptide” refers to a peptide wherein at least one amino acid residue, for example, Lys, or Cys has been conjugated with a PEG moiety. By “conjugated”, it is meant that the PEG moiety is either directly linked to said residue or is linked to the residue via a spacer moiety, for example a cross-linking agent. When said conjugation is at a lysine residue, that lysine residue is referred to herein as “PEGylated Lys”. A peptide that is conjugated to only one MPEG moiety is said to be “mono-PEGylated”.

As used herein, “Lys-PEG” and “Lys-PEGₘ” refer respectively to lysine residues which have been conjugated with PEG. “Lys(epsilon-SSA-PEGn)” refers to a lysine residue wherein the epsilon-amino group has been cross-linked with MPEG using a suitably functionalized SSA.

In the present specification, the term “human native GIP peptide” refers to the naturally occurring human GIP peptide. This human native GIP peptide (42 amino acids) has an amino acid sequence: YAEGTFISDYSIAMDKIHQ QDFVNWLLAQKGKKNDWKHNITQ (SEQ ID NO: 1) and is the functionally active molecule derived from the parent precursor described in National Center for Biotechnology Information (NCBI) Reference Sequence: NP_004114.1; REFSEQ: accession NM_004123.2 This full length precursor is encoded from the mRNA sequence of human gastric inhibitory polypeptide (GIP), mRNA; ACCESSION: NM_004123; VERSION; NM_004123.2.

“Percent (%) amino acid sequence identity” with respect to a reference polypeptide sequence is defined as the percentage of amino acid residues in a candidate polypeptide sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “treatment” (and variations such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of a condition, alleviation of symptoms, diminishment of any direct or indirect pathological consequences of the condition or treatment, preventing emesis, i.e., by preventing the occurrence of symptoms in whole or in part associated with a condition or side-effects known to accompany a specific treatment, decreasing the rate of progression, amelioration or palliation of the symptoms associated with emesis, such as nausea and/or vomiting, and remission or improved prognosis. In some embodiments, GIP receptor agonist peptides of the disclosure are used to inhibit or delay development of emesis, i.e. nausea or vomiting or to slow the progression of emesis or the symptoms associated with emesis, or to prevent, delay or inhibit the development of emesis, nausea and/or vomiting related to the treatment of a different disease being actively treated.

By “reduce” or “inhibit” is meant the ability to cause an overall decrease of 20%, 30%, 40%, 50%, 60%, 70%, 75%, 80%, 85%, 90%, 95%, or greater. In some embodiments, reduce or inhibit can refer to a relative reduction compared to a reference (e.g., reference level of biological activity (e.g., the number of episodes of nausea and/or vomiting after administration to a subject of a prescribed amount of chemotherapy, for example, a prescribed dose of a chemotherapeutic agent that is known to cause emesis). In some embodiments, reduce or inhibit can refer to the relative reduction of a side effect (i.e. nausea and/or vomiting) associated with a treatment for a condition or disease.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. MoI. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One illustrative example of an algorithm that is suitable for determining percent sequence identity and sequence similarity is the BLAST algorithm, which is described by Altschul et al. (J. MoI. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

Variants can also be synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine.

“Conservative amino acid substitutions” as referenced herein result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to penetrate the blood brain barrier (BBB)). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can also select conservative amino acid substitutions encompassed suitable for amino acids on the interior of a protein or peptide, for example one can use suitable conservative substitutions for amino acids is on the interior of a protein or peptide (i.e., the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

As is understood by one skilled in the art, reference to “about” a value or parameter herein includes (and describes) embodiments that are directed to that value or parameter per se or that have a variance plus or minus of that value ranging from less than 10%, or less than 9%, or less than 8%, or less 7%, or less than 6%, or less than 5%, or less than 4%, or less than 3%, or less than 2%, or less than 1%, or less than 0.1% than the stated value. For example, description referring to “about X” includes description of “X”.

It is understood that aspect and embodiments of the disclosure described herein include “consisting” and/or “consisting essentially” of aspects and embodiments. As used herein, the singular form “a”, “an”, and “the” includes plural references unless indicated otherwise.

### A. GIP RECEPTOR AGONIST PEPTIDES

In various embodiments of the present disclosure, GIP receptor agonist peptides are provided. In addition, methods are provided for the prevention and/or treatment of diabetes mellitus (e.g., type-2 diabetes mellitus) obesity, a metabolic syndrome and emesis in a subject in need thereof. In various embodiments, the methods provide administration of a therapeutically effective amount of a GIP receptor agonist peptide once per day or QD (for example, Q1D, used interchangeably herein) to the subject.

As used herein, GIPr agonist peptides of the present disclosure refer to peptides that preferentially bind to GIP receptors compared to other receptors, such as GLP receptors. In some embodiments, an exemplary GIPr agonist peptide of the present disclosure are GIPr agonist peptides that have a selectivity ratio as defined as the ratio of (EC₅₀ GLP1R/EC₅₀ GIPR) greater than 10, or greater than 100, or greater than 1,000, or greater than 10,000, or greater than 100,000. An exemplary GIP receptor agonist peptide is a GIPr agonist peptide when the peptide has a selectivity ratio of (EC₅₀ GLP1R/EC₅₀ GIPR) of greater than 10, or 100, or 1,000, or 10,000, or from about 100-1,000,000 or more.

As used herein, “Lys(R)” is synonymous with “Km” and are used interchangeably.

In some embodiments, a GIP receptor agonist peptide, or a salt thereof is provided.

In some embodiments, the GIP receptor agonist peptide is represented by formula (I): P¹-Tyr-A2-Glu-Gly-Thr-Phe-Ile-Ser-A9-Tyr-Ser-Ile-A13-A14-Asp-A16-A17-A18-Gln-A20-A21-Phe-Val-A24-Trp-A26-Leu-A28-Gln-A30-A31-A32-A33-A34-A35-A36-A37-A38-A39-A40-P², or a pharmaceutically acceptable salt thereof;

wherein
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Gly, or Pro;
A9: represents Asp or Leu;
A13: represents Aib, or Ala;
A14: represents Leu, Aib, Lys;
A16: represents Arg, Ser, or Lys;
A17: represents Aib, Gln, or Ile;
A18: represents Ala, His, or Lys;
A19: represents Gln, or Ala;
A20: represents Aib, Gln, Lys, or Ala;
A21: represents Asp, Asn, or Lys;
A24: represents Asn, or Glu;
A26: represents Leu or Lys;
A28: represents Ala, Lys, or Aib;
A29: represents Gln, Lys, Gly, or Aib;
A30: represents Arg, Gly, Ser, or Lys;
A31: represents Gly, Pro, or a deletion;
A32: represents Ser, Gly, or a deletion;
A33: represents Ser, Gly, or a deletion;
A34: represents Gly, Lys, Asn, or a deletion;
A35: represents Ala, Asp, Ser, Lys, or a deletion;
A36: represents Pro, Trp, Lys, or a deletion;
A37: represents Pro, Lys, Gly, or a deletion;
A38: represents Pro, His, Lys, or a deletion;
A39: represents Ser, Asn, Gly, Lys, or a deletion; and
A40: represents Ile, Lys or a deletion.

In related embodiments, the GIP receptor agonist peptide according to Formula (I) has an amino acid sequence of Formula (I), wherein A31 is Gly and A32-A39 are deletion, or A32 is Gly and 33-A39 are deletion.

In various embodiments, the GIP receptor agonist peptide of Formula (I) comprises a peptide wherein P² is —OH.

On other embodiments, the GIP receptor agonist peptide of Formula (I) comprises a peptide wherein P¹ is methyl, (Me).

In various embodiments, the GIP receptor agonist peptide of Formula (I) comprises a peptide wherein P¹ is methyl, (Me), and P² is —OH.

In some embodiments, a GIP receptor agonist peptide, or a salt thereof is provided. The GIP receptor agonist peptide is represented by formula (II): P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-A26-Leu-Ala-A29-A30-A31-A32-A33-A34-A35-A36-A37-A38-A39-A40-P², or a pharmaceutically acceptable salt thereof, wherein:

P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, Ser, Ala, D-Ala, or Gly;
A13: represents Aib, Tyr, or Ala;
A14: represents Leu, or Lys(R);
A16: represents Arg, Ser, or Lys;
A17: represents Aib, Ile, Gln, or Lys(R);
A18: represents Ala, His, or Lys(R);
A19: represents Gln or Ala;
A20: represents Aib, Gln, or Lys(R);
A21: represents Asn, Glu, Asp, or Lys(R);
A24: represents Asn, or Glu;
A26: represents Leu or Lys(R);
A28: represents Ala, Aib, or Lys(R);
A29: represents Gln, Aib, or Lys(R);
A30: represents Arg, Gly, Lys, Ser, or Lys(R);
A31: represents Gly, Pro, or a deletion;
A32: represents Ser, Lys, Pro, Gly, or a deletion;
A33: represents Ser, Lys, Gly, or a deletion;
A34: represents Gly, Lys, Asn, or a deletion;
A35: represents Ala, Asp, Ser, Lys, or a deletion;
A36: represents Pro, Trp, Lys, or a deletion;
A37: represents Pro, Lys, Gly, or a deletion;
A38: represents Pro, His, Lys, or a deletion;
A39: represents Ser, Asn, Lys, Gly, or a deletion;
A40: represents Ile, Lys(R), or a deletion;
wherein the residue Lys(R), the (R) portion represents X-L-, wherein L represents a linker, and is selected from the following group consisting of gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE; and X represents a lipid.

In some embodiments, a GIP receptor agonist peptide, or a salt thereof is provided. The GIP receptor agonist peptide is represented by formula (III): P¹-Tyr-A2-Glu-Gly-Thr-Phe-Ile-Ser-Asp-Tyr-Ser-Ile-A13-A14-Asp-A16-A17-A18-Gln-A20-A21-Phe-Val-A24-Trp-A26-Leu-A28-A29-A30-A31-A32-A33-A34-A35-A36-A37-A38-A39-A40-P², or a pharmaceutically acceptable salt thereof, wherein:

P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Ser, or Gly;
A13: represents Aib, Tyr, or Ala;
A14: represents Leu, or Lys(R);
A16: represents Arg, Ser, or Lys;
A17: represents Aib, Ile, Gln, or Lys(R);
A18: represents Ala, His, or Lys(R);
A20: represents Aib, Gln, or Lys(R);
A21: represents Asp, Asn, Glu, or Lys(R);
A24: represents Asn, or Glu;
A26: represents Leu, or Lys(R);
A28: represents Ala, Aib, or Lys(R);
A29: represents Gln, Aib, Gly, or Lys(R);
A30: represents Arg, Lys, Ser, or Lys(R);
A31: represents Gly, Pro, or a deletion;
A32: represents Ser, Gly, or a deletion;
A33: represents Ser, Gly, or a deletion;
A34: represents Gly, Lys, or a deletion;
A35: represents Ala, Lys, Ser, or a deletion;
A36: represents Pro, Lys, or a deletion;
A37: represents Pro, Lys, Gly, or a deletion;
A38: represents Pro, Lys, or a deletion;
A39: represents Ser, Lys, Gly, or a deletion;
A40: represents Lys(R) or a deletion; and
wherein the residue Lys(R), the (R) portion represents X-L-, wherein L represents a linker, and is selected from the following group consisting of 2OEGgEgE, OEGgEgE, 2OEGgE, 3OEGgEgE, G5gEgE, 2OEGgEgEgE, 2OEG and G5gEgE; and X represents a lipid.

In some embodiments, a GIP receptor agonist peptide, or a salt thereof is provided. The GIP receptor agonist peptide is represented by formula (IV): P¹-Tyr-A2-Glu-Gly-Thr-A6-A7-Ser-Asp-Tyr-Ser-Ile-A13-A14-Asp-A16-A17-A18-Gln-A20-A21-Phe-Val-Asn-Trp-Leu-Leu-A28-A29-A30-A31-A32-A33-A34-A35-A36-A37-A38-A39-P², or a pharmaceutically acceptable salt thereof;

wherein
P¹ represents H, C₁₋₆ alkyl, or absent;
P² represents —NH₂ or —OH;
A2 represents Aib, Gly, or Ser;
A6 represents Phe or Leu;
A7 represents Ile or Thr;
A13 represents Ala, Aib, or Tyr;
A14 represents Leu, Lys, or Lys(R);
A16 represents Lys, Arg, or Ser;
A17 represents Aib, Ile, Lys, or Lys(R);
A18 represents Ala, His, Lys, or Lys(R);
A20 represents Gln, Lys, Lys(R), or Aib;
A21 represents Asp, Lys, Lys(R), or Asn;
A28 represents Ala, Aib, or, Lys, Lys(R);
A29 represents Gln, Lys, Lys(R), or Aib;
A30 represents Lys, Ser, Arg, Lys(R), or Lys(Ac);
A31 represents Pro, Gly, or a deletion;
A32 represents Ser, Gly, or a deletion;
A33 represents Ser, Gly, or a deletion;
A34 represents Gly, Lys, or a deletion;
A35 represents Ala, Ser, Lys, or a deletion;
A36 represents Pro, Lys, or a deletion;
A37 represents Pro, Lys, Gly, or a deletion;
A38 represents Pro, Lys, or a deletion; and
A39 represents Ser, Gly, Lys, or a deletion,
wherein in the residue Lys(R), the (R) portion represents X-L-, wherein L represents a linker and is selected from the group consisting of 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE, and OEGgEgEgE; and X represents C₁₄-C₁₈ monoacid or C₁₄-C₁₈ diacid.

In some embodiments, A2 represents Aib.

In some embodiments, A6 represents Phe.

In some embodiments, A7 represents Ile.

In some embodiments, A13 represents Ala or Aib.

In some embodiments, A16 represents Arg.

In some embodiments, A31 represents Pro or Gly, and A32-A39 is deletion.

In some embodiments of formula (IV), A14 represents Leu or Lys(R).

In some embodiments of formula (IV), A17 represents Aib, Ile, or Lys(R).

In some embodiments of formula (IV), A17 represents Aib or Lys(R).

In some embodiments of formula (IV), A18 represents Ala, His, or Lys(R).

In some embodiments of formula (IV), A20 represents Gln, Lys(R), or Aib.

In some embodiments of formula (IV), A21 represents Asp, Lys(R), or Asn.

In some embodiments of formula (IV), A28 represents Ala, Aib, or Lys(R).

In some embodiments of formula (IV), A29 represents Gln, Lys(R), or Aib.

In some embodiments of formula (IV), A30 represents Lys, Ser, Arg, Lys(R), or Lys(Ac).

In some embodiments of formula (IV), A30 represents Ser, Arg, Lys(R), or Lys(Ac).

In some embodiments of formula (IV),

A14 represents Leu or Lys(R);
A17 represents Aib, Ile, or Lys(R);
A18 represents Ala, His, or Lys(R);
A20 represents Gln, Lys(R), or Aib;
A21 represents Asp, Lys(R), or Asn;
A28 represents Ala, Aib, or Lys(R);
A29 represents Gln, Lys(R), or Aib; and
A30 represents Lys, Ser, Arg, Lys(R), or Lys(Ac).

In some embodiments of formula (IV),

A2 represents Aib;
A17 represents Aib, Lys, or Lys(R);
A20 represents Aib; and
A28 represents Ala or Aib,
wherein L is selected from the group consisting of 2OEG, 2OEGgE, 2OEGgEgE, G2E3, G4gE, G4gEgE, G5, G5E, G5gE, G5gEgE, gEgEgE, GGEEE, GGPAPAP, OEGgEgE, and OEGgEgEgE.

In some embodiments of formula (IV),

A2 represents Aib;
A14 represents Leu or Lys(R);
A17 represents Aib or Lys(R);
A18 represents Ala, His, or Lys(R);
A20 represents Aib;
A21 represents Asp, Lys(R), or Asn;
A28 represents Ala or Aib;
A29 represents Gln, Lys(R), or Aib; and
A30 represents Lys, Ser, Arg, Lys(R), or Lys(Ac),
wherein L is selected from the group consisting of 2OEG, 2OEGgE, 2OEGgEgE, G2E3, G4gE, G4gEgE, G5, G5E, G5gE, G5gEgE, gEgEgE, GGEEE, GGPAPAP, OEGgEgE, and OEGgEgEgE.

In some embodiments, the GIP receptor agonist peptide comprises a peptide wherein P² is —OH. In some embodiments, the GIP receptor agonist peptide comprises a peptide wherein P² is —NH₂.

In some embodiments, the GIP receptor agonist peptide comprises a peptide wherein P¹ is a C₁₋₆ alkyl group. In some embodiments, the GIP receptor agonist peptide comprises a peptide wherein P¹ is methyl, (Me).

In some embodiments, the GIP receptor agonist peptide comprises a peptide wherein P¹ is Me and P² is —OH.

In some embodiments, the GIP receptor agonist peptide comprises a peptide wherein L is 2OEGgEgE or GGGGG.

In some embodiments, the GIP receptor agonist peptide comprises a peptide wherein X is C₁₅ diacid or C₁₆ diacid.

In some embodiments, the GIPR agonist peptide or the pharmaceutically acceptable salt thereof is represented by Formula (V): P¹-Tyr-Aib-Glu-Gly-The-Phe-Ile-Ser-Asp-Tyr-Ser-Ile-A13-Leu-Asp-Arg-Aib-A18-Gln-Aib-A21-Phe-Val-Asn-Trp-Leu-Leu-Ala-Gln-A30-A31-A32-P², wherein

P¹ is methyl;
P² is OH or NH₂;
A13 represents Ala or Aib;
A18 represents Ala, Lys, or Lys(R);
A21 represents Lys, Lys(R), or Asp;
A30 represents Lys or Ser;
A31 represents Gly or Pro; and
A32 represents Gly or deletion;
wherein (R) represents X-L-, L represents 2OEGgEgE or GGGGG; and X represents a C₁₅ diacid or C₁₆ diacid.

In some embodiments of formula (V), A18 represents Ala or Lys(R).

In some embodiments of formula (V), A21 represents Lys(R) or Asp.

In some embodiments of Formula (V), the GIPR agonist peptide or the pharmaceutically acceptable salt thereof is represented by the following formula: P¹-Tyr-Aib-Glu-Gly-The-Phe-Ile-Ser-Asp-Tyr-Ser-Ile-A13-Leu-Asp-Arg-Aib-A18-Gln-Aib-A21-Phe-Val-Asn-Trp-Leu-Leu-Ala-Gln-A30-A31-A32-P², wherein

P¹ is methyl;
P² is OH or NH₂;
A13 represents Ala or Aib;
A18 represents Ala or Lys(R);
A21 represents Lys(R) or Asp;
A30 represents Lys or Ser;
A31 represents Gly or Pro; and
A32 represents Gly or deletion;
wherein (R) represents X-L-, L represents 2OEGgEgE or GGGGG; and X represents a C₁₅ diacid or C₁₆ diacid.

In various embodiments, an illustrative GIP receptor agonist peptide for use in the methods, compositions and medicaments exemplified herein, has at least 80%, or at least 85%, or at least 90%, or at least 95%, or at least 96%, or at least 97%, or at least 98%, or at least 99%, or 100% sequence identity to any GIP receptor agonist peptide as defined by formulas (I), (II), (III), (IV), or (V).

In various embodiments, an illustrative GIP receptor agonist peptide for use in the methods, compositions and medicaments exemplified herein, has 100% sequence identity to any GIP receptor agonist peptide as defined by formulas (I), (II), (III), (IV), or (V).

In various embodiments, the GIP receptor agonist peptide as defined by formulas (I), (II), (III), (IV), or (V), has a P² defined by a hydroxyl (—OH) group. In various embodiments, the GIP receptor agonist peptide as defined by formulas (I), (II), (III), (IV), or (V), has a P² defined by an amino (—NH₂) group.

In various embodiments, the GIP receptor agonist peptide as defined by formulas (I), (II), (III), (IV), or (V), has a P¹ defined by a C₁₋₆ alkyl group. In some embodiments, P¹ is a methyl (Me) group.

With reference to the above GIP receptor agonist peptides as defined by formulas (I), (II), (III), (IV), and (V), in various embodiments, a GIP receptor agonist peptide has at least one amino acid having a bivalent substituent, covalently coupled to a side chain of an amino acid. For example, in some embodiments, a GIP receptor agonist peptide has an amino acid sequence having a side chain of at least one amino acid, or modified amino acid for example, a Lys residue of the GIP receptor agonist peptide being covalently attached to a substituent group (R). In various embodiments, a Lys residue of the GIP receptor agonist peptide may be covalently attached to a substituent (R) as shown in the present disclosure as Lys(R).

For example, a selective GIP receptor agonist peptide of the present disclosure may have a Lys residue substituted by an (R) group at an amino acid position A14-A30, for example, at amino acid position: A14, or A17, A18, A20, A21, A28, A29, or A30. In various embodiments, the (R) group represents X-L-, wherein L represents a bivalent linker. In some embodiments, the bivalent linker can include a PEG, Abu-, (Gly)(2-8)-, gGlu(1-3)-, gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE one to ten amino acids, for example, a glycine linker having two to ten glycine residues, two to six or from five to six glycines linked, or combinations of the foregoing linkers. In these embodiments, X represents a substituent group, for example, a lipid. In various embodiments, X represents a monoacid or diacid lipid having C₁₄ to C₁₆ carbons in length, for example, a C₁₄, a C₁₅, a C₁₆ monoacid or diacid lipid. In various embodiments, X represents a monoacid or diacid lipid having C₁₄ to C₁₈ carbons in length, for example, a C₁₅, a C₁₆, a C₁₈ monoacid or diacid lipid. In some embodiments, X is a C₁₅ diacid, C₁₆ diacid, or C₁₈ diacid. In some embodiments, X is a C₁₅ diacid or C₁₆ diacid. In some embodiments, X is a C₁₈ diacid.

In various embodiments, the GIP receptor agonist peptide may include one or two Lys residues is substituted with an X-L- substituent. In some embodiments, a Lys residue is substituted with an X-L- substituent, wherein L represents (PEG3)2-, Abu-, (Gly)(2-8)-, gGlu(1-3)-, or combinations thereof, for example, (PEG3)2-gGlu-, Abu-gGlu-, (Gly)s-gGlu-, or (Gly)₆-gGlu-, GGGGG-, (PEG3)2-, PEG3)2-(Gly)5-6-, gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, or combinations thereof.

In some embodiments, the GIP receptor agonist peptide has one, or two Lys residues having a substituted side chain. For example, a selective GIPr agonist peptide may have a Lys residue substituted by X-L-, wherein L represents a bivalent linker, as discussed herein, for example, L may represent a bond or a bivalent substituent group, and wherein X represents an optionally substituted hydrocarbon group, for example a monoacid or diacid lipid, or a salt thereof. In some embodiments, the bivalent substituent group comprises: an alkylene group, a carbonyl group, an oxycarbonyl group, an imino group, an alkylimino group, a sulfonyl group, an oxy group, a sulfide group, an ester bond, an amide bond, a carbonate bond or combinations thereof.

In various embodiments, the GIP receptor agonist peptide may include one, or two Lys residues which may be substituted with an (R) group defined as an X-L- substituent. In some embodiments, Lys(R) is a Lys residue having a side chain substituted with X-L-. In related embodiments, the GIP receptor agonist peptide, the X moiety can be an optionally substituted hydrocarbon. In some embodiments, the X moiety in the X-L- substituent can include a C₁₇-C₂₂ monoacid, a C₁₇-C₂₂ diacid, an acetyl group, or combinations thereof Some exemplary X moieties may include: (Teda:C14 diacid), (Peda:C15 diacid), (Heda:C16 diacid).

In various embodiments, a GIP receptor agonist peptide of formulas (I) to (V), the L moiety of the X-L- group can include, a bivalent linker. In some examples, the bivalent linker can include PEG, Abu-, (Gly)₍₂₋₈₎-, gGlu₍₁₋₃₎-, one to ten amino acids, or combinations thereof. In these examples of X-L, X may represents a substituent group.

In some embodiments, (R) represents X-L- wherein L represents (PEG3)2-, Abu-, (Gly)₍₂₋₈₎-, gGlu₍₁₋₃₎-, or combinations thereof. In some embodiments, L represents (PEG3)2-gGlu-, Abu-gGlu-, (Gly)₅-gGlu, (Gly)₆-gGlu-, GGGGG-, GGGGGG-, (PEG3)2-, or (PEG3)2-(Gly)₅₋₆-, GGGGG-, (PEG3)2-, PEG3)2-(Gly)5-6-, gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, or combinations thereof.

In some related embodiments, L represents a bond or a bivalent substituent group, and X represents an optionally substituted hydrocarbon group, or a salt thereof For example, an illustrative GIP receptor agonist peptide has a Lys(R) residue, wherein the (R) portion of the Lys(R) residue is represented as X-L-, wherein X is a bivalent substituent group comprising an alkylene group, a carbonyl group, an oxycarbonyl group, an imino group, an alkylimino group, a sulfonyl group, an oxy group, a sulfide group, an ester bond, an amide bond, a carbonate bond or combinations thereof.

In some embodiments, an illustrative Lys(R) can include an (R) group defined as X-L- group, wherein the bivalent substituent X is a C₁₄-C₁₆ monoacid, a C₁₄-C₁₈ diacid, a C₁₇-C₂₂ diacid or an acetyl group. Some exemplary X moieties may include: (Teda:C14 diacid), (Peda:C15 diacid), (Heda:C16 diacid).

In some embodiments, an illustrative GIP receptor agonist peptide of formulas (I), (II), (III), (IV), or (V), can include a peptide having one, to two Lys(R) lipidated amino acids positioned in the amino acid sequence of the peptide ranging from residue A14 to A30, wherein the Lys(R) residue has a substituted side chain defined by X-L-. In exemplary embodiments, the X-L- group of the Lys(R) residue in the illustrative GIP receptor agonist peptide of formulas (I), (II), (III), (IV), or (V), may include: -(g-Glu)₂-Oda, -(g-Glu)₂-Eda, -(g-Glu)₂-Heda, -(PEG3)2-gGlu-Eda, -(PEG3)2-gGlu-Heda, -(PEG3)2-gGlu-Oda, -(PEG3)2-gGlu-Ida, -(PEG3)-gGlu-Eda, -(PEG3)-gGlu-Heda, -(PEG3)-gGlu-Oda, -Abu-gGlu-Oda, -(Gly)₅-gGlu-Eda, -(Gly)₅-gGlu-Heda, -(Gly)₅-gGlu-Oda, -(Gly)₅-Heda, -(Gly)₅-Oda, -(Gly)₅-Eda, -(PEG3)2-Heda, -(PEG3)2-Eda, -(PEG3)2-Oda, 2OEGgEgE-Teda:C14 diacid, OEGgEgE-Teda:C14 diacid, 2OEGgE-Teda:C14 diacid, 3OEGgEgE-Teda:C14 diacid, G5gEgE-Teda:C14 diacid, 2OEGgEgEgE-Teda:C14 diacid, 2OEG-Teda:C14 diacid, G5gEgE-Teda:C14 diacid, 2OEGgEgE-Peda:C15 diacid, OEGgEgE-Peda:C15 diacid, 2OEGgE-Peda:C15 diacid, 3OEGgEgE-Peda:C15 diacid, G5gEgE-Peda:C15 diacid, 2OEGgEgEgE-Peda:C15 diacid, 2OEG-Peda:C15 diacid, G5gEgE-Peda:C15 diacid, 2OEGgEgE-Heda:C16 diacid, OEGgEgE-Heda:C16 diacid, 2OEGgE-Heda:C16 diacid, 3OEGgEgE-Heda:C16 diacid, G5gEgE-Heda:C16 diacid, 2OEGgEgEgE-Heda:C16 diacid, 2OEG-Heda:C16 diacid, G5gEgE-Heda:C16 diacid, or combinations thereof.

In some illustrative examples, the (R) group may be covalently linked to a side chain of a Lys amino acid. In some examples, an exemplary (R) group represents X-L-, wherein L represents a bivalent linker comprising PEG and/or two or more amino acids, and X represents a substituent group, or a salt thereof. In various embodiments, the GIP receptor agonist peptide of formulas (I)-(V) or a salt thereof, has one or two Lys(R), residues located at a position between A14 to A30, wherein (R) represents a substituent group.

In some embodiments, R represents X-L-, wherein L is one or a combination of more than one selected from 2OEGgEgE, OEGgEgE, 2OEGgE, 3OEGgEgE, G5gEgE, 2OEGgEgEgE, 2OEG, G5gEgE, and X represents C₁₄-C₁₆ monoacid or diacid lipid, or an acetyl group.

Alternatively, in some embodiments, (R) represents X-L-, wherein L represents a linker selected from 2OEGgEgE, OEGgEgE, 2OEGgE, 3OEGgEgE, G5gEgE, 2OEGgEgEgE, 2OEG, and G5gEgE, and X represents C₁₄-C₁₆ linear saturated dicarboxylic acid.

In various embodiments, in each of the examples of GIP receptor agonist peptides of formulas (I) to (V), at least one amino acid between A14 to A30, or from A14 to A21, or A14 or A21 is Lys(R), wherein (R) represents X-L-, wherein L represents a bivalent linker L, wherein L represents 2OEGgEgE, OEGgEgE, 2OEGgE, 3OEGgEgE, G5gEgE, 2OEGgEgEgE, 2OEG, or G5gEgE. In some related embodiments, (R) represents X-L-, wherein L represents a bond or a bivalent substituent group, and X represents an optionally substituted hydrocarbon group, or a salt thereof. In various embodiments related to the various L moiety exemplifications, (R) represents X-L, wherein L is discussed above and X is a C₁₄-C₁₆ monoacid, or a C₁₄-C₁₆ diacid or an acetyl group. For example, in some embodiments, X is (Teda:C14 diacid), (Peda:C15 diacid), (Heda:C16 diacid). In various embodiments, an exemplary GIP receptor agonist peptide of formulas (I) to (V), comprises a peptide having at least one Lys amino acid positioned between A14 to A30, or from A14 to A21, for example, at an amino acid position A14, or A17, A18, A20, A21, A26, A29, or A30 of the peptide. The (R) substituent portion of the Lys(R) residue, represents X-L-, wherein L represents a bivalent linker L, for example, L represents 2OEGgEgE, OEGgEgE, 2OEGgE, 3OEGgEgE, G5gEgE, 2OEGgEgEgE, 2OEG, or G5gEgE and X is a C₁₄-C₁₆ monoacid, or a C₁₄-C₁₆ diacid or an acetyl group, for example, a C₁₄ monoacid or a C₁₄ diacid or a C₁₅ monoacid or a C₁₅ diacid or a C₁₆ monoacid or a C₁₆ diacid. In various embodiments, an exemplary GIP receptor agonist peptide of formulas (I) to (V), comprises at least one Lys amino acid positioned between A14 to A30, or from A14 to A21, or A14 or A21, wherein (R) represents X-L-, wherein L represents a bivalent linker L, wherein L represents 2×γGlu-2×OEG (miniPEG), and X is a C₁₅ monoacid or C₁₅ diacid.

In some embodiments, (R) represents X-L-, wherein L represents a bivalent linker comprising PEG and/or amino acid or consisting of PEG and/or one or more amino acids, for example, a Gly₂₋₁₀-linker, and X represents a substituent group. A known PEG linker, an amino acid linker or combinations thereof may be used as illustrative examples of a bivalent linker, as long as it is able to link Lys to a substituent group. Alternatively, in some embodiments, R represents X-L-, wherein L represents a bond or a bivalent substituent group, and X represents an optionally substituted hydrocarbon group, or a salt thereof A known bivalent substituent group may include, but is not limited to, an alkylene group, a carbonyl group, an oxycarbonyl group, an imino group, an alkylimino group, a sulfonyl group, an oxy group, a sulfide group, an ester bond, an amide bond, a carbonate bond or combinations thereof may be used.

In some embodiments, L represents (PEG3)2-, Abu-, (Gly)₍₂₋₁₀₎-, gGlu₍₁₋₃₎-, or combinations thereof. In some embodiments, L represents (PEG3)2-gGlu-. In some examples, L represents Abu-gGlu-. In other examples, L represents (Gly)₅-gGlu-, or (Gly)₆-gGlu-. In some embodiments, L represents a glycine peptide having from about two to about ten glycines linked, or from about two to about seven glycines linked. In some examples, L represents (Gly)₅₋₆-, or (Gly)₅-, GGGGG-, or GGGGG-gGlu-. In some examples, L represents 2OEGgEgE, OEGgEgE, 2OEGgE, 3OEGgEgE, G5gEgE, 2OEGgEgEgE, 2OEG, or G5gEgE.

In some embodiments, L represents (PEG3)2-. In some embodiments, L represents (Gly)₂₋₁₀-, for example, (Gly)₍₅₋₆₎. In some further embodiments, L represents a combination of groups, such as one or more PEG molecules linked to a glycine peptide: Gly₂₋₁₀ for example, L may be (PEG3)2-(Gly)₅₋₆-, or (PEG3)2-(Gly)₅-.

In some embodiments, the (R) group attached to an amino acid, for example, a Lys residue represents X-L-, wherein L represents a bivalent linker comprising PEG and/or one or more amino acids or consisting of PEG and/or one or more amino acids, and X represents a substituent group. A known PEG linker, an amino acid linker or combinations thereof may be used as the bivalent linker as long as it is able to link, a Lys residue to a substituent group. Alternatively, R represents X-L-, wherein L represents a bond or a bivalent substituent group, and X represents an optionally substituted hydrocarbon group, or a salt thereof. A known bivalent substituent group including, but are not limited to, an alkylene group, a carbonyl group, an oxycarbonyl group, an imino group, an alkylimino group, a sulfonyl group, an oxy group, a sulfide group, an ester bond, an amide bond, a carbonate bond or combinations thereof may be used. In some embodiments, (R) represents X-L-, wherein L is one or a combination of more than one selected from:

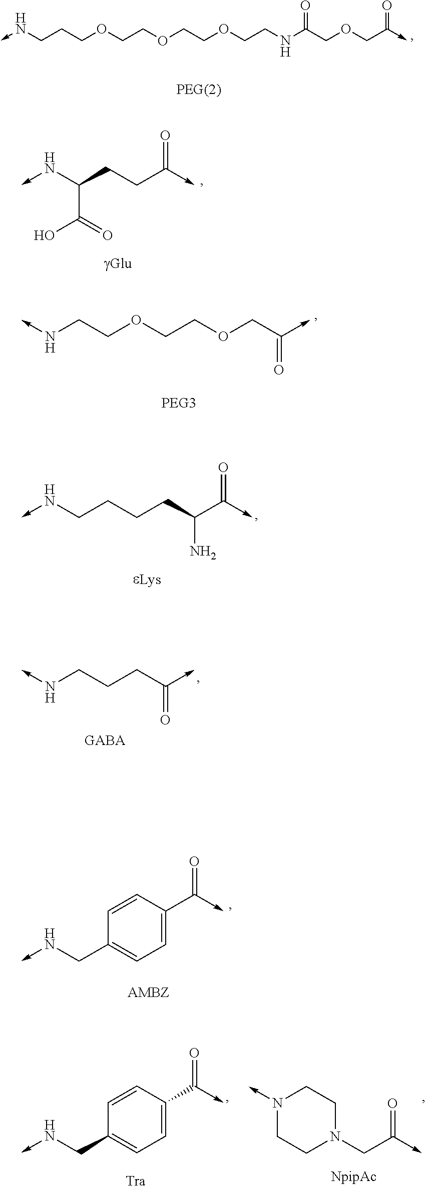

a glycine linker comprising one or two to nine-linked glycine(s) or a single bond, and X represents C₁₇-C₂₂ monoacid or diacid, or an acetyl group. In some embodiments, a linker L, can be coupled or linked covalently to a side chain of at least one amino acid, or modified amino acid for example, a Lys residue of the GIP receptor agonist peptide being covalently attached to a substituent group. In an embodiment, the selective GIP receptor agonist peptide is covalently attached to an (R) group, wherein the (R) group is a hydrophilic polymer, and the Lys(R) residue is positioned at an amino acid position ranging from A14 to A30. In an embodiment, the selective GIP receptor agonist peptide is covalently attached to a hydrophilic polymer, for example, the hydrophilic polymer is a polyethylene glycol (PEG) molecule or a variant thereof.

In some embodiments, the linker L is a PEG molecule, for example, PEG3(n), PEG(2)(n), or mPEG having a weight average molecular weight of about 5-30 kDa. In some embodiments, L can be any combination of PEG3(n), PEG(2)(n), gGlu(n), D-gGlu(n), AMBZ(n), GABA(n), G(x), NpipAc(n), Tra(n), eLya(n), where n=1-5 and x=1-10. Exemplary PEG linkers can be used as part of an (R) group in a substituted Lys residue, for example, located at one or more of A14-A30, for example, at an amino acid position: A14, A17, A18, A20, A21, AA26, A29, or A30, wherein the MPEG linker can include one or more of the following additional MPEG linkers:

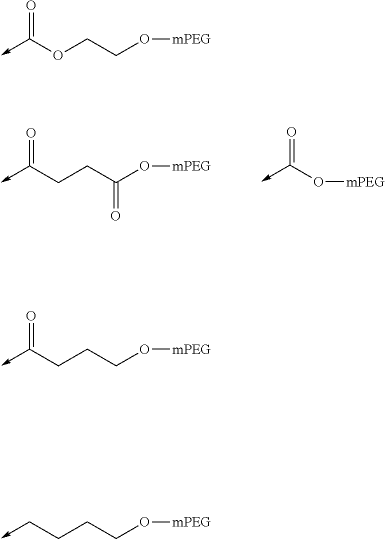

In some embodiments, exemplary MPEG linkers which may be used for coupling a substituent X to a Cys amino acid can include a MPEG molecule having an weight average molecular weight of about 5-30 kDa. In some embodiments, illustrative PEG linkers for attachment to a Cys side chain can include:

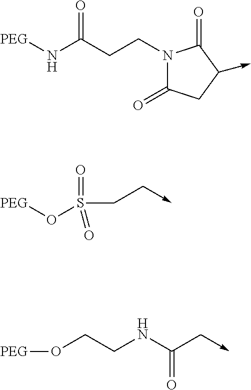

In various examples, R represents X-L-, wherein X-L- represents Teda-GGGG-(Teda:C14 diacid), Teda-GGGGG-, Teda-GGGGGG-, Peda-GGGG-(Peda:C15 diacid), Peda-GGGGG-, Peda-GGGGGG-, Heda-GGGG-(Heda:C16 diacid), Heda-GGGGG-, Heda-GGGGGG-, Heda-GGGGGGGGG-.

Alternatively, the (R) group represents X-L-, wherein L represents a glycine linker comprising five or six-linked glycines, and X represents C₁₄-C₁₆ linear saturated dicarboxylic acid.

Alternatively, the (R) group represents X-L-, wherein L represents a bond or a bivalent substituent group, and X represents an a C₁₄-C₁₆ fatty acid, or a C₁₄-C₁₆ acylated fatty acid or a C₁₄-C₁₆ dicarboxylic acid, or a salt thereof. In some embodiments, the X represents a palmitic fatty acid used to add a palmitoyl group to the epsilon amine side group of a Lys residue, for example, a Lys reside in the GIP receptor agonist peptide.

In other embodiments, the GIP receptor agonist peptide has one, or two modified lysine residues, i.e. Lys(R), wherein the (R) group represents X-L-, wherein L represents a glycine linker comprising three, four, five or six-linked glycines, and X represents C₁₄-C₁₆ linear saturated dicarboxylic acid. In an embodiment, the acyl group is a C₁₄ to C₁₆ fatty acyl group, for example a palmitoyl or myristoyl fatty acyl group.

In an embodiment, the GIP receptor agonist peptide is covalently attached to an (R) group, wherein the (R) group is a hydrophilic polymer at any amino acid position ranging from A14 to A30. In an embodiment, the GIP receptor agonist peptide is covalently attached to a hydrophilic polymer at amino acid position, A14, A17, A18, A20, A21, A26, A29, or A30, or combinations thereof, for example, at positions A14-A30 or from A14 to A21. For example, the hydrophilic polymer may be attached to the side chain of a Lys residue of the GIP receptor agonist peptide. In an embodiment, the hydrophilic polymer is a polyethylene glycol (mPEG). The mPEG polymer may also be further conjugated to a glycine linker, i.e. (Gly)₍₂₋₈₎-, or to one or more gGlu- residues, for example, gGlu₍₁₋₃₎-. In some examples, the mPEG has a weight average molecular weight of about 1,000 Daltons to about 60,000 Daltons, such as about 5,000 Daltons to about 40,000 Daltons, or about 1,000 Daltons, or 5,000 Daltons, or 10,000 Daltons, or 12,000 Daltons, or 14,000 Daltons to about 20,000 Daltons.

In some embodiments, methods for conjugating a polyethylene glycol (mPEG) polymer to a reactive amine or sulfhydryl group is well known in the art. For example, mPEG can be conjugated to a lysine amine sidechain using an amine-reactive pegylated crosslinker. A Bis(succinimidyl)penta-(ethylene glycol) spacer arm can be used as a homobifunctional, amine-to-amine crosslinker that contain N-hydroxy-succinimide (NHS) esters at both ends of a mPEG spacer arm. An amine-reactive crosslinker that contains a PEG spacer arm. A bis-succinimide ester-activated mPEG compound may be used for crosslinking between primary amines (—NH₂) in GIP receptor agonist peptides of the present disclosure. The N-hydroxysuccinimide ester (NHS) groups at either end of the mPEG spacer react specifically and efficiently with lysine and N-terminal amino groups at pH 7-9 to form stable amide bonds. Other homobifunctional, sulfhydryl-reactive crosslinkers that contain the maleimide group at either end of a PEG spacer may be used to couple PEG to a Cys amino acid of a GIP receptor agonist peptide. Heterofunctional crosslinking spacer arms may also be used when two different reactive groups are used as the linkage groups, e.g. an amine group and a sulfhydryl group. A sulfhydryl-reactive crosslinker that contains a PEG spacer arm, may be used to couple a PEG polymer to a GIP receptor agonist peptide. In some embodiments, a bismaleimide-activated PEG compound may be used for crosslinking between sulfhydryl (—SH) groups in proteins and other thiol molecules. The maleimide groups at either end of the PEG spacer may react specifically and efficiently with reduced sulfhydryls at pH 6.5-7.5 to form stable thioether bonds. In other embodiments, direct coupling of a PEG molecule to a GIP receptor agonist peptide may be accomplished using known methods in the art. For example, a well known technique whereby a peptide may be covalently modified with PEG groups requiring PEG compounds that contain a reactive or targetable functional group at one end. The simplest method to pegylate peptides, which are rich in surface primary amines, is to use a PEG compound that contains an NHS ester group at one end, for example, a methyl-(PEG)n-NHS ester. In a similar fashion, methyl-(PEG)n-maleimide (wherein n can be from 20-300) may be used to couple a PEG molecule to a Cys containing peptide of the present disclosure. Methods known in the art for conjugation of polyethylene glycol polymers of various lengths ranging from 1,000 Daltons to 20,000 Daltons or more are provided in 1. Hermanson, G. T. (2013). 3rd Edition. Bioconjugate Techniques, Academic Press, Veronese, F. and Harris, J. M. Eds. (2002). Peptide and protein PEGylation. Advanced Drug Delivery Review 54(4), 453-609, Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182 the disclosures of all of these references are hereby incorporated by reference herein in their entireties.

In various embodiments, the GIP receptor agonist peptide disclosed herein with the lipidated Lys(R) residues positioned between amino acids A14 and A30, for example, at amino acid positions A14, A17, A18, A20, A21, A28, A29, or A30, provide GIPR agonist peptides having enhanced ½ life of elimination, % remaining after 48 hours in serum, and solubility in various media, when compared to GIPR agonist peptides in the art. In some embodiments, the position of the lipidated lysine residue, the sequence of the GIPR peptide and the length of the lipid used in the (R) substituent on the Lys residue play a role in the improved half life and solubility of the GIPR peptide, that enables the GIPR agonist peptides to be dosed in a therapeutically effective way to a subject in need of antiemetic activity once per day (Q1D), for example, once per 24 hours. The enhanced 12 life of elimination, % remaining after 48 hours in serum, and solubility in various media are illustrated in the Examples section of the present disclosure.

In various embodiments, GIP receptor agonist peptides disclosed herein which are suitable for Q1D, or once per day dosing to treat emesis, including nausea and/or vomiting, have a human intravenous T½ life of elimination in human serum, ranging between 4-10 hours, or for example, ranging between 4-6 hours. In various embodiments, GIP receptor agonist peptides disclosed herein which are suitable for Q1D dosing, or once per day dosing to treat emesis, including nausea and/or vomiting, have a solubility of greater than 10 mg/mL, or greater than 15 mg/mL, or greater than 20 mg/mL, or greater than 30 mg/mL, or greater than 40 mg/mL, or greater than 50 mg/mL, or greater than 60 mg/mL, or greater than 75 mg/mL, or greater than 100 mg/mL, or greater than 125 mg/mL (for example, when tested in a dissolution test using phosphate buffer at pH 7.4 performed at 37° C.); and a human intravenous T½ life of elimination in human serum ranging between 5 to 20 hours, or for example, ranging between 8 to 16 hours, or from 10 to 15 hours. In various embodiments, GIP receptor agonist peptides disclosed herein which are suitable for Q1D dosing, or once per day dosing to treat emesis, including nausea and/or vomiting, in a mammal, for example, a human, have a solubility of 15 mg/mL, or greater; and a human intravenous T½ life of elimination ranging between 8-16 hours, or for example, ranging between 10-15 hours. In various embodiments, the GIPR agonist peptides of the present disclosure have a T½ life of elimination in humans ranging from 10 to 16 hours as determined with the methods of the Examples below, and a solubility greater than 25 mg/mL, for example, greater than 30 mg/mL, or greater than 40 mg/mL, or greater than 45 mg/ml, or greater than 50 mg/mL or higher.

In various embodiments, GIP receptor agonist peptides disclosed herein which are suitable for Q1D dosing, or once per day dosing to treat emesis, including nausea and/or vomiting, in a mammal, for example, a human, have a solubility of 15-100 mg/mL, or greater; and a human intravenous T½ life of elimination ranging from 10 to 16 hours as determined with the methods of the Examples below, and a an amino acid sequence length of 30-31 or 39 amino acids, a substituted (Lys(R) Lysine residue positioned in the amino acid position of 14 or 21, a lipid characterized as a C15 diacid and a linker selected from 2OEGgEgE or GGGGG.

Solubility of the GIPR peptides may be determined by dissolution in a phosphate buffer followed by quantitation using liquid chromatography, for example, High Performance Liquid Chromatography (HPLC). An illustrative method is provided. For determination of the solubility of the GIPr agonist peptides, 3 mg of peptides are weighted out in a small glass vial. 100 μL of 200 mM Phosphate buffer pH 7.4 are added and the vial is sonicated/vortexed as necessary for a maximum of 1 min. A visual inspection is performed, If the sample is fully dissolved, the solubility is recorded as 30 mg/mL. If insoluble material is observed in the tube the addition of 100 μL of buffer and mixing is repeated until complete dissolution. If the peptide is not soluble in 500 μL of buffer, it is labeled as solubility <6 mg/mL. The solubility can be confirmed by RP-HPLC after filtration on 0.2 μm filter on an Agilent 1200 system with a Kinetex column form Phenomenex® (2.6 μm EVO C18 100 Å, LC Column 50×3.0 mm) kept at 40° C., the eluent A is 0.05% TFA in Water, B is 0.035% TFA in Acetonitrile at a 0.6 ml/min flow rate. The gradient was from 20 to 70 over 5 min, the column is then washed for 1 min at 90% B. UV monitoring at 215 nm was used to monitor peptide concentration. Standards, may also be run on the same chromatographical experiment, to obtain standard measurements at 215 nm, from which a standard curve may be calculated and soluble peptide concentrations may be extrapolated from the standard curve.

In various embodiments, the GIP receptor agonist peptide disclosed herein, for example, as used in the preparation of a medicament, a composition, or for use in the prevention and/or treatment of a condition, or disorder, or in a method of prevention and/or treatment as disclosed herein, as represented by a GIP receptor agonist peptide has an amino acid sequence as provided in any one of formulas (I)-(V).

In various embodiments, suitable GIPR agonist peptides having the appropriate pharmacokinetics and pharmacodynamics required for therapeutically effective treatment of a subject with emesis or displaying one or more symptoms of emesis, or for use to prevent emesis by dosing Q1D, or once per day, for example, once per 24 hours have the following amino acid sequence and lipid-linker characteristics:

TABLE 1
Exemplary GIPR Agonist Peptides of the
Present Disclosure.

| Compound | Amino Acid Sequence | | |
|---|---|---|---|
| No. | (One Letter Residue) | Linker | Lipid |
| 14 | Me-Y-Aib-E-G-T-F-I-S-D-Y-S-I-A-L-D-R- | 2OEGgEgE | C15 |
| | Aib-A-Q-Aib-Km-F-V-N-W-L-L-A-Q-R-NH₂ | | diacid |
| 17 | Me-Y-Aib-E-G-T-F-I-S-D-Y-S-I-A-L-D-R- | 2OEGgEgE | C15 |
| | Aib-Km-Q-Aib-N-F-V-N-W-L-L-A-Q-S-P-S- | | diacid |
| | S-G-A-P-P-P-S-NH₂ | | |
| 25 | Me-Y-Aib-E-G-T-F-I-S-D-Y-S-I-A-L-D-R- | 2OEGgEgE | C15 |
| | Aib-A-Q-Aib-Km-F-V-N-W-L-L-A-Q-K-G-OH | | diacid |
| 142 | Me-Y-Aib-E-G-T-F-I-S-D-Y-S-I-Aib-Km-D- | GGGGG | C15 |
| | R-Aib-A-Q-Aib-D-F-V-N-W-L-L-A-Q-R-G-OH | | diacid |
| 21 | Me-Y-Aib-E-G-T-F-I-S-D-Y-S-I-Aib-L-D- | 2OEGgEgE | C15 |
| | R-Aib-A-Q-Aib-N-F-V-N-W-L-L-A-Q-Km-P- | | diacid |
| | S-S-G-A-P-P-P-S-NH₂ | | |
| 48 | Me-Y-Aib-E-G-T-F-I-S-D-Y-S-I-A-Km-D- | GGGGG | C15 |
| | R-Aib-A-Q-Aib-N-F-V-N-W-L-L-A-Q-S-P- | | diacid |
| | S-S-G-A-P-P-P-S-OH | | |
| 20 | Me-Y-Aib-E-G-T-F-I-S-D-Y-S-I-Aib-L- | 2OEGgEgE | C15 |
| | D-R-Km-A-Q-Aib-N-F-V-N-W-L-L-A-Q-R- | | diacid |
| | P-S-S-G-A-P-P-P-S-NH₂ | | |

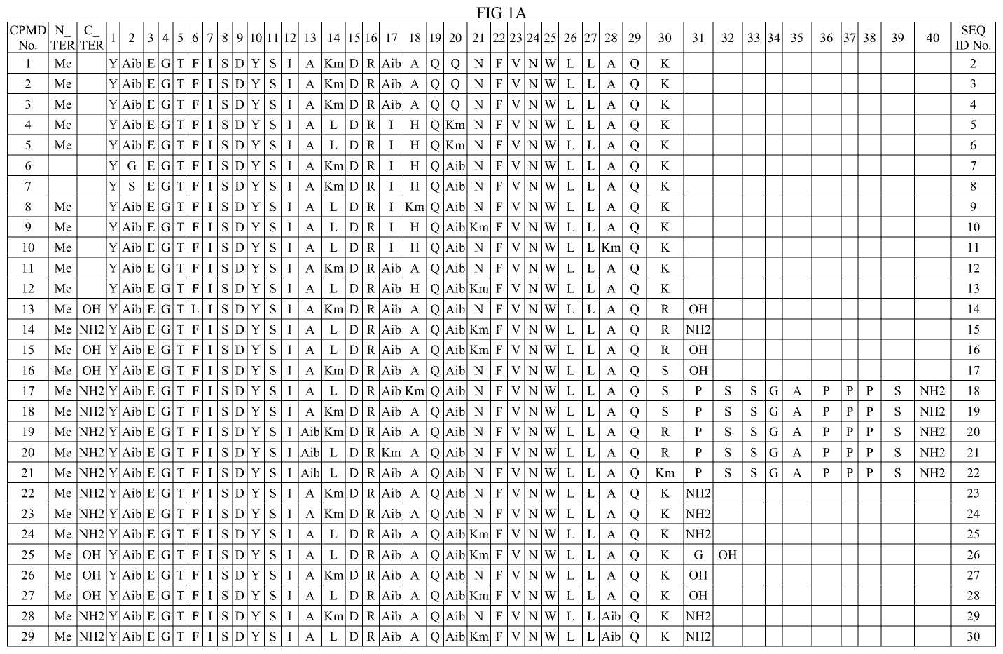

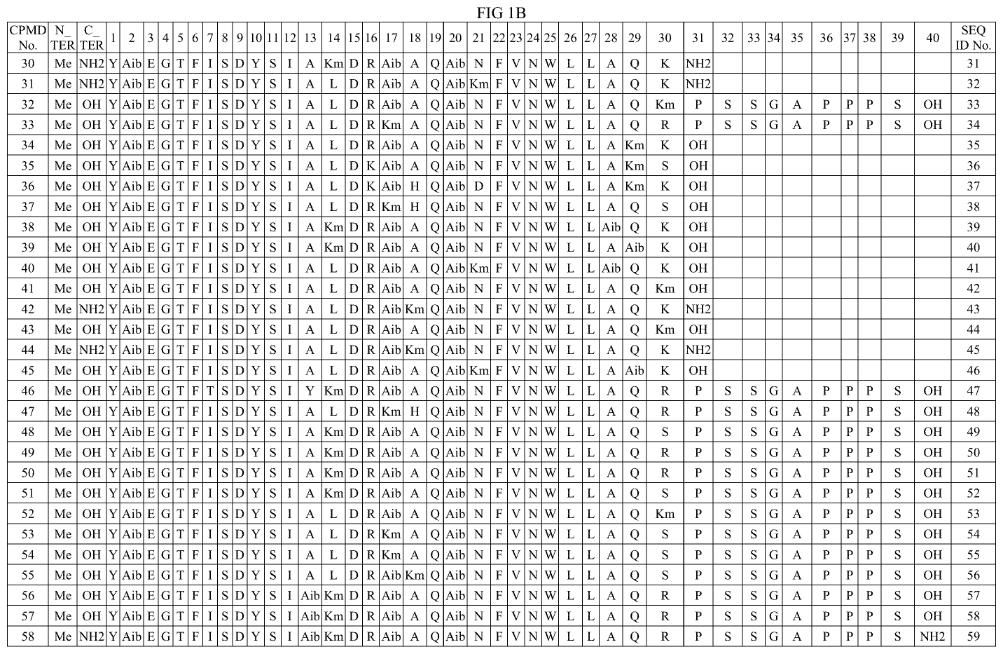

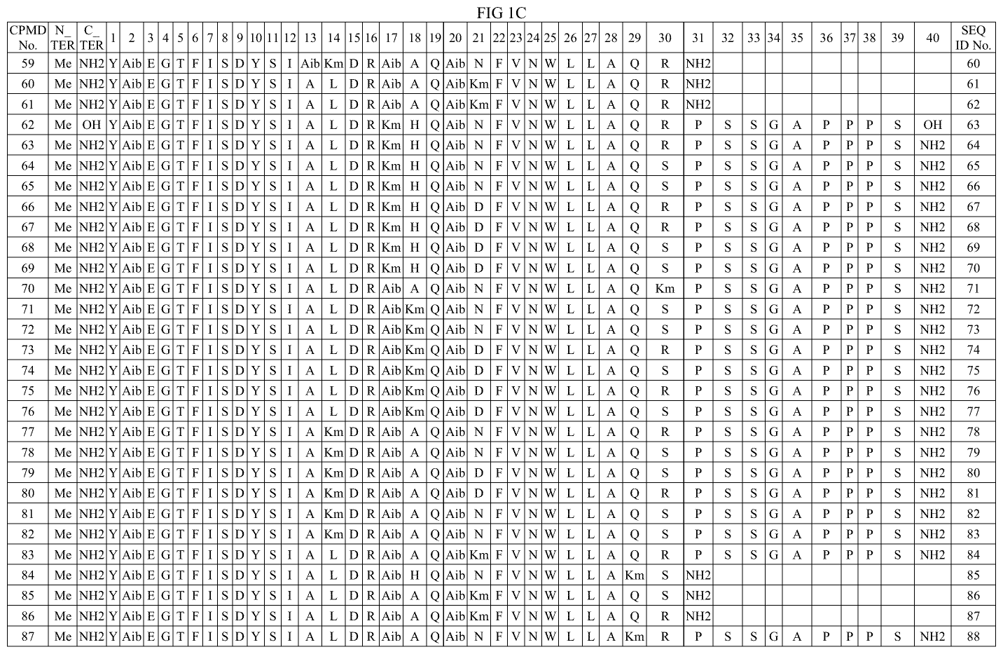

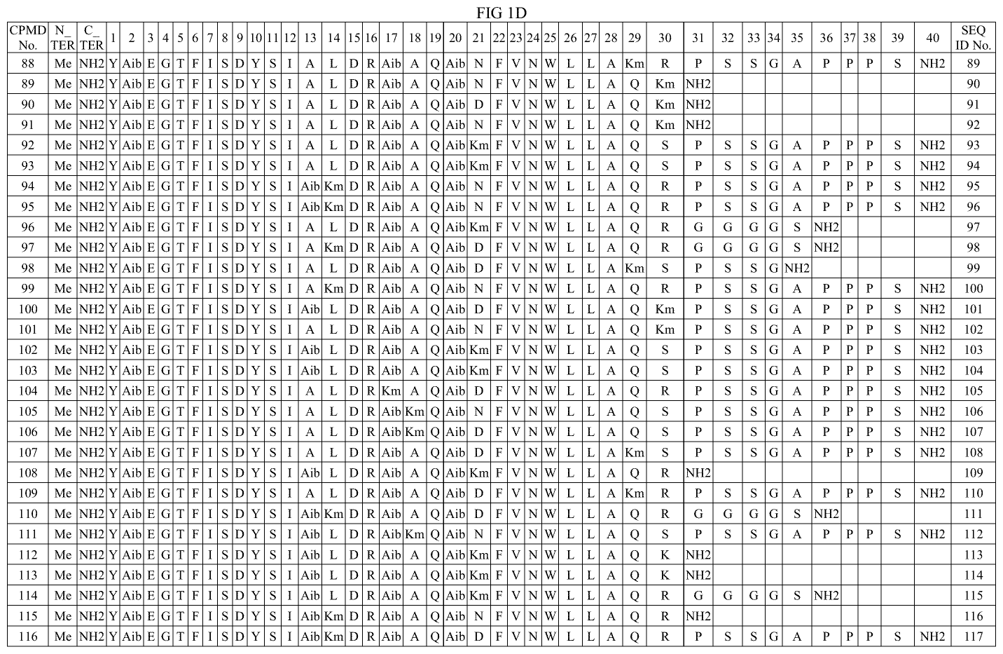

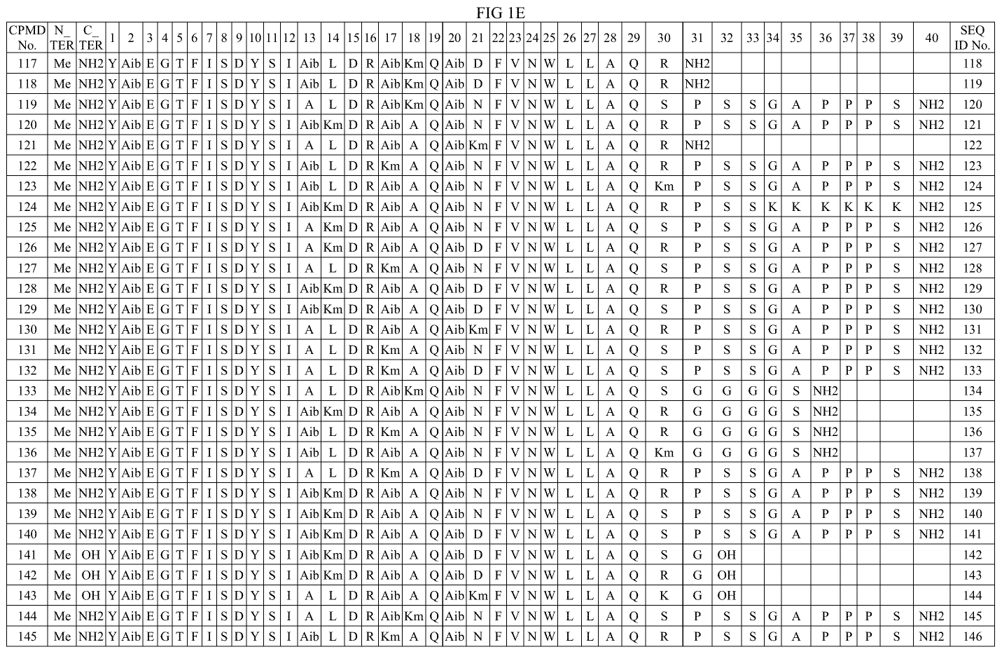

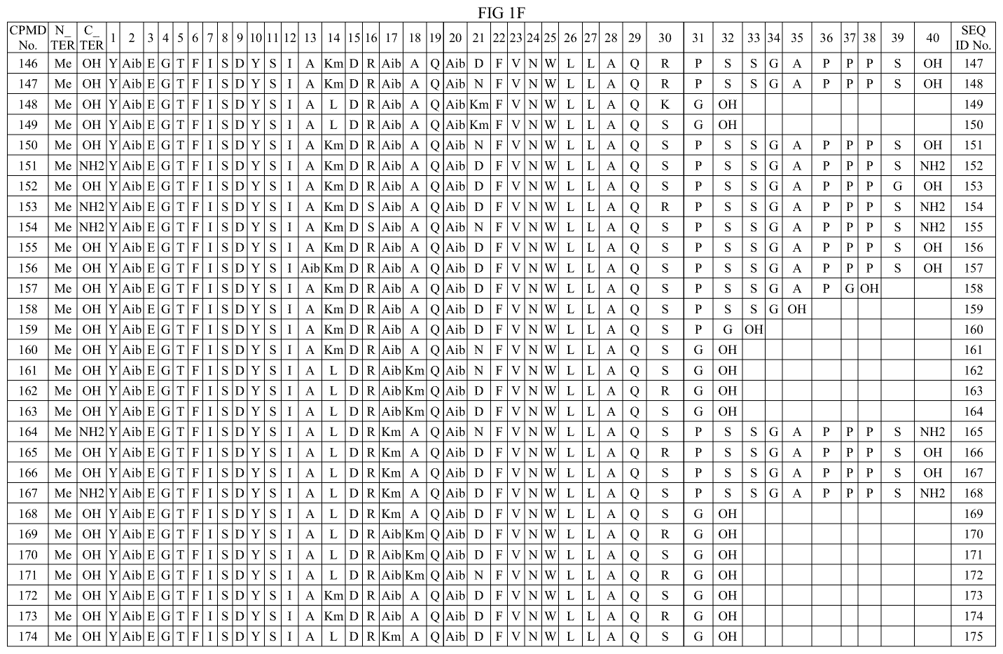

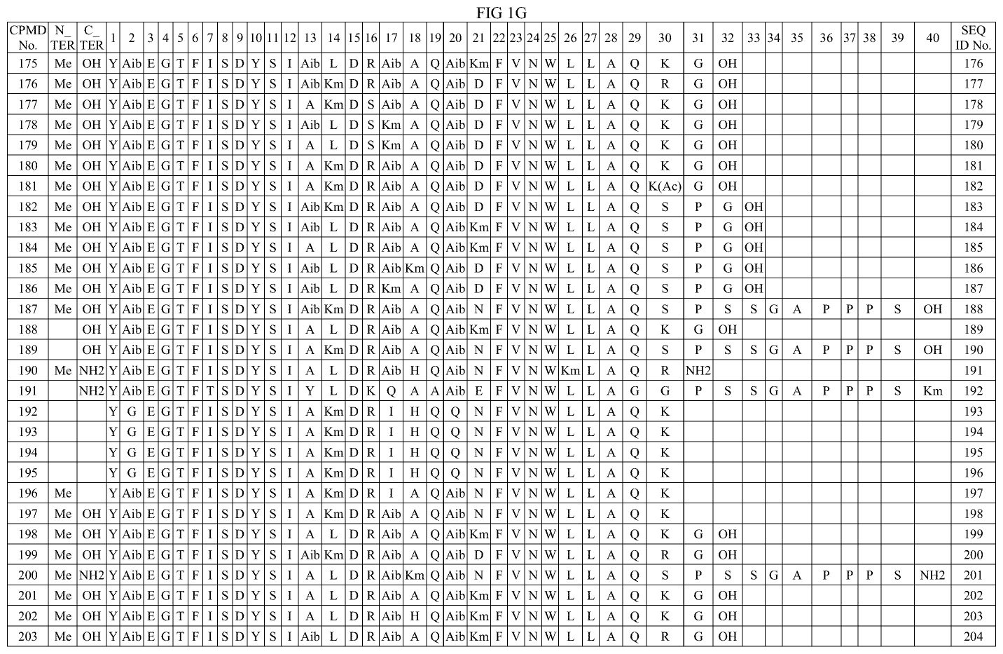

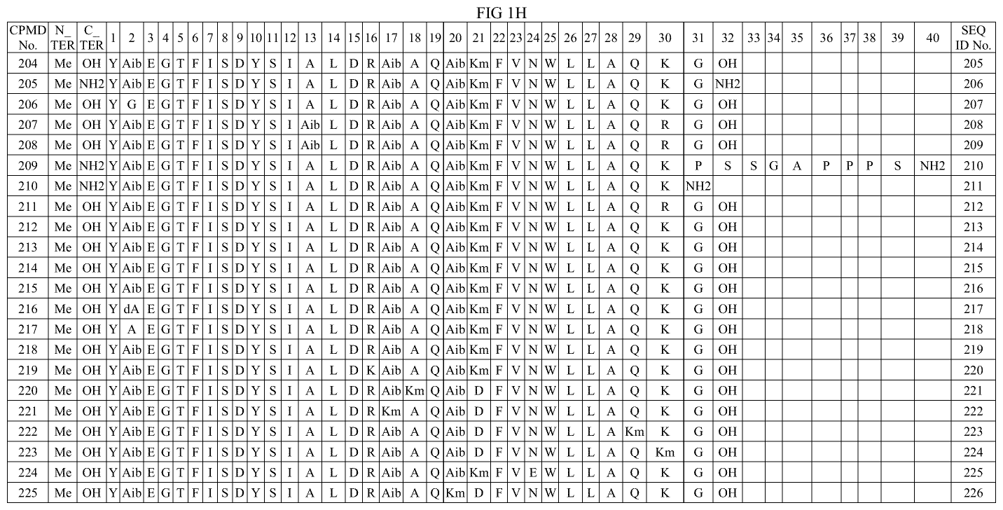

In various embodiments, exemplary GIP receptor agonist peptides having a structure as defined in any one of formulas (I)-(V), are provided in FIGS. 1A-1H.

### B. SYNTHESIS GIPR AGONIST PEPTIDES

The GIP receptor agonist peptide may be synthesized according to a peptide synthesis method known in the art. The peptide synthesis method may be any of, for example, a solid phase synthesis process and a liquid phase synthesis process. That is, the object GIP receptor agonist peptide can be produced by repeating condensation of a partial peptide or amino acid capable of constituting the GIP receptor agonist peptide, and the remaining portion (which may be constituted by two or more amino acids) according to a desired sequence. When a product having the desirable sequence has a protecting group, the object GIP receptor agonist peptide can be produced by eliminating a protecting group. Examples of the condensing method and eliminating method of a protecting group to be known include methods described in the following (1)-(5).
(1) M. Bodanszky and M. A. Ondetti: Peptide synthesis, Interscience Publishers, New York (1966)
(2) Schroeder and Luebke: The Peptide, Academic Press, New York (1965)
(3) Nobuo Izumiya, et al.: Peptide Gosei-no-Kiso to Jikken (Basics and experiments of peptide synthesis), published by Maruzen Co. (1975)
(4) Haruaki Yajima and Shunpei Sakakibara: Seikagaku Jikken Koza (Biochemical Experiment) 1, Tanpakushitsu no Kagaku (Chemistry of Proteins) IV, 205 (1977)
(5) Haruaki Yajima, ed.: Zoku Iyakuhin no Kaihatsu (A sequel to Development of Pharmaceuticals), Vol. 14, peptide synthesis, published by Hirokawa Shoten.

After the reaction, the GIP receptor agonist peptide can be purified and isolated using conventional methods of purification, such as solvent extraction, distillation, column chromatography, liquid chromatography, recrystallization, etc., in combination thereof When the peptide obtained by the above-mentioned method is in a free form, it can be converted to a suitable salt by a known method; conversely, when the peptide is obtained in the form of a salt, the salt can be converted to a free form or other salt by a known method.

The starting compound may also be a salt. Examples of such salt include those exemplified as salts of the exemplified selective GIPr agonists mentioned bellow.

For condensation of protected amino acid or peptide, various activation reagents usable for peptide synthesis can be used, which include trisphosphonium salts, tetramethyluronium salts, carbodiimides and the like. Examples of the trisphosphonium salt include benzotriazol-1-yloxytris(pyrrolizino)phosphoniumhexafluorophosphate (PyBOP), bromotris(pyrrolizino)phosphoniumhexafluorophosphate (PyBroP), 7-azabenzotriazol-1-yloxytris(pyrrolizino)phosphoniumhexafluorophosphate (PyAOP), examples of the tetramethyluronium salt include 2-(1H-benzotriazol-1-yl)-1,1,3,3-tetramethyluroniumhexafluorophosphate (HBTU), 2-(7-azabenzotriazol-1-yl)-1,1,3,3-tetramethyluroniumhexafluorophosphate (HATU), 2-(1H-benzotriazol-1-yl)-1,1,3,3-tetramethyluroniumtetrafluoroborate (TBTU), 2-(5-norbornane-2,3-dicarboxyimide)-1,1,3,3-tetramethyluroniumtetrafluoroborate (TNTU), O—(N-succimidyl)-1,1,3,3-tetramethyluroniumtetrafluoroborate (TSTU), and examples of the carbodiimide include N,N′-Dicyclohexylcarbodiimide (DCC), N,N′-diisopropylcarbodiimide (DIPCDI), N-ethyl-N′-(3-dimethylaminopropyl)carbodiimide hydrochloride (EDCI⋅HCl) and the like. For condensation using these, addition of a racemization inhibitor [e.g., N-hydroxy-5-norbornene-2,3-dicarboxylic imide (HONB), 1-hydroxybenzotriazole (HOBt), 1-Hydroxy-7-azabenzotriazole (HOAt), 3,4-Dihydro-3-hydroxy-4-oxo-1,2,3-benzotriazine (HOOBt), ethyl 2-cyano-2-(hydroxyimino)acetate (Oxyma) etc.] is example. A solvent to be used for the condensation can be appropriately selected from those known to be usable for peptide condensation reaction. For example, acid amides such as anhydrous or water-containing N,N-dimethylformamide, N,N-dimethylacetamide, N-methylpyrrolidone and the like, halogenated hydrocarbons such as methylene chloride, chloroform and the like, alcohols such as trifluoroethanol, phenol and the like, sulfoxides such as dimethylsulfoxide and the like, tertiary amines such as pyridine and the like, ethers such as dioxane, tetrahydrofuran and the like, nitriles such as acetonitrile, propionitrile and the like, esters such as methyl acetate, ethyl acetate and the like, an appropriate mixture of these and the like can be used. Reaction temperature is appropriately selected from the range known to be usable for peptide binding reactions, and is normally selected from the range of about −20° C. to 90° C. An activated amino acid derivative is normally used from 1.5 to 6 times in excess. In solid phase synthesis, when a test using the ninhydrin reaction reveals that the condensation is insufficient, sufficient condensation can be conducted by repeating the condensation reaction without elimination of protecting groups. If the condensation is yet insufficient even after repeating the reaction, unreacted amino acids can be acylated with acetic anhydride, acetylimidazole or the like so that an influence on the subsequent reactions can be avoided.

Examples of the protecting groups for the amino groups of the starting amino acid include benzyloxycarbonyl (Z), tert-butoxycarbonyl (Boc), tert-pentyloxycarbonyl, isobornyloxycarbonyl, 4-methoxybenzyloxycarbonyl, 2-chlorobenzyloxycarbonyl (Cl—Z), 2-bromobenzyloxycarbonyl (Br—Z), adamantyloxycarbonyl, trifluoroacetyl, phthaloyl, formyl, 2-nitrophenylsulphenyl, diphenylphosphinothioyl, 9-fluorenylmethyloxycarbonyl (Fmoc), trityl and the like.

Examples of the carboxyl-protecting group for the starting amino acid include aryl, 2-adamantyl, 4-nitrobenzyl, 4-methoxybenzyl, 4-chlorobenzyl, phenacyl and benzyloxycarbonylhydrazide, tert-butoxycarbonylhydrazide, tritylhydrazide and the like, in addition to the above-mentioned C₁₋₆ alkyl group, C₃₋₁₀ cycloalkyl group, C₇₋₁₄ aralkyl group.

The hydroxyl group of serine or threonine can be protected, for example, by esterification or etherification. Examples of the group suitable for the esterification include lower (C₂₋₄) alkanoyl groups such as an acetyl group and the like, aroyl groups such as a benzoyl group and the like, and the like, and a group derived from an organic acid and the like. In addition, examples of the group suitable for etherification include benzyl, tetrahydropyranyl, tert-butyl(Bu^{t}), trityl (Trt) and the like.

Examples of the protecting group for the phenolic hydroxyl group of tyrosine include Bzl, 2,6-dichlorobenzyl, 2-nitrobenzyl, Br—Z, tert-butyl and the like.

Examples of the protecting group for the imidazole of histidine include p-toluenesulfonyl (Tos), 4-methoxy-2,3,6-trimethylbenzenesulfonyl (Mtr), dinitrophenyl (DNP), benzyloxymethyl (Bom), tert-butoxymethyl (Bum), Boc, Trt, Fmoc and the like.

Examples of the protecting group for the guanidino group of arginine include Tos, Z, 4-methoxy-2,3,6-trimethylbenzenesulfonyl (Mtr), p-methoxybenzenesulfonyl (MBS), 2,2,5,7,8-pentamethylchromane-6-sulfonyl (Pmc), mesitylene-2-sulfonyl (Mts), 2,2,4,6,7-pentamethyldihydrobenzofuran-5-sulfonyl (Pbf), Boc, Z, NO₂ and the like.

Examples of the protecting group for a side chain amino group of lysine include Z, Cl—Z, trifluoroacetyl, Boc, Fmoc, Trt, Mtr, 4,4-dimethyl-2,6-dioxocyclohexylideneyl (Dde) and the like.

Examples of the protecting group for indolyl of tryptophan include formyl (For), Z, Boc, Mts, Mtr and the like.

Examples of the protecting group for asparagine and glutamine include Trt, xanthyl (Xan), 4,4′-dimethoxybenzhydryl (Mbh), 2,4,6-trimethoxybenzyl (Tmob) and the like.

Examples of activated carboxyl groups in the starting material include corresponding acid anhydride, azide, active esters [ester with alcohol (e.g., pentachlorophenol, 2,4,5-trichlorophenol, 2,4-dinitrophenol, cyanomethylalcohol, paranitrophenol, HONB, N-hydroxysuccimide, 1-hydroxybenzotriazole (HOBt), 1-hydroxy-7-azabenzotriazole(HOAt))] and the like. Examples of the activated amino group in the starting material include corresponding phosphorous amide.

Examples of the method for removing (eliminating) a protecting group include a catalytic reduction in a hydrogen stream in the presence of a catalyst such as Pd-black or Pd-carbon; an acid treatment using anhydrous hydrogen fluoride, methanesulfonic acid, trifluoromethanesulfonic acid, trifluoroacetic acid (TFA), trimethylsilyl bromide (TMSBr), trimethylsilyl trifluoromethanesulfonate, tetrafluoroboric acid, tris(trifluoro)boric acid, boron tribromide, or a mixture solution thereof; a base treatment using diisopropylethylamine, triethylamine, piperidine, piperazine or the like; and reduction with sodium in liquid ammonia, and the like. The elimination reaction by the above-described acid treatment is generally carried out at a temperature of −20° C. to 40° C.; the acid treatment is efficiently conducted by adding a cation scavenger such as anisole, phenol, thioanisole, metacresol and paracresol; dimethylsulfide, 1,4-butanedithiol, 1,2-ethanedithiol, triisopropylsilane and the like. Also, a 2,4-dinitrophenyl group used as a protecting group of the imidazole of histidine is removed by thiophenol treatment; a formyl group used as a protecting group of the indole of tryptophan is removed by deprotection by acid treatment in the presence of 1,2-ethanedithiol, 1,4-butanedithiol, or the like, as well as by alkali treatment with dilute sodium hydroxide, dilute ammonia, or the like.

Protection of a functional group that should not be involved in the reaction of a starting material and a protecting group, elimination of the protecting group, activation of a functional group involved in the reaction and the like can be appropriately selected from known protecting groups and known means.

In a method of preparing an amide of the peptide, it is formed by a solid phase synthesis using a resin for amide synthesis, or the α-carboxyl group of the carboxy terminal amino acid is amidated, and a peptide chain is elongated to a desired chain length toward the amino group side, thereafter a peptide wherein the protecting group for the N-terminal α-amino group of the peptide chain only removed and a peptide wherein the protecting group for the C-terminal carboxyl group only removed of the peptide chain are prepared, and the both peptides are condensed in a mixed solvent described above. For details about the condensation reaction, the same as above applies. After the protected peptide obtained by the condensation is purified, all protecting groups can be removed by the above-described method to yield a desired crude polypeptide. By purifying this crude peptide using various publicly known means of purification, and freeze-drying the main fraction, a desired amide of the peptide can be prepared.

When the GIP receptor agonist peptide is present as a configurational isomer such as enantiomer, diastereomer etc., a conformer or the like, they are also encompassed within the description of a GIP receptor agonist peptide and each can be isolated by a means known per se or the above separation and purification methods on demand. In addition, when the GIP receptor agonist peptide is in the form of a racemate, it can be separated into S- and R-forms by conventional optical resolution.

When a GIP receptor agonist peptide includes stereoisomers, both the isomers alone and mixtures of each isomers are also encompassed within the meaning of a GIP receptor agonist peptide. A GIP receptor agonist peptide can be chemically modified according to a method known per se and using substituent and polyethylene glycol. For example, a chemically modified GIP receptor agonist peptide can be produced by introducing substituent and/or conjugatedly binding polyethylene glycol to Cys residue, Asp residue, Glu residue, Lys residue and the like of a GIP receptor agonist peptide. Additionally, there may be a linker structure between the amino acid of the GIP receptor agonist peptide and substituent and polyethylene glycol.

A GIP receptor agonist peptide modified by a substituent and/or polyethylene glycol (PEG) produces for example, one or more effects related to promoting the biological activity, prolonging the blood circulation time, resistance to elimination, reducing the immunogenicity, enhancing the solubility, and enhancing the resistance to metabolism, of a therapeutically and diagnostically important peptide.

The molecular weight of PEG is not particularly limited and is normally about 1 K to about 1000 K daltons, or about 10 K to about 100 K daltons, or about 20 K to about 60 K Daltons.

Modifying a selective GIPr agonist of the present disclosure by adding an (R) substituent can be conducted by introducing the (R) substituent based on known oxidation reaction and reduction reaction.

A method well known in the art can be used as a method for modifying a GIP receptor agonist peptide by PEG, and, for example, in addition to the exemplary methods listed above, the methods described below can be used.

(1) A PEGylating reagent having an active ester (e.g., SUNBRIGHT MEGC-30TS (trade name), NOF Corp.) is bound to an amino group of the GIP receptor agonist peptide.
(2) A PEGylating reagent having an aldehyde (e.g., SUNBRIGHT ME-300AL (trade name), NOF Corp.) is bound to the amino group of the GIP receptor agonist peptide.
(3) A divalent cross-linking reagent (e.g., GMBS (Dojindo Laboratories), EMCS (Dojindo Laboratories), KMUS (Dojindo Laboratories), SMCC (Pierce)) is bound to an amino acid, (for example, a Lys and/or a Cys), of the GIP receptor agonist peptide, to which a PEGylating reagent having a thiol group (e.g., SUNBRIGHT ME-300-SH (trade name), NOF Corp.) is then bound.
(4) A thiol group is introduced to a GIP receptor agonist peptide through an SH-introducing agent (e.g., D-cysteine residue, L-cysteine residue, Traut's reagent), and this thiol group is reacted with a PEGylating reagent having a maleimide group (e.g., SUNBRIGHT ME-300MA (trade name), NOF Corp.).
(5) A thiol group is introduced to GIP receptor agonist peptide through an SH-introducing agent (e.g., D-cysteine residue, L-cysteine residue, Traut's reagent), and this thiol group is reacted with a PEGylating reagent having an iodoacetamide group (e.g., SUNBRIGHT ME-300MA (trade name), NOF Corp.).
(6) A ω-aminocarboxylic acid, an α-amino acid or the like is introduced as a linker to the N-terminal amino group of a GIP receptor agonist peptide, and an amino group derived from this linker is reacted with a PEGylating reagent having an active ester (e.g., SUNBRIGHT MEGC-30TS (trade name), NOF Corp.).
(7) A ω-aminocarboxylic acid, an α-amino acid or the like is introduced as a linker to the N-terminal amino group of a GIP receptor agonist peptide, and an amino group derived from this linker is reacted with a PEGylating reagent having an aldehyde group (e.g., SUNBRIGHT ME-300AL (trade name), NOF Corp.).

In addition, the GIP receptor agonist peptide may be a solvate (e.g., hydrate) or a non-solvate (e.g., non-hydrate).

The GIP receptor agonist peptide may be labeled with an isotope (e.g., ³H, ¹⁴C, ³⁵S, ¹²⁵I) or the like.

Furthermore, GIP receptor agonist peptide may be a deuterium conversion form wherein ¹H is converted to ²H(D).

In some embodiments, a GIP receptor agonist peptide labeled with or substituted with an isotope can be used as, for example, a tracer (PET tracer) for use in Positron Emission Tomography (PET), and is useful in the fields of medical diagnosis and the like.

For the GIP receptor agonist peptide mentioned herein, the left end is the N-terminal (amino terminal) and the right end is the C-terminal (carboxyl terminal) in accordance with the conventional peptide marking. The C-terminal of peptide may be any of an amide (—CONH₂), a carboxyl group (—COOH), a carboxylate (—COO—), an alkylamide (—CONHR^{a}), and an ester (—COOR^{a}). In some embodiments, the C-terminal is amide (—CONH₂).

A GIP receptor agonist peptide of the present disclosure may be in a salt form. Examples of such salt include metal salts, ammonium salts, salts with organic base, salts with inorganic acid, salts with organic acid, salts with basic or acidic amino acid, and the like.

Examples of the metal salt include alkali metal salts such as sodium salt, potassium salt and the like; alkaline earth metal salts such as calcium salt, magnesium salt, barium salt and the like; aluminum salt and the like.

Examples of the salt with organic base include salts with trimethylamine, triethylamine, pyridine, picoline, 2,6-lutidine, ethanolamine, diethanolamine, triethanolamine, cyclohexylamine, dicyclohexylamine, N,N-dibenzylethylenediamine and the like.

Examples of the salt with inorganic acid include salts with hydrochloric acid, hydrobromic acid, nitric acid, sulfuric acid, phosphoric acid and the like.

Examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Examples of the salt with basic amino acid include salts with arginine, lysine, omithine and the like. Examples of the salt with acidic amino acid include salts with aspartic acid, glutamic acid and the like.

Among the above-mentioned salts, a pharmaceutically acceptable salt is of interest. For example, when a compound has an acidic functional group, an inorganic salt such as alkali metal salt (e.g., sodium salt, potassium salt etc.), alkaline earth metal salt (e.g., calcium salt, magnesium salt, barium salt etc.) and the like, ammonium salt etc., and when a compound has a basic functional group, for example, a salt with inorganic acid such as hydrochloric acid, hydrobromic acid, nitric acid, sulfuric acid, phosphoric acid and the like, or a salt with organic acid such as acetic acid, phthalic acid, fumaric acid, oxalic acid, tartaric acid, maleic acid, citric acid, succinic acid, methanesulfonic acid, p-toluenesulfonic acid and the like are some examples.

In some embodiments, the GIP receptor agonist peptide may be synthesized and/or used in a prodrug form to treat or prevent a disease of the present disclosure, for example, diabetes, obesity and/or emesis. A prodrug means a compound which is converted to a GIP receptor agonist peptide with a reaction due to an enzyme, gastric acid, etc. under the physiological condition in the living body, that is, a compound which is converted to a GIP receptor agonist peptide with oxidation, reduction, hydrolysis, etc. according to an enzyme; a polypeptide which is converted to GIP receptor agonist peptide by hydrolysis etc. due to gastric acid, etc.

Examples of a prodrug of a GIP receptor agonist peptide may include a compound wherein an amino group of a GIP receptor agonist peptide is acylated, alkylated or phosphorylated (e.g., compound wherein amino group of a GIP receptor agonist peptide is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy group of a GIP receptor agonist peptide is acylated, alkylated, phosphorylated or borated (e.g., a compound wherein a hydroxy group of a GIP receptor agonist peptide is acetylated, palmitoylated, propanoylated, pivaloylated, succinylated, fumarylated, alanylated or dimethylaminomethylcarbonylated); a compound wherein a carboxy group of a GIP receptor agonist peptide is esterified or amidated (e.g., a compound wherein a carboxy group of a GIP receptor agonist peptide is C₁₋₆ alkyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. Among others, a compound wherein a carboxy group of a GIP receptor agonist peptide is esterified with C₁₋₆ alkyl such as methyl, ethyl, tert-butyl or the like may be used. These compounds, peptides and polypeptides can be produced from a GIP receptor agonist peptide by a method known per se.

A prodrug of a GIP receptor agonist peptide may also be one which is converted into a GIP receptor agonist peptide under a physiological condition, such as those described in IYAKUHIN no KAIHATSU (Development of Pharmaceuticals), Vol. 7, Design of Molecules, p. 163-198, Published by HIROKAWA SHOTEN (1990).

In the present specification, the prodrug may form a salt. Examples of such a salt include those exemplified as the salt of a GIP receptor agonist peptide.

In some embodiments, a GIP receptor agonist peptide of the present disclosure may be synthesized and/or used as a crystal. Crystals having a singular crystal form or a mixture of plural crystal forms are also encompassed by the examples of GIP receptor agonist peptides. Crystals can be produced by crystallizing a GIP receptor agonist peptide according to a crystallization method known per se.

In addition, a GIP receptor agonist peptide may be a pharmaceutically acceptable cocrystal or cocrystal salt. Here, the cocrystal or cocrystal salt means a crystalline substance consisting of two or more particular substances which are solids at room temperature, each having different physical properties (e.g., structure, melting point, heat of melting, hygroscopicity, solubility, stability etc.). The cocrystal and cocrystal salt can be produced by cocrystallization known per se.

The crystal of a GIP receptor agonist peptide of the present disclosure is superior in physicochemical properties (e.g., melting point, solubility, stability) and biological properties (e.g., pharmacokinetics (absorption, distribution, metabolism, excretion), efficacy expression), and thus it is extremely useful as a medicament.

In some embodiments, a GIP receptor agonist peptide and/or a prodrug thereof (hereinafter to be sometimes abbreviated as a GIP receptor agonist peptide of the present disclosure) have a GIP receptor activating action, and may have selectivity as agonists of the GIP receptor over other receptors such as the GLP1R. The compounds of the present disclosure have a high GIP receptor selective activation action in vivo.

### C. METHODS OF PROPHYLAXIS AND TREATMENT OF GIP MEDIATED CONDITIONS, DISEASES, AND DISORDERS

GIP is a gastrointestinal hormone called incretin and has a promoting action on insulin secretion from the pancreas. Incretin is closely related to glucose metabolism and thus the compound having a GIP receptor activation action is useful for preventing and treating symptoms related to abnormal glucose metabolism including diabetes and obesity. Additionally, the compounds of the present disclosure have a GIP receptor selective activation action and suppresses vomiting by activating GABAergic neurons in the area postrema.

More specifically, the GIP receptor agonist peptides of the present disclosure have a hypoglycemic action, an antiemetic action, and the like.

The GIP receptor agonist peptides of the present disclosure have a high chemical stability and excellent persistence of the effects in vivo.

The GIP receptor agonist peptides of the present disclosure may be used as a GIP receptor activator.

In the present disclosure, the GIP receptor activator (GIP receptor agonist) means an agent having a GIP receptor activation action. Additionally, the GIP receptor selective activator (GIP receptor peptide agonist) specifically means an agent having an EC₅₀ for the GIP receptor of 1/10 or less, or 1/100 or less, or 1/1000 or less, or 1/10000 or less, times the EC₅₀ for the GLP-1 receptor.

The GIP receptor agonist peptides of the present disclosure is low in its toxicity (e.g., acute toxicity, chronic toxicity, genetic toxicity, reproductive toxicity, cardiac toxicity, carcinogenicity), shows a few side effects, and can be safely administered to a mammal (e.g., human, bovine, horse, dog, cat, monkey, mouse, rat) as an agent for the prophylaxis or treatment of various diseases mentioned below and the like.

The GIP receptor agonist peptides of the present disclosure can be used as an agent for the treatment or prophylaxis of various diseases including diabetes and obesity, by virtue of the above-mentioned activating action on GIP receptors. The GIP receptor agonist peptides of the present disclosure can be used as an agent for the prophylaxis or treatment of, for example, symptomatic obesity, obesity based on simple obesity, disease state or disease associated with obesity, eating disorder, diabetes (e.g., type 1 diabetes, type 2 diabetes, gestational diabetes, obese diabetes), hyperlipidemia (e.g., hypertriglyceridemia, hypercholesterolemia, high LDL-cholesterolemia, low HDL-cholesterolemia, postprandial hyperlipemia), hypertension, cardiac failure, diabetic complications [e.g., neuropathy, nephropathy, retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], metabolic syndrome (disease states having 3 or more selected from hypertriglyceridemia, (TG), low HDL cholesterol(HDL-C)emia, hypertension, abdominal obesity and impaired glucose tolerance), sarcopenia and the like.

Examples of the symptomatic obesity include endocrine obesity (e.g., Cushing syndrome, hypothyroidism, insulinoma, obese type II diabetes, pseudohypoparathyroidism, hypogonadism), central obesity (e.g., hypothalamic obesity, frontal lobe syndrome, Kleine-Levin syndrome), hereditary obesity (e.g., Prader-Willi syndrome, Laurence-Moon-Biedl syndrome), drug-induced obesity (e.g., steroid, phenothiazine, insulin, sulfonylurea (SU) agent, β-blocker-induced obesity) and the like.

Examples of the disease state or disease associated with obesity include glucose tolerance disorders, diabetes (e.g., type 2 diabetes (T2DM), obese diabetes), lipid metabolism abnormality (synonymous with the above-mentioned hyperlipidemia), hypertension, cardiac failure, hyperuricemia.gout, fatty liver (including non-alcoholic steato-hepatitis), coronary heart disease (myocardial infarction, angina pectoris), cerebral infarction (brain thrombosis, transient cerebral ischemic attack), bone/articular disease (knee osteoarthritis, hip osteoarthritis, spondylitis deformans, lumbago), sleep apnea syndrome/Pickwick syndrome, menstrual disorder (abnormal menstrual cycle, abnormality of menstrual flow and cycle, amenorrhea, abnormal catamenial symptom), metabolic syndrome and the like.

New diagnostic criteria were reported by The Japan Diabetes Society in 1999 about the diagnostic criteria of diabetes.

According to this report, diabetes refers to a state that meets any of a fasting blood glucose level (glucose concentration in venous plasma) of 126 mg/dl or more, a 2-hr value (glucose concentration in venous plasma) of 200 mg/dl or more in the 75 g oral glucose tolerance test (75 g OGTT), and a casual blood glucose level (glucose concentration in venous plasma) of 200 mg/dl or more. Also, a state that does not apply to the above-mentioned diabetes, and is not a state exhibiting “a fasting blood glucose level (glucose concentration in venous plasma) less than 110 mg/dl or a 2-hr value (glucose concentration in venous plasma) less than 140 mg/dl in the 75 g oral glucose tolerance test (75 g OGTT)” (normal type) is called “borderline type”.

Moreover, new diagnostic criteria were reported by American Diabetes Association (ADA) in 1997 and by World Health Organization (WHO) in 1998 about the diagnostic criteria of diabetes.

According to these reports, diabetes refers to a state that meets a fasting blood glucose level (glucose concentration in venous plasma) of 126 mg/dl or more and a 2-hr value (glucose concentration in venous plasma) of 200 mg/dl or more in the 75 g oral glucose tolerance test.

According to the above-mentioned reports, impaired glucose tolerance refers to a state that meets a fasting blood glucose level (glucose concentration in venous plasma) less than 126 mg/dl and a 2-hr value (glucose concentration in venous plasma) of 140 mg/dl or more and less than 200 mg/dl in the 75 g oral glucose tolerance test. According to the report of ADA, a state exhibiting a fasting blood glucose level (glucose concentration in venous plasma) of 110 mg/dl or more and less than 126 mg/dl is called IFG (Impaired Fasting Glucose). On the other hand, according to the report of WHO, a state of the IFG (Impaired Fasting Glucose) exhibiting a 2-hr value (glucose concentration in venous plasma) less than 140 mg/dl in the 75 g oral glucose tolerance test is called IFG (Impaired Fasting Glycemia).

The GIP receptor agonist peptides of the present disclosure may also be used as an agent for the prophylaxis or treatment of diabetes determined according to the above-mentioned new diagnostic criteria, borderline type diabetes, impaired glucose tolerance, IFG (Impaired Fasting Glucose) and IFG (Impaired Fasting Glycemia). Moreover, the GIP receptor agonist peptides of the present disclosure can prevent progress of borderline type, impaired glucose tolerance, IFG (Impaired Fasting Glucose) or IFG (Impaired Fasting Glycemia) into diabetes.

The GIP receptor agonist peptides of the present disclosure are also useful as an agent for the prophylaxis or treatment of metabolic syndrome. The incidence of cardiovascular disease is significantly high in metabolic syndrome patients, compared with patients with a single lifestyle-related disease. Thus, the prophylaxis or treatment of metabolic syndrome is exceedingly important for preventing cardiovascular disease.

The diagnostic criteria of metabolic syndrome were announced by WHO in 1999 and by NCEP in 2001. According to the diagnostic criteria of WHO, an individual having hyperinsulinemia or abnormal glucose tolerance as a requirement and two or more of visceral obesity, dyslipidemia (high TG or low HDL) and hypertension is diagnosed as having metabolic syndrome (World Health Organization: Definition, Diagnosis and Classification of Diabetes Mellitus and Its Complications. Part I: Diagnosis and Classification of Diabetes Mellitus, World Health Organization, Geneva, 1999). According to the diagnostic criteria of the Adult Treatment Panel III of the National Cholesterol Education Program (guideline of ischemic heart disease) in USA, an individual having three or more of visceral obesity, hypertriglyceridemia, low HDL-cholesterolemia, hypertension and abnormal glucose tolerance is diagnosed as having metabolic syndrome (National Cholesterol Education Program: Executive Summary of the Third Report of National Cholesterol Education Program (NCEP) Expert Panel on Detection, Evaluation, and Treatment of High Blood Cholesterol in Adults (Adults Treatment Panel III). The Journal of the American Medical Association, Vol. 285, 2486-2497, 2001).

More specifically, the GIP receptor agonist peptides of the present disclosure have an antiemetic action, and may inhibit or reduce the number and severity of the occurrence of nausea, and/or vomiting when associated with various stimuli disclosed herein, for example, when a subject has cyclic vomiting syndrome or is administered a chemotherapeutic drug, for example, a chemotherapeutic drug with emetic potential, such as platinum based chemotherapeutics such as cisplatin, oxaliplatin, and carboplatin; irinotecan and other topo isomerase inhibitors used in the treatment of cancer. The GIP receptor agonist peptides of the present disclosure have a high chemical stability and excellent persistence of the effects in vivo.

The GIP receptor agonist peptides of the present disclosure may be used as a GIP receptor activator. In the present disclosure, the GIP receptor activator (GIP receptor agonist) means an agent having a GIP receptor activation action. Additionally, the GIP receptor selective activator (i.e. a GIP receptor agonist as used herein) specifically means an agent having an EC₅₀ for the GIP receptor of 1/1000 or less, or 1/10000 or less, times the EC₅₀ for the GLP-1 receptor, or in other words the ratio of EC₅₀ GLP1R/EC₅₀ GIPR is greater than 10, greater than 100, or greater than 1,000, or greater than 10,000, or from 100 to 1,000,000 or more.

The GIP receptor agonist peptides of the present disclosure have low toxicity (e.g., acute toxicity, chronic toxicity, genetic toxicity, reproductive toxicity, cardiac toxicity, carcinogenicity), shows a few side effects, and can be safely administered to a mammal (e.g., human, bovine, horse, dog, cat, monkey, mouse, rat) as an agent for the prophylaxis or treatment of emesis.

“Treatment,” in the context of treating emesis by administering at least one of the GIP receptor agonist peptides disclosed herein, includes both prophylactic treatment and the treatment of emesis after a subject experiences emesis. Prophylactic treatment includes administration of a GIP receptor agonist peptide before a subject experiences emesis, such as when the subject experiences nausea, as well as administration of the GIP receptor agonist peptide before the subject is exposed to a substance, agent, or event, or before the subject contracts a condition, which results in or is likely to result in the subject experiencing emesis. As used herein, “therapeutically effective amount” refers to an amount of the GIP receptor agonist peptide sufficient to elicit the desired biological response. In the present disclosure, the desired biological response is treating and/or preventing an abnormal glucose metabolism in a subject, for example, in a subject in need thereof, including diabetes and obesity, or the prevention and/or treatment of emesis in a subject in need thereof.

The compound of the present invention can also be used for secondary prevention or suppression of progression of the above-mentioned various diseases (e.g., cardiovascular events such as myocardial infarction and the like). In addition, the compound of the present invention is also useful as a feeding suppressant and a weight reducing agent. The compound of the present invention can also be used in combination with a diet therapy (e.g., diet therapy for diabetes), and an exercise therapy. The GIP receptor agonist peptides of the present disclosure can be used to treat or prevent diabetes and/or obesity, a pathophysiological condition related to diabetes and/or obesity, emesis, for example, when a subject experiences or is about to experience emesis, such as nausea and/or vomiting. In various embodiments, the subject, for example, a mammal, for example, humans, non-human primates, apes, monkeys, laboratory mammals for example, mice, rats, rabbits, guinea-pigs, ferrets, domesticated mammals, such as companion mammals, dogs, cats and horses, and farm mammals, such as cattle, pigs, sheep and goats purely as examples, but not intended to be an exhaustive list, may be treated with a GIP receptor agonist peptide of the present disclosure. In each of these cases, the methods of the present disclosure are provided to treat or prevent diabetes, obesity, or emesis in a subject in need thereof, to reduce or inhibit diabetes, obesity, or emesis, to reduce or inhibit a symptom associated with diabetes, obesity, or emesis, or to reduce or inhibit a pathological condition or symptom associated with diabetes, obesity, or emesis, for example, nausea and/or vomiting.

In order to prevent or treat emesis, an effective amount of one or more of the present compounds in a pharmaceutical composition is administered once per day to a subject/patient (used interchangeably herein) in need thereof A subject is determined to be in need of treatment with the present GIP receptor agonist peptide either through observation of vomiting by the subject, or through a subject's self-reporting of emesis (in the case of a human subject). A patient is determined to be in need of preventative therapy by assessing that the patient is at risk of experiencing emesis due to another medical condition or due to exposure to an agent known to be associated with emesis, such as an infection by a virus or bacteria or chemical agent or radiation.

The present GIP receptor agonist peptides are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, pain, opioid use and withdrawal, migraine, and variations in intracranial pressure. The uses of this invention are of benefit in the therapy of emesis induced by radiation, for example during the treatment of cancer, or radiation sickness, and in the treatment of post-operative nausea and vomiting. Most especially, use of the invention is beneficial in the therapy of emesis induced by antineoplastic (cytotoxic) agents including those routinely used in cancer chemotherapy, emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram.

Examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, especially page 188. Commonly used chemotherapeutic agents include cisplatin, carboplatin, oxaliplatin, cyclophosphamide, dacarbazine (DTIC), dactinomycin, mechlorethamine (nitrogen mustard), streptozocin, cyclophosphamide, carmustine (BCNU), irinotecan, and other topoisomerase inhibitors, lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil (R. J. Gralle et al. in Cancer Treatment Reports, 1984, 68, 163-172). Emesis due to other chemical agents, such as the toxins soman or sarin, or opiod drug usage and/or withdrawal, e.g. morphine, heroin, oxycodone, and the like can also be prevented and/or treated.

The present compounds are administered to a patient in a quantity sufficient to treat or prevent the symptoms and/or underlying etiology associated with emesis in the patient. In a preferred embodiment, the GIP receptor agonist peptides are administered prior to administration of an agent which is likely to cause emesis, such as one or more of the chemotherapeutic agents described above. The present GIP receptor agonist peptides can also be administered in combination with such agents, either in physical combination or in combined therapy through the administration of the present compounds and agents in succession (in any order). Although the present invention is useful in any mammal suffering from emesis, a preferred subject is a human.

In some embodiments, the selective GIPr agonists of the present disclosure may be administered to treat emesis when a subject is concomitantly being treated for diabetes and/or obesity. Several known anti-diabetic medicaments are known for causing emesis, for example, Metformin (Glucophage, Glumetza, others), sulfonylureas, meglitinides, thiazolidinediones, DPP-4 inhibitors, SGLT2 inhibitors, and GLP-1 receptor agonists. In some embodiments, methods for treating emesis in a subject, for example in a subject in need thereof, may include administering an effective amount of a GIP receptor agonist peptide to a subject that does not have type-2 diabetes mellitus or a subject that is not taking a medicament to treat type-2 diabetes mellitus while experiencing emesis.

Nausea is a subjective unpleasant feeling in the back of one's throat and stomach that may lead to vomiting. There are many words that describe nausea including, but not limited to: sick to my stomach, queasy, or upset stomach. Nausea can have other symptoms that happen at the same time, such as increased saliva (spit), dizziness, light-headedness, trouble swallowing, skin temperature changes, and a fast heart rate. Vomiting is also described as “throwing up.” When one vomits, one's stomach muscles contract (squeeze) and push the contents of one's stomach out through their mouth. One might or might not feel nauseated. Retching is when one tries to vomit without bringing anything up from one's stomach. Other words used to describe retching are gagging or dry heaves. Nausea and vomiting often happen at the same time, but they can be 2 different conditions that may be mutually exclusive or mutually associated. Some chemotherapy drugs are more likely to cause nausea and vomiting than others. Doctors classify chemotherapy drugs according to their emetogenic potential (how likely the drug will cause nausea or vomiting) as high, moderate, low, or minimal risk.

In various embodiments, the GIPR agonist peptide compounds may be dosed once per day to provide treatment and prophylactic treatment against emesis and emesis related symptoms. The peptide compounds of the present disclosure may be used to preferentially treat cyclic vomiting syndrome (CVS); chemotherapy induced nausea and vomiting (CINV) and post-operative nausea and vomiting (PONV). Cyclic vomiting syndrome (CVS) is a chronic functional gastrointestinal disorder that is being increasingly recognized in adults. It is characterized by episodic nausea and vomiting and is associated with significant morbidity.

An estimated 80% of patients with cancer will experience chemotherapy-induced nausea and vomiting (CINV). The term CINV includes emesis and nausea, which can involve a loss of appetite and result in decreased oral intake of fluids and calories. Five different types of CINV have been defined and include acute, delayed, breakthrough, anticipatory, and refractory CINV.

Postoperative nausea and vomiting (PONV) is the phenomenon of nausea, vomiting or retching experienced by a patient in the Post Anesthesia Care Unit (PACU) or 24-hours following a surgical procedure. It is an unpleasant complication that affects about 10% of the population undergoing general anaesthesia each year.

In an exemplary embodiment, the present disclosure provides for the prophylactic treatment or maintenance therapy for cyclic vomiting syndrome (CVS); chemotherapy induced nausea and vomiting (CINV) and post-operative nausea and vomiting (PONV), comprising administering one or more GIPR agonist peptide compounds of the present disclosure, for example, a GIPR agonist peptide compound selected from compound 17, 25, 21, 48, 142, 14 and 20, in a therapeutically effective amount to a subject in need thereof.

The GIP receptor agonist peptides of the present disclosure may be used as a preventive/therapeutic agent, ie. prophylactic treatment or maintenance therapy for vomiting and/or nausea caused, for example, by clinical pathological conditions or causes described in the following

The GIP receptor agonist peptides of the present disclosure may be used as a preventive/therapeutic agent for vomiting and/or nausea caused, for example, by clinical pathological conditions or causes described in the following (1) to (10). Additionally, the GIP receptor agonist peptide of the present disclosure may be used as a preventive/therapeutic agent for chronic unexplained nausea and vomiting. The vomiting or nausea also includes imminent unpleasant sensations of wanting to eject the contents of the stomach through the mouth such as feeling queasy and retching, and may also be accompanied by autonomic symptoms such as facial pallor, cold sweat, salivary secretion, tachycardia, and diarrhea. The vomiting also includes acute vomiting, protracted vomiting, and anticipatory vomiting.

(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea caused by a vestibular disorder such as motion sickness or dizziness
(7) Opioid withdrawal;
(8) Vomiting and nausea caused by post-operative nausea and vomiting;
(9) A vestibular disorder such as motion sickness or dizziness; and
(10) A physical injury causing local, systemic, acute or chronic pain.

These causes of emesis, or nausea, or vomiting are not meant to be exhaustive. Other conditions, activities, side effects may cause emesis, for example, nausea and/or vomiting. Nausea can be measured in ways known to the art, such as through the use of a visual analog scale (VAS).

The compound of the present invention can also be used for secondary prevention or suppression of progression of the above-mentioned various diseases (e.g., cardiovascular events such as myocardial infarction and the like). In addition, the compound of the present invention is also useful as a feeding suppressant and a weight reducing agent. The compound of the present invention can also be used in combination with a diet therapy (e.g., diet therapy for diabetes), and an exercise therapy.

### D. FORMULATIONS

A medicament containing a GIP receptor agonist peptide of the present disclosure shows low toxicity and is obtained using the compound of the present disclosure alone or in admixture with a pharmacologically acceptable carrier according to a method known per se (e.g., the method described in the Japanese Pharmacopoeia) generally used as production methods of pharmaceutical preparations, and safely administered orally or parenterally (e.g., topically, rectally, intravenously administered) as a pharmaceutical preparation, for example, tablets (inclusive of sugar-coated tablets, film-coated tablets, sublingual tablets, orally disintegrating tablets), powders, granules, capsules (inclusive of soft capsules, microcapsules), liquids, troches, syrups, emulsions, suspensions, injections (e.g., subcutaneous injections, intravenous injections, intramuscular injections, intraperitoneal injections etc.), external preparations (e.g., transnasal preparations, dermal preparations, ointments), suppository (e.g., rectal suppositories, vaginal suppositories), pellets, nasal preparations, pulmonary preparations (inhalants), transfusions and the like.

These preparations may be controlled release preparations such as a rapid release preparation, a sustained release preparation and the like (e.g., a sustained release microcapsule). The content of the compound of the present disclosure in a pharmaceutical preparation is about 0.01-about 100 wt % of the whole preparation.

The above-mentioned pharmaceutically acceptable carrier may be exemplified by various organic or inorganic carrier materials that are conventionally used as preparation materials, for example, excipient, lubricant, binding agent and disintegrant for solid preparations; or solvent, solubilizing agent, suspending agent, isotonic agent, buffering agent, soothing agent and the like for liquid preparations. Further, if necessary, general additives such as preservative, antioxidant, colorant, sweetening agent, adsorbing agent, wetting agent and the like can be also used appropriately in a suitable amount.

Examples of the excipient include lactose, sucrose, D-mannitol, starch, corn starch, crystalline cellulose, light anhydrous silicic acid and the like.

Examples of the lubricant include magnesium stearate, calcium stearate, talc, colloidal silica and the like.

Examples of the binding agent include crystalline cellulose, sucrose, D-mannitol, dextrin, hydroxypropylcellulose, hydroxypropylmethylcellulose, polyvinylpyrrolidone, starch, sucrose, gelatin, methylcellulose, carboxymethylcellulose sodium and the like.

Examples of the disintegrant include starch, carboxymethylcellulose, carboxymethylcellulose calcium, carboxymethylstarch sodium, L-hydroxypropylcellulose and the like.

Examples of the solvent include water for injection, alcohol, propylene glycol, Macrogol, sesame oil, corn oil, olive oil and the like.

Examples of the solubilizing agent include polyethylene glycol, propylene glycol, D-mannitol, benzyl benzoate, ethanol, trisaminomethane, cholesterol, triethanolamine, sodium carbonate, sodium citrate and the like.

Examples of the suspending agent include surfactants such as stearyl triethanolamine, sodium lauryl sulfate, laurylaminopropionic acid, lecithin, benzalkonium chloride, benzetonium chloride, glycerin monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; and the like.

Examples of the isotonic agent include glucose, D-sorbitol, sodium chloride, glycerin, D-mannitol and the like.

Examples of the buffering agent include buffer solutions such as phosphates, acetates, carbonates, citrates and the like.

Examples of the soothing agent include benzyl alcohol and the like.

Examples of the preservative include parahydroxybenzoic acid esters, chlorobutanol, benzyl alcohol, phenethyl alcohol, dehydroacetic acid, sorbic acid and the like.

Examples of the antioxidant include sulfites, ascorbic acid, α-tocopherol and the like.

Examples of the colorant include water-soluble food coal tar dyes (e.g., food dyes such as Food Red No. 2 and No. 3, Food Yellow No. 4 and No. 5, Food Blue No. 1 and No. 2, and the like), water-insoluble lake dyes (e.g., aluminum salts of the aforementioned water-soluble Food coal tar dyes), natural dyes (e.g., β-carotene, chlorophyll, ferric oxide red) and the like.

Examples of the sweetening agent include saccharin sodium, dipotassium glycyrrhizinate, aspartame, stevia and the like.

Examples of the adsorbing include porous starch, calcium silicate (trade name: Florite RE), magnesium alumino metasilicate (trade name: Neusilin) and light anhydrous silicic acid (trade name: Sylysia).

Examples of the wetting agent include propylene glycol monostearate, sorbitan monooleate, diethylene glycol monolaurate and polyoxyethylenelauryl ether.

During production of an oral preparation, coating may be applied as necessary for the purpose of masking of taste, enteric property or durability.

Examples of the coating base to be used for coating include sugar coating base, aqueous film coating base, enteric film coating base and sustained-release film coating base.

As the sugar coating base, sucrose is used. Moreover, one or more kinds selected from talc, precipitated calcium carbonate, gelatin, gum arabic, pullulan, carnauba wax and the like may be used in combination.

Examples of the aqueous film coating base include cellulose polymers such as hydroxypropyl cellulose, hydroxypropylmethyl cellulose, hydroxyethyl cellulose, methylhydroxyethyl cellulose etc.; synthetic polymers such as polyvinylacetal diethylaminoacetate, aminoalkyl methacrylate copolymer E [Eudragit E (trade name)], polyvinylpyrrolidone etc.; and polysaccharides such as pullulan etc.

Examples of the enteric film coating base include cellulose polymers such as hydroxypropylmethyl cellulose phthalate, hydroxypropylmethyl cellulose acetate succinate, carboxymethylethyl cellulose, cellulose acetate phthalate etc.; acrylic polymers such as methacrylic acid copolymer L [Eudragit L (trade name)], methacrylic acid copolymer LD [Eudragit L-30D55 (trade name)], methacrylic acid copolymer S [Eudragit S (trade name)] etc.; and naturally occurring substances such as shellac etc.

Examples of the sustained-release film coating base include cellulose polymers such as ethyl cellulose etc.; and acrylic polymers such as aminoalkyl methacrylate copolymer RS [Eudragit RS (trade name)], ethyl acrylate-methyl methacrylate copolymer suspension [Eudragit NE (trade name)] etc.

The above-mentioned coating bases may be used after mixing with two or more kinds thereof at appropriate ratios. For coating, for example, a light shielding agent such as titanium oxide, red ferric oxide and the like can be used.

### E. ADMINISTRATION

The therapeutically effective amount or dose of a composition or medicament containing a GIP receptor agonist peptide to be administered to a subject will depend on the age, sex and weight of the patient, and the current medical condition of the patient. The skilled artisan will be able to determine appropriate dosages depending on these and other factors to achieve the desired biological response.

The dosage of the GIP receptor agonist peptide of the present disclosure is appropriately determined according to the subject of administration, symptom, administration method and the like. For example, when the GIP receptor agonist peptide of the present disclosure is administered orally to a subject prior to engaging in an act that will likely cause emesis or after the onset of emesis in a human subject (body weight of approximately 60 kg), the daily dose of the compound of the present disclosure is about 0.01 to 100 mg, or about 1.0 to 50 mg, or about 1.0 to 20 mg. When the compound of the present disclosure is administered parenterally to an obesity or diabetes patient or a gastroparesis (body weight 60 kg), the daily dose of the compound of the present disclosure is about 0.001 to 30 mg, or about 0.01 to 20 mg, or about 0.1 to 10 mg. These amounts can be administered in about 1 to several portions a day. In some embodiments, a therapeutically effective amount of a GIP receptor agonist peptide to prevent and/or treat emesis in a subject in need thereof may range from about 0.01 to 0.5 mg/kg/day, 0.1 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

The GIP receptor agonist peptide of the present disclosure can be administered, for example, once per day, every 2 days, every 3 days, every 4 days, every 5 days, every 6 days, every week, twice per week, every other week, every 3 weeks, every month, every 2 months, every 3 months, every 4 months, every 5 months or every 6 months. In some embodiments, the GIP receptor agonist peptide of the present disclosure can be administered to the subject 1 times per day, QD, or 1-7 times per week, for 1-5 days, 1-5 weeks, 1-5 months, or 1-5 years.

The GIP receptor agonist peptide of the present disclosure can be used in combination with another drug that does not adversely influence the GIP receptor agonist peptide of the present disclosure, for the purpose of, for example, promoting the action (antiemetic action) of the GIP receptor agonist peptide of the present disclosure, reducing the dose of the GIP receptor agonist peptide of the present disclosure, and the like.

Examples of a drug that can be used in combination with the GIP receptor agonist peptide of the present disclosure (hereinafter sometimes to be abbreviated as a concomitant drug) include anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, therapeutic agents for osteoporosis, vitamins, antidementia drugs, erectile dysfunction drugs, therapeutic drugs for urinary frequency or urinary incontinence, therapeutic agents for dysuria, central D2 receptor antagonists, prokinetic agents, antihistamines, muscarine receptor antagonists, serotonin 5HT3 receptor antagonists, somatostatin analogues, corticosteroids, benzodiazepine anxiolytics, NK-1 receptor antagonists, hypercalcemia therapeutic drug and the like. Specific examples of the concomitant drug include those mentioned below.

Examples of the anti-obesity agent include monoamine uptake inhibitors (e.g., phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), neuropeptide Y antagonists (e.g., velneperit), cannabinoid receptor antagonists (e.g., rimonabant, taranabant), ghrelin antagonists, ghrelin receptor antagonists, ghrelinacylation enzyme inhibitors, opioid receptor antagonists (e.g., GSK-1521498), orexin receptor antagonists, melanocortin 4 receptor agonists, 110-hydroxysteroid dehydrogenase inhibitors (e.g., AZD-4017), pancreatic lipase inhibitors (e.g., orlistat, cetilistat), 03 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), Na-glucose cotransporter inhibitors (e.g., JNJ-28431754, remogliflozin), NFx inhibitory (e.g., HE-3286), PPAR agonists (e.g., GFT-505, DRF-11605), phosphotyrosine phosphatase inhibitors (e.g., sodium vanadate, Trodusquemin), GPR119 agonists (e.g., PSN-821, MBX-2982, APD597), glucokinase activators (e.g., AZD-1656), leptin, leptin derivatives (e.g., metreleptin), CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin preparations: FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), GLP-1 receptor agonist, GLP-1 receptor/GIP receptor coagonist, glucagon receptor/GLP-1 receptor/GIP receptor triagonist, and the like.

Here, as the therapeutic agent for diabetes, for example, insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (e.g., hydrochloride), rosiglitazone or a salt thereof (e.g., maleate), Metaglidasen, AMG-131, Balaglitazone, MBX-2044, Rivoglitazone, Aleglitazar, Chiglitazar, Lobeglitazone, PLX-204, PN-2034, GFT-505, THR-0921, compound described in WO007/013694, WO2007/018314, WO2008/093639 or WO2008/099794), α-glucosidase inhibitors (e.g., voglibose, acarbose, miglitol, emiglitate), biguanides (e.g., metformin, buformin or a salt thereof (e.g., hydrochloride, fumarate, succinate)), insulin secretagogues (e.g., sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrate thereof), dipeptidyl peptidase IV inhibitors (e.g., Alogliptin or a salt thereof (e.g., benzoate), Vildagliptin, Sitagliptin, Saxagliptin, BI1356, GRC8200, MP-513, PF-00734200, PHX1149, SK-0403, ALS2-0426, TA-6666, TS-021, KRP-104, Trelagliptin or a salt thereof (e.g., succinate)), 03 agonists (e.g., N-5984), GPR40 agonists (e.g., Fasiglifam or a hydrate thereof, compound described in WO2004/041266, WO2004/106276, WO2005/063729, WO2005/063725, WO2005/087710, WO2005/095338, WO2007/013689 or WO2008/001931), SGLT2 (sodium-glucose cotransporter 2) inhibitors (e.g., Dapagliflozin, AVE2268, TS-033, YM543, TA-7284, Remogliflozin, ASP1941), SGLT1 inhibitors, 11-hydroxysteroid dehydrogenase inhibitors (e.g., BVT-3498, INCB-13739), adiponectin or agonist thereof, IKK inhibitors (e.g., AS-2868), leptin resistance improving drugs, somatostatin receptor agonists, glucokinase activators (e.g., Piragliatin, AZD1656, AZD6370, TTP-355, compound described in WO006/112549, WO007/028135, WO008/047821, WO008/050821, WO008/136428 or WO008/156757), GPR119 agonists (e.g., PSN821, MBX-2982, APD597), FGF21, FGF analogue, ACC2 inhibitors, GLP-1 receptor agonist, GLP-1 receptor/GIP receptor coagonist, glucagon receptor/GLP-1 receptor/GIP receptor triagonist, and the like can be mentioned.

As the therapeutic agent for diabetic complications may include, aldose reductase inhibitors (e.g., tolrestat, epalrestat, zopolrestat, fidarestat, CT-112, ranirestat (AS-3201), lidorestat), neurotrophic factor and increasing agents thereof (e.g., NGF, NT-3, BDNF, neurotrophic production/secretion promoting agent described in WO01/14372 (e.g., 4-(4-chlorophenyl)-2-(2-methyl-1-imidazolyl)-5-[3-(2-methylphenoxy)propyl]oxazole), compound described in WO2004/039365), PKC inhibitors (e.g., ruboxistaurin mesylate), AGE inhibitors (e.g., ALT946, N-phenacylthiazolium bromide (ALT766), EXO-226, Pyridorin, pyridoxamine), GABA receptor agonists (e.g., gabapentin, pregabalin), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), apoptosis signal regulating kinase-1 (ASK-1) inhibitors, GLP-1 receptor agonist, GLP-1 receptor/GIP receptor coagonist, glucagon receptor/GLP-1 receptor/GIP receptor triagonist, and the like can be mentioned.

As the therapeutic agent for hyperlipidemia, HMG-CoA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compound described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), probucol, nicotinic acid drugs (e.g., nicomol, niceritrol, niaspan), ethyl icosapentate, phytosterol (e.g., soysterol, gamma oryzanol (γ-oryzanol)), cholesterol absorption inhibitors (e.g., zechia), CETP inhibitors (e.g., dalcetrapib, anacetrapib), ω-3 fatty acid preparations (e.g., ω-3-fatty acid ethyl esters 90 (ω-3-acid ethyl esters 90)) and the like can be mentioned.

Examples of the antihypertensive agent include angiotensin converting enzyme inhibitors (e.g., captopril, enalapril, delapril, etc.), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil, etc.), calcium antagonists (e.g., manidipine, nifedipine, amlodipine, efonidipine, nicardipine, cilnidipine, etc.), β blockers (e.g., metoprolol, atenolol, propranolol, carvedilol, pindolol, etc.), clonidine and the like.

As the diuretic, for example, xanthine derivatives (e.g., theobromine sodium salicylate, theobromine calcium salicylate and the like), thiazide preparations (e.g., ethiazide, cyclopenthiazide, trichloromethiazide, hydrochlorothiazide, hydroflumethiazide, benzylhydrochlorothiazide, penfluthiazide, poly5thiazide, methyclothiazide and the like), antialdosterone preparations (e.g., spironolactone, triamterene and the like), carbonic anhydrase inhibitors (e.g., acetazolamide and the like), chlorobenzenesulfonamide agents (e.g., chlortalidone, mefruside, indapamide and the like), azosemide, isosorbide, ethacrynic acid, piretanide, bumetanide, furosemide and the like can be mentioned.

Examples of the chemotherapeutic include alkylating agents (e.g., cyclophosphamide, ifosfamide), antimetabolites (e.g., methotrexate, 5-fluorouracil), anticancer antibiotics (e.g., mitomycin, adriamycin), plant-derived anticancer agents (e.g., vincristine, vindesine, Taxol), cisplatin, carboplatin, etoposide and the like. Among others, a 5-fluorouracil derivative Furtulon or Neofurtulon or the like may be used. Also a composition comprising a GIP receptor agonist peptide of the disclosure can be administered before, after or during the administration of the following anti-cancer agents: cisplatin, carboplatin. Oxaliplatin, cyclophosphamide, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil.

Examples of the immunotherapeutic include microbial or bacterial components (e.g., muramyl dipeptide derivative, Picibanil), polysaccharides having immunoenhancing activity (e.g., lentinan, sizofiran, Krestin), cytokines obtained by genetic engineering approaches (e.g., interferon, interleukin (IL)), colony-stimulating factors (e.g., granulocyte colony-stimulating factor, erythropoietin) and the like. Among others, interleukins such as IL-1, IL-2, IL-12 and the like are some examples.

Examples of the anti-inflammatory drug include nonsteroidal anti-inflammatory drugs such as aspirin, acetaminophen, indomethacin and the like.

As the antithrombotic agent, for example, heparin (e.g., heparin sodium, heparin calcium, enoxaparin sodium, dalteparin sodium), warfarin (e.g., warfarin potassium), anti-thrombin drugs (e.g., aragatroban, dabigatran), FXa inhibitors (e.g., rivaroxaban, apixaban, edoxaban, YM150, compound described in WO02/06234, WO2004/048363, WO2005/030740, WO2005/058823 or WO2005/113504), thrombolytic agents (e.g., urokinase, tisokinase, alteplase, nateplase, monteplase, pamiteplase), platelet aggregation inhibitors (e.g., ticlopidine hydrochloride, clopidogrel, prasugrel, E5555, SHC530348, cilostazol, ethyl icosapentate, beraprost sodium, sarpogrelate hydrochloride) and the like can be mentioned.

Examples of the therapeutic agent for osteoporosis include alfacalcidol, calcitriol, elcatonin, calcitonin salmon, estriol, ipriflavone, pamidronate disodium, alendronate sodium hydrate, incadronate disodium, risedronate disodium and the like.

Examples of the vitamin include vitamin B1, vitamin B12 and the like.

Examples of the antidementia drug include tacrine, donepezil, rivastigmine, galanthamine and the like.

Examples of the erectile dysfunction drug include apomorphine, sildenafil citrate and the like.

Examples of the therapeutic drug for urinary frequency or urinary incontinence include flavoxate hydrochloride, oxybutynin hydrochloride, propiverine hydrochloride and the like.

Examples of the therapeutic agent for dysuria include acetylcholine esterase inhibitors (e.g., distigmine) and the like.

Examples of the central D2 receptor antagonist include typical psychotropic drugs (prochlorperazine, haloperidol, chlorpromazine, and the like), serotonin dopamine antagonists (perospirone, risperidone, and the like), and multi-acting receptor targeted antipsychotic drugs (olanzapine and the like).

Examples of the prokinetic agent include peripheral D2 receptor antagonists (metoclopramide, domperidone, and the like) and 5HT4 receptor agonists (mosapride and the like).

Examples of the antihistamine include hydroxyzine, diphenhydramine, and chlorpheniramine.

Examples of the muscarinic receptor antagonist include central muscarinic receptor antagonists (scopolamine and the like) and peripheral muscarinic receptor antagonists (butylscopolamine and the like).

Examples of the serotonin 5HT3 receptor antagonist include granisetron, ondansetron, azasetron, indisetron, palonosetron, and ramosetron.

Examples of the somatostatin analogue include octreotide.

Examples of the corticosteroid include dexamethasone, betamethasone, and methylprednisolone.

Examples of the benzodiazepine anxiolytic include lorazepam and alprazolam, examples of the NK-1 receptor antagonist include aprepitant and fosaprepitant, and examples of the hypercalcemia therapeutic drug include bisphosphonate.

Moreover, a drug confirmed to have a cachexia-ameliorating action either in animal models or clinically, i.e., a cyclooxygenase inhibitor (e.g., indomethacin), a progesterone derivative (e.g., megestrol acetate), glucocorticoid (e.g., dexamethasone), a metoclopramide drug, a tetrahydrocannabinol drug, an agent for improving fat metabolism (e.g., eicosapentaenoic acid), growth hormone, IGF-1, or an antibody against a cachexia-inducing factor TNF-α, LIF, IL-6 or oncostatin M or the like can also be used in combination with the compound of the present disclosure.

Alternatively, a glycation inhibitor (e.g., ALT-711), a nerve regeneration-promoting drug (e.g., Y-128, VX853, prosaptide), an antidepressant (e.g., desipramine, amitriptyline, imipramine), an antiepileptic drug (e.g., lamotrigine, Trileptal, Keppra, Zonegran, Pregabalin, Harkoseride, carbamazepine), an antiarrhythmic drug (e.g., mexiletine), an acetylcholine receptor ligand (e.g., ABT-594), an endothelin receptor antagonist (e.g., ABT-627), a monoamine uptake inhibitor (e.g., tramadol), a narcotic analgesic (e.g., morphine), a GABA receptor agonist (e.g., gabapentin, MR preparation of gabapentin), an α2 receptor agonist (e.g., clonidine), a local analgesic (e.g., capsaicin), an antianxiety drug (e.g., benzothiazepine), a phosphodiesterase inhibitor (e.g., sildenafil), a dopamine receptor agonist (e.g., apomorphine), midazolam, ketoconazole or the like may be used in combination with the compound of the present disclosure.

The time of administration of the GIP receptor agonist peptide of the present disclosure and that of the concomitant drug are not limited, and they may be administered simultaneously or in a staggered manner to the administration subject.

Examples of such administration mode include the following:

(1) administration of a single preparation obtained by simultaneously processing the GIP receptor agonist peptide of the present disclosure and the concomitant drug, (2) simultaneous administration of two kinds of preparations of the GIP receptor agonist peptide of the present disclosure and the concomitant drug, which have been separately produced, by the same administration route, (3) administration of two kinds of preparations of the GIP receptor agonist peptide of the present disclosure and the concomitant drug, which have been separately produced, by the same administration route in a staggered manner, (4) simultaneous administration of two kinds of preparations of the GIP receptor agonist peptide of the present disclosure and the concomitant drug, which have been separately produced, by different administration routes, (5) administration of two kinds of preparations of the compound of the present disclosure and the concomitant drug, which have been separately produced, by different administration routes in a staggered manner (e.g., administration in the order of the GIP receptor agonist peptide of the present disclosure and the concomitant drug, or in the reverse order) and the like.

The dose of the concomitant drug can be appropriately determined based on the dose employed in clinical situations. The mixing ratio of the GIP receptor agonist peptide of the present disclosure and a concomitant drug can be appropriately determined depending on the administration subject, symptom, administration method, target disease, combination and the like. When the subject of administration is human, for example, a concomitant drug can be used in 0.01-100 parts by weight relative to 1 part by weight of the GIP receptor agonist peptide of the present disclosure.

By combining the GIP receptor agonist peptide of the present disclosure and concomitant drug: (1) the dose of the GIP receptor agonist peptide of the present disclosure or a concomitant drug can be reduced as compared to single administration of the GIP receptor agonist peptide of the present disclosure or a concomitant drug,

(2) the drug to be used in combination with the GIP receptor agonist peptide of the present disclosure can be selected depending on the condition of patients (mild, severe and the like),

(3) the period of treatment can be set longer by selecting a concomitant drug having different action and mechanism from those of the GIP receptor agonist peptide of the present disclosure,

(4) a sustained treatment effect can be designed by selecting a concomitant drug having different action and mechanism from those of the GIP receptor agonist peptide of the present disclosure, and

(5) a synergistic effect can be afforded by a combined use of the GIP receptor agonist peptide of the present disclosure and a concomitant drug, and the like, can be achieved.

### F. EXAMPLES

The abbreviations used in the present specification mean the following (Table 2). A hyphen in terms such as α-MePhe and the like as described herein may be omitted, and the event of omission also represents the same meaning.

In the amino acid sequences used in the present specification, the left terminal represents N terminal and the right terminal represents C terminal.

TABLE 2
Commonly used abbreviations in the present disclosure.

| | |
|---|---|
| Ac | acetyl |
| Aib | α-aminoisobutyric acid |
| Ambz(4) | 4-aminomethylbenzoyl |
| GABA | γ-aminobutyric acid |
| Iva | isovaline |
| Lys(Ac) | Nε-acetyllysine |
| α-MePhe | α-methylphenylalanine |
| MeTyr | N-Methyltyrosine |
| Hda | 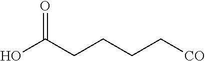 |
| Doda | 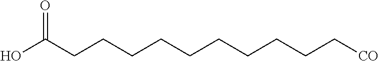 |
| Trda | 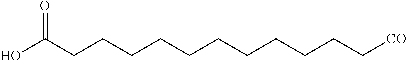 |
| Teda | 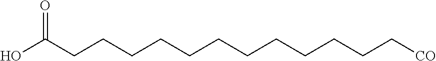 |
| Peda | 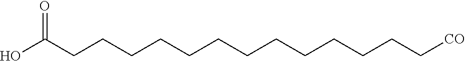 |
| Heda | 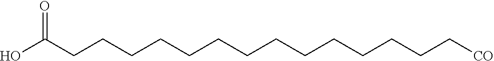 |
| Hepda | 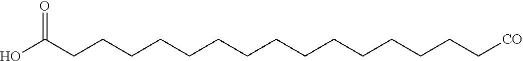 |
| Oda | 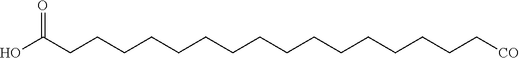 |
| Eda | 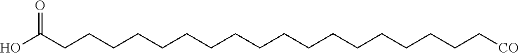 |
| Dda | 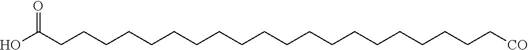 |
| Pal | 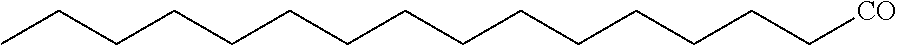 |
| PEG(2) | 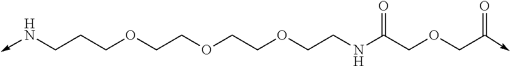 |
| PEG3 | 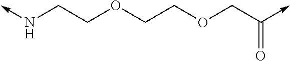 |
| (PEG3)2 | 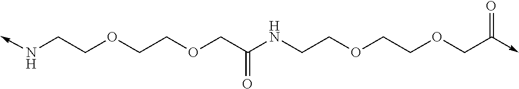 |
| (PEG3)3 | 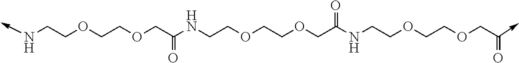 |
| (PEG3)4 | 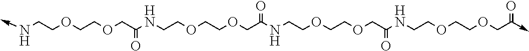 |
| (PEG3)5 | 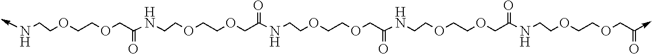 |
| PEG(4) | 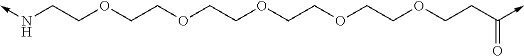 |
| γGlu | 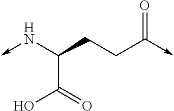 |
| (γGlu)2 | 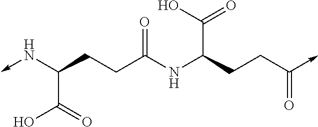 |
| (γGlu)3 | 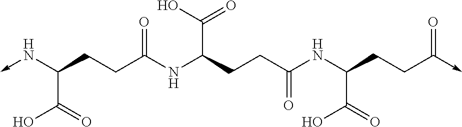 |
| (γGlu)2-PEG3 | 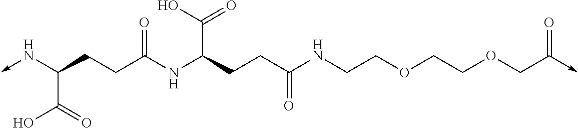 |
| AMBZ-PEG3 | 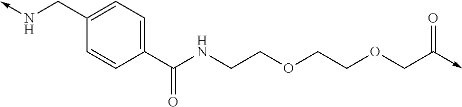 |
| GABA- (PEG3)2 | 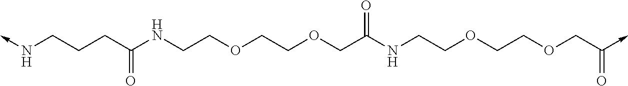 |
| GABA-GGG | 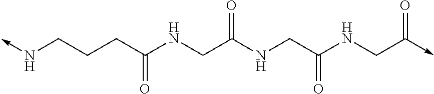 |
| GG | 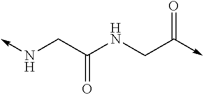 |
| GGG | 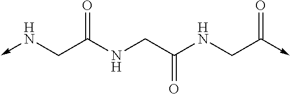 |
| GGGG | 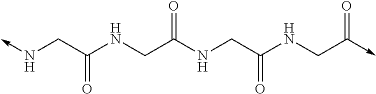 |
| GGGGG | 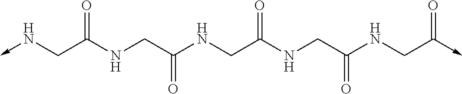 |
| GGGGGG | 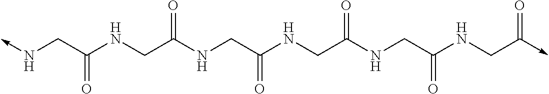 |
| G9 | 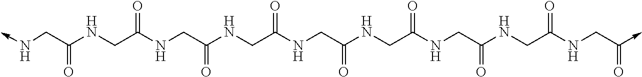 |
| NpipAc | 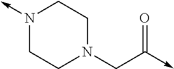 |
| NpipAc- PEG3 | 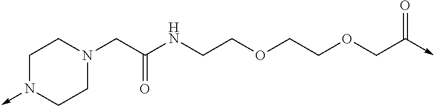 |
| Tra | 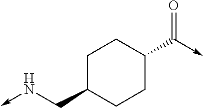 |
| Tra-GGG | 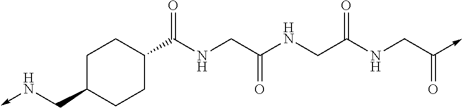 |
| Tra-PEG3 | 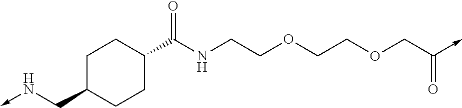 |
| γGlu-PEG3 | 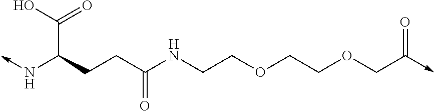 |
| γGlu- (PEG3)2 | 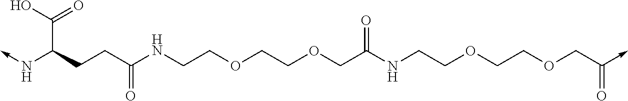 |
| γGlu-AMBZ- PEG3 | 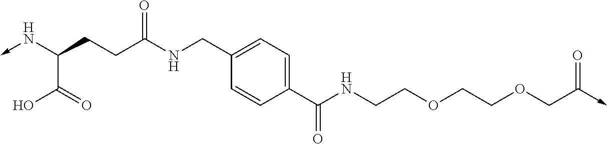 |
| γGlu-GGG | 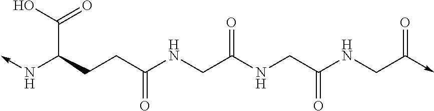 |
| εLys | 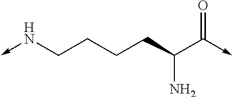 |
| εLys-GGG | 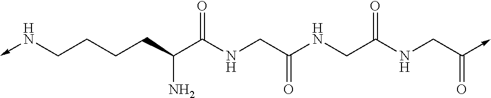 |
| εLys-PEG3 | 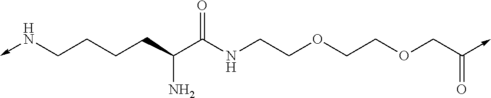 |
| gE | 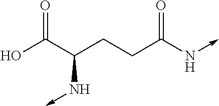 |
| OEG = AEEA = PEG3 | 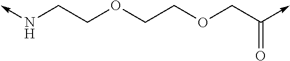 |
| GGGGG | 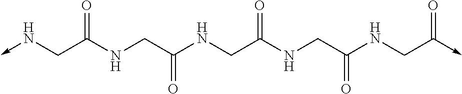 |
| GGEEE = G2E3 | 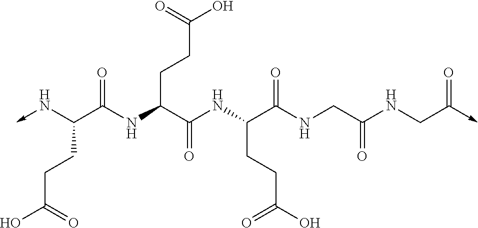 |
| G3gEgE | 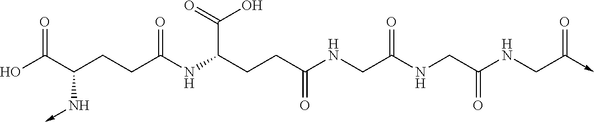 |
| 2OEGgEgE | 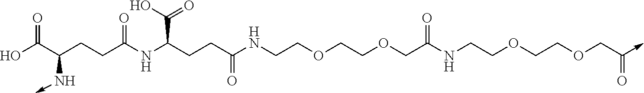 |
| OEGgEgE | 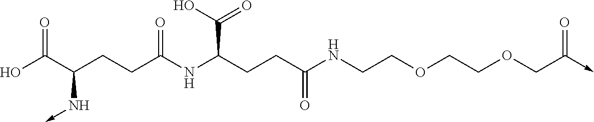 |
| GGPAPAP | 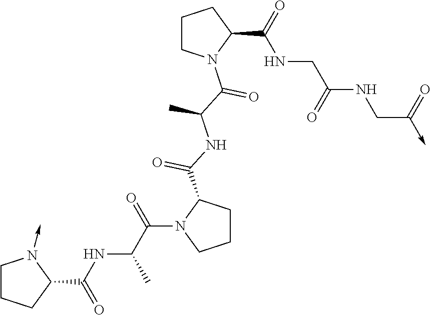 |
| 2OEGgE | 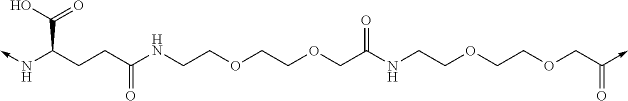 |
| 3OEGgEgE | 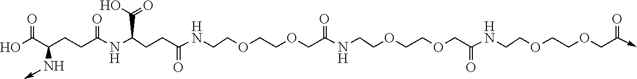 |
| G4gE | 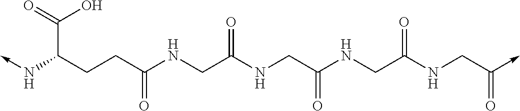 |
| G5gE | 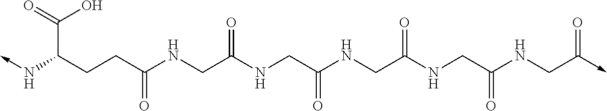 |
| 2OEGgEgEgE | 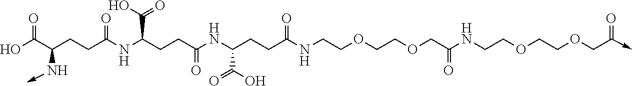 |
| 2OEG | 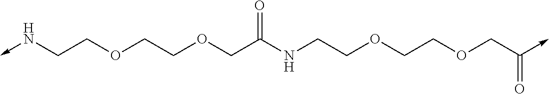 |
| G5gEgE | 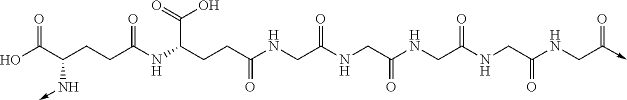 |
| gE = γGlu | 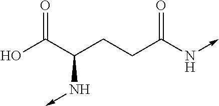 |
| C15DA = C₁₅ diacid | 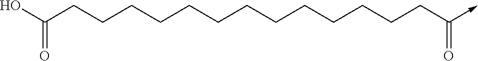 |
| C16DA = C₁₆ diacid | 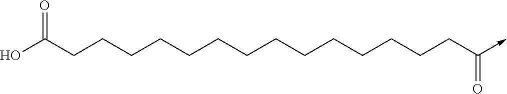 |
| C16 | 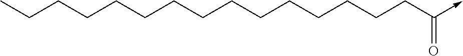 |
| C17DA = C₁₇ diacid | 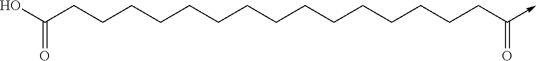 |
| C18DA = C₁₈ diacid | 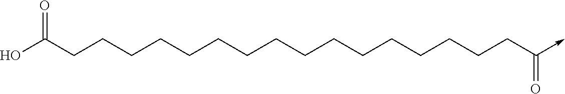 |
| C18 | 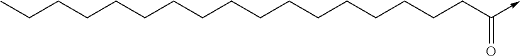 |
| C20DA = C₂₀ diacid | 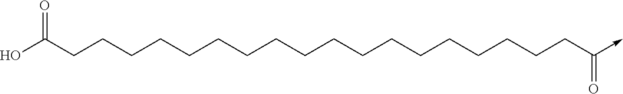 |
| Ac | 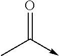 |
| Me-Tyr = MeTyr | 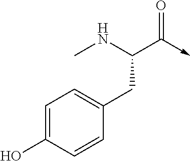 |
| Aib | 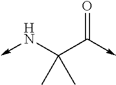 |

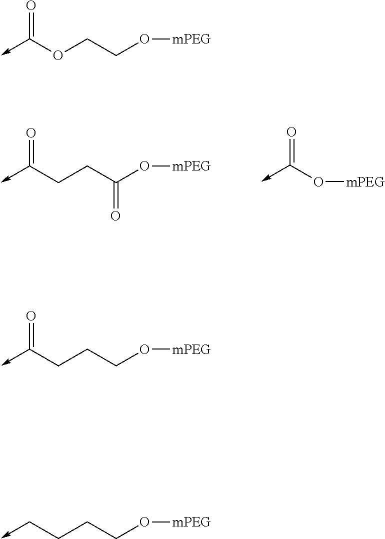

PEG linkers used for Cys. PEG=5-30 kDA PEG

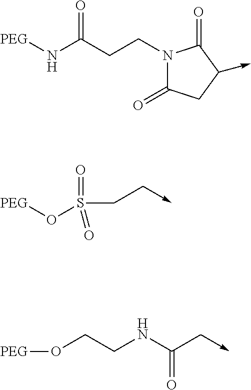

In the specification, where bases, amino acids, etc. are denoted by their codes, they are based on conventional codes in accordance with the IUPAC-IUB Commission on Biochemical Nomenclature or by the common codes in the art, examples of which are shown below. For amino acids that may have an optical isomer, L-form is presented unless otherwise indicated (e.g., “Ala” is L-form of Ala). In addition, “D-” means a D-form (e.g., “D-Ala” is D-form of Ala), and “DL-” means a racemate of a D-form and an L-form (e.g., “DL-Ala” is DL racemate of Ala).

The present disclosure is explained in detail in the following by referring to the following Reference Examples, Examples, Test Examples and Formulation Examples, which are mere embodiments and not to be construed as limitative. In addition, the present disclosure may be modified without departing from the scope of invention.

The term “room temperature” in the following Examples indicates the range of generally from about 10° C. to about 35° C. As for “%”, the yield is in mol/mol %, the solvent used for chromatography is in % by volume and other “%” is in % by weight.

NMP: methylpyrrolidone
THF: tetrahydrofuran

### DMF: N,N-dimethylformamide

WSC: 1-(3-dimethylaminopropyl)-3-ethylcarbodiimide hydrochloride

### DCC: N,N′-Dicyclohexylcarbodiimide

DIPCDI N,N′-diisopropylcarbodiimide
HOBt: 1-hydroxybenzotriazole monohydrate
Oxyma: ethyl 2-cyano-2-(hydroxyimino)acetate

### Example 1. Synthesis Schemes

Exemplary methods for synthesizing GIP receptor agonist peptides are disclosed for example in Applicant's International PCT Application No. PCT/JP2018/013540, filed on Mar. 30, 2018, ranging from pages 162 to 213, the disclosure of which is specifically incorporated herein by reference in its entirety.

### Example 2. Synthesis of Selective GIP Receptor Agonist Peptides of the Present Disclosure. Compound No. 25; SEQ ID NO: 26

The peptide compound 25 was synthesized using standard Fmoc chemistry.

1. Resin preparation: the 2-CTC Resin (100 g, 50.0 mmol, 1.00 eq, Sub 0.50 mmol/g) was added Fmoc-Gly-OH (14.9 g, 50.0 mmol, 1.00 eq) and DIEA (25.8 g, 200 mmol, 33.1 mL, 4.00 eq) in DCM (250 mL). The mixture was agitated with N₂ for 2 h at 25° C., then added MeOH (100 mL) agitated with N₂ for another 30 min. The resin was washed with DMF (900 mL times 5). Then 20% piperidine in DMF (900 mL) was added and the mixture was agitated with N₂ for 20 min at 25° C. Then the mixture was filtered to get the resin. The resin was washed with DMF (900 mL times 5) and filtered to get the resin.

2. Coupling: A solution of Fmoc-Lys(Boc)-OH (70.3 g, 150 mmol, 3.00 eq), DIEA (38.8 g, 300 mmol, 49.7 mL, 6.00 eq) and HBTU (54.1 g, 143 mmol, 2.85 eq) in DMF (250 m 3) was added to the resin and agitated with N₂ for 35 min at 30° C. The resin was then washed with DMF (900 mL times 5).

3. Deprotection: 20 piperidine in DMF (900 mL) was added to the resin and the mixture was agitated with N₂ for 20 min at 30′° C.

4. Repeat Step 2 and 3 for the coupling of following amino acids: (1-29):

| # | Materials | Coupling reagents |
|---|---|---|
| 1 | FMOC-GLN(TRT)-OH (3.00 eq) | HBTU (2.85 eq) and DIEA (6.00 eq) |
| 2 | FMOC-ALA-OH (3.00 eq) | HBTU (2.85 eq) and DIEA (6.00 eq) |
| 3 | FMOC-LEU-OH (3.00 eq) | HBTU (2.85 eq) and DIEA (6.00 eq) |
| 4 | FMOC-LEU-OH (3.00 eq) | HBTU (2.85 eq) and DIEA (6.00 eq) |
| 5 | FMOC-TRP(BOC)-OH (3.00 eq) | HBTU (2.85 eq) and DIEA (6.00 eq) |
| 6 | FMOC-ASN(TRT)-OH (3.00 eq) | HBTU (2.85 eq) and DIEA (6.00 eq) |
| 7 | FMOC-VAL-OH (3.00 eq) | HBTU (2.85 eq) and DIEA (6.00 eq) |
| 8 | FMOC-PHE-OH (3.00 eq) | HBTU (2.85 eq) and DIEA (6.00 eq) |
| 9 | FMOC-LYS(DDE)-OH (3.00 eq) | HBTU (2.85 eq) and DIEA (6.00 eq) |
| 10 | FMOC-AIB-OH (3.00 eq) | HBTU (2.85 eq) and DIEA (6.00 eq) |
| 11 | FMOC-GLN(TRT)-OH (6.00 eq) | HATU (5.70 eq) and DIEA (12.0 eq) |
| 12 | FMOC-ALA-OH (3.00 eq) | HBTU (2.85 eq) and DIEA (6.00 eq) |
| 13 | FMOC-AIB-OH (3.00 eq) | HBTU (2.85 eq) and DIEA (6.00 eq) |
| 14 | FMOC-ARG(PBF)-OH (6.00 eq) | HATU (2.85 eq) and DIEA (6.00 eq) |
| 15 | FMOC-ASP(OTBU)-OH (3.00 eq) | HBTU (2.85 eq) and DIEA (6.00 eq) |
| 16 | FMOC-LEU-OH (3.00 eq) | HBTU (2.85 eq) and DIEA (6.00 eq) |
| 17 | FMOC-ALA-OH (3.00 eq) | HBTU (2.85 eq) and DIEA (6.00 eq) |
| 18 | FMOC-ILE-OH (3.00 eq) | HBTU (2.85 eq) and DIEA (6.00 eq) |
| 19 | FMOC-SER(TBU)-OH (3.00 eq) | HBTU (2.85 eq) and DIEA (6.00 eq) |
| 20 | FMOC-TYR(TBU)-OH (3.00 eq) | HBTU (2.85 eq) and DIEA (6.00 eq) |
| 21 | FMOC-ASP(OTBU)-OH (4.00 eq) | HBTU (3.80 eq) and DIEA (8.00 eq) |
| 22 | FMOC-SER(TBU)-OH (4.00 eq) | HBTU (3.80 eq) and DIEA (8.00 eq) |
| 23 | FMOC-ILE-OH (4.00 eq) | HATU (3.80 eq) and DIEA (8.00 eq) |
| 24 | FMOC-PHE-OH (4.00 eq) | HATU (3.80 eq) and DIEA (8.00 eq) |
| 25 | FMOC-THR(TBU)-OH (4.00 eq) | HATU (3.80 eq) and DIEA (8.00 eq) |
| 26 | FMOC-GLY-OH (4.00 eq) | HATU (3.80 eq) and DIEA (8.00 eq) |
| 27 | FMOC-GLU(OTBU)-OH (6.00 eq) | HATU (5.70 eq) and DIEA (12.0 eq) |
| 28 | FMOC-AIB-OH (4.00 eq) | HATU (3.80 eq) and DIEA (8.00 eq) |
| 29 | FMOC-N-ME-TYR(TBU)-OH (3.00 eq) | HATU (2.85 eq) and DIEA (6.00 eq) |

5. Coupling: Boc₂/DIPEA/DMF (10/5/85) 1400 ma for 30 m, then the resin was washed with DMF (1600 mL times 5).

6. Deprotection: Dde was treated with Hydrazine hydrate/DMF (3/97) 1400 mL for 30 min, then the resin was washed with DMF (1600 mL times 5).

7. Repeat Step 2 and 3 for the coupling of following amino acids: (1-5):

| | | |
|---|---|---|
| 1 | Fmoc-AEEA-OH (3.00 eq) | HBTU (2.85 eq) and DIEA (6.00 eq) |
| 2 | Fmoc-AEEA-OH (3.00 eq) | HBTU (2.85 eq) and DIEA (6.00 eq) |
| 3 | Fmoc-Glu-OtBu (3.00 eq) | HBTU (2.85 eq) and DIEA (6.00 eq) |
| 4 | Fmoc-Glu-OtBu (3.00 eq) | HBTU (2.85 eq) and DIEA (6.00 eq) |
| 5 | pentadecanedioic | HOBt (4.00 eq) and DIC (4.00 eq) |
| | acid (4.00 eq) | |

Peptide Cleavage and Purification:

1. After coupling, the resin was washed with DMF for 5 times. After last step, the resin was washed with MeOH for 3 times and dried under vacuum to get 301 g peptide resin. Then 3000 mL of cleavage buffer (92.5% TFA/2.5%₀ 3-Mercaptopropionic acid/2.5% TIS/2.5% H2O) was added to the flask containing the side chain protected peptide resin at 25° C. and the mixture was stirred for 2.5 h. The cleavage buffer was concentrated under reduced pressure to give 1000 mL. The peptide was precipitated with cold tert-butyl methyl ether (7000 mL), then was filtered to give the filter cake, dried the filter cake over vacuum for 2 h to give the crude peptide (182 g) which was confirmed by LCMS (Rt=1.563 min).

2. The crude peptide was purified by prep-HPLC (TFA condition; A: 0.075% TFA in H2O, B: CH3CN) to give the peptide, then the peptide was purified by prep-HPLC (HOAC condition; A: 0.5% HOAC in H2O, B: ACN) to give the final product compound 25 (11.86 g, 5.23% yield, 96.23% purity, HOAC) was obtained as a white solid.

Purification Conditions:

First Purification condition

| | |
|---|---|
| Dissolution | Dissolve in 20% ACN in H₂O |
| condition | |
| Instrument | Gilson GX-281 |
| Mobile Phase | A: H₂O (0.075% TFA in H₂O) |
| | B: ACN |
| Gradient | 21-51-60 min. Retention time: 39 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

Second Purification condition

| | |
|---|---|
| Dissolution | The liquid is directly Purification |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 29-49-50 min. Retention time: 61 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

### Example 3. Synthesis of Selective GIP Receptor Agonist Peptides of the Present Disclosure. Compound No. 142; SEQ ID NO: 143

The peptide 142 was synthesized using standard Fmoc chemistry.

1. Resin preparation: the 2-CTC Resin (100 g, 50.0 mmol, 1.00 eq, Sub 0.50 mmol/g) was added Fmoc-GLY-OH (14.9 g, 50.0 mmol, 1.00 eq) and DIEA (25.85 g, 200.0 mmol, 33.14 mL, 4.00 eq) in DCM (280 mL). The mixture was agitated with N₂ for 2 h at 25° C., then added MeOH (100.0 mL) agitated with N₂ for another 30 min. The resin was washed with DMF (400 mL×5). Then 20% piperidine in DMF (400 mL) was added and the mixture was agitated with N₂ for 15 min at 25° C. Then the mixture was filtered to get the resin. The resin was washed with DMF (400 mL×5) and filtered to get the resin.

2. Coupling: a solution of FMOC-ARG(PBF)-OH (97.32 g, 150 mmol, 3.00 eq), HBTU (53.87 g, 142.5 mmol, 2.85 eq) and DIEA (38.772 g, 300 mmol, 49.707 mL, 6.00 eq) in DMF (300 mL) was added to the resin and agitated with N₂ for 40 min at 20° C. The resin was then washed with DMF (400 mL times 3).

3. Deprotection: 20% piperidine in DMF (400 mL) was added to the resin and the mixture was agitated with N₂ for 15 min at 20° C. The resin was washed with DMF (400 mL×5) and filtered to get the resin.

4. Repeat step 2 to 3 for next amino acid coupling:

| # | Materials | Coupling reagents |
|---|---|---|
| 1 | FMOC-GLN(TRT)-OH (3.00 eq) | HBTU (2.85 eq) and DIEA (6.00 eq) |
| 2 | FMOC-ALA-OH (3.00 eq) | HBTU (2.85 eq) and DIEA (6.00 eq) |
| 3 | FMOC- LEU -OH (3.00 eq) | HBTU (2.85 eq) and DIEA (6.00 eq) |
| 4 | FMOC-LEU-OH (3.00 eq) | HBTU (2.85 eq) and DIEA (6.00 eq) |
| 5 | FMOC-TRP(BOC)-OH (3.00 eq) | HBTU (2.85 eq) and DIEA (6.00 eq) |
| 6 | FMOC-ASN(TRT)-OH (3.00 eq) | HBTU (2.85 eq) and DIEA (6.00 eq) |
| 7 | FMOC-VAL-OH (3.00 eq) | HBTU (2.85 eq) and DIEA (6.00 eq) |
| 8 | FMOC-PHE-OH (3.00 eq) | HBTU (2.85 eq) and DIEA (6.00 eq) |
| 9 | FMOC-ASP(OTBU)-OH (3.00 eq) | HBTU (2.85 eq) and DIEA (6.00 eq) |
| 10 | FMOC-AIB-OH (3.00 eq) | HBTU (2.85 eq) and DIEA (6.00 eq) |
| 11 | FMOC-GLN(TRT)-OH (3.00 eq) | HATU (2.85 eq) and DIEA (6.00 eq) |
| 12 | FMOC-ALA-OH (3.00 eq) | HBTU (2.85 eq) and DIEA (6.00 eq) |
| 13 | FMOC-AIB-OH (3.00 eq) | HBTU (2.85 eq) and DIEA (6.00 eq) |
| 14 | FMOC-ARG(PBF)-OH (3.00 eq) | HATU (2.85 eq) and DIEA (6.00 eq) |
| 14 | FMOC-ARG(PBF)-OH (3.00 eq) | HATU (2.85 eq) and DIEA (6.00 eq) |
| 15 | FMOC-ASP(OTBU)-OH (3.00 eq) | HBTU (2.85 eq) and DIEA (6.00 eq) |
| 16 | FMOC-LYS(DDE)-OH (3.00 eq) | HBTU (2.85 eq) and DIEA (6.00 eq) |
| 17 | FMOC-AIB-OH (3.00 eq) | HBTU (2.85 eq) and DIEA (6.00 eq) |
| 18 | FMOC-ILE-OH (3.00 eq) | HATU (2.85 eq) and DIEA (6.00 eq) |
| 18 | FMOC-ILE-OH (5.00 eq) | HOAT (5.00 eq) and DIC (5.00 eq) |
| 19 | FMOC-SER(TBU)-OH (4.00 eq) | HBTU (3.80 eq) and DIEA (8.00 eq) |
| 20 | FMOC-TYR(TBU)-OH (4.00 eq) | HBTU (3.80 eq) and DIEA (8.00 eq) |
| 21 | FMOC-ASP(OTBU)-OH (4.00 eq) | HBTU (3.80 eq) and DIEA (8.00 eq) |
| 22 | FMOC-SER(TBU)-OH (4.00 eq) | HBTU (3.80 eq) and DIEA (8.00 eq) |
| 23 | FMOC-ILE-OH (4.00 eq) | HATU (3.80 eq) and DIEA (8.00 eq) |
| 24 | FMOC-PHE-OH (4.00 eq) | HATU (3.80 eq) and DIEA (8.00 eq) |
| 25 | FMOC-THR(TBU)-OH (4.00 eq) | HATU (3.80 eq) and DIEA (8.00 eq) |
| 26 | FMOC-GLY-OH (4.00 eq) | HATU (3.80 eq) and DIEA (8.00 eq) |
| 27 | FMOC-GLU(OTBU)-OH (4.00 eq) | HATU (3.80 eq) and DIEA (8.00 eq) |
| 27 | FMOC-GLU(OTBU)-OH (4.00 eq) | HATU (3.80 eq) and DIEA (8.00 eq) |
| 28 | FMOC-AIB-OH (4.00 eq) | HATU (3.80 eq) and DIEA (8.00 eq) |
| 29 | FMOC-N-ME-TYR(TBU)-OH (3.00 eq) | HATU (2.85 eq) and DIEA (6.00 eq) |

5. Coupling: Boc₂O/DIPEA/DMF (10/5/85) 500 mL for 30 min and then repeat it for one more time, then the resin was washed with DMF (500 mL×5).

6. Deprotection: Dde was treated with Hydrazine hydrate/DMF (3/97) 500 mL for 10 min and then repeat it for one more time, then the resin was washed with DMF (500 mL×5).

7. Repeat Step 2 and 3 for all other amino acids: (FMOC-GLY-GLY-GLY-OH, FMOC-GLY-GLY-OH, pentadecanedioic acid).

Peptide Cleavage and Purification:

1. The resin was washed with MeOH (500 mL×3) and dried under vacuum to get 270 g peptide resin. Then 2800 mL of cleavage buffer (92.5% TFA/2.5% 3-Mercaptopropionic acid/2.5% TIS/2.5% H2O) was added to the flask containing the side chainprotected peptide resin at 20° C. and the mixture was stirred for 2.5 h. The cleavage buffer was concentrated under reduced pressure to give 900 mL. The peptide was precipitated with cold tert-butyl methyl ether (7.20 L), then was filtered to give the filter cake, dried the filter cake over vacuum for 2 h to give the crude peptide (179.5 g) was obtained as a white solid and LCMS.

2. The crude peptide was purified by prep-HPLC (TFA condition; A: 0.075% TFA in H2O, B: CH3CN) to give the peptide, then the peptide was purified by prep-HPLC (HOAC condition; A: 0.5% HOAC in H2O, B: ACN) to give the final product. The product (8.39 g) and (3.56 g) was combined for lyophilization to give the final product Compound 142 (11.95 g, 98.49% purity, HOAC) was obtained as a white solid.

Purification Conditions:

First Purification condition

| | |
|---|---|
| Dissolution | Dissolve in 20% ACN in H₂O |
| condition | |
| Instrument | Gilson GX-281 |
| Mobile Phase | A: H₂O (0.075% TFA in H₂O) |
| | B: ACN |
| Gradient | 24-44-60 min. Retention time: 42 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 30-45-53 min. Retention time: 53 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

### Example 4. Synthesis of Selective GIP Receptor Agonist Peptides of the Present Disclosure. Compound No. 17; SEQ ID NO: 18

The peptide compound 17 was synthesized using standard Fmoc chemistry.

1. Resin preparation: The Rink Amine MBHA resin (6 mmol, 1.00 eq, 24 g, Sub 0.25 mmol/g) in DMF (250 mL) was agitated with N₂ for 2 hrs at 20° C. Then 20% piperidine in DMF (500 mL) was added and the mixture was agitated with N₂ for 15 mi at 20° C. Then the mixture was filtered to get the resin. The resin was washed with DMF (500 mL times 5) and filtered to get the resin.

2. Coupling: a solution of FMOC-SER(TBU)-OH (3.00 eq) and HBTU (2.85 eq), DIEA (6.00 eq) in DMF (250 mL) was added to the resin and agitated with N₂ for 30 min at 20° C. The resin was then washed with DMF (500 mL times 3).

3. Deprotection: 20% piperidine in DMF (500 mL) was added to the resin and the mixture was agitated with N₂ for 15 m at 20° C. The resin was washed with DMF (500 mL times 5) and filtered to get the resin.

4. Repeat step 2 to 3 for the coupling of following amino acids: (1-38)

| # | Materials | Coupling reagents |
|---|---|---|
| 1 | FMOC-PRO-OH(3.00 eq) | HBTU (2.85 eq) and DIEA (6.00 eq) |
| 2 | FMOC-PRO-OH(3.00 eq) | HBTU (2.85 eq) and DIEA (6.00 eq) |
| 3 | FMOC-PRO-OH(3.00 eq) | HBTU (2.85 eq) and DIEA (6.00 eq) |
| 4 | FMOC-ALA-OH(3.00 eq) | HBTU (2.85 eq) and DIEA (6.00 eq) |
| 5 | FMOC-GLY-OH(3.00 eq) | HBTU (2.85 eq) and DIEA (6.00 eq) |
| 6 | FMOC-SER(TBU)-OH(3.00 eq) | HBTU (2.85 eq) and DIEA (6.00 eq) |
| 7 | FMOC-SER(TBU)-OH(3.00 eq) | HBTU (2.85 eq) and DIEA (6.00 eq) |
| 8 | FMOC-PRO-OH(3.00 eq) | HBTU (2.85 eq) and DIEA (6.00 eq) |
| 9 | FMOC-SER(TBU)-OH(3.00 eq) | HBTU (2.85 eq) and DIEA (6.00 eq) |
| 10 | FMOC-GLN(TRT)-OH (3.00 eq) | HBTU (2.85 eq) and DIEA (6.00 eq) |
| 11 | FMOC-ALA-OH (3.00 eq) | HBTU (2.85 eq) and DIEA (6.00 eq) |
| 12 | FMOC-LEU-OH (3.00 eq) | HBTU (2.85 eq) and DIEA (6.00 eq) |
| 13 | FMOC-LEU-OH (3.00 eq) | HBTU (2.85 eq) and DIEA (6.00 eq) |
| 14 | FMOC-TRP(BOC)-OH (3.00 eq) | HBTU (2.85 eq) and DIEA (6.00 eq) |
| 15 | FMOC-ASN(TRT)-OH (3.00 eq) | HBTU (2.85 eq) and DIEA (6.00 eq) |
| 16 | FMOC-VAL-OH (3.00 eq) | HBTU (2.85 eq) and DIEA (6.00 eq) |
| 17 | FMOC-PHE-OH (3.00 eq) | HBTU (2.85 eq) and DIEA (6.00 eq) |
| 18 | FMOC-ASN(TRT)-OH (3.00 eq) | HBTU (2.85 eq) and DIEA (6.00 eq) |
| 19 | FMOC-AIB-OH (3.00 eq) | HBTU (2.85 eq) and DIEA (6.00 eq) |
| 20 | FMOC-GLN(TRT)-OH (3.00 eq) | HATU (2.85 eq) and DIEA (6.00 eq) |
| 21 | FMOC-LYS(DDE)-OH (3.00 eq) | HBTU (2.85 eq) and DIEA (6.00 eq) |
| 22 | FMOC-AIB-OH (3.00 eq) | HBTU (2.85 eq) and DIEA (6.00 eq) |
| 23 | FMOC-ARG(PBF)-OH (3.00 eq) | HATU (2.85 eq) and DIEA (6.00 eq) |
| 24 | FMOC-ASP(OTBU)-OH (3.00 eq) | HBTU (2.85 eq) and DIEA (6.00 eq) |
| 25 | FMOC-LEU-OH (3.00 eq) | HBTU (2.85 eq) and DIEA (6.00 eq) |
| 26 | FMOC-ALA-OH (3.00 eq) | HBTU (2.85 eq) and DIEA (6.00 eq) |
| 27 | FMOC-ILE-OH (3.00 eq) | HBTU (2.85 eq) and DIEA (6.00 eq) |
| 28 | FMOC-SER(TBU)-OH (3.00 eq) | HBTU (2.85 eq) and DIEA (6.00 eq) |
| 29 | FMOC-TYR(TBU)-OH (3.00 eq) | HBTU (2.85 eq) and DIEA (6.00 eq) |
| 30 | FMOC-ASP(OTBU)-OH (3.00 eq) | HBTU (2.85 eq) and DIEA (6.00 eq) |
| 31 | FMOC-SER(TBU)-OH (3.00 eq) | HBTU (2.85 eq) and DIEA (6.00 eq) |
| 32 | FMOC-ILE-OH (3.00 eq) | HBTU (2.85 eq) and DIEA (6.00 eq) |
| 33 | FMOC-PHE-OH (3.00 eq) | HBTU (2.85 eq) and DIEA (6.00 eq) |
| 34 | FMOC-THR(TBU)-OH (3.00 eq) | HBTU (2.85 eq) and DIEA (6.00 eq) |
| 35 | FMOC-GLY-OH (3.00 eq) | HBTU (2.85 eq) and DIEA (6.00 eq) |
| 36 | FMOC-GLU(OTBU)-OH (3.00 eq) | HBTU (2.85 eq) and DIEA (6.00 eq) |
| 37 | FMOC-AIB-OH (3.00 eq) | HBTU (2.85 eq) and DIEA (6.00 eq) |
| 38 | FMOC-N-ME-TYR(TBU)-OH (3.00 eq) | HATU (2.85 eq) and DIEA (6.00 eq) |

5. To a solution of DIEA (5.00 eq) and Boc₂O (10.00 eq) in DMF (300 mL) was added to the resin and agitated with N₂ for 1 hour at 20° C. Then the resin was washed with DMF (500 mLtimes3).

6. Add 3% N₂H4-H₂O/DMF and react on 20 mins and then repeat it for one more time. Drain and wash with DMF (500 mLtimes5).

7. Repeat step 2 to 3 for the coupling of following amino acids: (1-4)

| # | Materials | Coupling reagents |
|---|---|---|
| 1 | FMOC-AEEA-OH (3.00 eq) | HBTU (2.85 eq) and DIEA (6.00 eq) |
| 2 | FMOC-AEEA-OH (3.00 eq) | HBTU (2.85 eq) and DIEA (6.00 eq) |
| 3 | FMOC-GLU-OTBU (3.00 eq) | HBTU (2.85 eq) and DIEA (6.00 eq) |
| 4 | FMOC-GLU-OTBU (3.00 eq) | HBTU (2.85 eq) and DIEA (6.00 eq) |

8. Coupling: a solution of pentadecanedioic acid (2.00 eq) and HOBt (2.00 eq), DIC (2.00 eq) in DMF (250 mL) was added to the resin and agitated with N₂ for 12 hrs at 20° C. The resin was then washed with DMF (500 mLtimes3).

9. The coupling reaction was monitored by ninhydrin color reaction.

Peptide Cleavage and Purification:

1. After coupling, the resin was washed with DMF for 5 times. After last step, the resin was washed with MeOH for 3 times, and dried under vacuum. Then the 50 g peptide resin was treated with the cleavage cocktail (500 mL, 90% TFA/3% 3-Mercaptopropionic acid/3% TIS/4% H2O) for 2.5 hours. The peptide was concentrated under reduced pressure and precipitated with cold isopropyl ether, filtered and washed two times with isopropyl ether to give 22 g residue.

2. The crude peptide was purified by Prep-HPLC (A: 0.075% TFA in H2O, B: ACN) and then was second purified by Prep-HPLC (A: 0.5% HOAc in H2O, B: ACN) to give the compound 17 (1.23 g, 97.46% purity, HOAC) was obtained as a white solid, which was confirmed by LCMS (Rt=1.563 min) and HPLC.

Purification Conditions:

First Purification condition

| | |
|---|---|
| Dissolution | Dissolve in 20% ACN-H2O |
| condition | |
| Instrument | Gilson GX-281 |
| Mobile Phase | A: H₂O (0.075% TFA in H₂O) |
| | B: ACN |
| Gradient | 24-44-60 min. Retention time: 47.5 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 33-48-60 min. Retention time: 57 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

### Example 5. Synthesis of Selective GIP Receptor Agonist Peptides of the present disclosure. Compound No. 21; SEQ ID NO: 22

The peptide compound 21 was synthesized using standard Fmoc chemistry.

1. Resin preparation: The Rink Amine MBHA resin (0.300 mmol, 1.00 eq, 1.00 g, Sub 0.30 mmol/g) in DMF (5 mL) was agitated with N₂ for 2 hrs at 20° C. Then 20% piperidine in DMF (10 mL) was added and the mixture was agitated with N₂ for 15 min at 20° C. Then the mixture was filtered to get the resin. The resin was washed with DMF (20 mLtimes5) and filtered to get the resin.

2. Coupling: a solution of FMOC-SER(TBU)-OH (345 mg, 0.900 mmol, 3.00 eq) and HBTU (323 mg, 0.855 mmol, 2.85 eq), DIEA (233 mg, 1.80 mmol, 6.00 eq) in DMF (50 mL) was added to the resin and agitated with N₂ for 30 min at 20° C. The resin was then washed with DMF (20 mLtimes5).

3. Deprotection: 20% piperidine in DMF (20 mL) was added to the resin and the mixture was agitated with N₂ for 15 min at 20° C. The resin was washed with DMF (20 mLtimes5) and filtered to get the resin.

4. Repeat step 2 to 3 for the coupling of following amino acids: (1-38)

| # | Materials | Coupling reagents |
|---|---|---|
| 1 | FMOC-PRO-OH(3.00 eq) | HBTU (2.85 eq) and DIEA (6.00 eq) |
| 2 | FMOC-PRO-OH(3.00 eq) | HBTU (2.85 eq) and DIEA (6.00 eq) |
| 3 | FMOC-PRO-OH(3.00 eq) | HBTU (2.85 eq) and DIEA (6.00 eq) |
| 4 | FMOC-ALA-OH(3.00 eq) | HBTU (2.85 eq) and DIEA (6.00 eq) |
| 5 | FMOC-GLY-OH(3.00 eq) | HBTU (2.85 eq) and DIEA (6.00 eq) |
| 6 | FMOC-SER(TBU)-OH(3.00 eq) | HBTU (2.85 eq) and DIEA (6.00 eq) |
| 7 | FMOC-SER(TBU)-OH(3.00 eq) | HBTU (2.85 eq) and DIEA (6.00 eq) |
| 8 | FMOC-PRO-OH(3.00 eq) | HBTU (2.85 eq) and DIEA (6.00 eq) |
| 9 | FMOC-LYS(DDE)-OH(3.00 eq) | HBTU (2.85 eq) and DIEA (6.00 eq) |
| 10 | FMOC-GLN(TRT)-OH (3.00 eq) | HBTU (2.85 eq) and DIEA (6.00 eq) |
| 11 | FMOC-ALA-OH (3.00 eq) | HBTU (2.85 eq) and DIEA (6.00 eq) |
| 12 | FMOC-LEU-OH (3.00 eq) | HBTU (2.85 eq) and DIEA (6.00 eq) |
| 13 | FMOC-LEU-OH (3.00 eq) | HBTU (2.85 eq) and DIEA (6.00 eq) |
| 14 | FMOC-TRP(BOC)-OH (3.00 eq) | HBTU (2.85 eq) and DIEA (6.00 eq) |
| 15 | FMOC-ASN(TRT)-OH (3.00 eq) | HBTU (2.85 eq) and DIEA (6.00 eq) |
| 16 | FMOC-VAL-OH (3.00 eq) | HBTU (2.85 eq) and DIEA (6.00 eq) |
| 17 | FMOC-PHE-OH (3.00 eq) | HBTU (2.85 eq) and DIEA (6.00 eq) |
| 18 | FMOC-ASN(TRT)-OH (3.00 eq) | HBTU (2.85 eq) and DIEA (6.00 eq) |
| 19 | FMOC-AIB-OH (3.00 eq) | HBTU (2.85 eq) and DIEA (6.00 eq) |
| 20 | FMOC-GLN(TRT)-OH (4.00 eq) | HATU (3.80 eq) and DIEA (8.00 eq) |
| 21 | FMOC-ALA-OH (3.00 eq) | HBTU (2.85 eq) and DIEA (6.00 eq) |
| 22 | FMOC-AIB-OH (3.00 eq) | HBTU (2.85 eq) and DIEA (6.00 eq) |
| 23 | FMOC-ARG(PBF)-OH (6.00 eq) | HATU (5.70 eq) and DIEA (12.00 eq) |
| 24 | FMOC-ASP(OTBU)-OH (3.00 eq) | HBTU (2.85 eq) and DIEA (6.00 eq) |
| 25 | FMOC-LEU-OH (3.00 eq) | HBTU (2.85 eq) and DIEA (6.00 eq) |
| 26 | FMOC-AIB-OH (3.00 eq) | HBTU (2.85 eq) and DIEA (6.00 eq) |
| 27 | FMOC-ILE-OH (6.00 eq) | HATU (5.70 eq) and DIEA (12.00 eq) |
| 28 | FMOC-SER(TBU)-OH (3.00 eq) | HBTU (2.85 eq) and DIEA (6.00 eq) |
| 29 | FMOC-TYR(TBU)-OH (3.00 eq) | HBTU (2.85 eq) and DIEA (6.00 eq) |
| 30 | FMOC-ASP(OTBU)-OH (3.00 eq) | HBTU (2.85 eq) and DIEA (6.00 eq) |
| 31 | FMOC-SER(TBU)-OH (3.00 eq) | HATU (2.85 eq) and DIEA (6.00 eq) |
| 32 | FMOC-ILE-OH (3.00 eq) | HATU (2.85 eq) and DIEA (6.00 eq) |
| 33 | FMOC-PHE-OH (3.00 eq) | HATU (2.85 eq) and DIEA (6.00 eq) |
| 34 | FMOC-THR(TBU)-OH (3.00 eq) | HATU (2.85 eq) and DIEA (6.00 eq) |
| 35 | FMOC-GLY-OH (3.00 eq) | HATU (2.85 eq) and DIEA (6.00 eq) |
| 36 | FMOC-GLU(OTBU)-OH (4.00 eq) | HATU (3.80 eq) and DIEA (8.00 eq) |
| 37 | FMOC-AIB-OH (3.00 eq) | HATU (2.85 eq) and DIEA (6.00 eq) |
| 38 | FMOC-N-ME-TYR(TBU)-OH (3.00 eq) | HATU (2.85 eq) and DIEA (6.00 eq) |

5. Coupling: Boc₂O/DIPEA/DMF (10/5/85) 20 mL for 15 mintimes 2, then the resin was washed with DMF (20 mL times 5).

6. Add 3% N₂H₄.H₂O/DMF and react on 20 mins and then repeat it for one more time. Drain and wash with DMF (20 mLtimes5).

7. Repeat step 2 to 3 for the coupling of following amino acids: 01-4)

| # | Materials | Coupling reagents |
|---|---|---|
| 1 | FMOC-AEEA-OH (3.00 eq) | HBTU (2.85 eq) and DIEA (6.00 eq) |
| 2 | FMOC-AEEA-OH (3.00 eq) | HBTU (2.85 eq) and DIEA (6.00 eq) |
| 3 | FMOC-GLU-OTBU (3.00 eq) | HBTU (2.85 eq) and DIEA (6.00 eq) |
| 4 | FMOC-GLU-OTBU (3.00 eq) | HBTU (2.85 eq) and DIEA (6.00 eq) |

8. Coupling: a solution of pentadecanedioic acid (3.00 eq) and HOBt (3.00 eq), DIC (3.00 eq) in DMF (10 mL) was added to the resin and agitated with N₂ for 12 hrs at 20° C. The resin was then washed with DMF (20 mLtimes3).

9. The coupling reaction was monitored by ninhydrin color reaction.

Peptide Cleavage and Purification:

1. After coupling, the resin was washed with DMF for 5 times. After last step, the resin was washed with MeOH for 3 times, and dried under vacuum to get 1.5 g peptide resin. Then the peptide resin was treated with the cleavage cocktail (15 mL, 92.5% TFA/2.5% 3-Mercaptopropionic acid/2.5% TIS/2.5% H₂O) for 2.5 hours. The peptide was concentrated under reduced pressure and precipitated with cold isopropyl ether, filtered and washed two times with isopropyl ether to give 1.2 g residue.

2. The crude peptide was purified by Prep-HPLC (A: 0.075% TFA in H₂O, B: ACN) and then was second purified by Prep-HPLC (A: 0.5% HOAc in H₂O, B: ACN) to give the compound 21 (60.6 mg, 99.13% purity, HOAC) as a white solid which was confirmed by LCMS (Rt=1.533 min) and HPLC (Rt=11.392 min).

Purification Conditions:

First Purification condition

| | |
|---|---|
| Dissolution | Dissolve in 10% ACN-H₂O |
| condition | |
| Instrument | Gilson GX-281 |
| Mobile Phase | A: H₂O (0.075% TFA in H₂O) |
| | B: ACN |
| Gradient | 27-47-60 min. Retention time: 42 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

Second Purification condition

| | |
|---|---|
| Dissolution | Dissolve in 10% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 30-45-60 min. Retention time: 65 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

### Example 6. Synthesis of Selective GIP Receptor Agonist Peptides of the Present Disclosure. Compound No. 48; SEQ ID NO: 43

The peptide compound 48 was synthesized using standard Fmoc chemistry.

1. Resin preparation: the 2-CTC Resin (800 mg, 0.400 mmol, 1.00 eq, Sub 0.50 mmol/g) was added Fmoc-Ser(tBu)-OH (153 mg, 0.400 mmol, 1.00 eq) and DIEA (207 mg, 1.60 mmol, 0.279 mL, 4.00 eq) in DCM (5.00 mL). The mixture was agitated with N₂ for 2 h at 25° C., then added MeOH (0.800 mL) agitated with N₂ for another 30 min. The resin was washed with DMF (30.0 mL times 5). Then 20% piperidine in DMF (30.0 mL) was added and the mixture was agitated with N₂ for 15 min at 25° C. Then the mixture was filtered to get the resin. The resin was washed with DMF (30.0 mL times 5) and filtered to get the resin.

2. Coupling: A solution of Fmoc-Pro-OH (405 mg 1.20 mmol, 3.00 eq), DIEA (310 mg, 2.40 mmol, 0.418 mL, 6.00 eq) and HBTU (432 mg, 1.14 mmol, 2.85 eq) in DMF (5.00 mL) was added to the resin and agitated with N₂ for 30 min at 25° C. The resin was then washed with DMF (30.0 mL times 5).

3. Deprotection: 20% piperidine in DMF (30.0 mL) was added to the resin and the mixture was agitated with N₂ for 15 min at 25° C.

4. Repeat Step 2 and 3 for the coupling of following amino acids: (1-37):

| # | Materials | Coupling reagents |
|---|---|---|
| 1. | Fmoc-Pro-OH (3.00 eq) | HBTU (2.85 eq) and DIEA (6.00 eq) |
| 2. | Fmoc-Pro-OH (3.00 eq) | HBTU (2.85 eq) and DIEA (6.00 eq) |
| 3. | Fmoc-Ala-OH(3.0 eq) | HBTU (2.85 eq) and DIEA (6.00 eq) |
| 4. | Fmoc-Gly-OH (3.00 eq) | HBTU (2.85 eq) and DIEA (6.00 eq) |
| 5. | Fmoc-Ser(tBu)-OH (3.00 eq) | HBTU (2.85 eq) and DIEA (6.00 eq) |
| 6. | Fmoc-Ser(tBu)-OH (3.00 eq) | HBTU (2.85 eq) and DIEA (6.00 eq) |
| 7. | Fmoc-Pro-OH (3.00 eq) | HBTU (2.85 eq) and DIEA (6.00 eq) |
| 8. | Fmoc-Ser(tBu)-OH (3.00 eq) | HBTU (2.85 eq) and DIEA (6.00 eq) |
| 9. | Fmoc-Gln(Trt)-OH (3.00 eq) | HBTU (2.85 eq) and DIEA (6.00 eq) |
| 10. | Fmoc-Ala-OH (3.00 eq) | HBTU (2.85 eq) and DIEA (6.00 eq) |
| 11. | Fmoc-Leu-OH (3.00 eq) | HBTU (2.85 eq) and DIEA (6.00 eq) |
| 12. | Fmoc-Leu-OH (3.00 eq) | HBTU (2.85 eq) and DIEA (6.00 eq) |
| 13. | Fmoc-Trp(Boc)-OH (3.00 eq) | HBTU (2.85 eq) and DIEA (6.00 eq) |
| 14. | Fmoc-Asn(Trt)-OH (3.00 eq) | HBTU (2.85 eq) and DIEA (6.00 eq) |
| 15. | Fmoc-Val-OH (3.00 eq) | HBTU (2.85 eq) and DIEA (6.00 eq) |
| 16. | Fmoc-Phe-OH (3.00 eq) | HBTU (2.85 eq) and DIEA (6.00 eq) |
| 17. | Fmoc-Asn(Trt)-OH (3.00 eq) | HBTU (2.85 eq) and DIEA (6.00 eq) |
| 18. | Fmoc-Aib-OH (3.00 eq) | HBTU (2.85 eq) and DIEA (6.00 eq) |
| 19. | Fmoc-Gln(Trt)-OH (6.00 eq) | HATU (5.70 eq) and DIEA (12.0 eq) |
| 20. | Fmoc-Ala-OH (3.00 eq) | HBTU (2.85 eq) and DIEA (6.00 eq) |
| 21. | Fmoc-Aib-OH (3.00 eq) | HBTU (2.85 eq) and DIEA (6.00 eq) |
| 22. | Fmoc-Arg(pbf)-OH (6.00 eq) | HATU (5.70 eq) and DIEA (12.0 eq) |
| 23. | Fmoc-Asp(OtBu)-OH (3.00 eq) | HBTU (2.85 eq) and DIEA (6.00 eq) |
| 24. | Fmoc-Lys(Dde)-OH (3.00 eq) | HBTU (2.85 eq) and DIEA (6.00 eq) |
| 25. | Fmoc-Ala-OH (3.00 eq) | HBTU (2.85 eq) and DIEA (6.00 eq) |
| 26. | Fmoc-Ile-OH (3.00 eq) | HBTU (2.85 eq) and DIEA (6.00 eq) |
| 27. | Fmoc-Ser(tBu)-OH (3.00 eq) | HBTU (2.85 eq) and DIEA (6.00 eq) |
| 28. | Fmoc-Tyr(tBu)-OH (4.00 eq) | HBTU (3.80 eq) and DIEA (8.00 eq) |
| 29. | Fmoc-Asp(OtBu)-OH (4.00 eq) | HATU (3.80 eq) and DIEA (8.00 eq) |
| 30. | Fmoc-Ser(tBu)-OH (4.00 eq) | HATU (3.80 eq) and DIEA (8.00 eq) |
| 31. | Fmoc-Ile-OH (4.00 eq) | HATU (3.80 eq) and DIEA (8.00 eq) |
| 32. | Fmoc-Phe-OH (4.00 eq) | HATU (3.80 eq) and DIEA (8.00 eq) |
| 33. | Fmoc-Thr(tBu)-OH (4.00 eq) | HATU (3.80 eq) and DIEA (8.00 eq) |
| 34. | Fmoc-Gly-OH (4.00 eq) | HATU (3.80 eq) and DIEA (8.00 eq) |
| 35. | Fmoc-Glu(OtBu)-OH (4.00 eq) | HATU (3.80 eq) and DIEA (8.00 eq) |
| 36. | Fmoc-Aib-OH (4.00 eq) | HATU (3.80 eq) and DIEA (8.00 eq) |
| 37. | Fmoc-N-Me-Tyr(tBu)-OH (3.00 eq) | HATU (2.85 eq) and DIEA (6.00 eq) |

5. Coupling: Boc₂O/DIPEA/DMF (10/5/85) 50.0 mL for 30 min, then the resin was washed with DMF (30.0 mL times 5).

6. Deprotection: Dde was treated with Hydrazine hydrate/DMF (3/97) 50.0 mL for 30 min, then the resin was washed with DMF (30.0 mL times 5).

Repeat Step 2 and 3 for the coupling of following amino acids: (1-3):

| | | |
|---|---|---|
| 1 | Fmoc-Gly-Gly-Gly-OH (3.00 eq) | HBTU (2.85 eq) and DIEA (6.00 eq) |
| 2 | Fmoc-Gly-Gly-OH (3.00 eq) | HBTU (2.85 eq) and DIEA (6.00 eq) |
| 3 | pentadecanedioic | HOBt (4.00 eq) and DIC (4.00 eq) |
| | acid (4.00 eq) | |

Peptide Cleavage and Purification:

1. The resin was washed with MeOH (30 mLtimes3) and dried under vacuum to get 3.00 g peptide resin. Then 30.0 mL of cleavage buffer (92.5% TFA/2.5%₀ 3-Mercaptopropionic acid/2.5% TIS/2.5% H2O) was added to the flask containing the side chain protected peptide resin at 25° C. and the mixture was stirred for 2.5 h. The peptide was precipitated with cold isopropyl ether (200 mL) and centrifuged (3 min at 3000 rpm). Wash the peptide precipitation with tert-butyl methyl ether for two more times (200 mL). Dry the crude peptide over vacuum for 2 h to give the crude peptide (1.70 g).

2. The crude peptide was purified by prep-HPLC (TFA condition; A: 0.075% TFA in H₂O, B: CH₃CN) to give the peptide, then the peptide was purified by prep-HPLC (HOAC condition; A: 0.5% HOAC in H₂O, B: ACN) to give the final product compound 48 (152.7 mg, 8.08% yield, 97.1% purity, HOAC) was obtained as a white solid.

Purification Conditions:

First Purification condition

| | |
|---|---|
| Dissolution | Dissolve in 20% ACN in H₂O |
| condition | |
| Instrument | Gilson GX-281 |
| Mobile Phase | A: H₂O (0.075% TFA in H₂O) |
| | B: ACN |
| Gradient | 25-45-60 min. Retention time: 42.5 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 25-40-50 min. Retention time: 68 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

### Example 7. Synthesis of Selective GIP Receptor Agonist Peptides of the Present Disclosure. Compound No. 14; SEQ ID NO: 15

The peptide compound 14 was synthesized using standard Fmoc chemistry.

1. Resin preparation: To the Rink Amide MBHA resin (0.300 mmol, 1.00 eq, Sub 0.280 mmol/g) in DMF (5.00 mL) was agitated with N₂ for 2 heat 20° C. Then 20% piperidine in DMF (20.0 mL) was added and the mixture was agitated with N₂ for 30 min at 20° C. The resin was washed with DMF (20.0 mL times 5) and filtered to get the resin.

2. Coupling: A solution of FMOC-ARG(PBF)-OH (584 mg, 900 umol, 3.00 eq), DIEA (232 mg, 1.80 mmol, 314 uL, 6.00 eq) and HBTU (324 mg, 855 umol, 2.85 eq) in DMF (2.00 mL) was added to the resin and agitated with N₂ for 30 min at 20° C. The resin was then washed with DMF (20.0 mL times 3).

3. Deprotection: 200 piperidine in DMF (20.0 mL) was added to the resin and the mixture was agitated with N₂ for 30 min at 20° C. The resin was washed with DMF (20.0 mL times 5) and filtered to get the resin.

4. Repeat step 2 to 3 for the coupling of following amino acids: (1-29):

| # | Materials | Coupling reagents |
|---|---|---|
| 1 | FMOC-GLN(TRT)-OH (3.00 eq) | HBTU (2.85 eq) and DIEA (6.00 eq) |
| 2 | FMOC-ALA-OH (3.00 eq) | HBTU (2.85 eq) and DIEA (6.00 eq) |
| 3 | FMOC- LEU -OH (3.00 eq) | HBTU (2.85 eq) and DIEA (6.00 eq) |
| 4 | FMOC-LEU-OH (3.00 eq) | HBTU (2.85 eq) and DIEA (6.00 eq) |
| 5 | FMOC-TRP(BOC)-OH (3.00 eq) | HBTU (2.85 eq) and DIEA (6.00 eq) |
| 6 | FMOC-ASN(TRT)-OH (3.00 eq) | HBTU (2.85 eq) and DIEA (6.00 eq) |
| 7 | FMOC-VAL-OH (3.00 eq) | HBTU (2.85 eq) and DIEA (6.00 eq) |
| 8 | FMOC-PHE-OH (3.00 eq) | HBTU (2.85 eq) and DIEA (6.00 eq) |
| 9 | FMOC-LYS(DDE)-OH (3.00 eq) | HBTU (2.85 eq) and DIEA (6.00 eq) |
| 10 | FMOC-AIB-OH (3.00 eq) | HBTU (2.85 eq) and DIEA (6.00 eq) |
| 11 | FMOC-GLN(TRT)-OH (4.00 eq) | HATU (3.80 eq) and DIEA (8.00 eq) |
| 12 | FMOC-ALA-OH (3.00 eq) | HBTU (2.85 eq) and DIEA (6.00 eq) |
| 13 | FMOC-AIB-OH (3.00 eq) | HBTU (2.85 eq) and DIEA (6.00 eq) |
| 14 | FMOC-ARG(PBF)-OH (4.00 eq) | HATU (3.80 eq) and DIEA (8.00 eq) |
| 15 | FMOC-ASP(OTBU)-OH (3.00 eq) | HBTU (2.85 eq) and DIEA (6.00 eq) |
| 16 | FMOC-LEU-OH (3.00 eq) | HBTU (2.85 eq) and DIEA (6.00 eq) |
| 17 | FMOC-ALA-OH (3.00 eq) | HBTU (2.85 eq) and DIEA (6.00 eq) |
| 18 | FMOC-ILE-OH (3.00 eq) | HBTU (2.85 eq) and DIEA (6.00 eq) |
| 19 | FMOC-SER(TBU)-OH (3.00 eq) | HBTU (2.85 eq) and DIEA (6.00 eq) |
| 20 | FMOC-TYR(TBU)-OH (3.00 eq) | HATU (2.85 eq) and DIEA (6.00 eq) |
| 21 | FMOC-ASP(OTBU)-OH (4.00 eq) | HATU (3.80 eq) and DIEA (8.00 eq) |
| 22 | FMOC-SER(TBU)-OH (4.00 eq) | HATU (3.80 eq) and DIEA (8.00 eq) |
| 23 | FMOC-ILE-OH (4.00 eq) | HATU (3.80 eq) and DIEA (8.00 eq) |
| 24 | FMOC-PHE-OH (4.00 eq) | HATU (3.80 eq) and DIEA (8.00 eq) |
| 25 | FMOC-THR(TBU)-OH (4.00 eq) | HATU (3.80 eq) and DIEA (8.00 eq) |
| 26 | FMOC-GLY-OH (4.00 eq) | HATU (3.80 eq) and DIEA (8.00 eq) |
| 27 | FMOC-GLU(OTBU)-OH (4.00 eq) | HATU (3.80 eq) and DIEA (8.00 eq) |
| 28 | FMOC-AIB-OH (4.00 eq) | HATU (3.80 eq) and DIEA (8.00 eq) |
| 29 | FMOC-N-ME-TYR(TBU)-OH (3.00 eq) | HATU (2.85 eq) and DIEA (6.00 eq) |

5. Coupling: Boc₂O/DIPEA/DMF (10/5/85) 20.0 mL for 30 min, then the resin was washed with DMF (20.0 mL times 5).

6. Deprotection: Dde was treated with Hydrazine hydrate/DMF (3/97) 20.0 mL for 30 min, then the resin was washed with DMF (30.0 mL times 5).

7. Repeat step 2 to 3 for the coupling of following amino acids: (1-5):

| | | |
|---|---|---|
| 1 | FMOC-AEEA-OH (3.00 eq) | HBTU (2.85 eq) and DIEA (6.00 eq) |
| 2 | FMOC-AEEA-OH (3.00 eq) | HBTU (2.85 eq) and DIEA (6.00 eq) |
| 3 | FMOC-GLU-OTBU (3.00 eq) | HBTU (2.85 eq) and DIEA (6.00 eq) |
| 4 | FMOC-GLU-OTBU (3.00 eq) | HBTU (2.85 eq) and DIEA (6.00 eq) |
| 5 | pentadecanedioic | HOBT (4.00 eq) and DIC (4.00 eq) |
| | acid (4.00 eq) | |

Peptide Cleavage and Purification:

1. The resin was washed with MeOH (30 mLtimes2) and dried under vacuum to get 3.5 g peptide resin. Then 30 mL of cleavage buffer (92.5% TFA/2.5%₀ 3-Mercaptopropionic acid/2.5% TIS/2.5% H2O) was added to the flask containing the side chainprotected peptide resin at 20° C. and the mixture was stirred for 2 h. The peptide was precipitated with cold tert-butyl methylether (250 mL) and centrifuged (3 min at 3000 rpm). Wash the peptide precipitation with tert-butyl methyl ether for two more times (120 mL). Dry the crude peptide (1.4 g) over vacuum for 2 h.

2. The residue was purified by prep-HPLC (TFA condition; 30° C., A: 0.07500 TFA/1H2O, B: CH3CN) and then was second purified by prep-HPLC (HOAc condition; 30° C., A: 0.5% HOAc/H2O, B: CH3CN) to give the product to give the compound 14 (79.8 mg, 6.22% yield, 96.4% purity, HOAC) as a white solid.

Purification Conditions:

First Purification condition

| | |
|---|---|
| Dissolution | Dissolve in 20% TFA, 10% CH₃CN and 70% H₂O |
| condition | |
| Instrument | Gilson GX-281 |
| Mobile Phase | A: H₂O (0.075% TFA in H₂O) |
| | B: ACN |
| Gradient | 24-44-60 min. Retention time: 43 min |
| Column | luna, c18, 10 um, 100 A + Gemini, 5 um, c18, 110 A |
| Flow Rate | 20 mL/Min |
| Wavelength | 214/254 nm |
| Oven Tem. | 20° C. |

Second Purification condition

| | |
|---|---|
| Dissolution | The liquid is directly Purification |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 28-38-60 min. Retention time: 52 min |
| Column | luna, c18, 10 um, 100 A + Gemini, 5 um, c18, 110 A |
| Flow Rate | 20 mL/Min |
| Wavelength | 214/254 nm |
| Oven Tem. | Room temperature |

Table 3 below lists exemplary GIP receptor agonist peptides made according to methods described in Example 1-7.

TABLE 3
Exemplary GIP receptor agonist peptides made according to methods described in Examples 1-7

| CPMD No. | LINKER | LIPID | N_TER | C_TER | 1 | 2 | 3 | 4 | 5 | 6 | 7 | 8 | 9 | 10 | 11 | 12 |
|---|---|---|---|---|---|---|---|---|---|---|---|---|---|---|---|---|
| 1 | 2OEGgE | C16DA | M [symbol] | | Y | Aib | E | G | T | F | I | S | D | Y | S | I |
| 2 | 2OEGgE | C16DA | M [symbol] | | Y | Aib | E | G | T | F | I | S | D | Y | S | I |
| 3 | 2OEGgE | C16DA | M [symbol] | | Y | Aib | E | G | T | F | I | S | D | Y | S | I |
| 4 | 2OEGgE | C16DA | M [symbol] | | Y | Aib | E | G | T | F | I | S | D | Y | S | I |
| 5 | 2OEGgE | C16DA | M [symbol] | | Y | G | E | G | T | F | I | S | D | Y | S | I |
| 6 | 2OEGgE | C16DA | M [symbol] | | Y | S | E | G | T | F | I | S | D | Y | S | I |
| 7 | 2OEGgE | C16DA | M [symbol] | | Y | Aib | E | G | T | F | I | S | D | Y | S | I |
| 8 | 2OEGgE | C16DA | M [symbol] | | Y | Aib | E | G | T | F | I | S | D | Y | S | I |
| 9 | 2OEGgE | C16DA | M [symbol] | | Y | Aib | E | G | T | F | I | S | D | Y | S | I |
| 10 | 2OEGgE | C16DA | M [symbol] | | Y | Aib | E | G | T | F | I | S | D | Y | S | I |
| 11 | 2OEGgE | C16DA | M [symbol] | | Y | Aib | E | G | T | F | I | S | D | Y | S | I |
| 12 | 2OEGgE | C15DA | M [symbol] | OH | Y | Aib | E | G | T | F | I | S | D | Y | S | I |
| 13 | 2OEGgEgE | C15DA | M [symbol] | NH2 | Y | Aib | E | G | T | F | I | S | D | Y | S | I |
| 14 | 2OEGgEgE | C15DA | M [symbol] | OH | Y | Aib | E | G | T | F | I | S | D | Y | S | I |
| 15 | 2OEGgEgE | C15DA | M [symbol] | OH | Y | Aib | E | G | T | F | I | S | D | Y | S | I |
| 16 | 2OEGgEgE | C15DA | M [symbol] | NH2 | Y | Aib | E | G | T | F | I | S | D | Y | S | I |
| 17 | 2OEGgEgE | C15DA | M [symbol] | NH2 | Y | Aib | E | G | T | F | I | S | D | Y | S | I |
| 18 | gEgEgE | C15DA | M [symbol] | NH2 | Y | Aib | E | G | T | F | I | S | D | Y | S | I |
| 19 | 2OEGgEgE | C15DA | M [symbol] | NH2 | Y | Aib | E | G | T | F | I | S | D | Y | S | I |
| 20 | 2OEGgEgE | C15DA | M [symbol] | NH2 | Y | Aib | E | G | T | F | I | S | D | Y | S | I |
| 21 | 2OEGgEgE | C15DA | M [symbol] | NH2 | Y | Aib | E | G | T | F | I | S | D | Y | S | I |
| 22 | 2OEGgEgE | C15DA | M [symbol] | NH2 | Y | Aib | E | G | T | F | I | S | D | Y | S | I |
| 23 | OEGgEgEgE | C15DA | M [symbol] | NH2 | Y | Aib | E | G | T | F | I | S | D | Y | S | I |
| 24 | OEGgEgEgE | C15DA | M [symbol] | NH2 | Y | Aib | E | G | T | F | I | S | D | Y | S | I |
| 25 | 2OEGgEgE | C15DA | M [symbol] | OH | Y | Aib | E | G | T | F | I | S | D | Y | S | I |
| 26 | 2OEGgEgE | C15DA | M [symbol] | OH | Y | Aib | E | G | T | F | I | S | D | Y | S | I |
| 27 | 2OEGgEgE | C15DA | M [symbol] | OH | Y | Aib | E | G | T | F | I | S | D | Y | S | I |
| 28 | OEGgEgEgE | C15DA | M [symbol] | NH2 | Y | Aib | E | G | T | F | I | S | D | Y | S | I |
| 29 | OEGgEgEgE | C15DA | M [symbol] | NH2 | Y | Aib | E | G | T | F | I | S | D | Y | S | I |
| 30 | G2E3 | C15DA | M [symbol] | NH2 | Y | Aib | E | G | T | F | I | S | D | Y | S | I |
| 31 | G2E3 | C15DA | M [symbol] | NH2 | Y | Aib | E | G | T | F | I | S | D | Y | S | I |
| 32 | G2E3 | C15DA | M [symbol] | OH | Y | Aib | E | G | T | F | I | S | D | Y | S | I |
| 33 | 2OEGgE | C15DA | M [symbol] | OH | Y | Aib | E | G | T | F | I | S | D | Y | S | I |
| 34 | 2OEGgE | C15DA | M [symbol] | OH | Y | Aib | E | G | T | F | I | S | D | Y | S | I |
| 35 | 2OEGgE | C15DA | M [symbol] | OH | Y | Aib | E | G | T | F | I | S | D | Y | S | I |
| 36 | 2OEGgE | C15DA | M [symbol] | OH | Y | Aib | E | G | T | F | I | S | D | Y | S | I |
| 37 | 2OEGgE | C15DA | M [symbol] | OH | Y | Aib | E | G | T | F | I | S | D | Y | S | I |
| 38 | OEGgEgEgE | C15DA | M [symbol] | OH | Y | Aib | E | G | T | F | I | S | D | Y | S | I |
| 39 | OEGgEgEgE | C15DA | M [symbol] | OH | Y | Aib | E | G | T | F | I | S | D | Y | S | I |
| 40 | OEGgEgEgE | C15DA | M [symbol] | OH | Y | Aib | E | G | T | F | I | S | D | Y | S | I |
| 41 | G2E3 | C15DA | M [symbol] | NH2 | Y | Aib | E | G | T | F | I | S | D | Y | S | I |
| 42 | G2E3 | C15DA | M [symbol] | OH | Y | Aib | E | G | T | F | I | S | D | Y | S | I |
| 43 | OEGgEgEgE | C15DA | M [symbol] | NH2 | Y | Aib | E | G | T | F | I | S | D | Y | S | I |
| 44 | OEGgEgEgE | C15DA | M [symbol] | OH | Y | Aib | E | G | T | F | I | S | D | Y | S | I |
| 45 | OEGgEgEgE | C15DA | M [symbol] | OH | Y | Aib | E | G | T | F | I | S | D | Y | S | I |
| 46 | 2OEGgE | C15DA | M [symbol] | OH | Y | Aib | E | G | T | F | I | S | D | Y | S | I |
| 47 | 2OEGgE | C15DA | M [symbol] | OH | Y | Aib | E | G | T | F | I | S | D | Y | S | I |
| 48 | GS | C15DA | M [symbol] | OH | Y | Aib | E | G | T | F | I | S | D | Y | S | I |
| 49 | G2E3 | C15DA | M [symbol] | OH | Y | Aib | E | G | T | F | I | S | D | Y | S | I |
| 50 | [symbol] E3 | C15DA | M [symbol] | OH | Y | Aib | E | G | T | F | I | S | D | Y | S | I |
| 51 | [symbol] E3 | C15DA | M [symbol] | OH | Y | Aib | E | G | T | F | I | S | D | Y | S | I |
| 52 | OEGgEgEgE | C15DA | M [symbol] | OH | Y | Aib | E | G | T | F | I | S | D | Y | S | I |
| 53 | G2E3 | C15DA | M [symbol] | OH | Y | Aib | E | G | T | F | I | S | D | Y | S | I |
| 54 | OEGgEgEgE | C15DA | M [symbol] | OH | Y | Aib | E | G | T | F | I | S | D | Y | S | I |
| 55 | G2E3 | C15DA | M [symbol] | OH | Y | Aib | E | G | T | F | I | S | D | Y | S | I |
| 56 | [symbol] | C15DA | M [symbol] | OH | Y | Aib | E | G | T | F | I | S | D | Y | S | I |
| 57 | [symbol] | C15DA | M [symbol] | OH | Y | Aib | E | G | T | F | I | S | D | Y | S | I |
| 58 | [symbol] gEgE | C15DA | M [symbol] | NH2 | Y | Aib | E | G | T | F | I | S | D | Y | S | I |
| 59 | [symbol] gEgE | C15DA | M [symbol] | NH2 | Y | Aib | E | G | T | F | I | S | D | Y | S | I |
| 60 | [symbol] gE | C15DA | M [symbol] | NH2 | Y | Aib | E | G | T | F | I | S | D | Y | S | I |
| 61 | [symbol] E | C15DA | M [symbol] | NH2 | Y | Aib | E | G | T | F | I | S | D | Y | S | I |
| 62 | [symbol] gE | C15DA | M [symbol] | OH | Y | Aib | E | G | T | F | I | S | D | Y | S | I |
| 63 | [symbol] gEgE | C15DA | M [symbol] | NH2 | Y | Aib | E | G | T | F | I | S | D | Y | S | I |
| 64 | [symbol] gEgE | C15DA | M [symbol] | NH2 | Y | Aib | E | G | T | F | I | S | D | Y | S | I |
| 65 | [symbol] gE | C15DA | M [symbol] | NH2 | Y | Aib | E | G | T | F | I | S | D | Y | S | I |
| 66 | [symbol] gEgE | C15DA | M [symbol] | NH2 | Y | Aib | E | G | T | F | I | S | D | Y | S | I |
| 67 | [symbol] gE | C15DA | M [symbol] | NH2 | Y | Aib | E | G | T | F | I | S | D | Y | S | I |
| 68 | GS | C15DA | M [symbol] | NH2 | Y | Aib | E | G | T | F | I | S | D | Y | S | I |
| 69 | [symbol] gE | C15DA | M [symbol] | NH2 | Y | Aib | E | G | T | F | I | S | D | Y | S | I |
| 70 | OEGgEgE | C15DA | M [symbol] | NH2 | Y | Aib | E | G | T | F | I | S | D | Y | S | I |
| 71 | G5gE | C15DA | M [symbol] | NH2 | Y | Aib | E | G | T | F | I | S | D | Y | S | I |
| 72 | [symbol] gE | C15DA | M [symbol] | NH2 | Y | Aib | E | G | T | F | I | S | D | Y | S | I |
| 73 | G5gE | C15DA | M [symbol] | NH2 | Y | Aib | E | G | T | F | I | S | D | Y | S | I |
| 74 | G5gE | C15DA | M [symbol] | NH2 | Y | Aib | E | G | T | F | I | S | D | Y | S | I |
| 75 | 2OEGgE | C15DA | M [symbol] | NH2 | Y | Aib | E | G | T | F | I | S | D | Y | S | I |
| 76 | 2OEGgE | C15DA | M [symbol] | NH2 | Y | Aib | E | G | T | F | I | S | D | Y | S | I |
| 77 | [symbol] gEgE | C15DA | M [symbol] | NH2 | Y | Aib | E | G | T | F | I | S | D | Y | S | I |
| 78 | G4gE | C15DA | M [symbol] | NH2 | Y | Aib | E | G | T | F | I | S | D | Y | S | I |
| 79 | G5gE | C15DA | M [symbol] | NH2 | Y | Aib | E | G | T | F | I | S | D | Y | S | I |
| 80 | G5gE | C15DA | M [symbol] | NH2 | Y | Aib | E | G | T | F | I | S | D | Y | S | I |
| 81 | G5gEgE | C15DA | M [symbol] | NH2 | Y | Aib | E | G | T | F | I | S | D | Y | S | I |
| 82 | G5gE | C15DA | M [symbol] | NH2 | Y | Aib | E | G | T | F | I | S | D | Y | S | I |
| 83 | [symbol] gEgE | C15DA | M [symbol] | NH2 | Y | Aib | E | G | T | F | I | S | D | Y | S | I |
| 84 | [symbol] gEgE | C15DA | M [symbol] | NH2 | Y | Aib | E | G | T | F | I | S | D | Y | S | I |
| 85 | [symbol] gEgE | C15DA | M [symbol] | NH2 | Y | Aib | E | G | T | F | I | S | D | Y | S | I |
| 86 | OEGgEgE | C15DA | M [symbol] | NH2 | Y | Aib | E | G | T | F | I | S | D | Y | S | I |
| 87 | [symbol] GgE | C15DA | M [symbol] | NH2 | Y | Aib | E | G | T | F | I | S | D | Y | S | I |
| 88 | 2OEGgEgE | C15DA | M [symbol] | NH2 | Y | Aib | E | G | T | F | I | S | D | Y | S | I |
| 89 | [symbol] gEgE | C15DA | M [symbol] | NH2 | Y | Aib | E | G | T | F | I | S | D | Y | S | I |
| 90 | [symbol] gEgE | C15DA | M [symbol] | NH2 | Y | Aib | E | G | T | F | I | S | D | Y | S | I |
| 91 | 2OEGgEgE | C15DA | M [symbol] | NH2 | Y | Aib | E | G | T | F | I | S | D | Y | S | I |
| 92 | 2OEGgE | C15DA | M [symbol] | NH2 | Y | Aib | E | G | T | F | I | S | D | Y | S | I |
| 93 | 2OEGgEgE | C15DA | M [symbol] | NH2 | Y | Aib | E | G | T | F | I | S | D | Y | S | I |
| 94 | OEGgEgE | C15DA | M [symbol] | NH2 | Y | Aib | E | G | T | F | I | S | D | Y | S | I |
| 95 | GGEEE | C15DA | M [symbol] | NH2 | Y | Aib | E | G | T | F | I | S | D | Y | S | I |
| 96 | 2OEGgE | C15DA | M [symbol] | NH2 | Y | Aib | E | G | T | F | I | S | D | Y | S | I |
| 97 | G5gE | C15DA | M [symbol] | NH2 | Y | Aib | E | G | T | F | I | S | D | Y | S | I |
| 98 | 2OEGgE | C15DA | M [symbol] | NH2 | Y | Aib | E | G | T | F | I | S | D | Y | S | I |
| 99 | GGEEE | C15DA | M [symbol] | NH2 | Y | Aib | E | G | T | F | I | S | D | Y | S | I |
| 100 | 2OEGgE | C15DA | M [symbol] | NH2 | Y | Aib | E | G | T | F | I | S | D | Y | S | I |
| 101 | 2OEGgEgE | C15DA | M [symbol] | NH2 | Y | Aib | E | G | T | F | I | S | D | Y | S | I |
| 102 | 2OEGgE | C15DA | M [symbol] | NH2 | Y | Aib | E | G | T | F | I | S | D | Y | S | I |
| 103 | 2OEGgEgE | C15DA | M [symbol] | NH2 | Y | Aib | E | G | T | F | I | S | D | Y | S | I |
| 104 | 2OEGgE | C15DA | M [symbol] | NH2 | Y | Aib | E | G | T | F | I | S | D | Y | S | I |
| 105 | GGEEE | C15DA | M [symbol] | NH2 | Y | Aib | E | G | T | F | I | S | D | Y | S | I |
| 106 | [symbol] PAPAP | C15DA | M [symbol] | NH2 | Y | Aib | E | G | T | F | I | S | D | Y | S | I |
| 107 | [symbol] PAPAP | C15DA | M [symbol] | NH2 | Y | Aib | E | G | T | F | I | S | D | Y | S | I |
| 108 | 2OEGgEgE | C15DA | M [symbol] | NH2 | Y | Aib | E | G | T | F | I | S | D | Y | S | I |
| 109 | 2OEGgE | C15DA | M [symbol] | NH2 | Y | Aib | E | G | T | F | I | S | D | Y | S | I |
| 110 | G5gE | C15DA | M [symbol] | NH2 | Y | Aib | E | G | T | F | I | S | D | Y | S | I |
| 111 | 2OEGgEgE | C15DA | M [symbol] | NH2 | Y | Aib | E | G | T | F | I | S | D | Y | S | I |
| 112 | OEGgEgEgE | C15DA | M [symbol] | NH2 | Y | Aib | E | G | T | F | I | S | D | Y | S | I |
| 113 | GG-EEE | C15DA | M [symbol] | NH2 | Y | Aib | E | G | T | F | I | S | D | Y | S | I |
| 114 | 2OEGgEgE | C15DA | M [symbol] | NH2 | Y | Aib | E | G | T | F | I | S | D | Y | S | I |
| 115 | OEGgEgEgE | C15DA | M [symbol] | NH2 | Y | Aib | E | G | T | F | I | S | D | Y | S | I |
| 116 | 2OEGgE | C15DA | M [symbol] | NH2 | Y | Aib | E | G | T | F | I | S | D | Y | S | I |
| 117 | 2OEGgEgE | C15DA | M [symbol] | NH2 | Y | Aib | E | G | T | F | I | S | D | Y | S | I |
| 118 | 2OEGgE | C15DA | M [symbol] | NH2 | Y | Aib | E | G | T | F | I | S | D | Y | S | I |
| 119 | 2OEGgEgE | C15DA | M [symbol] | NH2 | Y | Aib | E | G | T | F | I | S | D | Y | S | I |
| 120 | 2OEGgEgE | C15DA | M [symbol] | NH2 | Y | Aib | E | G | T | F | I | S | D | Y | S | I |
| 121 | 2OEGgEgE | C15DA | M [symbol] | NH2 | Y | Aib | E | G | T | F | I | S | D | Y | S | I |
| 122 | 2OEGgEgE | C15DA | M [symbol] | NH2 | Y | Aib | E | G | T | F | I | S | D | Y | S | I |
| 123 | 2OEGgEgE | C15DA | M [symbol] | NH2 | Y | Aib | E | G | T | F | I | S | D | Y | S | I |
| 124 | GS | C15DA | M [symbol] | NH2 | Y | Aib | E | G | T | F | I | S | D | Y | S | I |
| 125 | G5gE | C15DA | M [symbol] | NH2 | Y | Aib | E | G | T | F | I | S | D | Y | S | I |
| 126 | G5gE | C15DA | M [symbol] | NH2 | Y | Aib | E | G | T | F | I | S | D | Y | S | I |
| 127 | 2OEGgE | C15DA | M [symbol] | NH2 | Y | Aib | E | G | T | F | I | S | D | Y | S | I |
| 128 | 2OEGgE | C15DA | M [symbol] | NH2 | Y | Aib | E | G | T | F | I | S | D | Y | S | I |
| 129 | 2OEGgE | C15DA | M [symbol] | NH2 | Y | Aib | E | G | T | F | I | S | D | Y | S | I |
| 130 | 2OEGgEgE | C15DA | M [symbol] | NH2 | Y | Aib | E | G | T | F | I | S | D | Y | S | I |
| 131 | G5gE | C15DA | M [symbol] | NH2 | Y | Aib | E | G | T | F | I | S | D | Y | S | I |
| 132 | G5gE | C15DA | M [symbol] | NH2 | Y | Aib | E | G | T | F | I | S | D | Y | S | I |
| 133 | 2OEGgEgE | C15DA | M [symbol] | NH2 | Y | Aib | E | G | T | F | I | S | D | Y | S | I |
| 134 | 2OEGgEgE | C15DA | M [symbol] | NH2 | Y | Aib | E | G | T | F | I | S | D | Y | S | I |
| 135 | 2OEGgEgE | C15DA | M [symbol] | NH2 | Y | Aib | E | G | T | F | I | S | D | Y | S | I |
| 136 | 2OEGgEgE | C15DA | M [symbol] | NH2 | Y | Aib | E | G | T | F | I | S | D | Y | S | I |
| 137 | G5gE | C15DA | M [symbol] | NH2 | Y | Aib | E | G | T | F | I | S | D | Y | S | I |
| 138 | G2E3 | C15DA | M [symbol] | NH2 | Y | Aib | E | G | T | F | I | S | D | Y | S | I |
| 139 | 2OEGgEgE | C15DA | M [symbol] | NH2 | Y | Aib | E | G | T | F | I | S | D | Y | S | I |
| 140 | GS | C15DA | M [symbol] | NH2 | Y | Aib | E | G | T | F | I | S | D | Y | S | I |
| 141 | GS | C15DA | M [symbol] | OH | Y | Aib | E | G | T | F | I | S | D | Y | S | I |
| 142 | GS | C15DA | M [symbol] | OH | Y | Aib | E | G | T | F | I | S | D | Y | S | I |
| 143 | 2OEGgEgE | C15DA | M [symbol] | OH | Y | Aib | E | G | T | F | I | S | D | Y | S | I |
| 144 | 2OEGgEgE | C15DA | M [symbol] | NH2 | Y | Aib | E | G | T | F | I | S | D | Y | S | I |
| 145 | 2OEGgEgE | C15DA | M [symbol] | NH2 | Y | Aib | E | G | T | F | I | S | D | Y | S | I |
| 146 | GS | C15DA | M [symbol] | OH | Y | Aib | E | G | T | F | I | S | D | Y | S | I |
| 147 | GS | C15DA | M [symbol] | OH | Y | Aib | E | G | T | F | I | S | D | Y | S | I |
| 148 | 2OEGgEgE | C15DA | M [symbol] | OH | Y | Aib | E | G | T | F | I | S | D | Y | S | I |
| 149 | 2OEGgEgE | C15DA | M [symbol] | OH | Y | Aib | E | G | T | F | I | S | D | Y | S | I |
| 150 | GS | C15DA | M [symbol] | OH | Y | Aib | E | G | T | F | I | S | D | Y | S | I |
| 151 | GS | C15DA | M [symbol] | NH2 | Y | Aib | E | G | T | F | I | S | D | Y | S | I |
| 152 | GS | C15DA | M [symbol] | OH | Y | Aib | E | G | T | F | I | S | D | Y | S | I |
| 153 | GS | C15DA | M [symbol] | NH2 | Y | Aib | E | G | T | F | I | S | D | Y | S | I |
| 154 | GS | C15DA | M [symbol] | NH2 | Y | Aib | E | G | T | F | I | S | D | Y | S | I |
| 155 | GS | C15DA | M [symbol] | OH | Y | Aib | E | G | T | F | I | S | D | Y | S | I |
| 156 | GS | C15DA | M [symbol] | OH | Y | Aib | E | G | T | F | I | S | D | Y | S | I |
| 157 | GS | C15DA | M [symbol] | OH | Y | Aib | E | G | T | F | I | S | D | Y | S | I |
| 158 | GS | C15DA | M [symbol] | OH | Y | Aib | E | G | T | F | I | S | D | Y | S | I |
| 159 | GS | C15DA | M [symbol] | OH | Y | Aib | E | G | T | F | I | S | D | Y | S | I |
| 160 | GS | C15DA | M [symbol] | OH | Y | Aib | E | G | T | F | I | S | D | Y | S | I |
| 161 | 2OEGgEgE | C15DA | M [symbol] | OH | Y | Aib | E | G | T | F | I | S | D | Y | S | I |
| 162 | 2OEGgEgE | C15DA | M [symbol] | OH | Y | Aib | E | G | T | F | I | S | D | Y | S | I |
| 163 | 2OEGgEgE | C15DA | M [symbol] | OH | Y | Aib | E | G | T | F | I | S | D | Y | S | I |
| 164 | GS | C15DA | M [symbol] | NH2 | Y | Aib | E | G | T | F | I | S | D | Y | S | I |
| 165 | GS | C15DA | M [symbol] | OH | Y | Aib | E | G | T | F | I | S | D | Y | S | I |
| 166 | GS | C15DA | M [symbol] | OH | Y | Aib | E | G | T | F | I | S | D | Y | S | I |
| 167 | GS | C15DA | M [symbol] | NH2 | Y | Aib | E | G | T | F | I | S | D | Y | S | I |
| 168 | GS | C15DA | M [symbol] | OH | Y | Aib | E | G | T | F | I | S | D | Y | S | I |
| 169 | 2OEGgEgE | C15DA | M [symbol] | OH | Y | Aib | E | G | T | F | I | S | D | Y | S | I |
| 170 | 2OEGgEgE | C15DA | M [symbol] | OH | Y | Aib | E | G | T | F | I | S | D | Y | S | I |
| 171 | 2OEGgEgE | C15DA | M [symbol] | OH | Y | Aib | E | G | T | F | I | S | D | Y | S | I |
| 172 | GS | C15DA | M [symbol] | OH | Y | Aib | E | G | T | F | I | S | D | Y | S | I |
| 173 | GS | C15DA | M [symbol] | OH | Y | Aib | E | G | T | F | I | S | D | Y | S | I |
| 174 | GS | C15DA | M [symbol] | OH | Y | Aib | E | G | T | F | I | S | D | Y | S | I |
| 175 | 2OEGgEgE | C15DA | M [symbol] | OH | Y | Aib | E | G | T | F | I | S | D | Y | S | I |
| 176 | GS | C15DA | M [symbol] | OH | Y | Aib | E | G | T | F | I | S | D | Y | S | I |
| 177 | GS | C15DA | M [symbol] | OH | Y | Aib | E | G | T | F | I | S | D | Y | S | I |
| 178 | GS | C15DA | M [symbol] | OH | Y | Aib | E | G | T | F | I | S | D | Y | S | I |
| 179 | GS | C15DA | M [symbol] | OH | Y | Aib | E | G | T | F | I | S | D | Y | S | I |
| 180 | G4gE | C15DA | M [symbol] | OH | Y | Aib | E | G | T | F | I | S | D | Y | S | I |
| 181 | GS | C15DA | M [symbol] | OH | Y | Aib | E | G | T | F | I | S | D | Y | S | I |
| 182 | GS | C15DA | M [symbol] | OH | Y | Aib | E | G | T | F | I | S | D | Y | S | I |
| 183 | 2OEGgEgE | C15DA | M [symbol] | OH | Y | Aib | E | G | T | F | I | S | D | Y | S | I |
| 184 | 2OEGgEgE | C15DA | M [symbol] | OH | Y | Aib | E | G | T | F | I | S | D | Y | S | I |
| 185 | 2OEGgEgE | C15DA | M [symbol] | OH | Y | Aib | E | G | T | F | I | S | D | Y | S | I |
| 186 | GS | C15DA | M [symbol] | OH | Y | Aib | E | G | T | F | I | S | D | Y | S | I |
| 187 | GS | C15DA | M [symbol] | OH | Y | Aib | E | G | T | F | I | S | D | Y | S | I |
| 188 | 2OEGgEgE | C15DA | | OH | Y | Aib | E | G | T | F | I | S | D | Y | S | I |
| 189 | GS | C15DA | | OH | Y | Aib | E | G | T | F | I | S | D | Y | S | I |
| 190 | gE | C16 | M [symbol] | NH2 | Y | Aib | E | G | T | F | I | S | D | Y | S | I |
| 191 | | C16 | | NH2 | Y | Aib | E | G | T | F | I | S | D | Y | S | I |
| 192 | 2OEGgE | C16DA | | | Y | G | E | G | T | F | I | S | D | Y | S | I |
| 193 | PEG [symbol] gE | C16DA | | | Y | G | E | G | T | F | I | S | D | Y | S | I |
| 194 | PEG [symbol] gE | C16DA | | | Y | G | E | G | T | F | I | S | D | Y | S | I |
| 195 | PEG [symbol] gE | C16DA | | | Y | G | E | G | T | F | I | S | D | Y | S | I |
| 196 | 2OEGgE | C16DA | M [symbol] | | Y | Aib | E | G | T | F | I | S | D | Y | S | I |
| 197 | gEgEgE | C15DA | M [symbol] | OH | Y | Aib | E | G | T | F | I | S | D | Y | S | I |
| 198 | 2OEGgEgE | C15DA | M [symbol] | OH | Y | Aib | E | G | T | F | I | S | D | Y | S | I |
| 199 | GS | C15DA | M [symbol] | OH | Y | Aib | E | G | T | F | I | S | D | Y | S | I |
| 200 | 2OEGgEgE | C15DA | M [symbol] | NH2 | Y | Aib | E | G | T | F | I | S | D | Y | S | I |
| 201 | 2OEGgEgEgE | C15DA | M [symbol] | OH | Y | Aib | E | G | T | F | I | S | D | Y | S | I |
| 202 | 2OEGgEgE | C16 ( [symbol] ) | M [symbol] | OH | Y | Aib | E | G | T | F | I | S | D | Y | S | I |
| 203 | 2OEGgEgE | C16 ( [symbol] ) | M [symbol] | OH | Y | Aib | E | G | T | F | I | S | D | Y | S | I |
| 204 | 2OEGgEgE | C15DA | M [symbol] | OH | Y | Aib | E | G | T | F | I | S | D | Y | S | I |
| 205 | 2OEGgEgE | C15DA | M [symbol] | NH2 | Y | Aib | E | G | T | F | I | S | D | Y | S | I |
| 206 | 2OEGgEgE | C15DA | M [symbol] | OH | Y | G | E | G | T | F | I | S | D | Y | S | I |
| 207 | 2OEGgEgE | C15DA | M [symbol] | OH | Y | Aib | E | G | T | F | I | S | D | Y | S | I |
| 208 | 2OEGgEgE | C15DA | M [symbol] | OH | Y | Aib | E | G | T | F | I | S | D | Y | S | I |
| 209 | 2OEGgEgE | C15DA | M [symbol] | NH2 | Y | Aib | E | G | T | F | I | S | D | Y | S | I |
| 210 | 2OEGgEgE | C15DA | M [symbol] | NH2 | Y | Aib | E | G | T | F | I | S | D | Y | S | I |
| 211 | 2OEGgEgE | C15DA | M [symbol] | OH | Y | Aib | E | G | T | F | I | S | D | Y | S | I |
| 212 | OEGgEgEgE | C15DA | M [symbol] | OH | Y | Aib | E | G | T | F | I | S | D | Y | S | I |
| 213 | 2OEGgE | C15DA | M [symbol] | OH | Y | Aib | E | G | T | F | I | S | D | Y | S | I |
| 214 | 2OEGgEgE | C15DA | M [symbol] | OH | Y | Aib | E | G | T | F | I | S | D | Y | S | I |
| 215 | GSgEgE | C15DA | M [symbol] | OH | Y | Aib | E | G | T | F | I | S | D | Y | S | I |
| 216 | 2OEGgEgE | C15DA | M [symbol] | OH | Y | [symbol] | E | G | T | F | I | S | D | Y | S | I |
| 217 | 2OEGgEgE | C15DA | M [symbol] | OH | Y | A | E | G | T | F | I | S | D | Y | S | I |
| 218 | 2OEG | C15DA | M [symbol] | OH | Y | Aib | E | G | T | F | I | S | D | Y | S | I |
| 219 | 2OEGgEgE | C15DA | M [symbol] | OH | Y | Aib | E | G | T | F | I | S | D | Y | S | I |
| 220 | 2OEGgEgE | C15DA | M [symbol] | OH | Y | Aib | E | G | T | F | I | S | D | Y | S | I |
| 221 | 2OEGgEgE | C15DA | M [symbol] | OH | Y | Aib | E | G | T | F | I | S | D | Y | S | I |
| 222 | 2OEGgEgE | C15DA | M [symbol] | OH | Y | Aib | E | G | T | F | I | S | D | Y | S | I |
| 223 | 2OEGgEgE | C15DA | M [symbol] | OH | Y | Aib | E | G | T | F | I | S | D | Y | S | I |
| 224 | 2OEGgEgE | C15DA | M [symbol] | OH | Y | Aib | E | G | T | F | I | S | D | Y | S | I |
| 225 | 2OEGgEgE | C15DA | M [symbol] | OH | Y | Aib | E | G | T | F | I | S | D | Y | S | I |
| 226 | 2OEGgE | C15DA | M [symbol] | | Y | Aib | E | G | T | F | I | S | D | Y | S | I |
| 227 | 2OEGgE | C15DA | M [symbol] | | Y | Aib | E | G | T | F | I | S | D | Y | S | I |
| 228 | 2OEGgE | C15DA | M [symbol] | | Y | Aib | E | G | T | F | I | S | D | Y | S | I |
| 229 | 2OEGgE | C15DA | M [symbol] | | Y | Aib | E | G | T | F | I | S | D | Y | S | I |
| 230 | 2OEGgE | C15DA | M [symbol] | | Y | Aib | E | G | T | F | I | S | D | Y | S | I |
| 231 | GS | C15DA | M [symbol] | NH2 | Y | Aib | E | G | T | F | I | S | D | Y | S | I |
| 232 | GS | C15DA | M [symbol] | NH2 | Y | Aib | E | G | T | F | I | S | D | Y | S | I |
| 233 | 2OEGgE | C15DA | M [symbol] | NH2 | Y | Aib | E | G | T | F | I | S | D | Y | S | I |
| 234 | 2OEGgE | C15DA | M [symbol] | NH2 | Y | Aib | E | G | T | F | I | S | D | Y | S | I |
| 235 | 2OEGgE | C15DA | M [symbol] | NH2 | Y | Aib | E | G | T | F | I | S | D | Y | S | I |
| 236 | 2OEGgE | C15DA | M [symbol] | NH2 | Y | Aib | E | G | T | F | I | S | D | Y | S | I |
| 237 | 2OEGgE | C15DA | M [symbol] | | Y | Aib | E | G | T | F | I | S | D | Y | S | I |
| 238 | 2OEGgE | C15DA | M [symbol] | | Y | Aib | E | G | T | F | I | S | D | Y | S | I |
| 239 | 2OEGgE | C15DA | M [symbol] | | Y | Aib | E | G | T | F | I | S | D | Y | S | I |
| 240 | 2OEGgE | C15DA | M [symbol] | | Y | Aib | E | G | T | F | I | S | D | Y | S | I |
| 241 | 2OEGgE | C15DA | | | Y | A | E | G | T | F | I | S | D | Y | S | I |
| 242 | 2OEGgE | C15DA | M [symbol] | | Y | Aib | E | G | T | F | I | S | D | Y | S | I |
| 243 | 2OEGgE | C15DA | M [symbol] | | Y | G | E | G | T | F | I | S | D | Y | S | I |
| 244 | 2OEGgE | C15DA | | | Y | S | E | G | T | F | I | S | D | Y | S | I |
| 245 | 2OEGgE | C15DA | | | Y | Aib | E | G | T | F | I | S | D | Y | S | I |
| 246 | 2OEGgEgE | C15DA | M [symbol] | NH2 | Y | Aib | E | G | T | F | I | S | D | Y | S | I |
| 247 | 2OEGgEgE | C15DA | M [symbol] | OH | Y | Aib | E | G | T | F | I | S | D | Y | S | I |
| 248 | OEGgEgEgE | C15DA | M [symbol] | OH | Y | Aib | E | G | T | F | I | S | D | Y | S | I |
| 249 | G2E3 | C15DA | M [symbol] | OH | Y | Aib | E | G | T | F | I | S | D | Y | S | I |
| 250 | GS | C15DA | M [symbol] | OH | Y | Aib | E | G | T | F | I | S | D | Y | S | I |
| 251 | GS | C15DA | M [symbol] | OH | Y | Aib | E | G | T | F | I | S | D | Y | S | I |
| 252 | [symbol] PAPAP | C15DA | M [symbol] | NH2 | Y | Aib | E | G | T | F | I | S | D | Y | S | I |
| 253 | 2OEGgE | C15DA | M [symbol] | OH | Y | Aib | E | G | T | F | I | S | D | Y | S | I |
| 254 | OEGgEgE | C15DA | M [symbol] | NH2 | Y | Aib | E | G | T | F | I | S | D | Y | S | I |
| 255 | GGPAPAP | C15DA | M [symbol] | NH2 | Y | Aib | E | G | T | F | I | S | D | Y | S | I |
| 256 | GGPAPAP | C15DA | M [symbol] | NH2 | Y | Aib | E | G | T | F | I | S | D | Y | S | I |
| 257 | G3gEgE | C15DA | M [symbol] | NH2 | Y | Aib | E | G | T | F | I | S | D | Y | S | I |
| 258 | G3gEgE | C15DA | M [symbol] | OH | Y | Aib | E | G | T | F | I | S | D | Y | S | I |
| 259 | G5gE | C15DA | M [symbol] | NH2 | Y | Aib | E | G | T | F | I | S | D | Y | S | I |
| 260 | G5gEgE | C15DA | M [symbol] | NH2 | Y | Aib | E | G | T | F | I | S | D | Y | S | I |
| 261 | G3E2 | C15DA | M [symbol] | NH2 | Y | Aib | E | G | T | F | I | S | D | Y | S | I |
| 262 | G3E2 | C15DA | M [symbol] | NH2 | Y | Aib | E | G | T | F | I | S | L | Y | S | I |
| 263 | GSgEgE | C15DA | M [symbol] | NH2 | Y | Aib | E | G | T | F | I | S | D | Y | S | I |
| 264 | 2OEGgEgE | C15DA | M [symbol] | NH2 | Y | Aib | E | G | T | F | I | S | D | Y | S | I |
| 265 | 2OEGgEgE | C15DA | M [symbol] | NH2 | Y | Aib | E | G | T | F | I | S | D | Y | S | I |
| 266 | 2OEGgEgE | C15DA | M [symbol] | OH | Y | Aib | E | G | T | F | I | S | D | Y | S | I |
| 267 | 2OEGgEgE | C15DA | M [symbol] | NH2 | Y | Aib | E | G | T | F | I | S | D | Y | S | I |
| 268 | 2OEGgEgE | C15DA | M [symbol] | NH2 | Y | Aib | E | G | T | F | I | S | L | Y | S | I |
| 269 | GS | C15DA | M [symbol] | OH | Y | Aib | E | G | T | F | I | S | D | Y | S | I |
| 270 | GSgEgE | C15DA | M [symbol] | NH2 | Y | Aib | E | G | T | F | I | S | D | Y | S | I |
| 271 | GSgE | C15DA | M [symbol] | NH2 | Y | Aib | E | G | T | F | I | S | D | Y | S | I |
| 272 | GSgE | C15DA | M [symbol] | NH2 | Y | Aib | E | G | T | F | I | S | D | Y | S | I |
| 273 | 2OEGgE | C15DA | M [symbol] | NH2 | Y | Aib | E | G | T | F | I | S | D | Y | S | I |
| 274 | GSgE | C15DA | M [symbol] | NH2 | Y | Aib | E | G | T | F | I | S | D | Y | S | I |
| 275 | GSgEgE | C15DA | M [symbol] | NH2 | Y | Aib | E | G | T | F | I | S | D | Y | S | I |
| 276 | GSgE | C15DA | M [symbol] | NH2 | Y | Aib | E | G | T | F | I | S | D | Y | S | I |
| 277 | 2OEGgE | C15DA | M [symbol] | NH2 | Y | Aib | E | G | T | F | I | S | D | Y | S | I |
| 278 | 2OEGgEgE | C15DA | M [symbol] | NH2 | Y | Aib | E | G | T | F | I | S | D | Y | S | I |
| 279 | GSgE | C15DA | M [symbol] | OH | Y | Aib | E | G | T | F | I | S | D | Y | S | I |
| 280 | 2OEGgE | C15DA | M [symbol] | NH2 | Y | Aib | E | G | T | F | I | S | D | Y | S | I |
| 281 | GSgE | C15DA | M [symbol] | NH2 | Y | Aib | E | G | T | F | I | S | D | Y | S | I |
| 282 | 2OEGgE | C15DA | M [symbol] | NH2 | Y | Aib | E | G | T | F | I | S | D | Y | S | I |
| 283 | GSgE | C15DA | M [symbol] | NH2 | Y | Aib | E | G | T | F | I | S | D | Y | S | I |
| 284 | 2OEGgE | C15DA | M [symbol] | NH2 | Y | Aib | E | G | T | F | I | S | D | Y | S | I |
| 285 | 2OEGgE | C15DA | M [symbol] | NH2 | Y | Aib | E | G | T | F | I | S | D | Y | S | I |
| 286 | 2OEGgE | C15DA | M [symbol] | NH2 | Y | Aib | E | G | T | F | I | S | D | Y | S | I |
| 287 | GSgE | C15DA | M [symbol] | NH2 | Y | Aib | E | G | T | F | I | S | D | Y | S | I |
| 288 | GSgE | C15DA | M [symbol] | NH2 | Y | Aib | E | G | T | F | I | S | D | Y | S | I |
| 289 | OEGgEgEgE | C15DA | M [symbol] | NH2 | Y | Aib | E | G | T | F | I | S | D | Y | S | I |
| 290 | 2OEGgEgE | C15DA | M [symbol] | NH2 | Y | Aib | E | G | T | F | I | S | D | Y | S | I |
| 291 | GS | C15DA | M [symbol] | NH2 | Y | Aib | E | G | T | F | I | S | D | Y | S | I |
| 292 | 2OEG | C15DA | M [symbol] | NH2 | Y | Aib | E | G | T | F | I | S | D | Y | S | I |
| 293 | 2OEGgE | C15DA | M [symbol] | NH2 | Y | Aib | E | G | T | F | I | S | D | Y | S | I |
| 294 | 2OEGgEgE | C15DA | M [symbol] | NH2 | Y | Aib | E | G | T | F | I | S | D | Y | S | I |
| 295 | 2OEGgEgE | C15DA | M [symbol] | NH2 | Y | Aib | E | G | T | F | I | S | D | Y | S | I |
| 296 | GS | C15DA | M [symbol] | NH2 | Y | Aib | E | G | T | F | I | S | D | Y | S | I |
| 297 | GS | C15DA | M [symbol] | NH2 | Y | Aib | E | G | T | F | I | S | D | Y | S | I |
| 298 | GS | C15DA | M [symbol] | NH2 | Y | Aib | E | G | T | F | I | S | D | Y | S | I |
| 299 | GS | C15DA | M [symbol] | OH | Y | Aib | E | G | T | F | I | S | D | Y | S | I |
| 300 | GS | C15DA | M [symbol] | NH2 | Y | Aib | E | G | T | F | I | S | D | Y | S | I |
| 301 | GS | C15DA | M [symbol] | OH | Y | Aib | E | G | T | F | I | S | D | Y | S | I |
| 302 | GS | C15DA | M [symbol] | OH | Y | Aib | E | G | T | F | I | S | D | Y | S | I |
| 303 | gE | C16 | | | Y | Aib | E | G | T | F | I | S | D | Y | S | I |
| 304 | Ac | | M [symbol] | OH | Y | Aib | E | G | T | F | I | S | D | Y | S | I |
| 305 | Ac | | M [symbol] | OH | Y | Aib | E | G | T | F | I | S | D | Y | S | I |

| CPMD No. | 13 | 14 | 15 | 16 | 17 | 18 | 19 | 20 | 21 | 22 | 23 | 24 | 25 | 26 | 27 | 28 |
|---|---|---|---|---|---|---|---|---|---|---|---|---|---|---|---|---|
| 1 | A | Km | D | R | Aib | A | Q | Q | N | F | V | N | W | L | L | A |
| 2 | A | Km | D | R | Aib | A | Q | Q | N | F | V | N | W | L | L | A |
| 3 | A | Km | D | R | Aib | A | Q | Q | N | F | V | N | W | L | L | A |
| 4 | A | L | D | R | | H | Q | Km | N | F | V | N | W | L | L | A |
| 5 | A | L | D | R | | H | Q | Km | N | F | V | N | W | L | L | A |
| 6 | A | Km | D | R | | H | Q | Aib | N | F | V | N | W | L | L | A |
| 7 | A | Km | D | R | | H | Q | Aib | N | F | V | N | W | L | L | A |
| 8 | A | L | D | R | | Km | Q | Aib | N | F | V | N | W | L | L | A |
| 9 | A | L | D | R | | H | Q | Aib | Km | F | V | N | W | L | L | A |
| 10 | A | L | D | R | | H | Q | Aib | N | F | V | N | W | L | L | Km |
| 11 | A | Km | D | R | Aib | A | Q | Aib | N | F | V | N | W | L | L | A |
| 12 | A | L | D | R | Aib | H | Q | Aib | Km | F | V | N | W | L | L | A |
| 13 | A | Km | D | R | Aib | A | Q | Aib | N | F | V | N | W | L | L | A |
| 14 | A | L | D | R | Aib | A | Q | Aib | Km | F | V | N | W | L | L | A |
| 15 | A | L | D | R | Aib | A | Q | Aib | Km | F | V | N | W | L | L | A |
| 16 | A | Km | D | R | Aib | A | Q | Aib | N | F | V | N | W | L | L | A |
| 17 | A | L | D | R | Aib | Km | Q | Aib | N | F | V | N | W | L | L | A |
| 18 | A | Km | D | R | Aib | A | Q | Aib | N | F | V | N | W | L | L | A |
| 19 | Aib | Km | D | R | Aib | A | Q | Aib | N | F | V | N | W | L | L | A |
| 20 | Aib | L | D | R | Aib | A | Q | Aib | N | F | V | N | W | L | L | A |
| 21 | Aib | L | D | R | Aib | A | Q | Aib | N | F | V | N | W | L | L | A |
| 22 | A | Km | D | R | Aib | A | Q | Aib | N | F | V | N | W | L | L | A |
| 23 | A | Km | D | R | Aib | A | Q | Aib | N | F | V | N | W | L | L | A |
| 24 | A | L | D | R | Aib | A | Q | Aib | Km | F | V | N | W | L | L | A |
| 25 | A | L | D | R | Aib | A | Q | Aib | Km | F | V | N | W | L | L | A |
| 26 | A | Km | D | R | Aib | A | Q | Aib | N | F | V | N | W | L | L | A |
| 27 | A | L | D | R | Aib | A | Q | Aib | Km | F | V | N | W | L | L | A |
| 28 | A | Km | D | R | Aib | A | Q | Aib | N | F | V | N | W | L | L | Aib |
| 29 | A | L | D | R | Aib | A | Q | Aib | Km | F | V | N | W | L | L | Aib |
| 30 | A | Km | D | R | Aib | A | Q | Aib | N | F | V | N | W | L | L | A |
| 31 | A | L | D | R | Aib | A | Q | Aib | Km | F | V | N | W | L | L | A |
| 32 | A | L | D | R | Aib | A | Q | Aib | N | F | V | N | W | L | L | A |
| 33 | A | L | D | R | Aib | A | Q | Aib | N | F | V | N | W | L | L | A |
| 34 | A | L | D | R | Aib | A | Q | Aib | N | F | V | N | W | L | L | A |
| 35 | A | L | D | R | Aib | A | Q | Aib | N | F | V | N | W | L | L | A |
| 36 | A | L | D | R | Aib | A | Q | Aib | O | F | V | N | W | L | L | A |
| 37 | A | L | D | R | Aib | A | Q | Aib | N | F | V | N | W | L | L | A |
| 38 | A | Km | D | R | Aib | A | Q | Aib | N | F | V | N | W | L | L | Aib |
| 39 | A | Km | D | R | Aib | A | Q | Aib | N | F | V | N | W | L | L | A |
| 40 | A | L | D | R | Aib | A | Q | Aib | Km | F | V | N | W | L | L | Aib |
| 41 | A | L | D | R | Aib | A | Q | Aib | N | F | V | N | W | L | L | A |
| 42 | A | L | D | R | Aib | A | Q | Aib | N | F | V | N | W | L | L | A |
| 43 | A | L | D | R | Aib | A | Q | Aib | N | F | V | N | W | L | L | A |
| 44 | A | L | D | R | Aib | A | Q | Aib | N | F | V | N | W | L | L | A |
| 45 | Y | L | D | R | Aib | A | Q | Aib | Km | F | V | N | W | L | L | A |
| 46 | A | Km | D | R | Aib | A | Q | Aib | N | F | V | N | W | L | L | A |
| 47 | A | L | D | R | Aib | A | Q | Aib | N | F | V | N | W | L | L | A |
| 48 | A | Km | D | R | Aib | A | Q | Aib | N | F | V | N | W | L | L | A |
| 49 | A | Km | D | R | Aib | A | Q | Aib | N | F | V | N | W | L | L | A |
| 50 | A | Km | D | R | Aib | A | Q | Aib | N | F | V | N | W | L | L | A |
| 51 | A | Km | D | R | Aib | A | Q | Aib | N | F | V | N | W | L | L | A |
| 52 | A | L | D | R | Aib | A | Q | Aib | N | F | V | N | W | L | L | A |
| 53 | A | L | D | R | Aib | A | Q | Aib | N | F | V | N | W | L | L | A |
| 54 | A | L | D | R | Km | A | Q | Aib | N | F | V | N | W | L | L | A |
| 55 | A | L | D | R | Aib | Km | Q | Aib | N | F | V | N | W | L | L | A |
| 56 | Aib | Km | D | R | Aib | A | Q | Aib | N | F | V | N | W | L | L | A |
| 57 | Aib | Km | D | R | Aib | A | Q | Aib | N | F | V | N | W | L | L | A |
| 58 | Aib | Km | D | R | Aib | A | Q | Aib | N | F | V | N | W | L | L | A |
| 59 | Aib | Km | D | R | Aib | A | Q | Aib | N | F | V | N | W | L | L | A |
| 60 | A | L | D | R | Aib | A | Q | Aib | Km | F | V | N | W | L | L | A |
| 61 | A | L | D | R | Aib | A | Q | Aib | Km | F | V | N | W | L | L | A |
| 62 | A | L | D | R | Km | H | Q | Aib | N | F | V | N | W | L | L | A |
| 63 | A | L | D | R | Km | H | Q | Aib | N | F | V | N | W | L | L | A |
| 64 | A | L | D | R | Km | H | Q | Aib | N | F | V | N | W | L | L | A |
| 65 | A | L | D | R | Km | H | Q | Aib | N | F | V | N | W | L | L | A |
| 66 | A | L | D | R | Km | H | Q | Aib | D | F | V | N | W | L | L | A |
| 67 | A | L | D | R | Km | H | Q | Aib | D | F | V | N | W | L | L | A |
| 68 | A | L | D | R | Km | H | Q | Aib | D | F | V | N | W | L | L | A |
| 69 | A | L | D | R | Km | H | Q | Aib | D | F | V | N | W | L | L | A |
| 70 | A | L | D | R | Aib | A | Q | Aib | N | F | V | N | W | L | L | A |
| 71 | A | L | D | R | Aib | Km | Q | Aib | N | F | V | N | W | L | L | A |
| 72 | A | L | D | R | Aib | Km | Q | Aib | N | F | V | N | W | L | L | A |
| 73 | A | L | D | R | Aib | Km | Q | Aib | D | F | V | N | W | L | L | A |
| 74 | A | L | D | R | Aib | Km | Q | Aib | D | F | V | N | W | L | L | A |
| 75 | A | L | D | R | Aib | Km | Q | Aib | D | F | V | N | W | L | L | A |
| 76 | A | L | D | R | Aib | Km | Q | Aib | D | F | V | N | W | L | L | A |
| 77 | A | Km | D | R | Aib | A | Q | Aib | N | F | V | N | W | L | L | A |
| 78 | A | Km | D | R | Aib | A | Q | Aib | N | F | V | N | W | L | L | A |
| 79 | A | Km | D | R | Aib | A | Q | Aib | D | F | V | N | W | L | L | A |
| 80 | A | Km | D | R | Aib | A | Q | Aib | D | F | V | N | W | L | L | A |
| 81 | A | Km | D | R | Aib | A | Q | Aib | N | F | V | N | W | L | L | A |
| 82 | A | Km | D | R | Aib | A | Q | Aib | N | F | V | N | W | L | L | A |
| 83 | A | L | D | R | Aib | A | Q | Aib | Km | F | V | N | W | L | L | A |
| 84 | A | L | D | R | Aib | H | Q | Aib | N | F | V | N | W | L | L | A |
| 85 | A | L | D | R | Aib | A | Q | Aib | Km | F | V | N | W | L | L | A |
| 86 | A | L | D | R | Aib | A | Q | Aib | Km | F | V | N | W | L | L | A |
| 87 | A | L | D | R | Aib | A | Q | Aib | N | F | V | N | W | L | L | A |
| 88 | A | L | D | R | Aib | A | Q | Aib | N | F | V | N | W | L | L | A |
| 89 | A | L | D | R | Aib | A | Q | Aib | N | F | V | N | W | L | L | A |
| 90 | A | L | D | R | Aib | A | Q | Aib | D | F | V | N | W | L | L | A |
| 91 | A | L | D | R | Aib | A | Q | Aib | N | F | V | N | W | L | L | A |
| 92 | A | L | D | R | Aib | A | Q | Aib | Km | F | V | N | W | L | L | A |
| 93 | A | L | D | R | Aib | A | Q | Aib | Km | F | V | N | W | L | L | A |
| 94 | Aib | Km | D | R | Aib | A | Q | Aib | N | F | V | N | W | L | L | A |
| 95 | Aib | Km | D | R | Aib | A | Q | Aib | N | F | V | N | W | L | L | A |
| 96 | A | L | D | R | Aib | A | Q | Aib | Km | F | V | N | W | L | L | A |
| 97 | A | Km | D | R | Aib | A | Q | Aib | D | F | V | N | W | L | L | A |
| 98 | A | L | D | R | Aib | A | Q | Aib | D | F | V | N | W | L | L | A |
| 99 | A | Km | D | R | Aib | A | Q | Aib | N | F | V | N | W | L | L | A |
| 100 | Aib | L | D | R | Aib | A | Q | Aib | D | F | V | N | W | L | L | A |
| 101 | A | L | D | R | Aib | A | Q | Aib | N | F | V | N | W | L | L | A |
| 102 | Aib | L | D | R | Aib | A | Q | Aib | Km | F | V | N | W | L | L | A |
| 103 | Aib | L | D | R | Aib | A | Q | Aib | Km | F | V | N | W | L | L | A |
| 104 | A | L | D | R | Aib | A | Q | Aib | D | F | V | N | W | L | L | A |
| 105 | A | L | D | R | Aib | Km | Q | Aib | N | F | V | N | W | L | L | A |
| 106 | A | L | D | R | Aib | Km | Q | Aib | D | F | V | N | W | L | L | A |
| 107 | A | L | D | R | Aib | A | Q | Aib | D | F | V | N | W | L | L | A |
| 108 | Aib | L | D | R | Aib | A | Q | Aib | Km | F | V | N | W | L | L | A |
| 109 | A | L | D | R | Aib | A | Q | Aib | D | F | V | N | W | L | L | A |
| 110 | Aib | Km | D | R | Aib | A | Q | Aib | D | F | V | N | W | L | L | A |
| 111 | Aib | L | D | R | Aib | Km | Q | Aib | N | F | V | N | W | L | L | A |
| 112 | Aib | L | D | R | Aib | A | Q | Aib | Km | F | V | N | W | L | L | A |
| 113 | Aib | L | D | R | Aib | A | Q | Aib | Km | F | V | N | W | L | L | A |
| 114 | Aib | L | D | R | Aib | A | Q | Aib | Km | F | V | N | W | L | L | A |
| 115 | Aib | Km | D | R | Aib | A | Q | Aib | N | F | V | N | W | L | L | A |
| 116 | Aib | Km | D | R | Aib | A | Q | Aib | D | F | V | N | W | L | L | A |
| 117 | Aib | L | D | R | Aib | Km | Q | Aib | D | F | V | N | W | L | L | A |
| 118 | Aib | L | D | R | Aib | Km | Q | Aib | D | F | V | N | W | L | L | A |
| 119 | Aib | L | D | R | Aib | Km | Q | Aib | N | F | V | N | W | L | L | A |
| 120 | Aib | Km | D | R | Aib | A | Q | Aib | N | F | V | N | W | L | L | A |
| 121 | A | L | D | R | Aib | A | Q | Aib | Km | F | V | N | W | L | L | A |
| 122 | Aib | L | D | R | Km | A | Q | Aib | N | F | V | N | W | L | L | A |
| 123 | Aib | L | D | R | Aib | A | Q | Aib | N | F | V | N | W | L | L | A |
| 124 | Aib | Km | D | R | Aib | A | Q | Aib | N | F | V | N | W | L | L | A |
| 125 | A | Km | D | R | Aib | A | Q | Aib | N | F | V | N | W | L | L | A |
| 126 | A | Km | D | R | Aib | A | Q | Aib | D | F | V | N | W | L | L | A |
| 127 | A | L | D | R | Km | A | Q | Aib | N | F | V | N | W | L | L | A |
| 128 | Aib | Km | D | R | Aib | A | Q | Aib | D | F | V | N | W | L | L | A |
| 129 | Aib | Km | D | R | Aib | A | Q | Aib | D | F | V | N | W | L | L | A |
| 130 | A | L | D | R | Aib | A | Q | Aib | Km | F | V | N | W | L | L | A |
| 131 | A | L | D | R | Km | A | Q | Aib | N | F | V | N | W | L | L | A |
| 132 | A | L | D | R | Km | A | Q | Aib | D | F | V | N | W | L | L | A |
| 133 | A | L | D | R | Aib | Km | Q | Aib | N | F | V | N | W | L | L | A |
| 134 | Aib | Km | D | R | Aib | A | Q | Aib | N | F | V | N | W | L | L | A |
| 135 | Aib | L | D | R | Aib | A | Q | Aib | N | F | V | N | W | L | L | A |
| 136 | Aib | L | D | R | Aib | A | Q | Aib | N | F | V | N | W | L | L | A |
| 137 | A | L | D | R | Aib | A | Q | Aib | D | F | V | N | W | L | L | A |
| 138 | Aib | Km | D | R | Aib | A | Q | Aib | N | F | V | N | W | L | L | A |
| 139 | Aib | Km | D | R | Aib | A | Q | Aib | N | F | V | N | W | L | L | A |
| 140 | Aib | Km | D | R | Aib | A | Q | Aib | D | F | V | N | W | L | L | A |
| 141 | Aib | Km | D | R | Aib | A | Q | Aib | D | F | V | N | W | L | L | A |
| 142 | Aib | Km | D | R | Aib | A | Q | Aib | D | F | V | N | W | L | L | A |
| 143 | A | L | D | R | Aib | A | Q | Aib | Km | F | V | N | W | L | L | A |
| 144 | A | L | D | R | Aib | Km | Q | Aib | N | F | V | N | W | L | L | A |
| 145 | Aib | L | D | R | Aib | A | Q | Aib | N | F | V | N | W | L | L | A |
| 146 | A | Km | D | R | Aib | A | Q | Aib | D | F | V | N | W | L | L | A |
| 147 | A | Km | D | R | Aib | A | Q | Aib | N | F | V | N | W | L | L | A |
| 148 | A | L | D | R | Aib | A | Q | Aib | Km | F | V | N | W | L | L | A |
| 149 | A | L | D | R | Aib | A | Q | Aib | Km | F | V | N | W | L | L | A |
| 150 | A | Km | D | R | Aib | A | Q | Aib | N | F | V | N | W | L | L | A |
| 151 | A | Km | D | R | Aib | A | Q | Aib | D | F | V | N | W | L | L | A |
| 152 | A | Km | D | R | Aib | A | Q | Aib | D | F | V | N | W | L | L | A |
| 153 | A | Km | D | S | Aib | A | Q | Aib | D | F | V | N | W | L | L | A |
| 154 | A | Km | D | S | Aib | A | Q | Aib | N | F | V | N | W | L | L | A |
| 155 | A | Km | D | R | Aib | A | Q | Aib | D | F | V | N | W | L | L | A |
| 156 | Aib | Km | D | R | Aib | A | Q | Aib | D | F | V | N | W | L | L | A |
| 157 | A | Km | D | R | Aib | A | Q | Aib | D | F | V | N | W | L | L | A |
| 158 | A | Km | D | R | Aib | A | Q | Aib | D | F | V | N | W | L | L | A |
| 159 | A | Km | D | R | Aib | A | Q | Aib | D | F | V | N | W | L | L | A |
| 160 | A | Km | D | R | Aib | A | Q | Aib | N | F | V | N | W | L | L | A |
| 161 | A | L | D | R | Aib | Km | Q | Aib | N | F | V | N | W | L | L | A |
| 162 | A | L | D | R | Aib | Km | Q | Aib | D | F | V | N | W | L | L | A |
| 163 | A | L | D | R | Aib | Km | Q | Aib | D | F | V | N | W | L | L | A |
| 164 | A | L | D | R | Km | A | Q | Aib | N | F | V | N | W | L | L | A |
| 165 | A | L | D | R | Km | A | Q | Aib | D | F | V | N | W | L | L | A |
| 166 | A | L | D | R | Km | A | Q | Aib | D | F | V | N | W | L | L | A |
| 167 | A | L | D | R | Km | A | Q | Aib | D | F | V | N | W | L | L | A |
| 168 | A | L | D | R | Km | A | Q | Aib | D | F | V | N | W | L | L | A |
| 169 | A | L | D | R | Aib | Km | Q | Aib | D | F | V | N | W | L | L | A |
| 170 | A | L | D | R | Aib | Km | Q | Aib | D | F | V | N | W | L | L | A |
| 171 | A | L | D | R | Aib | Km | Q | Aib | N | F | V | N | W | L | L | A |
| 172 | A | Km | D | R | Aib | A | Q | Aib | D | F | V | N | W | L | L | A |
| 173 | A | Km | D | R | Aib | A | Q | Aib | D | F | V | N | W | L | L | A |
| 174 | A | L | D | R | Km | A | Q | Aib | D | F | V | N | W | L | L | A |
| 175 | Aib | L | D | R | Aib | A | Q | Aib | Km | F | V | N | W | L | L | A |
| 176 | Aib | Km | D | R | Aib | A | Q | Aib | D | F | V | N | W | L | L | A |
| 177 | A | Km | D | S | Aib | A | Q | Aib | D | F | V | N | W | L | L | A |
| 178 | Aib | L | D | S | Km | A | Q | Aib | D | F | V | N | W | L | L | A |
| 179 | A | L | D | S | Km | A | Q | Aib | D | F | V | N | W | L | L | A |
| 180 | A | Km | D | R | Aib | A | Q | Aib | D | F | V | N | W | L | L | A |
| 181 | A | Km | D | R | Aib | A | Q | Aib | D | F | V | N | W | L | L | A |
| 182 | Aib | Km | D | R | Aib | A | Q | Aib | D | F | V | N | W | L | L | A |
| 183 | Aib | L | D | R | Aib | A | Q | Aib | Km | F | V | N | W | L | L | A |
| 184 | A | L | D | R | Aib | A | Q | Aib | Km | F | V | N | W | L | L | A |
| 185 | Aib | L | D | R | Aib | Km | Q | Aib | D | F | V | N | W | L | L | A |
| 186 | Aib | L | D | R | Km | A | Q | Aib | D | F | V | N | W | L | L | A |
| 187 | Aib | Km | D | R | Aib | A | Q | Aib | N | F | V | N | W | L | L | A |
| 188 | A | L | D | R | Aib | A | Q | Aib | Km | F | V | N | W | L | L | A |
| 189 | A | Km | D | R | Aib | A | Q | Aib | N | F | V | N | W | L | L | A |
| 190 | A | L | D | R | Aib | H | Q | Aib | N | F | V | N | W | Km | L | A |
| 191 | V | L | D | R | Q | A | A | Aib | E | F | V | N | W | L | L | A |
| 192 | A | Km | D | R | I | H | Q | Q | N | F | V | N | W | L | L | A |
| 193 | A | Km | D | R | I | H | Q | Q | N | F | V | N | W | L | L | A |
| 194 | A | Km | D | R | I | H | Q | Q | N | F | V | N | W | L | L | A |
| 195 | A | Km | D | R | I | H | Q | Q | N | F | V | N | W | L | L | A |
| 196 | A | Km | D | R | I | A | Q | Aib | N | F | V | N | W | L | L | A |
| 197 | A | Km | D | R | Aib | A | Q | Aib | N | F | V | N | W | L | L | A |
| 198 | A | L | D | R | Aib | A | Q | Aib | Km | F | V | N | W | L | L | A |
| 199 | Aib | Km | D | R | Aib | A | Q | Aib | D | F | V | N | W | L | L | A |
| 200 | A | L | D | R | Aib | Km | Q | Aib | N | F | V | N | W | L | L | A |
| 201 | A | L | D | R | Aib | A | Q | Aib | Km | F | V | N | W | L | L | A |
| 202 | A | L | D | R | Aib | H | Q | Aib | Km | F | V | N | W | L | L | A |
| 203 | Aib | L | D | R | Aib | A | Q | Aib | Km | F | V | N | W | L | L | A |
| 204 | A | L | D | R | Aib | A | Q | Aib | Km | F | V | N | W | L | L | A |
| 205 | A | L | D | R | Aib | A | Q | Aib | Km | F | V | N | W | L | L | A |
| 206 | A | L | D | R | Aib | A | Q | Aib | Km | F | V | N | W | L | L | A |
| 207 | Aib | L | D | R | Aib | A | Q | Aib | Km | F | V | N | W | L | L | A |
| 208 | Aib | L | D | R | Aib | A | Q | Aib | Km | F | V | N | W | L | L | A |
| 209 | A | L | D | R | Aib | A | Q | Aib | Km | F | V | N | W | L | L | A |
| 210 | A | L | D | R | Aib | A | Q | Aib | Km | F | V | N | W | L | L | A |
| 211 | A | L | D | R | Aib | A | Q | Aib | Km | F | V | N | W | L | L | A |
| 212 | A | L | D | R | Aib | A | Q | Aib | Km | F | V | N | W | L | L | A |
| 213 | A | L | D | R | Aib | A | Q | Aib | Km | F | V | N | W | L | L | A |
| 214 | A | L | D | R | Aib | A | Q | Aib | Km | F | V | N | W | L | L | A |
| 215 | A | L | D | R | Aib | A | Q | Aib | Km | F | V | N | W | L | L | A |
| 216 | A | L | D | R | Aib | A | Q | Aib | Km | F | V | N | W | L | L | A |
| 217 | A | L | D | R | Aib | A | Q | Aib | Km | F | V | N | W | L | L | A |
| 218 | A | L | D | R | Aib | A | Q | Aib | Km | F | V | N | W | L | L | A |
| 219 | A | L | D | R | Aib | A | Q | Aib | Km | F | V | N | W | L | L | A |
| 220 | A | L | D | R | Aib | Km | Q | Aib | D | F | V | N | W | L | L | A |
| 221 | A | L | D | R | Km | A | Q | Aib | D | F | V | N | W | L | L | A |
| 222 | A | L | D | R | Aib | A | Q | Aib | D | F | V | N | W | L | L | A |
| 223 | A | L | D | R | Aib | A | Q | Aib | D | F | V | N | W | L | L | A |
| 224 | A | L | D | R | Aib | A | Q | Aib | Km | F | V | N | W | L | L | A |
| 225 | A | L | D | R | Aib | A | Q | Km | D | F | V | N | W | L | L | A |
| 226 | A | L | D | R | I | H | Q | Q | N | F | V | N | W | L | L | A |
| 227 | A | L | D | R | I | H | Q | Q | N | F | V | N | W | L | L | A |
| 228 | A | L | D | R | Km | H | Q | Q | N | F | V | N | W | L | L | A |
| 229 | A | L | D | R | I | H | Q | Q | N | F | V | N | W | L | L | A |
| 230 | A | L | D | R | I | H | Q | Q | N | F | V | N | W | L | L | A |
| 231 | A | L | D | R | Aib | A | Q | Aib | N | F | V | N | W | L | L | A |
| 232 | A | L | D | R | I | A | Q | Q | N | F | V | N | W | L | L | A |
| 233 | A | L | D | R | Aib | A | Q | Aib | N | F | V | N | W | L | L | A |
| 234 | A | L | D | R | Aib | A | Q | Q | N | F | V | N | W | L | L | A |
| 235 | A | L | D | R | I | A | Q | Aib | N | F | V | N | W | L | L | A |
| 236 | A | L | D | R | I | A | Q | Q | N | F | V | N | W | L | L | A |
| 237 | A | L | D | R | I | A | Q | Q | N | F | V | N | W | L | L | A |
| 238 | A | L | D | R | Aib | A | Q | Q | N | F | V | N | W | L | L | A |
| 239 | A | L | D | R | I | H | Q | Q | N | F | V | N | W | L | L | A |
| 240 | A | L | D | R | Km | H | Q | Q | N | F | V | N | W | L | L | A |
| 241 | A | L | D | R | I | H | Q | Aib | N | F | V | N | W | L | L | A |
| 242 | A | L | D | R | Aib | A | Q | Aib | N | F | V | N | W | L | L | A |
| 243 | A | Km | D | R | Aib | A | Q | Aib | N | F | V | N | W | L | L | A |
| 244 | A | Km | D | R | Aib | H | Q | Aib | N | F | V | N | W | L | L | A |
| 245 | A | Km | D | R | Aib | H | Q | Aib | N | F | V | N | W | L | L | A |
| 246 | Aib | L | D | R | Aib | A | Q | Aib | Km | F | V | N | W | L | L | A |
| 247 | A | Km | D | R | Aib | A | Q | Aib | N | F | V | N | W | L | L | A |
| 248 | A | L | D | R | Aib | Km | Q | Aib | N | F | V | N | W | L | L | A |
| 249 | A | Km | D | R | Aib | A | Q | Aib | N | F | V | N | W | L | L | A |
| 250 | A | Km | D | R | Aib | A | Q | Aib | N | F | V | N | W | L | L | A |
| 251 | A | L | D | R | Aib | H | Q | Aib | Km | F | V | N | W | L | L | A |
| 252 | A | L | D | R | Aib | A | Q | Aib | Km | F | V | N | W | L | L | A |
| 253 | A | L | D | K | Aib | A | Q | Aib | D | F | V | N | W | L | L | A |
| 254 | A | L | D | K | Aib | A | Q | Aib | N | F | V | N | W | L | L | A |
| 255 | A | L | D | R | Aib | Km | Q | Aib | N | F | V | N | W | L | L | A |
| 256 | A | Km | D | R | Aib | A | Q | Aib | N | F | V | N | W | L | L | A |
| 257 | Aib | Km | D | R | Aib | A | Q | Aib | N | F | V | N | W | L | L | A |
| 258 | Aib | Km | D | R | Aib | A | Q | Aib | N | F | V | N | W | L | L | A |
| 259 | Aib | Km | D | R | Aib | A | Q | Aib | N | F | V | N | W | L | L | A |
| 260 | Aib | Km | D | R | Aib | A | Q | Aib | N | F | V | N | W | L | L | A |
| 261 | Aib | Km | D | R | Aib | A | Q | Aib | N | F | V | N | W | L | L | A |
| 262 | Aib | Km | D | R | Aib | A | Q | Aib | N | F | V | N | W | L | L | A |
| 263 | A | L | D | R | Aib | A | Q | Aib | Km | F | V | N | W | L | L | A |
| 264 | A | L | D | R | Aib | H | Q | Aib | Km | F | V | N | W | L | L | A |
| 265 | A | L | D | R | Aib | H | Q | Aib | Km | F | V | N | W | L | L | A |
| 266 | A | L | D | R | Aib | A | Q | Aib | Km | F | V | N | W | L | L | A |
| 267 | A | L | D | R | Aib | A | Q | Aib | Km | F | V | N | W | L | L | A |
| 268 | A | L | D | R | Aib | A | Q | Aib | Km | F | V | N | W | L | L | A |
| 269 | A | L | D | R | Aib | H | Q | Aib | N | F | V | N | W | L | L | A |
| 270 | A | L | D | R | Aib | A | Q | Aib | N | F | V | N | W | L | L | A |
| 271 | A | L | D | R | Aib | A | Q | Aib | N | F | V | N | W | L | L | A |
| 272 | A | L | D | R | Aib | Km | Q | Aib | N | F | V | N | W | L | L | A |
| 273 | A | L | D | R | Aib | Km | Q | Aib | N | F | V | N | W | L | L | A |
| 274 | A | Km | D | R | Aib | A | Q | Aib | D | F | V | N | W | L | L | A |
| 275 | A | Km | D | R | Aib | A | Q | Aib | N | F | V | N | W | L | L | A |
| 276 | A | L | D | R | Km | H | Q | Aib | N | F | V | N | W | L | L | A |
| 277 | A | L | D | R | Aib | A | Q | Aib | Km | F | V | N | W | L | L | A |
| 278 | A | L | D | R | Aib | H | Q | Aib | N | F | V | N | W | L | L | A |
| 279 | A | L | D | R | Aib | A | Q | Aib | Km | F | V | N | W | L | L | A |
| 280 | A | L | D | R | Aib | A | Q | Aib | N | F | V | N | W | L | L | A |
| 281 | A | L | D | R | Aib | Km | Q | Aib | N | F | V | N | W | L | L | A |
| 282 | A | L | D | R | Aib | A | Q | Aib | Km | F | V | N | W | L | L | A |
| 283 | A | L | D | R | Aib | A | Q | Aib | Km | F | V | N | W | L | L | A |
| 284 | A | L | D | R | Aib | A | Q | Aib | Km | F | V | N | W | L | L | A |
| 285 | A | L | D | R | Km | A | Q | Aib | N | F | V | N | W | L | L | A |
| 286 | Aib | L | D | R | Km | A | Q | Aib | N | F | V | N | W | L | L | A |
| 287 | A | Km | D | R | Aib | A | Q | Aib | N | F | V | N | W | L | L | A |
| 288 | Aib | Km | D | R | Aib | A | Q | Aib | N | F | V | N | W | L | L | A |
| 289 | Aib | Km | D | R | Aib | A | Q | Aib | N | F | V | N | W | L | L | A |
| 290 | Aib | L | D | R | Aib | Km | Q | Aib | N | F | V | N | W | L | L | A |
| 291 | Aib | L | D | R | Km | A | Q | Aib | N | F | V | N | W | L | L | A |
| 292 | Aib | L | D | R | Aib | A | Q | Aib | Km | F | V | N | W | L | L | A |
| 293 | A | L | D | R | Km | A | Q | Aib | N | F | V | N | W | L | L | A |
| 294 | A | L | D | R | Aib | A | Q | Aib | Km | F | V | N | W | L | L | A |
| 295 | Aib | L | D | R | Km | A | Q | Aib | N | F | V | N | W | L | L | A |
| 296 | A | Km | D | R | Aib | A | Q | Aib | N | F | V | N | W | L | L | A |
| 297 | A | Km | D | R | Aib | A | Q | Aib | D | F | V | N | W | L | L | A |
| 298 | A | Km | D | S | Aib | A | Q | Aib | [symbol] | F | V | N | W | L | L | A |
| 299 | A | L | D | R | Km | A | Q | Aib | D | F | V | N | W | L | L | A |
| 300 | A | L | D | R | Km | A | Q | Aib | D | F | V | N | W | L | L | A |
| 301 | A | L | D | R | Km | A | Q | Aib | D | F | V | N | W | L | L | A |
| 302 | A | Km | D | R | Aib | A | Q | Aib | D | F | V | N | W | L | L | A |
| 303 | A | M | D | K | Km | H | Q | Q | D | F | V | N | W | L | L | A |
| 304 | A | L | D | R | Aib | A | Q | Aib | Km | F | V | N | W | L | L | A |
| 305 | Aib | L | D | R | Aib | A | Q | Aib | Km | F | V | N | W | L | L | A |

| | | | | | | | | | | | | | SEQ |
|---|---|---|---|---|---|---|---|---|---|---|---|---|---|
| | | | | | | | | | | | | | ID |
| CPMD No. | 29 | 30 | 31 | 32 | 33 | 34 | 35 | 36 | 37 | 38 | 39 | 40 | NO. |
| 1 | Q | K | | | | | | | | | | | 2 |
| 2 | Q | K | | | | | | | | | | | 3 |
| 3 | Q | K | | | | | | | | | | | 4 |
| 4 | Q | K | | | | | | | | | | | 5 |
| 5 | Q | K | | | | | | | | | | | 6 |
| 6 | Q | K | | | | | | | | | | | 7 |
| 7 | Q | K | | | | | | | | | | | 8 |
| 8 | Q | K | | | | | | | | | | | 9 |
| 9 | Q | K | | | | | | | | | | | 10 |
| 10 | Q | K | | | | | | | | | | | 11 |
| 11 | Q | K | | | | | | | | | | | 12 |
| 12 | Q | K | | | | | | | | | | | 13 |
| 13 | Q | R | OH | | | | | | | | | | 14 |
| 14 | Q | R | NH2 | | | | | | | | | | 15 |
| 15 | Q | R | OH | | | | | | | | | | 16 |
| 16 | Q | S | OH | | | | | | | | | | 17 |
| 17 | Q | S | P | S | S | G | A | P | P | P | S | NH2 | 18 |
| 18 | Q | S | P | S | S | G | A | P | P | P | S | NH2 | 19 |
| 19 | Q | R | P | S | S | G | A | P | P | P | S | NH2 | 20 |
| 20 | Q | R | P | S | S | G | A | P | P | P | S | NH2 | 21 |
| 21 | Q | Km | P | S | S | G | A | P | P | P | S | NH2 | 22 |
| 22 | Q | K | NH2 | | | | | | | | | | 23 |
| 23 | Q | K | NH2 | | | | | | | | | | 24 |
| 24 | Q | K | NH2 | | | | | | | | | | 25 |
| 25 | Q | K | O | OH | | | | | | | | | 26 |
| 26 | Q | K | OH | | | | | | | | | | 27 |
| 27 | Q | K | OH | | | | | | | | | | 28 |
| 28 | Q | E | NH2 | | | | | | | | | | 29 |
| 29 | Q | K | NH2 | | | | | | | | | | 30 |
| 30 | Q | K | NH2 | | | | | | | | | | 31 |
| 31 | Q | E | NH2 | | | | | | | | | | 32 |
| 32 | Q | Km | P | | | | | | | | | | 33 |
| 33 | Q | [symbol] | P | | | | | | | | | | 34 |
| 34 | Km | [symbol] | OH | | | | | | | | | | 35 |
| 35 | Km | S | OH | | | | | | | | | | 36 |
| 36 | Km | K | OH | | | | | | | | | | 37 |
| 37 | Q | S | OH | | | | | | | | | | 38 |
| 38 | Q | K | OH | | | | | | | | | | 39 |
| 39 | Aib | K | OH | | | | | | | | | | 40 |
| 40 | Q | [symbol] | OH | | | | | | | | | | 41 |
| 41 | Q | Km | OH | | | | | | | | | | 42 |
| 42 | Q | K | [symbol] | | | | | | | | | | 43 |
| 43 | Q | Km | OH | | | | | | | | | | 44 |
| 44 | Q | K | NH2 | | | | | | | | | | 45 |
| 45 | Aib | K | OH | | | | | | | | | | 46 |
| 46 | Q | [symbol] | P | S | S | G | A | P | P | P | S | OH | 47 |
| 47 | Q | [symbol] | P | S | S | G | A | P | P | P | S | OH | 48 |
| 48 | Q | [symbol] | P | S | S | G | A | P | P | P | S | OH | 49 |
| 49 | Q | [symbol] | P | S | S | G | A | P | P | P | S | OH | 50 |
| 50 | Q | [symbol] | P | S | S | G | A | P | P | P | S | OH | 51 |
| 51 | Q | [symbol] | P | S | S | G | A | P | P | P | S | OH | 52 |
| 52 | Q | Km | P | S | S | G | A | P | P | P | S | OH | 53 |
| 53 | Q | S | P | S | S | G | A | P | P | P | S | OH | 54 |
| 54 | Q | S | P | S | S | G | A | P | P | P | S | OH | 55 |
| 55 | Q | S | P | S | S | G | A | P | P | P | S | OH | 56 |
| 56 | Q | R | P | S | S | G | A | P | P | P | S | OH | 57 |
| 57 | Q | R | P | S | S | G | A | P | P | P | S | OH | 58 |
| 58 | Q | R | P | S | S | G | A | P | P | P | S | NH2 | 59 |
| 59 | Q | R | NH2 | | | | | | | | | | 60 |
| 60 | Q | R | NH2 | | | | | | | | | | 61 |
| 61 | Q | R | NH2 | | | | | | | | | | 62 |
| 62 | Q | R | P | S | S | G | A | P | P | P | S | OH | 63 |
| 63 | Q | R | P | S | S | G | A | P | P | P | S | NH2 | 64 |
| 64 | Q | S | P | S | S | G | A | P | P | P | S | NH2 | 65 |
| 65 | Q | S | P | S | S | G | A | P | P | P | S | NH2 | 66 |
| 66 | Q | R | P | S | S | G | A | P | P | P | S | NH2 | 67 |
| 67 | Q | R | P | S | S | G | A | P | P | P | S | NH2 | 68 |
| 68 | Q | S | P | S | S | G | A | P | P | P | S | NH2 | 69 |
| 69 | Q | S | P | S | S | G | A | P | P | P | S | NH2 | 70 |
| 70 | Q | Km | P | S | S | G | A | P | P | P | S | NH2 | 71 |
| 71 | Q | S | P | S | S | G | A | P | P | P | S | NH2 | 72 |
| 72 | Q | S | P | S | S | G | A | P | P | P | S | NH2 | 73 |
| 73 | Q | R | P | S | S | G | A | P | P | P | S | NH2 | 74 |
| 74 | Q | S | P | S | S | G | A | P | P | P | S | NH2 | 75 |
| 75 | Q | R | P | S | S | G | A | P | P | P | S | NH2 | 76 |
| 76 | Q | S | P | S | S | G | A | P | P | P | S | NH2 | 77 |
| 77 | Q | R | P | S | S | G | A | P | P | P | S | NH2 | 78 |
| 78 | Q | S | P | S | S | G | A | P | P | P | S | NH2 | 79 |
| 79 | Q | S | P | S | S | G | A | P | P | P | S | NH2 | 80 |
| 80 | Q | R | P | S | S | G | A | P | P | P | S | NH2 | 81 |
| 81 | Q | S | P | S | S | G | A | P | P | P | S | NH2 | 82 |
| 82 | Q | S | P | S | S | G | A | P | P | P | S | NH2 | 83 |
| 83 | Q | R | P | S | S | G | A | P | P | P | S | NH2 | 84 |
| 84 | Km | S | NH2 | | | | | | | | | | 85 |
| 85 | Q | S | NH2 | | | | | | | | | | 86 |
| 86 | Q | R | NH2 | | | | | | | | | | 87 |
| 87 | Km | R | P | S | S | G | A | P | P | P | S | NH2 | 88 |
| 88 | Km | R | P | S | S | G | A | P | P | P | S | NH2 | 89 |
| 89 | Q | Km | NH2 | | | | | | | | | | 90 |
| 90 | Q | Km | NH2 | | | | | | | | | | 91 |
| 91 | Q | Km | NH2 | | | | | | | | | | 92 |
| 92 | Q | S | P | S | S | G | A | P | P | P | S | NH2 | 93 |
| 93 | Q | S | P | S | S | G | A | P | P | P | S | NH2 | 94 |
| 94 | Q | R | P | S | S | G | A | P | P | P | S | NH2 | 95 |
| 95 | Q | R | P | S | S | G | A | P | P | P | S | NH2 | 96 |
| 96 | Q | R | G | G | G | G | S | NH2 | | | | | 97 |
| 97 | Q | R | G | G | G | G | S | NH2 | | | | | 98 |
| 98 | Km | S | P | S | S | G | NH2 | | | | | | 99 |
| 99 | Q | R | P | S | S | G | A | P | P | P | S | NH2 | 100 |
| 100 | Q | Km | P | S | S | G | A | P | P | P | S | NH2 | 101 |
| 101 | Q | Km | P | S | S | G | A | P | P | P | S | NH2 | 102 |
| 102 | Q | S | P | S | S | G | A | P | P | P | S | NH2 | 103 |
| 103 | Q | S | P | S | S | G | A | P | P | P | S | NH2 | 104 |
| 104 | Q | R | P | S | S | G | A | P | P | P | S | NH2 | 105 |
| 105 | Q | S | P | S | S | G | A | P | P | P | S | NH2 | 106 |
| 106 | Q | S | P | S | S | G | A | P | P | P | S | NH2 | 107 |
| 107 | Km | S | P | S | S | G | A | P | P | P | S | NH2 | 108 |
| 108 | Q | R | NH2 | | | | | | | | | | 109 |
| 109 | Km | R | P | S | S | G | A | P | P | P | S | NH2 | 110 |
| 110 | Q | R | G | G | G | G | S | NH2 | P | P | S | NH2 | 111 |
| 111 | Q | S | P | S | S | G | A | P | P | P | S | NH2 | 112 |
| 112 | Q | R | NH2 | | | | | | | | | | 113 |
| 113 | Q | R | NH2 | | | | | | | | | | 114 |
| 114 | Q | R | G | G | G | G | S | NH2 | | | | | 115 |
| 115 | Q | R | NH2 | | | | | | | | | | 116 |
| 116 | Q | R | P | S | S | G | A | P | P | P | S | NH2 | 117 |
| 117 | Q | R | NH2 | | | | | | | | | | 118 |
| 118 | Q | R | NH2 | | | | | | | | | | 119 |
| 119 | Q | S | P | S | S | G | A | P | P | P | S | NH2 | 120 |
| 120 | Q | R | P | S | S | G | A | P | P | P | S | NH2 | 121 |
| 121 | Q | R | NH2 | | | | | | | | | | 122 |
| 122 | Q | R | P | S | S | G | A | P | P | P | S | NH2 | 123 |
| 123 | Q | Km | P | S | S | G | A | P | P | P | S | NH2 | 124 |
| 124 | Q | R | P | S | S | G | E | P | K | K | K | NH2 | 125 |
| 125 | Q | S | P | S | S | G | A | P | P | P | S | NH2 | 126 |
| 126 | Q | R | P | S | S | G | A | P | P | P | S | NH2 | 127 |
| 127 | Q | S | P | S | S | G | A | P | P | P | S | NH2 | 128 |
| 128 | Q | R | P | S | S | G | A | P | P | P | S | NH2 | 129 |
| 129 | Q | S | P | S | S | G | A | P | P | P | S | NH2 | 130 |
| 130 | Q | R | P | S | S | G | A | P | P | P | S | NH2 | 131 |
| 131 | Q | S | P | S | S | G | A | P | P | P | S | NH2 | 132 |
| 132 | Q | S | P | S | S | G | A | P | P | P | S | NH2 | 133 |
| 133 | Q | S | G | G | G | G | S | NH2 | | | | | 134 |
| 134 | Q | R | G | G | G | G | S | NH2 | | | | | 135 |
| 135 | Q | R | G | G | G | G | S | NH2 | | | | | 136 |
| 136 | Q | Km | G | G | G | G | S | NH2 | | | | | 137 |
| 137 | Q | R | P | S | S | G | A | P | P | P | S | NH2 | 138 |
| 138 | Q | R | P | S | S | G | A | P | P | P | S | NH2 | 139 |
| 139 | Q | S | P | S | S | G | A | P | P | P | S | NH2 | 140 |
| 140 | Q | S | P | S | S | G | A | P | P | P | S | NH2 | 141 |
| 141 | Q | S | G | OH | | | | | | | | | 142 |
| 142 | Q | R | G | OH | | | | | | | | | 143 |
| 143 | Q | R | G | OH | | | | | | | | | 144 |
| 144 | Q | S | P | S | S | G | A | P | P | P | S | NH2 | 145 |
| 145 | Q | R | P | S | S | G | A | P | P | P | S | NH2 | 146 |
| 146 | Q | R | P | S | S | G | A | P | P | P | S | OH | 147 |
| 147 | Q | R | P | S | S | G | A | P | P | P | S | OH | 148 |
| 148 | Q | R | G | OH | | | | | | | | | 149 |
| 149 | Q | S | G | OH | | | | | | | | | 150 |
| 150 | Q | S | P | S | S | G | A | P | P | P | S | OH | 151 |
| 151 | Q | S | P | S | S | G | A | P | P | P | S | NH2 | 152 |
| 152 | Q | S | P | S | S | G | A | P | P | P | G | OH | 153 |
| 153 | Q | R | P | S | S | G | A | P | P | P | S | NH2 | 154 |
| 154 | Q | S | P | S | S | G | A | P | P | P | S | NH2 | 155 |
| 155 | Q | S | P | S | S | G | A | P | P | P | S | OH | 156 |
| 156 | Q | S | P | S | S | G | A | P | P | P | S | OH | 157 |
| 157 | Q | S | P | S | S | G | A | P | G | OH | | | 158 |
| 158 | Q | S | P | S | S | G | OH | | | | | | 159 |
| 159 | Q | S | P | G | OH | | | | | | | | 160 |
| 160 | Q | S | G | OH | | | | | | | | | 161 |
| 161 | Q | S | G | OH | | | | | | | | | 162 |
| 162 | Q | R | G | OH | | | | | | | | | 163 |
| 163 | Q | S | G | OH | | | | | | | | | 164 |
| 164 | Q | S | P | S | S | G | A | P | P | P | S | NH2 | 165 |
| 165 | Q | R | P | S | S | G | A | P | P | P | S | OH | 166 |
| 166 | Q | S | P | S | S | G | A | P | P | P | S | OH | 167 |
| 167 | Q | S | P | S | S | G | A | P | P | P | S | NH2 | 168 |
| 168 | Q | S | G | OH | | | | | | | | | 169 |
| 169 | Q | R | G | OH | | | | | | | | | 170 |
| 170 | Q | S | G | OH | | | | | | | | | 171 |
| 171 | Q | R | G | OH | | | | | | | | | 172 |
| 172 | Q | S | G | OH | | | | | | | | | 173 |
| 173 | Q | R | G | OH | | | | | | | | | 174 |
| 174 | Q | S | G | OH | | | | | | | | | 175 |
| 175 | Q | R | G | OH | | | | | | | | | 176 |
| 176 | Q | R | G | OH | | | | | | | | | 177 |
| 177 | Q | R | G | OH | | | | | | | | | 178 |
| 178 | Q | R | G | OH | | | | | | | | | 179 |
| 179 | Q | R | G | OH | | | | | | | | | 180 |
| 180 | Q | R | G | OH | | | | | | | | | 181 |
| 181 | Q | K(Ac) | G | OH | | | | | | | | | 182 |
| 182 | Q | S | P | G | OH | | | | | | | | 183 |
| 183 | Q | S | P | G | OH | | | | | | | | 184 |
| 184 | Q | S | P | G | OH | | | | | | | | 185 |
| 185 | Q | S | P | G | OH | | | | | | | | 186 |
| 186 | Q | S | P | G | OH | | | | | | | | 187 |
| 187 | Q | S | P | S | S | G | A | P | P | P | S | OH | 188 |
| 188 | Q | R | G | OH | | | | | | | | | 189 |
| 189 | Q | S | P | S | S | G | A | P | P | P | S | OH | 190 |
| 190 | Q | R | NH2 | | | | | | | | | | 191 |
| 191 | G | G | P | S | S | G | A | P | P | P | S | Km | 192 |
| 192 | Q | K | | | | | | | | | | | 193 |
| 193 | Q | K | | | | | | | | | | | 194 |
| 194 | Q | K | | | | | | | | | | | 195 |
| 195 | Q | K | | | | | | | | | | | 196 |
| 196 | Q | K | | | | | | | | | | | 197 |
| 197 | Q | K | | | | | | | | | | | 198 |
| 198 | Q | K | G | OH | | | | | | | | | 199 |
| 199 | Q | R | G | OH | | | | | | | | | 200 |
| 200 | Q | K | P | S | S | G | A | P | P | P | S | OH | 201 |
| 201 | Q | K | G | OH | | | | | | | | | 202 |
| 202 | Q | K | G | OH | | | | | | | | | 203 |
| 203 | Q | R | G | OH | | | | | | | | | 204 |
| 204 | Q | K | G | OH | | | | | | | | | 205 |
| 205 | Q | K | G | NH2 | | | | | | | | | 206 |
| 206 | Q | K | G | OH | | | | | | | | | 207 |
| 207 | Q | R | G | OH | | | | | | | | | 208 |
| 208 | Q | R | G | OH | | | | | | | | | 209 |
| 209 | Q | K | P | S | S | G | A | P | P | P | S | NH2 | 210 |
| 210 | Q | K | NH2 | | | | | | | | | | 211 |
| 211 | Q | R | G | OH | | | | | | | | | 212 |
| 212 | Q | K | G | OH | | | | | | | | | 213 |
| 213 | Q | K | G | OH | | | | | | | | | 214 |
| 214 | Q | K | G | OH | | | | | | | | | 215 |
| 215 | Q | K | G | OH | | | | | | | | | 216 |
| 216 | Q | K | G | OH | | | | | | | | | 217 |
| 217 | Q | K | G | OH | | | | | | | | | 218 |
| 218 | Q | K | G | OH | | | | | | | | | 219 |
| 219 | Q | K | G | OH | | | | | | | | | 220 |
| 220 | Q | K | G | OH | | | | | | | | | 221 |
| 221 | Q | K | G | OH | | | | | | | | | 222 |
| 222 | Km | K | G | OH | | | | | | | | | 223 |
| 223 | Q | Km | G | OH | | | | | | | | | 224 |
| 224 | Q | K | G | OH | | | | | | | | | 225 |
| 225 | Q | K | G | OH | | | | | | | | | 226 |
| 226 | Q | Km | | | | | | | | | | | 227 |
| 227 | Q | K | | | | | | | | | | | 228 |
| 228 | Q | K | | | | | | | | | | | 229 |
| 229 | Q | Km | | | | | | | | | | | 230 |
| 230 | Q | Km | | | | | | | | | | | 231 |
| 231 | Q | K | NH2 | | | | | | | | | | 232 |
| 232 | Q | K | NH2 | | | | | | | | | | 233 |
| 233 | Q | K | NH2 | | | | | | | | | | 234 |
| 234 | Q | K | NH2 | | | | | | | | | | 235 |
| 235 | Q | K | NH2 | | | | | | | | | | 236 |
| 236 | Q | K | NH2 | | | | | | | | | | 237 |
| 237 | Q | K | | | | | | | | | | | 238 |
| 238 | Q | K | | | | | | | | | | | 239 |
| 239 | Q | K | | | | | | | | | | | 240 |
| 240 | Q | K | | | | | | | | | | | 241 |
| 241 | Q | K | | | | | | | | | | | 242 |
| 242 | Q | K | | | | | | | | | | | 243 |
| 243 | Q | K | | | | | | | | | | | 244 |
| 244 | Q | K | | | | | | | | | | | 245 |
| 245 | Q | K | | | | | | | | | | | 246 |
| 246 | Q | H | P | S | S | G | A | P | P | P | S | NH2 | 247 |
| 247 | Q | K | G | OH | | | | | | | | | 248 |
| 248 | Q | S | P | S | S | G | A | P | P | P | S | OH | 249 |
| 249 | Q | S | OH | | | | | | | | | | 250 |
| 250 | Q | S | OH | | | | | | | | | | 251 |
| 251 | Q | S | OH | | | | | | | | | | 252 |
| 252 | Q | K | NH2 | | | | | | | | | | 253 |
| 253 | Km | K | OH | | | | | | | | | | 254 |
| 254 | Km | S | NH2 | | | | | | | | | | 255 |
| 255 | Q | K | NH2 | | | | | | | | | | 256 |
| 256 | Q | K | NH2 | | | | | | | | | | 257 |
| 257 | Q | R | P | S | S | G | A | P | P | P | S | NH2 | 258 |
| 258 | Q | R | P | S | S | G | A | P | P | P | S | OH | 259 |
| 259 | Q | S | NH2 | | | | | | | | | | 260 |
| 260 | Q | R | NH2 | | | | | | | | | | 261 |
| 261 | Q | R | NH2 | | | | | | | | | | 262 |
| 262 | Q | R | NH2 | | | | | | | | | | 263 |
| 263 | Q | R | NH2 | | | | | | | | | | 264 |
| 264 | Q | R | NH2 | | | | | | | | | | 265 |
| 265 | Q | S | NH2 | | | | | | | | | | 266 |
| 266 | Q | S | OH | | | | | | | | | | 267 |
| 267 | Q | S | NH2 | | | | | | | | | | 268 |
| 268 | Q | S | NH2 | | | | | | | | | | 269 |
| 269 | Q | R | P | S | S | G | A | P | P | P | S | OH | 270 |
| 270 | Q | Km | P | S | S | G | A | P | P | P | S | NH2 | 271 |
| 271 | Q | Km | P | S | S | G | A | P | P | P | S | NH2 | 272 |
| 272 | Q | R | P | S | S | G | A | P | P | P | S | NH2 | 273 |
| 273 | Q | R | P | S | S | G | A | P | P | P | S | NH2 | 274 |
| 274 | Q | S | P | S | S | G | A | P | P | P | S | NH2 | 275 |
| 275 | Q | R | P | S | S | G | A | P | P | P | S | NH2 | 276 |
| 276 | Q | R | P | S | S | G | A | P | P | P | S | NH2 | 277 |
| 277 | Q | R | P | S | S | G | A | P | P | P | S | NH2 | 278 |
| 278 | Km | R | NH2 | | | | | | | | | | 279 |
| 279 | Q | R | G | OH | NH2 | | | | | | | | 280 |
| 280 | Km | S | P | S | S | G | A | P | P | P | S | NH2 | 281 |
| 281 | Q | R | NH2 | | | | | | | | | | 282 |
| 282 | Q | R | NH2 | | | | | | | | | | 283 |
| 283 | Q | S | NH2 | | | | | | | | | | 284 |
| 284 | Q | S | NH2 | | | | | | | | | | 285 |
| 285 | Q | R | P | S | S | G | A | P | P | P | S | NH2 | 286 |
| 286 | Q | R | P | S | S | G | A | P | P | P | S | NH2 | 287 |
| 287 | Q | R | P | S | S | G | A | P | P | P | S | NH2 | 288 |
| 288 | Q | R | P | S | S | G | A | P | P | P | S | NH2 | 289 |
| 289 | Q | K | NH2 | | | | | | | | | | 290 |
| 290 | Q | R | NH2 | | | | | | | | | | 291 |
| 291 | Q | R | P | S | S | K | K | K | K | K | K | NH2 | 292 |
| 292 | Q | R | P | S | S | K | K | K | K | K | K | NH2 | 293 |
| 293 | Q | R | P | S | S | G | A | P | P | P | S | NH2 | 294 |
| 294 | Q | N | G | G | G | G | G | [symbol] | NH2 | | | | 295 |
| 295 | Q | S | P | S | S | G | A | P | P | P | S | NH2 | 296 |
| 296 | Q | S | P | S | S | G | A | P | P | P | S | NH2 | 297 |
| 297 | Q | R | P | S | S | G | A | P | P | P | S | NH2 | 298 |
| 298 | Q | R | P | S | S | G | A | P | P | P | S | NH2 | 299 |
| 299 | Q | R | G | OH | | | | | | | | | 300 |
| 300 | Q | R | P | S | S | G | A | P | P | P | S | NH2 | 301 |
| 301 | Q | R | G | OH | | | | | | | | | 302 |
| 302 | Q | K | G | OH | | | | | | | | | 303 |
| 303 | Q | K | G | K | K | N | D | W | K | N | N | I | 304 |
| 304 | Q | K | G | OH | | | | | | | | | |
| 305 | Q | R | G | OH | | | | | | | | | |

N-TER means N-terminus;
C-TER means C-terminus;
C15DA means C₁₅ diacid;
C16DA means C₁₆ diacid [symbol]
indicates data missing or illegible when filed

### Biological Examples

Methods for performing GIP and GLP receptor binding assays, assays for inhibition of emesis, vomiting and nausea, caused by various stimuli, including from drug or chemotherapy induced emesis are specifically described in Applicant's International PCT Application No. PCT/JP2018/013540, filed on Mar. 30, 2018, ranging from pages 213 to 255, and are specifically incorporated herein by reference in their entirety.

### Example 8-Evaluation of Peptide Agonist Activity on Human GIPR and Human GLP1R by Measuring Intracellular cAMP Accumulation

GIPR Assay

HEK-293T cells overexpressing full-length human GIPR with a sequence identical to GenBank accession number NM_000164 with an N-terminal FLAG tag are purchased from Multispan, Inc (Hayward, Calif.). Cells are cultured per the manufacturer's protocol in DMEM with 10% fetal bovine serum and 1 μg/mL puromycin and stored in frozen aliquots to be used as assay ready cells. On the day of the assay, cells are removed from frozen storage, washed two times in 1× Kreb's Ringer Buffer (Zenbio, Research Triangle Park, N.C.), and re-suspended to a concentration of 4×10⁵ cells/mL in 1× Kreb's Ringer Buffer. 50 nL of test compound in 100% DMSO spanning a final concentration range of 3×10⁻¹⁰-5.08×10⁻¹⁵ M are acoustically dispensed in low volume, white, 384-well polypropylene plates (Corning, Tewksbury, Mass.), followed by the addition of 4×10³ cells per well in total volume of 10 μL. Cells are incubated with test compound for 1 hr at room temperature in the dark, and cAMP accumulation is measured using the Cisbio HiRange cAMP assay kit (Bedford, Mass.) per the manufacturer's protocol. Anti-cAMP antibody and d2-cAMP tracer reagents diluted in lysis/detection buffer are incubated in the dark for 1 hr, and results are measured on an Envision plate reader (Perkin Elmer, Waltham, Mass.). Data is normalized using 1 nM GIP as 100% activity, and DMSO alone as 0% activity.

HEK-293T cells overexpressing full-length human GLP-1R with a sequence identical to GenBank accession number NM_002062 with an N-terminal FLAG tag may be purchased from Multispan, Inc (Hayward, Calif.). Cells are cultured per the manufacturer's protocol in DMEM with 100 fetal bovine serum and 1 μg/mL puromycin and stored in frozen aliquots to be used as assay ready cells. On the day of the assay, cells are removed from frozen storage, washed two times in 1× Kreb's Ringer Buffer (Zenbio, Research Triangle Park, N.C.), and re-suspended to a concentration of 4×10⁵ cells/mL in 1× Kreb's Ringer Buffer. 50 nL of test compound in 100% DMSO spanning a final concentration range of 1×10⁻⁶-1.69×10⁻¹¹ M are acoustically dispensed in low volume, white, 384-well polypropylene plates (Corning, Tewksbury, Mass.), followed by the addition of 4×10³ cells per well in total volume of 10 μL. Cells are incubated with test compound for 1 hr at room temperature in the dark, and cAMP accumulation is measured using the Cisbio HiRange cAMP assay kit (Bedford, Mass.) per the manufacturer's protocol. Anti-cAMP antibody and d2-cAMP tracer reagents diluted in lysis/detection buffer are incubated in the dark for 1 hr, and results are measured on an Envision plate reader (Perkin Elmer, Waltham, Mass.). Data is normalized using 1 nM GLP-1 as 100% activity, and DMSO alone as 0% activity.

TABLE 4
GIP receptor selective activation of various GIP
receptor agonist peptides of the disclosure

| Compound | Sequence | Human GIPR cAMP, | Human GLP1R cAMP |
|---|---|---|---|
| No. | ID No. | HEK293T EC50 (nM) | HEK293T EC50 (nM) |
| 1 | 2 | 0.0002 | 870.9636 |
| 2 | 3 | 0.0001 | 1000 |
| 3 | 4 | 0.0004 | 691.831 |
| 4 | 5 | 0.0005 | 691.831 |
| 5 | 6 | 0.0008 | 954.9926 |
| 6 | 7 | 0.0537 | 691.831 |
| 7 | 8 | 0.0016 | 257.0396 |
| 8 | 9 | 0.0001 | 100 |
| 9 | 10 | 0.0002 | 95.4993 |
| 10 | 11 | 0.0006 | 954.9926 |
| 11 | 12 | 0.0003 | 144.544 |
| 12 | 13 | 0.0002 | 363.0781 |
| 13 | 14 | 0.0002 | 142.8894 |
| 14 | 15 | 0.0004 | 61.0942 |
| 15 | 16 | 0.0003 | 154.8817 |
| 16 | 17 | 0.0003 | 263.0268 |
| 17 | 18 | 0.0004 | 160.3245 |
| 18 | 19 | 0.0002 | 1000 |
| 19 | 20 | 0.0004 | 119.3072 |
| 20 | 21 | 0.0003 | 116.1449 |
| 21 | 22 | 0.0003 | 152.5223 |
| 22 | 23 | 0.0003 | 630.9573 |
| 23 | 24 | 0.0004 | 363.0781 |
| 24 | 25 | 0.0004 | 16.8526 |
| 25 | 26 | 0.0005 | 37.368 |
| 26 | 27 | 0.0003 | 1000 |
| 27 | 28 | 0.0006 | 144.544 |
| 28 | 29 | 0.0001 | 60.256 |
| 29 | 30 | 0.0001 | 47.1339 |
| 30 | 31 | 0.0001 | 441.5704 |
| 31 | 32 | 0.0002 | 0.7762 |
| 32 | 33 | 0.0002 | 4.7315 |
| 33 | 34 | 0.0002 | 37.5837 |
| 34 | 35 | 0.0003 | 117.4898 |
| 35 | 36 | 0.0003 | 67.6083 |
| 36 | 37 | 0.001 | 25.1189 |
| 37 | 38 | 0.0002 | 43.6516 |
| 38 | 39 | 0.0002 | 524.8075 |
| 39 | 40 | 0.0005 | 398.1072 |
| 40 | 41 | 0.0008 | 114.8154 |
| 41 | 42 | 0.0004 | 112.2018 |
| 42 | 43 | 0.0006 | 237.1374 |
| 43 | 44 | 0.0003 | 173.7801 |
| 44 | 45 | 0.0004 | 630.9573 |
| 45 | 46 | 0.0003 | 48.4172 |
| 46 | 47 | 0.0002 | 0.1445 |
| 47 | 48 | 0.0003 | 109.6478 |
| 48 | 49 | 0.0002 | 47.863 |
| 49 | 50 | 0.0002 | 794.3282 |
| 50 | 51 | 0.0004 | 44.6684 |
| 51 | 52 | 0.0003 | 1000 |
| 52 | 53 | 0.0003 | 239.8833 |
| 53 | 54 | 0.0001 | 616.595 |
| 54 | 55 | 0.0001 | 446.6836 |
| 55 | 56 | 0.0002 | 239.8833 |
| 56 | 57 | 0.0003 | 263.0268 |
| 59 | 60 | 0.0004 | 56.2341 |
| 63 | 64 | 0.0002 | 141.2538 |
| 64 | 65 | 0.0005 | 1000 |
| 65 | 66 | 0.0003 | 128.825 |
| 66 | 67 | 0.0004 | 1000 |
| 67 | 68 | 0.0006 | 524.8075 |
| 68 | 69 | 0.0007 | 199.5262 |
| 69 | 70 | 0.0004 | 1000 |
| 70 | 71 | 0.0009 | 91.2011 |
| 71 | 72 | 0.0006 | 954.9926 |
| 72 | 73 | 0.0006 | 190.5461 |
| 73 | 74 | 0.0005 | 1000 |
| 74 | 75 | 0.0004 | 549.5409 |
| 75 | 76 | 0.0011 | 1000 |
| 76 | 77 | 0.0005 | 295.1209 |
| 77 | 78 | 0.0002 | 331.1311 |
| 78 | 79 | 0.0003 | 112.2018 |
| 79 | 80 | 0.0002 | 707.9458 |
| 80 | 81 | 0.0004 | 467.7351 |
| 81 | 82 | 0.0002 | 1000 |
| 82 | 83 | 0.0003 | 295.1209 |
| 84 | 85 | 0.0012 | 371.5352 |
| 85 | 86 | 0.0009 | 63.0957 |
| 86 | 87 | 0.0014 | 93.3254 |
| 87 | 88 | 0.0009 | 199.5262 |
| 88 | 89 | 0.0006 | 165.9587 |
| 89 | 90 | 0.0006 | 83.1764 |
| 90 | 91 | 0.0007 | 295.1209 |
| 91 | 92 | 0.0008 | 125.8925 |
| 92 | 93 | 0.0013 | 74.131 |
| 93 | 94 | 0.0002 | 489.7788 |
| 94 | 95 | 0.0006 | 501.1872 |
| 95 | 96 | 0.0002 | 537.0318 |
| 96 | 97 | 0.0004 | 89.1251 |
| 97 | 98 | 0.0002 | 489.7788 |
| 98 | 99 | 0.0006 | 1000 |
| 99 | 100 | 0.0002 | 1000 |
| 100 | 101 | 0.0007 | 1000 |
| 101 | 102 | 0.0002 | 549.5409 |
| 102 | 103 | 0.0005 | 31.6228 |
| 103 | 104 | 0.0005 | 147.9108 |
| 104 | 105 | 0.0004 | 1000 |
| 105 | 106 | 0.0003 | 1000 |
| 106 | 107 | 0.0005 | 120.2264 |
| 107 | 108 | 0.0008 | 891.2509 |
| 108 | 109 | 0.0005 | 41.6869 |
| 109 | 110 | 0.0005 | 831.7638 |
| 110 | 111 | 0.0004 | 323.5937 |
| 111 | 112 | 0.0004 | 7.0795 |
| 112 | 113 | 0.0004 | 12.0226 |
| 113 | 114 | 0.0004 | 44.6684 |
| 114 | 115 | 0.0007 | 114.8154 |
| 115 | 116 | 0.0005 | 436.5158 |
| 116 | 117 | 0.0003 | 338.8442 |
| 117 | 118 | 0.0006 | 1000 |
| 118 | 119 | 0.0009 | 524.8075 |
| 119 | 120 | 0.0005 | 1000 |
| 120 | 121 | 0.0004 | 5.2481 |
| 121 | 122 | 0.0003 | 5.4954 |
| 122 | 123 | 0.0004 | 20.4174 |
| 123 | 124 | 0.0004 | 22.9087 |
| 124 | 125 | 0.004 | 125.8925 |
| 125 | 126 | 0.0002 | 165.9587 |
| 126 | 127 | 0.0002 | 588.8437 |
| 127 | 128 | 0.0005 | 66.0693 |
| 128 | 129 | 0.0004 | 457.0882 |
| 129 | 130 | 0.0005 | 1000 |
| 130 | 131 | 0.0004 | 91.2011 |
| 131 | 132 | 0.0003 | 75.8578 |
| 132 | 133 | 0.0001 | 21.8776 |
| 133 | 134 | 0.0005 | 1000 |
| 134 | 135 | 0.0004 | 1000 |
| 135 | 136 | 0.0005 | 645.6542 |
| 136 | 137 | 0.0009 | 407.3803 |
| 137 | 138 | 0.0002 | 478.6301 |
| 138 | 139 | 0.0004 | 1000 |
| 139 | 140 | 0.0004 | 1000 |
| 140 | 141 | 0.0004 | 177.8279 |
| 141 | 142 | 0.0003 | 64.5654 |
| 142 | 143 | 0.0001 | 84.1395 |
| 143 | 144 | 0.0004 | 141.2538 |
| 144 | 145 | 0.0001 | 18.8365 |
| 145 | 146 | 0.0004 | 912.0108 |
| 146 | 147 | 0.0005 | 112.2018 |
| 147 | 148 | 0.0011 | 16.5959 |
| 148 | 149 | 0.0011 | 13.8038 |
| 149 | 150 | 0.0007 | 25.704 |
| 150 | 151 | 0.0003 | 30.1995 |
| 151 | 152 | 0.0004 | 144.544 |
| 152 | 153 | 0.0003 | 301.9952 |
| 153 | 154 | 0.0002 | 389.0451 |
| 154 | 155 | 0.0003 | 288.4032 |
| 155 | 156 | 0.0003 | 295.1209 |
| 156 | 157 | 0.0002 | 89.1251 |
| 157 | 158 | 0.0001 | 346.7368 |
| 158 | 159 | 0.0002 | 245.4709 |
| 159 | 160 | 0.0002 | 144.544 |
| 160 | 161 | 0.0003 | 23.9883 |
| 161 | 162 | 0.0003 | >1000.0000 |
| 162 | 163 | 0.0002 | 512.8614 |
| 163 | 164 | 0.0003 | >1000.0000 |
| 164 | 165 | 0.0002 | 9.7724 |
| 165 | 166 | 0.0004 | 85.1138 |
| 166 | 167 | 0.0004 | 89.1251 |
| 167 | 168 | 0.0004 | 41.6869 |
| 168 | 169 | 0.0003 | 17.378 |
| 169 | 170 | 0.0006 | >1000.0000 |
| 170 | 171 | 0.0004 | >1000.0000 |
| 171 | 172 | 0.0005 | >1000.0000 |
| 172 | 173 | 0.0004 | 64.5654 |
| 173 | 174 | 0.0005 | >1000.0000 |
| 174 | 175 | 0.0003 | 10.7152 |
| 175 | 176 | 0.0004 | 44.6684 |
| 176 | 177 | 0.0004 | 89.1251 |
| 177 | 178 | 0.0002 | 1000 |
| 180 | 181 | 0.0003 | >1000.0000 |
| 181 | 182 | 0.0004 | 162.181 |
| 182 | 183 | 0.0002 | 154.8817 |
| 183 | 184 | 0.0005 | 89.1251 |
| 184 | 185 | 0.0003 | >1000.0000 |
| 185 | 186 | 0.0004 | >1000.0000 |
| 186 | 187 | 0.0003 | 26.9153 |
| 187 | 188 | 0.0002 | 51.2861 |
| 188 | 189 | 0.0003 | 22.9087 |
| 189 | 190 | 0.0003 | 15.4882 |
| 190 | 191 | 0.0158 | 1000 |
| 191 | 192 | 0.0049 | 0.0058 |
| 192 | 193 | 0.1479 | 741.3102 |
| 193 | 194 | 0.0741 | 17.7828 |
| 194 | 195 | 0.0389 | 125.8925 |
| 195 | 196 | 0.0708 | 1000 |
| 196 | 197 | 0.0002 | 691.831 |
| 201 | 202 | 0.0002 | 9.1201 |
| 202 | 203 | 0.0003 | 14.7911 |
| 203 | 204 | 0.003 | 2.6303 |
| 204 | 205 | 0.0008 | 3.0903 |
| 205 | 206 | 0.0002 | 13.4896 |
| 206 | 207 | 0.0013 | 144.544 |
| 207 | 208 | 0.0004 | 9.3325 |
| 208 | 209 | 0.0003 | 57.544 |
| 209 | 210 | 0.0003 | 478.6301 |
| 210 | 211 | 0.0003 | 128.825 |
| 211 | 212 | 0.0002 | 173.7801 |
| 212 | 213 | 0.0002 | 66.0693 |
| 213 | 214 | 0.0003 | 16.9824 |
| 214 | 215 | 0.0003 | 56.2341 |
| 215 | 216 | 0.0002 | 45.7088 |
| 216 | 217 | 0.0003 | 34.6737 |
| 217 | 218 | 0.0002 | 4.8978 |
| 218 | 219 | 0.0006 | 89.1251 |
| 219 | 220 | 0.0004 | 6.7608 |

Table 4 provides the selective binding activity of the GIPR agonist peptides of the present disclosure. As can be seen, the peptide compounds provided here have a human GLP1R cAMP EC₅₀/human GIPR cAMP EC₅₀ ratios ranging from about 800 to about 10,000,000, thus indicating incredibly selective GIPR agonist binding activity. Most of the GIPR agonist peptide compounds display Human GLP1R cAMP EC₅₀/Human GIPR cAMP EC₅₀ ratios of greater than 1,000, or greater than 5,000, or greater than 10,000, or greater than 50,000, or greater than 100,000, or greater than 500,000.

### Example 9. Oral Glucose Tolerance Test

An oral glucose tolerance test (OGTT) was carried out using C57BL/6J mice with a glucose load of 2.5 g/kg by oral administration. Testing concentrations of 0.1, 0.3 or 3 nmol/kg were selected depending on the peptide. Each peptide or a vehicle (control group) was subcutaneously administered 30 mi before glucose loading and the blood glucose levels were measured at 15, 30, 60 and 120 min post oral glucose administration to evaluate the action of the compound. The action of the compound was calculated by the calculation formula below and expressed as the 0% drop in glucose as measured over 120 min using AUC.

% inhibition=(1−(AUC cpd/AUC vehicle))×100.

Results are shown in Table 5. As shown in Table 5, it is verified that the compounds of the present invention suppress increase in blood glucose level caused by oral glucose loading.

TABLE 5
oral glucose tolerance test

| | | Oral Glucose Tolerance Test |
|---|---|---|
| | | Percent decrease of blood |
| Compound | Sequence | glucose AUC (%) over 120 mins |
| No. | ID No. | at different compound doses (nmol/kg) |
| 14 | 15 | 23% (0.1 nmol/kg), |
| | | 37% (3 nmol/kg) |
| 17 | 18 | 24% (0.1 nmol/kg), |
| | | 39% (3 nmol/kg) |
| 18 | 19 | 29% (3 nmol/kg) |
| 19 | 20 | 38% (3 nmol/kg) |
| 20 | 21 | 42% (3 nmol/kg) |
| 21 | 22 | 29% (0.1 nmol/kg), |
| | | 36% (0.3 nmol/kg), |
| | | 44% (3 nmol/kg) |
| 24 | 25 | 40% (3 nmol/kg) |
| 25 | 26 | 28% (0.1 nmol/kg), |
| | | 33% (0.3 nmol/kg)/kg, |
| | | 41% (3 nmol/kg) |
| 29 | 30 | 20% (0.1 nmol/kg) |
| 30 | 31 | 30% (3 nmol/kg) |
| 31 | 32 | 20% (0.1 nmol/kg) |
| 32 | 33 | 35% (3 nmol/kg) |
| 33 | 34 | 34% (3 nmol/kg) |
| 34 | 35 | 38% (3 nmol/kg) |
| 37 | 38 | 44% (3 nmol/kg) |
| 41 | 42 | 12% (0.1 nmol/kg) |
| 42 | 43 | 35% (3 nmol/kg) |
| 43 | 44 | 39% (3 nmol/kg) |
| 44 | 45 | 42% (3 nmol/kg) |
| 45 | 46 | 39% (3 nmol/kg) |
| 48 | 49 | 22% (0.1 nmol/kg), |
| | | 35% (0.3 nmol/kg), |
| | | 43% (3 nmol/kg) |
| 49 | 50 | 29% (3 nmol/kg) |
| 50 | 51 | 41% (3 nmol/kg) |
| 52 | 53 | 34% (3 nmol/kg) |
| 54 | 55 | 14% (0.1 nmol/kg) |
| 55 | 56 | 36% (3 nmol/kg) |
| 58 | 59 | 17% (0.1 nmol/kg) |
| 60 | 61 | 06% (0.1 nmol/kg) |
| 63 | 64 | 10% (0.1 nmol/kg) |
| 65 | 66 | 21% (0.1 nmol/kg) |
| 68 | 69 | 24% (0.1 nmol/kg) |
| 69 | 70 | 18% (0.1 nmol/kg) |
| 70 | 71 | 23% (0.1 nmol/kg) |
| 71 | 72 | 24% (0.1 nmol/kg) |
| 72 | 73 | 28% (0.1 nmol/kg) |
| 74 | 75 | 27% (0.1 nmol/kg) |
| 75 | 76 | 24% (0.1 nmol/kg) |
| 78 | 79 | 28% (0.1 nmol/kg) |
| 83 | 84 | 26% (0.1 nmol/kg) |
| 84 | 85 | 14% (0.1 nmol/kg) |
| 87 | 88 | 19% (0.1 nmol/kg) |
| 88 | 89 | 14% (0.1 nmol/kg) |
| 90 | 91 | 06% (0.1 nmol/kg) |
| 91 | 92 | 00% (0.1 nmol/kg) |
| 92 | 93 | 18% (0.1 nmol/kg) |
| 93 | 94 | 36% (0.1 nmol/kg) |
| | | 39% (0.3 nmol/kg) |
| 96 | 97 | 31% (0.1 nmol/kg) |
| 98 | 99 | 09% (0.1 nmol/kg) |
| 100 | 101 | 11% (0.1 nmol/kg) |
| 101 | 102 | 27% (0.1 nmol/kg) |
| 102 | 103 | 37% (0.1 nmol/kg) |
| 103 | 104 | 32% (0.1 nmol/kg) |
| 106 | 107 | 26% (0.1 nmol/kg) |
| 107 | 108 | 25% (0.1 nmol/kg) |
| 108 | 109 | 28% (0.1 nmol/kg) |
| 109 | 110 | 13% (0.1 nmol/kg) |
| 111 | 112 | 31% (0.1 nmol/kg) |
| 114 | 115 | 28% (0.1 nmol/kg) |
| 117 | 118 | 11% (0.1 nmol/kg) |
| 121 | 122 | 01% (0.1 nmol/kg) |
| 123 | 124 | 10% (0.1 nmol/kg) |
| 124 | 125 | 12% (0.1 nmol/kg) |
| 131 | 132 | 36% (0.1 nmol/kg) |
| 132 | 133 | 38% (0.1 nmol/kg) |
| | | 33% (0.3 nmol/kg) |
| 133 | 134 | 35% (0.1 nmol/kg) |
| 136 | 137 | 23% (0.1 nmol/kg) |
| 140 | 141 | 21% (0.1 nmol/kg) |
| 141 | 142 | 10% (0.1 nmol/kg) |
| 142 | 143 | 33% (0.1 nmol/kg), |
| | | 21 and 28% (0.3 nmol/kg) |
| 143 | 144 | 13% (0.1 nmol/kg) |
| 144 | 145 | 26% (0.1 nmol/kg), |
| | | 25% (0.3 nmol/kg) |
| 146 | 147 | 30% (0.1 nmol/kg) |
| 147 | 148 | 28% (0.1 nmol/kg) |
| 148 | 149 | 045 and 042% (3 nmol/kg) |
| 149 | 150 | 20% (0.1 nmol/kg) |
| 150 | 151 | 23% (0.1 nmol/kg) |
| 151 | 152 | 24% (0.1 nmol/kg) |
| 152 | 153 | 18% (0.1 nmol/kg) |
| 153 | 154 | 21% (0.1 nmol/kg) |
| 154 | 155 | 18% (0.1 nmol/kg) |
| 155 | 156 | 18% (0.1 nmol/kg) |
| 156 | 157 | 31% (0.1 nmol/kg) |
| 157 | 158 | 20% (0.1 nmol/kg) |
| 158 | 159 | 20% (0.1 nmol/kg) |
| 159 | 160 | 32% (0.1 nmol/kg) |
| | | 18% (0.3 nmol/kg) |
| 160 | 161 | 20% (0.1 nmol/kg) |
| 162 | 163 | 00% (0.1 nmol/kg) |
| 164 | 165 | 34% (0.1 nmol/kg) |
| 165 | 166 | 29% (0.1 nmol/kg) |
| 166 | 167 | 12% (0.1 nmol/kg) |
| 167 | 168 | 34% (0.1 nmol/kg) |
| 169 | 170 | 10% (0.1 nmol/kg) |
| 171 | 172 | 23% (0.1 nmol/kg) |
| 173 | 174 | 14% (0.1 nmol/kg) |
| 175 | 176 | 17% (0.1 nmol/kg), |
| | | 25% (0.3 nmol/kg) |
| 176 | 177 | 14% (0.1 nmol/kg), |
| | | 16% (0.3 nmol/kg) |
| 177 | 178 | 18% (0.1 nmol/kg) |
| 178 | 179 | 11% (0.1 nmol/kg) |
| 180 | 181 | 18% (0.1 nmol/kg) |
| 181 | 182 | 02% (0.1 nmol/kg) |
| 182 | 183 | 09% (0.1 nmol/kg) |
| 183 | 184 | 24% (0.1 nmol/kg) |
| 184 | 185 | 32% (0.1 nmol/kg) |
| 185 | 186 | 08% (0.1 nmol/kg) |
| 186 | 187 | 07% (0.1 nmol/kg) |
| 187 | 188 | 30% (0.1 nmol/kg), |
| | | 38% (0.3 nmol/kg) |

As shown in Table 5, the GIPR agonist peptide compounds of the present invention with a 20% or greater decrease in blood glucose suppress increase in blood glucose level caused by oral glucose loading.

### Example 10: PYY-1119-Induced Vomiting in Dogs

Effects of single subcutaneous administration of the GIPR agonist compounds of the present disclosure on Neuropeptide Y2 receptor (Y2R) agonist compound PYY-1119 (4-imidazolecarbonyl-Ser-D-Hyp-Iva-Pya(4)-Cha-Leu(Me)-Asn-Lys-Aib-Thr-Arg-Gln-Arg-Cha-NH2) (10 μg/kg [about 5 nmol/kg], s.c.) induced emesis were evaluated in dogs. The GIPR agonist peptide compounds of the present disclosure or vehicle (0.09% [w/v] Tween 80/10% DMSO/PBS) was administered subcutaneously (sc) at different doses to female beagle dogs (10 months old), followed by sc injections with Y2R agonist ((4-imidazolecarbonyl-Ser-D-Hyp-Iva-Pya(4)-Cha-Leu(Me)-Asn-Lys-Aib-Thr-Arg-Gln-Arg-Cha-NH2), 10 μg/kg), 10 μg/kg) at 1 hour or specified hours in the table postdose. Emetic episodes were counted for 2 hours after administration (by blinded analysis).

Table 6 shows the compounds suppressed the PYY-1119-induced emetic symptoms. In the below table, results are shown as percent inhibition (%) at the dose of compound (nmol/kg) shown, at the hour(s) postdose of PYY-1119, calculated as (1−(number of emetic episodes with peptide compound/number of emetic episodes with vehicle))×100.

TABLE 6
Percent inhibition of emetic episodes in dogs
treated with the peptide compounds of the present
disclosure when challenged with PYY-1119.

| | | Inhibition percent (%) of |
|---|---|---|
| | | PYY-1119-induced emetic |
| Compound | SEQ | symptoms at 1 hour (or *72 hours) |
| No. | ID No. | postdose at different compound dose |
| 14 | 15 | 91% (3 nmol/kg) |
| 17 | 18 | 100% (3 nmol/kg) |
| 19 | 20 | 86% (3 nmol/kg) |
| 20 | 21 | 96% (3 nmol/kg) |
| | | 82% (3 nmol/kg) *72 hr |
| 21 | 22 | 96% (3 nmol/kg) |
| 24 | 25 | 81% (3 nmol/kg) |
| 25 | 26 | 100% (3 nmol/kg) |
| | | 92% (1 nmol/kg) |
| | | 58% (0.3 nmol/kg) |
| 43 | 44 | 83% (3 nmol/kg) |
| 44 | 45 | 90% (10 nmol/kg) |
| 48 | 49 | 96.3% (3 nmol/kg) |
| 50 | 51 | 100% (10 nmol/kg) |
| 54 | 55 | 43% (1 nmol/kg) |
| 55 | 56 | 86% (3 nmol/kg) |
| 58 | 59 | 93.8% (3 nmol/kg) |
| 60 | 61 | 57% (1 nmol/kg) |
| 72 | 73 | 98% (1 nmol/kg) |
| 78 | 79 | 96% (1 nmol/kg) |
| 96 | 97 | 90% (1 nmol/kg) |
| 132 | 133 | 68% (1 nmol/kg) |
| 140 | 141 | 63% (1 nmol/kg) |
| 142 | 143 | 63% (1 nmol/kg) |
| | | 92% (3 nmol/kg) |
| 144 | 145 | 89% (3 nmol/kg); |
| | | 70% (1 nmol/kg); |
| | | 62% (3 nmol/kg) *72 h |
| 148 | 149 | 88% (3 nmol/kg) |
| | | 78% (3 nmol/kg) *72 hr, |
| 159 | 160 | 59% (1 nmol/kg) |
| 182 | 183 | 71% (1 nmol/kg) |
| 183 | 184 | 79% (1 nmol/kg) |
| 184 | 185 | 79% (1 nmol/kg) |

As shown in Table 6, it is verified that the compounds of the present invention inhibited PYY-1119 induced emesis, including symptoms of vomiting.

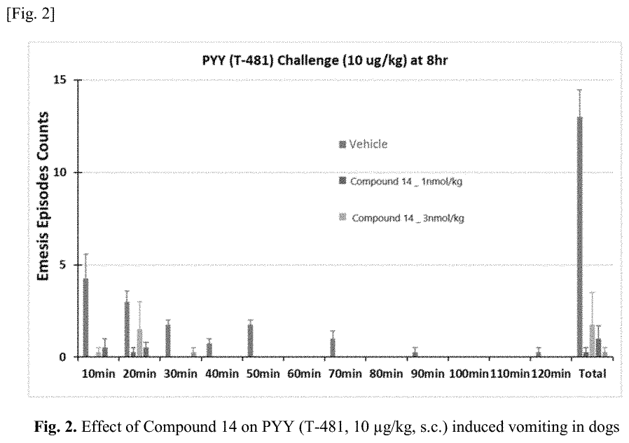

Table 7 shows the effect of Compound 14 on PYY (T-481, 10 μg/kg, s.c.) induced vomiting in dogs. The results are also shown in FIG. 2.

TABLE 7
Effect of Test Peptides on T-481 (10 μg/kg, s.c.)-induced vomiting in

| | | Doses | No. of | Emesis | | | |
|---|---|---|---|---|---|---|---|
| T-481 was | Compound | (nmol/kg, | animals | response^{a} | Latency^{b} | Duration^{c} | Inhibition |
| administered | No. | s.c.) | (responder/used) | (counts) | (min) | (min) | (%) |
| 8 h after | Vehicle^{d} | — | 4/4 | 13.0 ± 1.5 | 6.5 ± 0.9 | 67.3 ± 14.1 | |
| | 14 | 1 | 1/4 | 0.3 ± 0.3 *** | 93.7 ± 26.3 * | 0.1 ± 0.1 *** | 97.7 |
| | 14 | 3 | 1/4 | 1.8 ± 1.8 *** | 92.3 ± 27.7 * | 2.5 ± 2.5 *** | 86.2 |

* P < 0.05,
** P < 0.01,
*** P < 0.001 vs Vehicle.
Each value represents the mean ± SE.
^{a}The cumulative total counts of vomiting response.
^{b}The latency of dog that did not show emetic response was considered as 120 min.
^{c}The cumulative total duration of vomiting response and the duration of dog that show once emetic response was considered as 0.5 min.
^{d}0.09 w/v % Polysorbate 80/10% DMSO/saline 1 mL/kg, s.c.

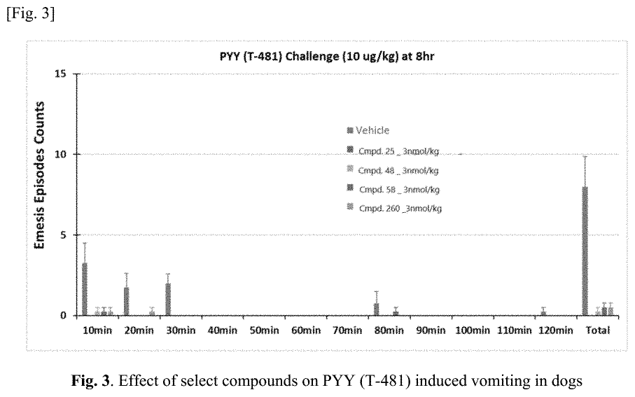

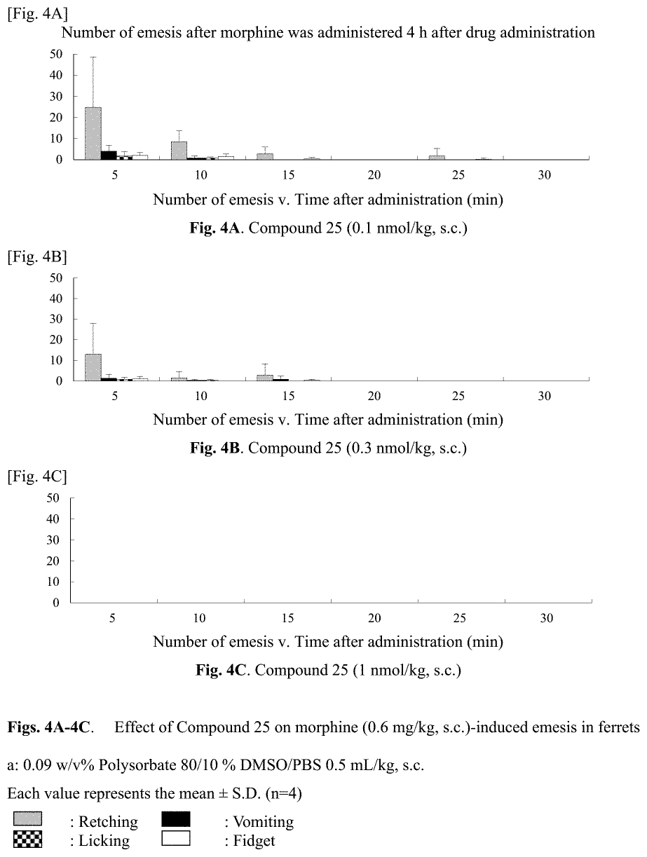

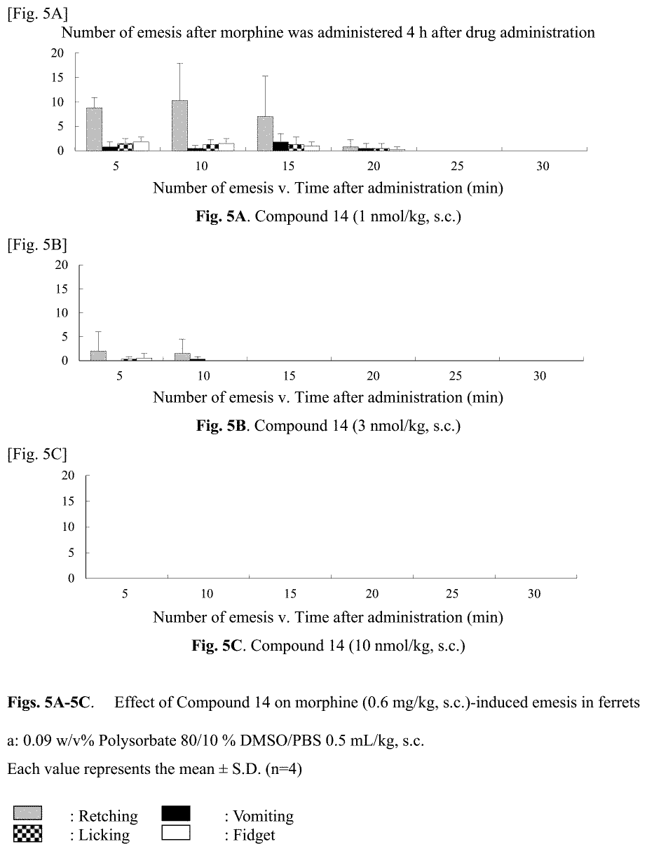

Table 8 shows the effect of Compound 25, Compound 48, Compound 58, and Compound 260 on PYY (T-481, 10 μg/kg, s.c.) induced vomiting in dogs. The results are also shown in FIG. 3.

TABLE 8
Effect of Test Peptides on T-481 (10 μg/kg, s.c.)-induced vomiting in dogs

| | | Doses | No. of | Emesis | | |
|---|---|---|---|---|---|---|
| T-481 was | Compound | (nmol/kg, | animals | response^{a} | Latency^{b} | Duration^{c} |
| administered | No. | s.c.) | (responder/used) | (counts) | (min) | (min) |
| 1 h after | Vehicle^{d} | — | 4/4 | 8.0 ± 1.9 | 5.0 ± 0.9 | 55.7 ± 22.4 |
| | 25 | 3 | 0/4 | 0.0 ± 0.0 *** | 120.0 ± 0.0 *** | 0.0 ± 0.0 ** |
| | 48 | 3 | 1/4 | 0.3 ± 0.3 *** | 91.6 ± 28.4 * | 0.1 ± 0.1 ** |
| | 58 | 3 | 2/4 | 0.5 ± 0.3 *** | 81.7 ± 25.8 * | 0.3 ± 0.1 ** |
| | 260 | 3 | 2/4 | 0.5 ± 0.3 *** | 65.6 ± 31.4 | 0.3 ± 0.1 ** |

* P < 0.05,
** P < 0.01,
*** P < 0.001 vs Vehicle.
Each value represents the mean ± SE.
^{a}The cumulative total counts of vomiting response.
^{b}The latency of dog that did not show emetic response was considered as 120 min.
^{c}The cumulative total duration of vomiting response and the duration of dog that show once emetic response was considered as 0.5 min.
^{d}0.09 w/v % Polysorbate 80/10% DMSO/saline 1 mL/kg, s.c.

As shown in Table 7 and Table 8, it is verified that the compounds of the present invention inhibited PYY (T-481) induced emesis, including symptoms of vomiting.

### Example 11: Y2R Agonist-Induced Emesis in Dogs

To evaluate the Y2R agonist-induced emesis in dogs, test compounds or vehicle (0.09% [w/v] Tween 80/10% DMSO/PBS) was administered subcutaneously (sc) to female beagle dogs (11 months old), followed by sc injections with Y2R agonist (T-3127481, 10 μg/kg) at 8 and 72 hours postdose. Emetic episodes were counted for 2 hours after each Y2R agonist administration (by blinded analysis).

| | | | | | No. of | | | |
|---|---|---|---|---|---|---|---|---|
| | | | | No. of | emetic | Latency | | |
| Day | | | | animals that | episodes | period of | Duration | |
| [after | Cmpd. | Dose | | did not | [% | emesis | of emesis | PK ^{b)} |
| dosing] | No. | (nmol/kg) | N | vomit | Inhibition ^{a)}] | (min) | (min) | (nmol/L) |
| 1 | Vehicle | 0 | 4 | 0 | 6.5 ± 0.6 | 6.3 ± 0.5 | 43.8 ± 35.3 | ND |
| [1 hr] | 142 | 3 | 4 | 3 | 0.5 ± 1.0** | 93.0 ± 54.0^{#} | 0.3 ± 0.5 | 3.8 ± 1.4 |
| | | | | | [92.3%] | | | |
| | 25 | 0.3 | 4 | 0 | 2.8 ± 2.1^{#} | 11.5 ± 3.1^{#} | 6.0 ± 8.5 | 0.47 ± 0.03 |
| | | | | | [57.7%] | | | |
| | 143 | 3 | 4 | 1 | 4.3 ± 5.9 | 39.5 ± 53.8 | 8.8 ± 16.2 | 2.4 ± 0.9 |
| | | | | | [34.6%] | | | |
| 4 | Vehicle | 0 | 4 | 0 | 5.5 ± 1.3 | 6.0 ± 1.2 | 40.3 ± 33.5 | ND |
| [72 hr] | 142 | 3 | 4 | 0 | 3.8 ± 2.5 | 7.8 ± 2.8 | 7.3 ± 7.1 | 0 ± 0 |
| | | | | | [31.8%] | | | |
| | 25 | 0.3 | 4 | 0 | 8.3 ± 2.6 | 7.3 ± 3.0 | 56.3 ± 24.0 | 0 ± 0 |
| | | | | | [−50%] | | | |
| | 143 | 3 | 4 | 1 | 1.8 ± 1.7* | 37.5 ± 55.0 | 7.5 ± 14.3 | 2.6 ± 2.1 |
| | | | | | [68.2%] | | | |

Significantly different from the vehicle group;
*p ≤ 0.05,
**p ≤ 0.01 (Student's t test),
^{#}p ≤ 0.05,
^{##}p ≤ 0.01 (Aspin & Welch t test),
Data is expressed as mean ± S.D.,
ND: Not determined
^{a)} Decreased ratio of the mean emetic episodes compared to those in the vehicle group
^{b)} Plasma test article concentration at 8 and 72 hours after dosing of Compound 142, Compound 25, and Compound 143 (immediately before Y2R agonist administration).

As shown in Table 9, it is verified that the compounds of the present invention inhibited Y2R agonist-induced emesis, including symptoms of vomiting.

### Example 12: Vomiting Suppression Test in Ferrets

1. Effect of subcutaneously administered GIP receptor agonist peptide in morphine-induced acute emetic model.

To evaluate the antiemetic effect, the GIP receptor agonist peptides compounds 25, 14, 142, 48, 17 and 20 other than natural human GIP are subcutaneously administered into male ferrets 30 minutes before morphine administration. Up to 60 minutes after morphine administration, the condition of the ferrets is monitored to record the frequencies and time points of abdominal contraction motions, vomiting behaviors, licking with the tongue, and fidgety behavior occurring.

GIP receptor agonist peptide compounds of the present disclosure are dosed at 0.1-10 nmol/kg to attenuate the morphine (0.6 mg/kg, s.c.)-induced emesis in the ferrets.

GIP receptor agonist peptides are dissolved with a vehicle (0.09 w/v % tween 80/10% DMSO/saline), respectively, to prepare test solutions. 0.5 mg/kg of the test solutions and the vehicle are subcutaneously administered to ferrets (4 in each group), respectively. At the time of each of 4 hours, after administration, 0.6 mg/kg of morphine is subcutaneously administered. Up to 60 minutes after morphine administration, the condition of the ferrets is monitored to record the number of animals that did not vomit, the number of emetic episodes, the latency period in minutes to observe the emetic episodes, the duration of the observed emesis if any.

TABLE 10
Percent inhibition of emetic symptoms induced by morphine in ferrets
treated with GIPr agonist peptides of the present disclosure.

| | | % inhibition of emetic events at |
|---|---|---|
| Compound | SEQ | doses (nmol/kg) shown in ferret |
| No. | ID No. | morphine model |
| 25 | 26 | 54% (0.1 nmol/kg); |
| | | 100% (0.3 nmol/kg); |
| | | 100% (1 nmol/kg) |
| 14 | 15 | 87% (1 nmol/kg); |
| | | 100% (3 nmol/kg); |
| | | 100% (10 nmol/kg) |
| 142 | 143 | 71% (0.3 nmol/kg) |
| 48 | 49 | 75% (0.3 nmol/kg) |
| 17 | 18 | 100% (3 nmol/kg) |
| 20 | 21 | 83% (3 nmol/kg) |

Results from the above example, clearly illustrate that multiple GIPr agonist peptides 25, 14, 142, 48, 17 and 20 were effective in strongly suppressing emesis induced by morphine in ferrets.

TABLE 11
Effect of Compound 25 on morphine (0.6 mg/kg, s.c.)-induced emesis in ferrets

| Morphine | | | Doses | No. of | | | |
|---|---|---|---|---|---|---|---|
| was | | Compound | (nmol/kg, | animals | Latency^{a} | Duration^{b} | |
| administered | Signs | No. | s.c.) | (responder/used) | (min) | (min) | Frequency |
| 4 h after | Retching | Vehicle^{c} | — | 4/4 | 1.8 ± 1.0 | 10.5 ± 5.6 | 37.8 ± 20.1 |
| | | 25 | 0.1 | 2/4 | 16.5 ± 15.6 | 3.5 ± 4.7 | 17.3 ± 20.0 |
| | | | 0.3 | 0/4# | 30.0 ± 0.0** | 0.0 ± 0.0* | 0.0 ± 0.0* |
| | | | 1 | 0/4# | 30.0 ± 0.0** | 0.0 ± 0.0* | 0.0 ± 0.0* |
| | Vomiting | Vehicle^{c} | — | 4/4 | 1.8 ± 1.0 | 3.8 ± 2.1 | 4.8 ± 3.9 |
| | | 25 | 0.1 | 2/4 | 16.5 ± 15.6 | 2.8 ± 4.9 | 2.3 ± 3.9 |
| | | | 0.3 | 0/4# | 30.0 ± 0.0** | 0.0 ± 0.0* | 0.0 ± 0.0* |
| | | | 1 | 0/4# | 30.0 ± 0.0** | 0.0 ± 0.0* | 0.0 ± 0.0* |
| | Emesis | Vehicle^{c} | — | 4/4 | 1.8 ± 1.0 | 10.8 ± 5.3 | 42.5 ± 24.0 |
| | | 25 | 0.1 | 2/4 | 16.5 ± 15.6 | 3.5 ± 4.7* | 19.5 ± 22.9 |
| | | | 0.3 | 0/4# | 30.0 ± 0.0** | 0.0 ± 0.0* | 0.0 ± 0.0* |
| | | | 1 | 0/4# | 30.0 ± 0.0** | 0.0 ± 0.0* | 0.0 ± 0.0* |
| | Licking | Vehicle^{c} | — | 3/4 | 9.8 ± 13.7 | 2.5 ± 2.4 | 2.5 ± 2.4 |
| | | 25 | 0.1 | 2/4 | 16.8 ± 15.3 | 1.0 ± 1.4 | 1.0 ± 1.4 |
| | | | 0.3 | 0/4 | 30.0 ± 0.0* | 0.0 ± 0.0 | 0.0 ± 0.0 |
| | | | 1 | 0/4 | 30.0 ± 0.0* | 0.0 ± 0.0 | 0.0 ± 0.0 |
| | Fidget | Vehicle^{c} | — | 4/4 | 1.8 ± 1.0 | 10.3 ± 5.9 | 4.3 ± 2.6 |
| | | 25 | 0.1 | 2/4 | 16.5 ± 15.6 | 2.5 ± 4.4* | 1.3 ± 1.5* |
| | | | 0.3 | 0/4# | 30.0 ± 0.0** | 0.0 ± 0.0* | 0.0 ± 0.0* |
| | | | 1 | 0/4# | 30.0 ± 0.0** | 0.0 ± 0.0* | 0.0 ± 0.0* |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/PBS 0.5 mL/kg, s.c.
^{#}Significant difference from vehicle treatment p < 0.05 (Fisher's exact test)
* and **Significant difference from vehicle treatment p < 0.05 and p < 0.01, respectively (Student's t-test or Aspin-Welch's t-test, One-side)

TABLE 12
Effect of Compound 14 on morphine (0.6 mg/kg, s.c.)-induced emesis in ferrets

| | | | | No. of | | | |
|---|---|---|---|---|---|---|---|
| Morphine | | | Doses | animals | | | |
| was | | Compound | (nmol/kg, | (responder/ | Latency^{a} | Duration^{b} | |
| administered | Signs | No. | s.c.) | used) | (min) | (min) | Frequency |
| 4 h after | Retching | Vehicle^{c} | — | 4/4 | 4.0 ± 0.8 | 9.3 ± 5.9 | 26.8 ± 11.4 |
| | | 14 | 1 | 1/4 | 23.8 ± 12.5* | 0.3 ± 0.5* | 3.5 ± 7.0** |
| | | | 3 | 0/4# | 30.0 ± 0.0** | 0.0 ± 0.0* | 0.0 ± 0.0** |
| | | | 10 | 0/4# | 30.0 ± 0.0** | 0.0 ± 0.0* | 0 0 ± 0.0** |
| | Vomiting | Vehicle^{c} | — | 4/4 | 5.5 ± 2.1 | 8.3 ± 5.0 | 3.5 ± 1.7 |
| | | 14 | 1 | 1/4 | 24.0 ± 12.0* | 0.3 ± 0.5* | 0.3 ± 0.5** |
| | | | 3 | 0/4# | 30.0 ± 0.0** | 0.0 ± 0.0* | 0.0 ± 0.0* |
| | | | 10 | 0/4# | 30.0 ± 0.0** | 0.0 ± 0.0* | 0.0 ± 0.0* |
| | Emesis | Vehicle^{c} | — | 4/4 | 4.0 ± 0.8 | 9.8 ± 6.3 | 30.3 ± 13.1 |
| | | 14 | 1 | 1/4 | 23.8 ± 12.5* | 0.3 ± 0.5* | 3.8 ± 7.8** |
| | | | 3 | 0/4# | 30.0 ± 0.0** | 0.0 ± 0.0* | 0.0 ± 0.0** |
| | | | 10 | 0/4# | 30.0 ± 0.0** | 0.0 ± 0.0* | 0.0 ± 0.0** |
| | Licking | Vehicle^{c} | — | 4/4 | 4.0 ± 0.8 | 8.3 ± 6.6 | 4.5 ± 3.7 |
| | | 14 | 1 | 1/4 | 23.8 ± 12.5* | 0.3 ± 0.5* | 0.3 ± 0.5 |
| | | | 3 | 0/4# | 30.0 ± 0.0** | 0.0 ± 0.0* | 0.0 ± 0.0* |
| | | | 10 | 0/4# | 30.0 ± 0.0** | 0.0 ± 0.0* | 0.0 ± 0.0* |
| | Fidget | Vehicle^{c} | — | 4/4 | 4.0 ± 0.8 | 9.3 ± 5.9 | 4.5 ± 2.6 |
| | | 14 | 1 | 1/4 | 23.8 ± 12.5* | 0.3 ± 0.5* | 0.5 ± 1.0* |
| | | | 3 | 0/4# | 30.0 ± 0.0** | 0.0 ± 0.0* | 0.0 ± 0.0* |
| | | | 10 | 0/4# | 30.0 ± 0.0** | 0.0 ± 0.0* | 0.0 ± 0.0* |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/PBS 0.5 mL/kg, s.c.
#Significant difference from vehicle treatment p < 0.05 (.Fisher's exact test)
* and **Significant difference frost vehicle treatment p < 0.05 and p < 0.01, respectively (Student's t-test or Aspin-Welch's t-test, One-side)

TABLE 13
Effect of Compound 142 and Compound 48 on morphine (0.6 mg/kg, s.c.)-induced emesis in ferrets

| | | | | No. of | | | |
|---|---|---|---|---|---|---|---|
| Morphine | | | Doses | animals | | | |
| was | | Compound | (nmol/kg, | (responder/ | Latency^{a} | Duration^{b} | |
| administered | Signs | No. | s.c.) | used) | (min) | (min) | Frequency |
| 4 h after | Retching | Vehicle^{c} | — | 4/4 | 4.3 ± 2.6 | 9.8 ± 3.1 | 28.8 ± 9.9 |
| | | 142 | 0.3 | 3/4 | 9.8 ± 13.6 | 1.5 ± 1.7** | 9.0 ± 11.7* |
| | | 48 | 0.1 | 4/4 | 3.5 ± 0.6 | 2.3 ± 2.5** | 8.3 ± 5.1** |
| | | 48 | 0.3 | 1/4 | 23.3 ± 13.5* | 0.8 ± 1.5** | 7.5 ± 15.0* |
| | Vomiting | Vehicle^{c} | — | 4/4 | 5.3 ± 4.6 | 8.5 ± 2.4 | 4.5 ± 2.1 |
| | | 142 | 0.3 | 1/4 | 23.0 ± 14.0* | 1.0 ± 2.0** | 0.5 ± 1.0** |
| | | 48 | 0.1 | 2/4 | 16.8 ± 15.3 | 0.5 ± 0.6** | 0.5 ± 0.6** |
| | | 48 | 0.3 | 1/4 | 23.3 ± 13.5* | 0.8 ± 1.5** | 0.8 ± 1.5* |
| | Emesis | Vehicle^{c} | — | 4/4 | 4.3 ± 2.6 | 10.0 ± 3.2 | 33.3 ± 8.2 |
| | | 142 | 0.3 | 3/4 | 9.8 ± 13.6 | 1.5 ± 1.7** | 9.5 ± 12.7** |
| | | 48 | 0.1 | 4/4 | 3.5 ± 0.6 | 2.3 ± 2.5** | 8.8 ± 5.6** |
| | | 48 | 0.3 | 1/4 | 23.3 ± 13.5* | 0.8 ± 1.5** | 8.3 ± 16.5* |
| | Licking | Vehicle^{c} | — | 4/4 | 6.5 ± 3.0 | 5.3 ± 5.3 | 3.8 ± 3.4 |
| | | 142 | 0.3 | 1/4 | 24.0 ± 12.0* | 0.3 ± 0.5 | 0.3 ± 0.5 |
| | | 48 | 0.1 | 1/4 | 24.8 ± 10.5** | 0.3 ± 0.5 | 0.3 ± 0.5 |
| | | 48 | 0.3 | 1/4 | 24.0 ± 12.0* | 0.3 ± 0.5 | 0.3 ± 0.5 |
| | Fidget | Vehicle^{c} | — | 4/4 | 4.5 ± 2.4 | 6.5 ± 2.9 | 3.3 ± 1.3 |
| | | 142 | 0.3 | 3/4 | 9.8 ± 13.6 | 1.5 ± 1.7* | 1.3 ± 1.3* |
| | | 48 | 0.1 | 4/4 | 3.5 ± 0.6 | 1.3 ± 0.5* | 1.5 ± 0.6* |
| | | 48 | 0.3 | 1/4 | 23.3 ± 13.5* | 0.8 ± 1.5** | 0.5 ± 1.0** |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/PBS 0.5 mL/kg, s.c.
No Significant difference from vehicle treatment (Fisher's exact test)
* and **Significant difference from vehicle treatment p < 0.05 and p < 0.01, respectively (Student's t-test or Aspin-Welch's t-test, One-side)

Results from the above morphine induced emesis example, clearly illustrate that Compounds 14 (SEQ ID NO: 15), Compound 48 (SEQ ID NO:), Compound 25 (SEQ ID NO: 26), and Compound 142 (SEQ ID NO: 143) are effective in inhibiting the frequency of emetic events, including frequency of both retching and vomiting events in ferrets dosed with morphine.

### Example 13: Apomorphine Induced Emesis in Dog

Dogs are transferred to an observation cage (700 mm W×700 mm D×700 mm H [W×D×H], without food) on 1 day before each apomorphine challenge. The dogs are weighed by using an electronic balance then test articles will be administrated via the subcutaneous route. Apomorphine is challenged at 8 hr after the administration and emetic events will be monitored for 1 h by video recording. The second apomorphine challenge will be 72 hr after the administration and emetic events will be recorded by the same protocol. Emesis symptoms are continuously recorded using a video camera and stored on a blue ray disc. Observations of symptoms include retching (a rhythmic contraction of the abdomen) and vomiting (vomiting behavior, including the elimination of vomitus or similar behavior). Besides, the combination of retching and vomiting is defined as emesis, and the number of episodes, latency (time elapsed from morphine administration until the onset of the first emesis symptom), duration (time elapsed between the onset of the first and final episodes of emesis), and frequency (number of animals showing emesis/number of experimental animals) of each of these symptoms is calculated. The latency in cases where emesis symptoms are not noted is taken as the maximum value (1 h for apomorphine challenge) at the end of observation. When the duration of the emesis symptoms is less than 1 min, the duration is recorded, for convenience, as 1 min.

TABLE 14
shows the results of the apomorphine testing:

| | | % inhibition of emetic events at |
|---|---|---|
| | | doses (nmol/kg) shown in dog |
| Compound | Sequence | Apomorphine model after 8 hrs |
| No. | ID No. | (or *72 hours) |
| 14 | 15 | 92% (30 nmol/kg) |
| 17 | 18 | 92% (30 nmol/kg); |
| | | 68% (10 nmol/kg) |
| 20 | 21 | 69% (30 nmol/kg) |
| 21 | 22 | 71% (10 nmol/kg) |
| 25 | 26 | 78% (30 nmol/kg); |
| | | 70% (10 nmol/kg); |
| | | 39% (3 nmol/kg) |
| 48 | 49 | 71% (10 nmol/kg); |
| | | 38% (3 nmol/kg) |
| 140 | 141 | 76% (10 nmol/kg) |
| 141 | 142 | 52% (10 nmol/kg) |
| 142 | 143 | 76% (10 nmol/kg); |
| | | 77% (30 nmol/kg); |
| | | 54% (3 nmol/kg) |
| 148 | 149 | 55% (30 nmol/kg), |
| | | 69% (60nmol/kg*) |

Results from the above example, clearly illustrate that Compounds 14, 17, 20, 21, 25, 48, 140, 142 and 148 are effective in inhibiting the frequency of emetic events in ferrets dosed with morphine in a dose dependent manner.

### Example 14: Serum Half-Life and Percentage Remaining at 48 Hours

Serum Half-Life Analysis

Human plasma (mixed gender: sodium heparin is used as anti-coagulant; pre-adjusted to pH 7.4—NB alternative species may be used) is spiked with each test peptide (500 nM) and incubated ·cn=3) at 37° C. for 48 hours in a 5% CO2 environment. Aliquots are taken at 0. 1, 2, 4, 7, 24 and 46 hours and pH adjusted to pH 3 with 20% formic acid prior to analysis. Appropriate positive control compounds will be incubated in parallel, in addition to a no plasma control, sampled at 0 and 8 hours. All samples will be treated with ice-cold acetonitrile/methanol (4:1 (v/v)) containing internal standard prior to centrifugation at 2000 g and 4° C. for 10 minutes and subjected to LC-MS/MS analysis.

Sample Analysis

The samples are analyzed by LC-MS/MS using a 6500 (or equivalent) triple quadrupole mass spectrometer (AB Sciex) coupled to an appropriate Liquid Chromatography (LC) system. Protein binding and stability values are determined via peak area ratios using multiple reaction monitoring (MRM) parameters following compound optimisation. Multiple reaction monitoring (MRM) is a highly sensitive method of targeted mass spectrometry (MS) that can be used to selectively detect and quantify peptides based on the screening of specified precursor peptide-to-fragment ion transitions.

TABLE 15
shows the results of the Serum half-life of the compounds
and the percentage remaining at 48 hours:

| Compound | Sequence | Serum t_{1/2} | % Remaining |
|---|---|---|---|
| No. | ID No. | (hours) | at 48 hr |
| 5 | 6 | 0.6 | 0 |
| 11 | 12 | 1.99 | 0 |
| 12 | 13 | 0.97 | 0 |
| 16 | 17 | >48 | 107.1 |
| 17 | 18 | >48 | 116 |
| 18 | 19 | >48 | 145.3 |
| 19 | 20 | >48 | 131.5 |
| 20 | 21 | >48 | 122 |
| 21 | 22 | >48 | 116.5 |
| 22 | 23 | 11.82 | 5.6 |
| 23 | 24 | 13.33 | 6.5 |
| 24 | 25 | 16.31 | 12.1 |
| 25 | 26 | 18.14 | 14.2 |
| 26 | 27 | 0.67 | 0 |
| 27 | 28 | 1.13 | 0 |
| 28 | 29 | 37.88 | 41.5 |
| 29 | 30 | 30.65 | 34.1 |
| 30 | 31 | 13.49 | 8 |
| 31 | 32 | 16.84 | 12.5 |

Table 15 provides two data points related to the pharmacokinetic activity of the GIPR agonist peptides of the present disclosure. Optimum values for the use of the GIPR agonist peptides of the present disclosure range between a serum T½ (half life) of 10-20 hours for once daily dosing. As can be seen from Table 15, when the T½ in serum approaches 30 hours and greater, the amount remaining after 48 hours exceeds 30%, which indicates that the peptide is accumulating and not being made available to exert its pharmacological activity.

### Example 15: Human Plasma Protein Binding (PPB)

Stock Solutions

Stock solutions: (1000 μM) of the peptides are prepared in DMSO.

Plasma Protein Binding (PPB) Analysis

Human plasma (mixed gender; containing K2-EDTA as anti-coagulant; pre-adjusted to pH 7.4—NB alternative species may be used) is spiked individually with each test peptide (1000 nmol/L), sampled for analysis and then incubated (n=4) at 37° C. in a water bath for 30 minutes. Following the incubation period, the plasma is sampled for analysis, then transferred to ultracentrifugation tubes and centrifuged (n=3) at −450,000 g and 4° C. for 3 hours, after which the supernatant is sampled for analysis. An additional aliquot of the supernatant is taken at the end of the centrifugation period to determine the total protein concentration. An aliquot of the incubated plasma will be stored at 4° C. for 3 hours and then sampled for analysis. At the point of sampling, all samples are matrix-matched, treated with ice-cold acetonitrile/methanol (4:1 (vfv)) containing internal standard, centrifuged al 2000 g and 4° C. for 10 minutes and stored prior to LC-MS/MS analysis. An appropriate positive control compound control will be incubated and centrifuged in parallel; control plasma is also centrifuged to generate samples for matrix-matching. Fraction unbound (Fu) values is determined by comparison of the analyte response in plasma to the analyte response in the supernatant, determined via peak area response ratios.

Plasma Stability Analysis

Human plasma (mixed gender: sodium heparin is used as anti-coagulant; pre-adjusted to pH 7.4—NB alternative species may be used) is spiked with each test peptide (500 nM) and incubated ·cn=3) at 37° C. for 48 hours in a 5% CO2 environment. Aliquots are taken at 0. 1, 2, 4, 7, 24 and 46 hours and pH adjusted to pH 3 with 20% formic acid prior to analysis. Appropriate positive control compounds will be incubated in parallel, in addition to a no plasma control, sampled at 0 and 8 hours. All samples will be treated with ice-cold acetonitrile/methanol (4:1 (v/v)) containing internal standard prior to centrifugation at 2000 g and 4° C. for 10 minutes and subjected to LC-MS/MS analysis.

Sample Analysis

The samples are analyzed by LC-MS/MS using a 6500 (or equivalent) triple quadrupole mass spectrometer (AB Sciex) coupled to an appropriate Liquid Chromatography (LC) system. Protein binding and stability values are determined via peak area ratios using multiple reaction monitoring (MRM) parameters following compound optimisation. Multiple reaction monitoring (MRM) is a highly sensitive method of targeted mass spectrometry (MS) that can be used to selectively detect and quantify peptides based on the screening of specified precursor peptide-to-fragment ion transitions.

Dog PPB values presented below are obtained essentially as described for Human PPB samples, with the difference being that dog serum is used instead of human serum. Table 16 is provided with the values of (Fu, plasma) as fraction unbound expressed as a percentage compared to the percent bound. i.e. if the value is 0.0123, then the fraction unbound is (0.0123/100)%, which is 1.23% of the peptide is unbound and 98.77% is bound in plasma.

TABLE 16
shows the results of the dog PPB and Human PPB:

| Compound | SEQ | Human PPB | Dog PPB |
|---|---|---|---|
| No. | ID No. | (Fu,ₚₗₐₛₘₐ) | (Fu,ₚₗₐₛₘₐ) |
| 14 | 15 | 0.0533 | 0.0439 |
| 16 | 17 | 0.0441 | 0.0151 |
| 17 | 18 | 0.064 | 0.0675 |
| 18 | 19 | 0.0588 | 0.0517 |
| 19 | 20 | 0.0321 | 0.0361 |
| 20 | 21 | 0.0013 | 0.0021 |
| 21 | 22 | 0.0414 | 0.037 |
| 22 | 23 | 0.0413 | |
| 23 | 24 | 0.0734 | |
| 24 | 25 | 0.0512 | |
| 25 | 26 | 0.0459 | 0.0401 |
| 29 | 30 | 0.018 | |
| 30 | 31 | 0.0407 | |
| 119 | 120 | 0.0185 | 0.0105 |
| 142 | 143 | 0.0111 | 0.003 |

As can be seen in Table 16, the GIPR agonist peptides of the present disclosure provide a percent of unbound or active drug for antiemetic activity, which ranges from about 0.1% to about 7.3%. The efficacy of the GIPR agonist peptide will be related to the exposure to the amount of unbound drug in plasma, i.e. the proportion free peptide to penetrate into surrounding tissues. The bound peptide in plasma can also serve as a reservoir for free peptide removed by various elimination processes thus prolonging the duration of action. These GIPR agonist peptides also demonstrate that due to the high proportion of the drug being bound (98.9% to 92.7%), the duration of action can be extended for longer periods of time. GIPR agonist peptides of the present disclosure provide an optimum range of unbound to plasma protein for once daily dosing to human subjects between 1-5% unbound. It is believed that GIPR agonist peptides of the present disclosure having a free fraction of about 1% to about 5% translates to a peptide having a desirable pK profile, demonstrating fast absorption and fast elimination to prevent excessive accumulation. Several compounds in Table 16 demonstrate optimum free unbound peptide, for example, compounds 14, 16, 18, 19, 21, 22, 24, 25, and 30.

### Example 16: Solubility of the GIPR Agonist Compounds

3 mg of peptides are weighted out in a small glass vial. 100 uL of 200 mM Phosphate buffer pH 7.4 are added and the vial is sonicated/vortexed as necessary for a maximum of 1 min. A visual inspection is performed, If the sample is fully dissolved, the solubility is recorded as 30 mg/mL. If insoluble material is observed in the tube the addition of 100 uL of buffer and mixing is repeated until complete dissolution. If the peptide is not soluble in 500 uL of buffer, it is labeled as solubility <6 mg/mL. The solubility can be confirmed by RP-HPLC after filtration on 0.2 μm filter on an Agilent 1200 system with a Kinetex column form Phenomenex® (2.6 μm EVO C18 100 Å, LC Column 50×3.0 mm) kept at 40° C., the eluent A is 0.05% TFA in Water, B is 0.035% TFA in Acetonitrile at a 0.6 ml/min flow rate. The gradient was from 20 to 70 over 5 min, the column is then washed for 1 min at 90% B. UV monitoring at 215 nm was used to monitor peptide concentration.

TABLE 17
shows the results of the solubility of the
compounds in phosphate buffer at pH 7.4:

| | | pH 7.4 Phosphate |
|---|---|---|
| Compound | SEQ | Buffer - Solubility |
| No. | ID No. | (mg/mL) |
| 1 | 2 | 15 |
| 2 | 3 | 15 |
| 3 | 4 | 15 |
| 4 | 5 | 15 |
| 5 | 6 | 15 |
| 6 | 7 | 15 |
| 7 | 8 | 15 |
| 8 | 9 | 15 |
| 9 | 10 | 15 |
| 10 | 11 | 15 |
| 11 | 12 | 15 |
| 12 | 13 | 15 |
| 13 | 14 | 30 |
| 14 | 15 | 15 |
| 15 | 16 | 30 |
| 16 | 17 | 30 |
| 17 | 18 | 30 |
| 18 | 19 | 30 |
| 19 | 20 | 30 |
| 20 | 21 | 30 |
| 21 | 22 | 30 |
| 22 | 23 | 30 |
| 23 | 24 | 30 |
| 24 | 25 | 30 |
| 25 | 26 | 30 |
| 26 | 27 | 30 |
| 27 | 28 | 30 |
| 28 | 29 | 30 |
| 29 | 30 | 30 |
| 30 | 31 | 30 |
| 31 | 32 | 30 |
| 32 | 33 | 30 |
| 33 | 34 | 30 |
| 34 | 35 | 30 |
| 35 | 36 | 30 |
| 36 | 37 | 30 |
| 37 | 38 | 15 |
| 38 | 39 | 30 |
| 39 | 40 | 30 |
| 40 | 41 | 30 |
| 41 | 42 | 30 |
| 42 | 43 | 30 |
| 43 | 44 | 30 |
| 44 | 45 | 30 |
| 45 | 46 | 30 |
| 46 | 47 | 30 |
| 47 | 48 | 30 |
| 48 | 49 | 30 |
| 49 | 50 | 30 |
| 50 | 51 | 30 |
| 51 | 52 | 30 |
| 52 | 53 | 30 |
| 53 | 54 | 30 |
| 54 | 55 | 30 |
| 55 | 56 | 30 |
| 56 | 57 | 15 |
| 57 | 58 | 15 |
| 58 | 59 | 15 |
| 59 | 60 | 15 |
| 60 | 61 | 30 |
| 61 | 62 | 30 |
| 62 | 63 | 30 |
| 63 | 64 | 15 |
| 64 | 65 | 30 |
| 65 | 66 | 15 |
| 66 | 67 | 15 |
| 67 | 68 | 15 |
| 68 | 69 | 15 |
| 69 | 70 | 15 |
| 70 | 71 | 15 |
| 71 | 72 | 15 |
| 72 | 73 | 15 |
| 73 | 74 | 30 |
| 74 | 75 | 30 |
| 75 | 76 | 15 |
| 76 | 77 | 15 |
| 77 | 78 | 30 |
| 78 | 79 | 30 |
| 79 | 80 | 15 |
| 80 | 81 | 30 |
| 81 | 82 | 30 |
| 82 | 83 | 30 |
| 83 | 84 | 30 |
| 84 | 85 | 30 |
| 85 | 86 | 30 |
| 86 | 87 | 30 |
| 87 | 88 | 30 |
| 88 | 89 | 30 |
| 89 | 90 | 15 |
| 90 | 91 | 15 |
| 91 | 92 | 15 |
| 92 | 93 | 15 |
| 93 | 94 | 30 |
| 94 | 95 | 30 |
| 95 | 96 | 30 |
| 96 | 97 | 30 |
| 97 | 98 | 30 |
| 98 | 99 | 30 |
| 99 | 100 | 30 |
| 100 | 101 | 30 |
| 101 | 102 | 30 |
| 102 | 103 | 30 |
| 103 | 104 | 30 |
| 104 | 105 | 30 |
| 105 | 106 | 30 |
| 106 | 107 | 30 |
| 107 | 108 | 30 |
| 108 | 109 | 15 |
| 109 | 110 | 30 |
| 110 | 111 | 30 |
| 111 | 112 | 30 |
| 112 | 113 | 30 |
| 113 | 114 | 30 |
| 114 | 115 | 30 |
| 115 | 116 | 15 |
| 116 | 117 | 30 |
| 117 | 118 | 30 |
| 118 | 119 | 30 |
| 119 | 120 | 30 |
| 120 | 121 | 30 |
| 121 | 122 | 30 |
| 122 | 123 | 30 |
| 123 | 124 | 30 |
| 124 | 125 | 30 |
| 125 | 126 | 30 |
| 126 | 127 | 30 |
| 127 | 128 | 30 |
| 128 | 129 | 30 |
| 129 | 130 | 30 |
| 130 | 131 | 30 |
| 131 | 132 | 30 |
| 132 | 133 | 30 |
| 133 | 134 | 30 |
| 134 | 135 | 30 |
| 135 | 136 | 30 |
| 136 | 137 | 30 |
| 137 | 138 | 30 |
| 138 | 139 | 30 |
| 139 | 140 | 30 |
| 140 | 141 | 30 |
| 141 | 142 | 30 |
| 142 | 143 | 30 |
| 143 | 144 | 30 |
| 144 | 145 | 30 |
| 145 | 146 | 30 |
| 146 | 147 | 30 |
| 147 | 148 | 30 |
| 148 | 149 | 30 |
| 149 | 150 | 30 |
| 150 | 151 | 30 |
| 151 | 152 | 30 |
| 152 | 153 | 30 |
| 153 | 154 | 30 |
| 154 | 155 | 30 |
| 155 | 156 | 30 |
| 156 | 157 | 30 |
| 157 | 158 | 30 |
| 158 | 159 | 30 |
| 159 | 160 | 30 |
| 160 | 161 | 30 |
| 161 | 162 | 30 |
| 162 | 163 | 30 |
| 163 | 164 | 30 |
| 164 | 165 | <16 |
| 165 | 166 | 30 |
| 166 | 167 | 30 |
| 167 | 168 | 30 |
| 168 | 169 | 30 |
| 169 | 170 | 30 |
| 170 | 171 | 30 |
| 171 | 172 | 30 |
| 172 | 173 | 30 |
| 173 | 174 | 30 |
| 174 | 175 | 30 |
| 175 | 176 | 30 |
| 176 | 177 | 30 |
| 177 | 178 | 30 |
| 178 | 179 | 30 |
| 179 | 180 | 30 |
| 180 | 181 | 30 |
| 181 | 182 | 30 |
| 182 | 183 | 30 |
| 183 | 184 | 30 |
| 184 | 185 | 30 |
| 185 | 186 | 30 |
| 186 | 187 | 30 |
| 187 | 188 | 60 |
| 188 | 189 | 30 |
| 189 | 190 | 30 |
| 226 | 227 | 0.8 |
| 227 | 228 | 0.7 |
| 228 | 229 | 0.7 |
| 229 | 230 | 7.7 |
| 230 | 231 | 6.8 |
| 231 | 232 | 0.7 |
| 232 | 233 | 0.7 |
| 233 | 234 | 7.1 |
| 234 | 235 | 7 |
| 235 | 236 | 0.8 |
| 236 | 237 | 0.7 |
| 237 | 238 | 0.8 |
| 238 | 239 | 13.7 |
| 239 | 240 | 6 |
| 240 | 241 | 6 |
| 241 | 242 | 0.9 |
| 242 | 243 | 7.5 |
| 243 | 244 | 10 |
| 244 | 245 | 10 |
| 245 | 246 | 7.5 |
| 246 | 247 | 6 |
| 247 | 248 | 10 |
| 248 | 249 | 7.5 |
| 249 | 250 | 7.5 |
| 250 | 251 | 10 |
| 251 | 252 | <6.00 |
| 252 | 253 | <6.00 |
| 253 | 254 | 10 |
| 254 | 255 | 10 |
| 255 | 256 | <6.00 |
| 256 | 257 | <6.00 |
| 257 | 258 | 6 |
| 258 | 259 | 6 |
| 259 | 260 | <6.00 |
| 260 | 261 | 6 |
| 261 | 262 | 6 |
| 262 | 263 | 6 |
| 263 | 264 | 6 |
| 264 | 265 | <6.00 |
| 265 | 266 | 6 |
| 266 | 267 | 6 |
| 267 | 268 | 6 |
| 268 | 269 | 6 |
| 269 | 270 | 6 |
| 270 | 271 | 10 |
| 271 | 272 | 7.5 |
| 272 | 273 | <6.00 |
| 273 | 274 | 6 |
| 274 | 275 | <6.00 |
| 275 | 276 | <6.00 |
| 276 | 277 | <6.00 |
| 277 | 278 | <6.00 |
| 278 | 279 | <6.00 |
| 279 | 280 | 7.5 |
| 280 | 281 | <6.00 |
| 281 | 282 | <6.00 |
| 282 | 283 | 6 |
| 283 | 284 | 6 |
| 284 | 285 | 10 |
| 285 | 286 | <6.00 |
| 286 | 287 | <6.00 |
| 287 | 288 | 7.5 |
| 288 | 289 | 7.5 |
| 289 | 290 | <6.00 |
| 290 | 291 | 7.5 |
| 291 | 292 | <6.00 |
| 292 | 293 | <6.00 |
| 293 | 294 | 6 |
| 294 | 295 | 7.5 |
| 295 | 296 | 7.5 |
| 296 | 297 | 6 |
| 297 | 298 | 10 |
| 298 | 299 | 6 |
| 299 | 300 | <15 |
| 300 | 301 | <15 |
| 301 | 302 | <15 |
| 302 | 303 | <6 |
| 303 | 304 | <6 |

As shown in Table 17, several of the tested GIPR agonist peptides demonstrate high solubility in physiological buffer (Phosphate buffer at pH 7.4) of 15 mg/mL and above. Compounds 1-189 exhibit a solubility in phosphate buffer at pH 7.4 of 15 mg/mL or greater, which are the preferred compounds for dosing in volumes that facilitate once per day or QD dosing. Compounds having a solubility of less than 15 mg/mL, for example less than 15 mg/mL, or from 10 mg/mL to 15 mg/mL are less preferred, and peptide compounds having less than 10 mg/mL solubility as described in Example 16 are excluded from the GIPR agonist peptides that are suitable for QD dosing. In some embodiments, GIPR agonist peptide compounds of the present disclosure having less than 15 mg/mL solubility as described in Example 16 are excluded from the GIPR agonist peptides that are suitable for QD dosing.

### Example 17: Summary of Pharmacokinetic (PK) and Pharmacodynamic (PD) Studies of Selective GIP Receptor Agonist Peptides

Pharmacokinetic (PK) were conducted in dog in order to determine the half-life after IV and SC dosing. The peptide was dissolved in 10% DMSO/0.09% Polysorbate/PBS pH 7.4 to a concentration of 3 nmol/mL and the animal were dose with a volume of 1 mL/kg SC or IV. Blood sample were collected at 0, 0.0330, 0.0830, 0.250, 0.500, 1.00, 2.00, 4.00, 6.00, 8.00, 12.0, 24.0, 48.0 hours for IV dosing and 0.250, 0.500, 1.00, 2.00, 4.00, 6.00, 8.00, 12.0, 24.0, 48.0 for SC dosing, EDTA-K2 was used as anticoagulant. The plasma concentration of the peptide was measured using LCMS. Allometric scaling of lipidated peptide pharmacokinetics including T½ and MRT is known in the art for rodent to dog and mini pig and to humans. In one illustrative embodiment, a lipidated peptide was shown to have MRT=16.5 hrs following s.c. dosing in dog and is dosed QD in humans. See for example, Discovery and Development of Liraglutide and Semaglutide. Knudsen, L. B.; Lau, J. Frontiers in Endocrinology, 2019, vol 10, Article 155.

TABLE 18
shows PK data of selective compounds

| | | Dog PK | |
|---|---|---|---|
| | | IV & SC 3 nmol/kg | |
| Cmpd | SEQ | IV T½ | |
| No. | ID No. | (SC MRT 0-last) in hours | |
| 17 | 18 | 2.6 | (7.3) |
| 25 | 26 | 5.3 | (9.9) |
| 21 | 22 | 4.6 | (8.7) |
| 48 | 49 | 7.4 | (15.2) |
| 142 | 143 | 8.4 | (13.8) |
| 14 | 15 | 4.5 | (8.1) |
| 20 | 21 | 20.6 | (21.8) |

As shown above in Table 18, peptide compounds 14, 17, 20, 21, 25, 48 and 142 all demonstrate exemplary pharmacokinetic activity providing the optimal exposure for once per day dosing. As shown in Table 18, the IV T½ life (data provided for dogs) can be extrapolated to human exposure ranging from IV T½ lives ranging from 6 to 16 hours when dosed at 3 nmol/kg.

### Formulation Example 1

| | | |
|---|---|---|
| (1) | Compound 10 | 10.0 mg |
| (2) | Lactose | 70.0 mg |
| (3) | Cornstarch | 50.0 mg |
| (4) | Soluble starch | 7.0 mg |
| (5) | Magnesium stearate | 3.0 mg |

Compound 10 (10.0 mg) and magnesium stearate (3.0 mg) are granulated with an aqueous soluble starch solution (0.07 mL) (7.0 mg as soluble starch), dried and mixed with lactose (70.0 mg) and cornstarch (50.0 mg). The mixture is compressed to give a tablet.

### Formulation Example 2

| | | | |
|---|---|---|---|
| (1) | Compound 5 | 5.0 | mg |
| (2) | Sodium chloride | 20.0 | mg |
| (3) | Distilled water to total amount | 2 | mL |

Compound 5 (5.0 mg) and sodium chloride (20.0 mg) are dissolved in distilled water, and water is added to a total amount of 2.0 ml. The solution is filtered, and filled in a 2 ml ampoule under aseptic conditions. The ampoule is sterilized and tightly sealed to give a solution for injection.

### INDUSTRIAL APPLICABILITY

The GIP receptor agonist peptides of the present disclosure have superior GIP receptor selective agonist activity, and are useful as a drug for the prophylaxis or treatment of emesis and conditions caused by associated with GIP receptor activity, for example, emesis and diseases associated with vomiting or nausea and the like. In one embodiment, the selective GIP receptor agonist peptides are useful as a drug or medicament, or for use in the prophylaxis or treatment of emesis and conditions caused by associated with GIP receptor activity, for example cyclic vomiting syndrome, and nausea and/or vomiting associated with administration of a chemotherapeutic or anti-cancer agent as illustrated herein.

All the publications, patents, and the patent applications cited herein are incorporated herein by reference in their entireties.

### [Free Text for Sequence Listing]

SEQ ID NO: 1: Natural human GIP (1-42 peptide)
SEQ ID NO: 2 to 305 Synthetic peptides (Formulas (I)-(III))

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

1. A GIP receptor agonist peptide represented by formula (I): P¹-Tyr-A2-Glu-Gly-Thr-Phe-Ile-Ser-A9-Tyr-Ser-Ile-A13-A14-Asp-A16-A17-A18-Gln-A20-A21-Phe-Val-A24-Trp-A26-Leu-A28-Gln-A30-A31-A32-A33-A34-A35-A36-A37-A38-A39-A40-P², or a pharmaceutically acceptable salt thereof;
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Gly, or Pro;
A9: represents Asp or Leu;
A13: represents Aib, or Ala;
A14: represents Leu, Aib, Lys;
A16: represents Arg, Ser, or Lys;
A17: represents Aib, Gln, or Ile;
A18: represents Ala, His, or Lys;
A19: represents Gln, or Ala;
A20: represents Aib, Gln, Lys, or Ala;
A21: represents Asp, Asn, or Lys;
A24: represents Asn, or Glu;
A26: represents Leu or Lys;
A28: represents Ala, Lys, or Aib;
A29: represents Gln, Lys, Gly, or Aib;
A30: represents Arg, Gly, Ser, or Lys;
A31: represents Gly, Pro, or a deletion;
A32: represents Ser, Gly, or a deletion;
A33: represents Ser, Gly, or a deletion;
A34: represents Gly, Lys, Asn, or a deletion;
A35: represents Ala, Asp, Ser, Lys, or a deletion;
A36: represents Pro, Trp, Lys, or a deletion;
A37: represents Pro, Lys, Gly, or a deletion;
A38: represents Pro, His, Lys, or a deletion;
A39: represents Ser, Asn, Gly, Lys, or a deletion; and
A40: represents Ile, Lys or a deletion.

2. The GIP receptor agonist peptide according to claim 1 or the pharmaceutically acceptable salt thereof, wherein A31 is Gly, A32-A39 are deletion; or A32 is Gly, A33-A39 are deletion.

3. The GIP receptor agonist peptide according to claim 1 or the pharmaceutically acceptable salt thereof, wherein A31 is Pro and A32 is Gly, and A33-A39 are deletion.

4. The GIP receptor agonist peptide according to any one of claims 1-3 or the pharmaceutically acceptable salt thereof, wherein P² is OH.

5. A GIP receptor agonist peptide represented by formula (II): P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-A26-Leu-Ala-A29-A30-A31-A32-A33-A34-A35-A36-A37-A38-A39-A40-P², or a pharmaceutically acceptable salt thereof, wherein:
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, Ser, Ala, D-Ala, or Gly;
A13: represents Aib, Tyr, or Ala;
A14: represents Leu, or Lys(R);
A16: represents Arg, Ser, or Lys;
A17: represents Aib, Ile, Gln, or Lys(R);
A18: represents Ala, His, or Lys(R);
A19: represents Gln or Ala;
A20: represents Aib, Gln, or Lys(R);
A21: represents Asn, Glu, Asp, or Lys(R);
A24: represents Asn, or Glu;
A26: represents Leu or Lys(R);
A28: represents Ala, Aib, or Lys(R);
A29: represents Gln, Aib, or Lys(R);
A30: represents Arg, Gly, Lys, Ser, or Lys(R);
A31: represents Gly, Pro, or a deletion;
A32: represents Ser, Lys, Pro, Gly, or a deletion;
A33: represents Ser, Lys, Gly, or a deletion;
A34: represents Gly, Lys, Asn, or a deletion;
A35: represents Ala, Asp, Ser, Lys, or a deletion;
A36: represents Pro, Trp, Lys, or a deletion;
A37: represents Pro, Lys, Gly, or a deletion;
A38: represents Pro, His, Lys, or a deletion;
A39: represents Ser, Asn, Lys, Gly, or a deletion;
A40: represents Ile, Lys(R), or a deletion;
wherein in the residue Lys(R), the (R) portion represents X-L-, wherein L represents a linker, and is selected from the following group consisting of gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE; and X represents a lipid.

6. A GIP receptor agonist peptide represented by formula (IV): P¹-Tyr-A2-Glu-Gly-Thr-A6-A7-Ser-Asp-Tyr-Ser-Ile-A13-A14-Asp-A16-A17-A18-Gln-A20-A21-Phe-Val-Asn-Trp-Leu-Leu-A28-A29-A30-A31-A32-A33-A34-A35-A36-A37-A38-A39-P², or a pharmaceutically acceptable salt thereof;
wherein
P¹ represents H, C₁₋₆ alkyl, or absent;
P² represents —NH₂ or —OH;
A2 represents Aib, Gly, or Ser;
A6 represents Phe or Leu;
A7 represents Ile or Thr;
A13 represents Ala, Aib, or Tyr;
A14 represents Leu, Lys, or Lys(R);
A16 represents Lys, Arg, or Ser;
A17 represents Aib, Ile, Lys, or Lys(R);
A18 represents Ala, His, Lys, or Lys(R);
A20 represents Gln, Lys, Lys(R), or Aib;
A21 represents Asp, Lys, Lys(R), or Asn;
A28 represents Ala, Aib, or, Lys, Lys(R);
A29 represents Gln, Lys, Lys(R), or Aib;
A30 represents Lys, Ser, Arg, Lys(R), or Lys(Ac);
A31 represents Pro, Gly, or a deletion;
A32 represents Ser, Gly, or a deletion;
A33 represents Ser, Gly, or a deletion;
A34 represents Gly, Lys, or a deletion;
A35 represents Ala, Ser, Lys, or a deletion;
A36 represents Pro, Lys, or a deletion;
A37 represents Pro, Lys, Gly, or a deletion;
A38 represents Pro, Lys, or a deletion; and
A39 represents Ser, Gly, Lys, or a deletion,
wherein in the residue Lys(R), the (R) portion represents X-L-, wherein L represents a linker and is selected from the group consisting of 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE, and OEGgEgEgE; and X represents C₁₄-C₁₈ monoacid or C₁₄-C₁₈ diacid.

7. The GIP receptor agonist peptide according to claim 6 or the pharmaceutically acceptable salt thereof, wherein
A14 represents Leu or Lys(R);
A17 represents Aib, Ile, or Lys(R);
A18 represents Ala, His, or Lys(R);
A20 represents Gln, Lys(R), or Aib;
A21 represents Asp, Lys(R), or Asn;
A28 represents Ala, Aib, or Lys(R);
A29 represents Gln, Lys(R), or Aib; and
A30 represents Lys, Ser, Arg, Lys(R), or Lys(Ac).

8. The GIP receptor agonist peptide according to claim 6 or the pharmaceutically acceptable salt thereof, wherein
A2 represents Aib;
A17 represents Aib, Lys, or Lys(R);
A20 represents Aib; and
A28 represents Ala or Aib,
wherein L is selected from the group consisting of 2OEG, 2OEGgE, 2OEGgEgE, G2E3, G4gE, G4gEgE, G5, G5E, G5gE, G5gEgE, gEgEgE, GGEEE, GGPAPAP, OEGgEgE, and OEGgEgEgE.

9. The GIP receptor agonist peptide according to claim 8 or the pharmaceutically acceptable salt thereof, wherein
A14 represents Leu or Lys(R);
A17 represents Aib or Lys(R).
A18 represents Ala, His, or Lys(R);
A21 represents Asp, Lys(R), or Asn;
A29 represents Gln, Lys(R), or Aib; and
A30 represents Lys, Ser, Arg, Lys(R), or Lys(Ac).

10. The GIPR agonist peptide of any one of claims 5-9 or the pharmaceutically acceptable salt thereof, wherein the lipid X is C₁₄-C₁₆ monoacid or diacid.

11. The GIPR agonist peptide of claim 10 or the pharmaceutically acceptable salt thereof, wherein the lipid X is a C₁₅ diacid or C₁₆ diacid.

12. The GIPR agonist peptide of any one of claims 5-11 or the pharmaceutically acceptable salt thereof, wherein the linker L is 2OEGgEgE or GGGGG.

13. The GIPR agonist peptide of any one of claims 5-12 or the pharmaceutically acceptable salt thereof, wherein (R) is 2OEGgEgE-C₁₅ diacid or 2OEGgEgE-C₁₆ diacid.

14. The GIPR agonist peptide of any one of claims 5-13 or the pharmaceutically acceptable salt thereof, wherein the peptide has a Lys(R) amino acid residue at amino acid position A14 and (R) is 2OEGgEgE-C16 diacid.

15. The GIPR agonist peptide of any one of claims 5-13 or the pharmaceutically acceptable salt thereof, wherein the peptide has a Lys(R) amino acid residue at amino acid position A21 and (R) is 2OEGgEgE-C₁₅ diacid

16. The GIPR agonist peptide of any one of claims 5-12 or the pharmaceutically acceptable salt thereof, represented by formula (V): P¹-Tyr-Aib-Glu-Gly-The-Phe-Ile-Ser-Asp-Tyr-Ser-Ile-A13-Leu-Asp-Arg-Aib-A18-Gln-Aib-A21-Phe-Val-Asn-Trp-Leu-Leu-Ala-Gln-A30-A31-A32-P², wherein
P¹ is methyl;
P² is OH or NH₂;
A13 represents Ala or Aib;
A18 represents Ala, Lys, or Lys(R);
A21 represents Lys, Lys(R), or Asp;
A30 represents Lys or Ser;
A31 represents Gly or Pro; and
A32 represents Gly or deletion;
wherein (R) represents X-L-, L represents 2OEGgEgE or GGGGG; and X represents a C₁₅ diacid or C₁₆ diacid.

17. The GIPR agonist peptide of claim 16 or the pharmaceutically acceptable salt thereof, wherein
A18 represents Ala or Lys(R); and
A21 represents Lys(R) or Asp.

18. The GIPR agonist peptide of any one of claims 5-13 and 16-17, or the pharmaceutically acceptable salt thereof, represented by the formula: P¹-Y-Aib-E-G-T-F-I-S-D-Y-S-I-A-L-D-R-Aib-A-Q-Aib-Km-F-V-N-W-L-L-A-Q-K-G-P²; wherein Km is Lys-2OEGgEgE-C₁₆ diacid, P¹-Y-Aib-E-G-T-F-I-S-D-Y-S-I-Aib-L-D-R-Aib-Km-Q-Aib-D-F-V-N-W-L-L-A-Q-S-P-G-P²; wherein Km is Lys-2OEGgEgE-C₁₆ diacid, or P¹-Y-Aib-E-G-T-F-I-S-D-Y-S-I-A-L-D-R-Aib-A-Q-Aib-Km-F-V-N-W-L-L-A-Q-K-G-P²; wherein Km is Lys-2OEGgEgE-C₁₅ diacid.

19. The GIPR agonist peptide of any one of claims 16-18 or the pharmaceutically acceptable salt thereof, represented by the formula: Me-Tyr-Aib-Glu-Gly-Thr-Phe-Ile-Ser-Asp-Tyr-Ser-Ile-Ala-Leu-Asp-Arg-Aib-Ala-Gln-Aib-Lys(R)-Phe-Val-Asn-Trp-Leu-Leu-Ala-Gln-Lys-Gly-OH; wherein Lys(R) is Lys-2OEGgEgE-C₁₆ diacid.

20. The GIPR agonist peptide of any one of claims 16-18 or the pharmaceutically acceptable salt thereof, represented by the formula: Me-Tyr-Aib-Glu-Gly-Thr-Phe-Ile-Ser-Asp-Tyr-Ser-Ile-Aib-Leu-Asp-Arg-Aib-Lys(R)-Gln-Aib-Asp-Phe-Val-Asn-Trp-Leu-Leu-Ala-Gln-Ser-Pro-Gly-OH; wherein Lys(R) is Lys-2OEGgEgE-C₁₆ diacid.

21. The GIPR agonist peptide of any one of claims 16-18 or the pharmaceutically acceptable salt thereof, represented by the formula: Me-Tyr-Aib-Glu-Gly-Thr-Phe-Ile-Ser-Asp-Tyr-Ser-Ile-Ala-Leu-Asp-Arg-Aib-Ala-Gln-Aib-Lys(R)-Phe-Val-Asn-Trp-Leu-Leu-Ala-Gln-Lys-Gly-OH; wherein Lys(R) is Lys-2OEGgEgE-C₁₅ diacid.

22. The GIP receptor agonist peptide according to any one of claims 5-7 or the pharmaceutically acceptable salt thereof, wherein the amino acid sequence comprises: Me-Tyr-Aib-Glu-Gly-Thr-Phe-Ile-Ser-Asp-Tyr-Ser-Ile-Ala-Leu-Asp-Arg-Aib-Ala-Gln-Aib-Lys(R)-Phe-Val-Asn-Trp-Leu-Leu-Ala-Gln-Arg-NH2; wherein Lys(R) is Lys-2OEGgEgE-C₁₅ diacid.

23. The GIP receptor agonist peptide according to any one of claims 5-9 or the pharmaceutically acceptable salt thereof, represented by: Me-Tyr-Aib-Glu-Gly-Thr-Phe-Ile-Ser-Asp-Tyr-Ser-Ile-Aib-Lys(R)-Asp-Arg-Aib-Ala-Gln-Aib-Asn-Phe-Val-Asn-Trp-Leu-Leu-Ala-Gln-Ser-Pro-Ser-Ser-Gly-Ala-Pro-Pro-Pro-Ser-OH; wherein Lys(R) is Lys-GGGGG-C₁₅ diacid.

24. The GIP receptor agonist peptide according to any one of claims 1-23 or a salt thereof, wherein the GIP receptor agonist peptide has a selectivity ratio, expressed as a ratio of (GLP1R EC50/GIPR EC50) of greater than 10, or greater than 100, or greater than 1,000, or greater than 100,000.

25. The GIP receptor agonist peptide according to any one of claims 1-23 or a salt thereof, wherein the GIP receptor agonist peptide has an IV T½ life of elimination of ranges between 4-10 hours.

26. The GIP receptor agonist peptide according to claim 7 or 9, or a salt thereof, wherein the GIP receptor agonist peptide has a solubility of 15 mg/mL or greater.

27. A medicament comprising the GIP receptor agonist peptide according to any one of claims 1-26, or a pharmaceutically acceptable salt thereof.

28. A pharmaceutical composition comprising the GIP receptor agonist peptide according to any one of claims 1-26, or a pharmaceutically acceptable salt thereof.

29. The GIP receptor agonist peptide according to any one of claims 1-26 or a salt thereof, or the medicament according to claim 27, or the pharmaceutical composition according to claim 28, which is administered to treat emesis as a monotherapy.

30. The GIP receptor agonist peptide according to any one of claims 1-26 or a salt thereof, or the medicament according to claim 27, or the pharmaceutical composition according to claim 28, which is administered to a subject Q1D, or once per 24 hours to treat or prevent emesis, including vomiting and/or nausea.

31. The medicament according to claim 27, which is an activator of a GIP receptor.

32. The medicament according to claim 27, which is a suppressant for vomiting or nausea.

33. Use of the GIP receptor agonist peptide of any one of claims 1-26, or a salt thereof, or the medicament according to claim 27, or the pharmaceutical composition according to claim 28, for the manufacture of a suppressant for vomiting or nausea.

34. The GIP receptor agonist peptide of any one of claims 1-26 or a salt thereof, or the medicament according to claim 27, or the pharmaceutical composition according to claim 28, for use in suppressing vomiting or nausea.

35. A method for preventing or treating emesis in a subject, comprising administering an effective amount of the peptide of any one of claims 1-26 or a salt thereof, or the medicament according to claim 27, or the pharmaceutical composition according to claim 28, to the subject.

36. The method according to claim 35, wherein the emesis is nausea and/or vomiting.

37. The medicament according to claim 32, the use according to claim 33, the peptide or a salt thereof, the medicament, or the pharmaceutical composition according to claim 34, or the method according to claim 36, where the emesis, vomiting or the nausea is caused by one or more conditions or causes selected from the following (1) to (10):
(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea caused by a vestibular disorder such as motion sickness or dizziness
(7) Opioid withdrawal;
(8) Pregnancy including hyperemesis gravidarium;
(9) A vestibular disorder such as motion sickness or dizziness; or
(10) A physical injury causing local, systemic, acute or chronic pain.

38. The method according to claim 35, wherein the emesis is a result of cyclic vomiting syndrome or chemotherapy.

39. The method of claim 35, wherein the subject is a non-type 2 diabetes mellitus subject.

40. The method according to claim 35, wherein the emesis is delayed emesis or anticipatory emesis.

41. The method according to any one of claims 35-40, wherein emesis is treated in the subject without inducing anxiety or sedation in the subject.

42. The method according to any one of claims 35-41, wherein emesis is treated in the subject without inducing suppression of glucagon secretion when plasma glucose levels are above fasting levels.

43. The method according to any one of claims 35-42, wherein emesis is treated in the subject without substantially activating the GLP-1 receptor.

44. The method according to claim 42 or 43, wherein emesis is treated in the subject without concomitant, subsequent, or prior administration of a GLP-1 receptor agonist.

45. The method according to any one of claims 35-44, wherein emesis is treated in a subject not taking a medicament to control a metabolic syndrome disorder.

46. The method according to any one of claims 35-45, wherein emesis is treated in a subject taking a medicament to control a metabolic syndrome disorder.

47. The method according to claim 46, wherein the metabolic syndrome disorder is type 2 diabetes mellitus or obesity.

48. The method according to any one of claims 35-47, wherein the emesis is caused by or causes cyclic vomiting syndrome, or nausea or vomiting associated with chemotherapy.

49. The method according to claim 38 or 48, wherein the chemotherapy or chemotherapeutic agent comprises: (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome

50. The method according to claim 35, wherein the subject has type 2 diabetes mellitus.

51. The method according to any one of claims 35-50, wherein the GIP receptor agonist peptide or medicament is administered subcutaneously, intravenously, intramuscularly, intraperitonealy, orally or via inhalation.

52. The method according to any one of claims 35-51, wherein the effective amount of the GIP receptor agonist peptide administered to the subject is about 0.01 to 0.5 mg/kg/day, 0.1 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

53. The method according to any one of claims 35-52, wherein the subject is human.

54. The method according to any one of claims 35-53, wherein the GIP receptor agonist peptide or medicament is administered to the subject before, during, or after the subject develops the disease-state.

55. The method according to any one of claims 35-54, wherein the GIP receptor agonist peptide or medicament is administered to the subject 1 times per day, or 1 times per 24 hours.

56. The method according to any one of claims 35-55, wherein the GIP receptor agonist peptide or medicament is administered to the subject for 1-5 days, 1-5 weeks, 1-5 months, or 1-5 years.


SEQUENCE LISTING (355 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
  MOD_RES (17)..(17): Aib
YXEGTFISDYSIAXDRXAQQNFVNWLLAQK

SEQ ID NO: 3 - protein, 30 aa, Artificial Sequence; identical to SEQ ID NO: 2
  source: /note=“Description of Artificial Sequence Synthetic polypeptide”
  MOD_RES (2)..(2): Aib
  MOD_RES (14)..(14): Lys, wherein the side chain of Lys is connected to the sequence 1OEGgE-C16DA
  MOD_RES (17)..(17): Aib

SEQ ID NO: 4 - protein, 30 aa, Artificial Sequence; identical to SEQ ID NO: 2
  source: /note=“Description of Artificial Sequence Synthetic polypeptide”
  MOD_RES (2)..(2): Aib
  MOD_RES (14)..(14): Lys, wherein the side chain of Lys is connected to the sequence 3OEGgE-C16DA
  MOD_RES (17)..(17): Aib

SEQ ID NO: 5 - protein, 30 aa, Artificial Sequence
  source: /note=“Description of Artificial Sequence Synthetic polypeptide”
  MOD_RES (2)..(2): Aib
  MOD_RES (20)..(20): Lys, wherein the side chain of Lys is connected to the sequence 2OEGgE-C16DA
YXEGTFISDYSIALDRIHQXNFVNWLLAQK

SEQ ID NO: 6 - protein, 30 aa, Artificial Sequence; identical to SEQ ID NO: 5
  source: /note=“Description of Artificial Sequence Synthetic polypeptide”
  MOD_RES (2)..(2): Aib
  MOD_RES (20)..(20): Lys, wherein the side chain of Lys is connected to the sequence 2OEGgE-C15DA

SEQ ID NO: 7 - protein, 30 aa, Artificial Sequence
  source: /note=“Description of Artificial Sequence Synthetic polypeptide”
  MOD_RES (14)..(14): Lys, wherein the side chain of Lys is connected to the sequence 2OEGgE-C16DA
  MOD_RES (20)..(20): Aib
YGEGTFISDYSIAXDRIHQXNFVNWLLAQK

SEQ ID NO: 8 - protein, 30 aa, Artificial Sequence
  source: /note=“Description of Artificial Sequence Synthetic polypeptide”
  MOD_RES (14)..(14): Lys, wherein the side chain of Lys is connected to the sequence 2OEGgE-C16DA
  MOD_RES (20)..(20): Aib
YSEGTFISDYSIAXDRIHQXNFVNWLLAQK

SEQ ID NO: 9 - protein, 30 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgE-C16DA
  MOD_RES (20)..(20): Aib
YXEGTFISDYSIALDRIXQXNFVNWLLAQK

SEQ ID NO: 10 - protein, 30 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgE-C16DA
YXEGTFISDYSIALDRIHQXXFVNWLLAQK

SEQ ID NO: 11 - protein, 30 aa, Artificial Sequence
  source: /note=“Description of Artificial Sequence Synthetic polypeptide”
  MOD_RES (2)..(2): Aib
  MOD_RES (20)..(20): Aib
  MOD_RES (28)..(28): Lys, wherein the side chain of Lys is connected to the sequence 2OEGgE-C16DA
YXEGTFISDYSIALDRIHQXNFVNWLLXQK

SEQ ID NO: 12 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
  MOD_RES (17)..(17): Aib
  MOD_RES (20)..(20): Aib
YXEGTFISDYSIAXDRXAQXNFVNWLLAQK

SEQ ID NO: 13 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA
YXEGTFISDYSIALDRXHQXXFVNWLLAQK

SEQ ID NO: 14 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib
YXEGTLISDYSIAXDRXAQXNFVNWLLAQR

SEQ ID NO: 15 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQR

SEQ ID NO: 16 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 17 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib
YXEGTFISDYSIAXDRXAQXNFVNWLLAQS

SEQ ID NO: 18 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXNFVNWLLAQSPSSGAPPPS

SEQ ID NO: 19 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence gEgEgE-C15DA
  MOD_RES (17)..(17): Aib
  MOD_RES (20)..(20): Aib
YXEGTFISDYSIAXDRXAQXNFVNWLLAQSPSSGAPPPS

SEQ ID NO: 20 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib
YXEGTFISDYSIXXDRXAQXNFVNWLLAQRPSSGAPPPS

SEQ ID NO: 21 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C15DA
  MOD_RES (20)..(20): Aib
YXEGTFISDYSIXLDRXAQXNFVNWLLAQRPSSGAPPPS

SEQ ID NO: 22 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15DA
YXEGTFISDYSIXLDRXAQXNFVNWLLAQXPSSGAPPPS

SEQ ID NO: 23 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib

SEQ ID NO: 24 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence OEGgEgEgE-C15DA
  MOD_RES (17)..(17): Aib
  MOD_RES (20)..(20): Aib

SEQ ID NO: 25 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence OEGgEgEgE-C15DA
YXEGTFISDYSIALDRXAQXXFVNWLLAQK

SEQ ID NO: 26 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQKG

SEQ ID NO: 27 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib

SEQ ID NO: 28 - protein, 30 aa, Artificial Sequence; identical to SEQ ID NO: 25
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 29 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence OEGgEgEgE-C15DA
  MOD_RES (17)..(17): Aib
  MOD_RES (20)..(20): Aib
  MOD_RES (28)..(28): Aib
YXEGTFISDYSIAXDRXAQXNFVNWLLXQK

SEQ ID NO: 30 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence OEGgEgEgE-C15DA
  MOD_RES (28)..(28): Aib
YXEGTFISDYSIALDRXAQXXFVNWLLXQK

SEQ ID NO: 31 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence G2E3-C15DA
  MOD_RES (17)..(17): Aib
  MOD_RES (20)..(20): Aib

SEQ ID NO: 32 - protein, 30 aa, Artificial Sequence; identical to SEQ ID NO: 25
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G2E3-C15DA

SEQ ID NO: 33 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence G2E3-C15DA
YXEGTFISDYSIALDRXAQXNFVNWLLAQXPSSGAPPPS

SEQ ID NO: 34 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA
  MOD_RES (20)..(20): Aib
YXEGTFISDYSIALDRXAQXNFVNWLLAQRPSSGAPPPS

SEQ ID NO: 35 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDRXAQXNFVNWLLAXK

SEQ ID NO: 36 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDKXAQXNFVNWLLAXS

SEQ ID NO: 37 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDKXHQXDFVNWLLAXK

SEQ ID NO: 38 - protein, 30 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA
  MOD_RES (20)..(20): Aib
YXEGTFISDYSIALDRXHQXNFVNWLLAQS

SEQ ID NO: 39 - protein, 30 aa, Artificial Sequence; identical to SEQ ID NO: 29
  source: /note=“Description of Artificial Sequence Synthetic polypeptide”
  MOD_RES (2)..(2): Aib
  MOD_RES (14)..(14): Lys, wherein the side chain of Lys is connected to the sequence OEGgEgEgE-C15DA
  MOD_RES (17)..(17): Aib
  MOD_RES (20)..(20): Aib
  MOD_RES (28)..(28): Aib

SEQ ID NO: 40 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence OEGgEgEgE-C15DA
  MOD_RES (17)..(17): Aib
  MOD_RES (20)..(20): Aib
  MOD_RES (29)..(29): Aib
YXEGTFISDYSIAXDRXAQXNFVNWLLAXK

SEQ ID NO: 41 - protein, 30 aa, Artificial Sequence; identical to SEQ ID NO: 30
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence OEGgEgEgE-C15DA
  MOD_RES (28)..(28): Aib

SEQ ID NO: 42 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence G2E3-C15DA
YXEGTFISDYSIALDRXAQXNFVNWLLAQX

SEQ ID NO: 43 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G2E3-C15DA
  MOD_RES (20)..(20): Aib
YXEGTFISDYSIALDRXXQXNFVNWLLAQK

SEQ ID NO: 44 - protein, 30 aa, Artificial Sequence; identical to SEQ ID NO: 42
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence OEGgEgEgE-C15DA

SEQ ID NO: 45 - protein, 30 aa, Artificial Sequence; identical to SEQ ID NO: 43
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence OEGgEgEgE-C15DA
  MOD_RES (20)..(20): Aib

SEQ ID NO: 46 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence OEGgEgEgE-C15DA
  MOD_RES (29)..(29): Aib
YXEGTFISDYSIALDRXAQXXFVNWLLAXK

SEQ ID NO: 47 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C15DA
  MOD_RES (17)..(17): Aib
  MOD_RES (20)..(20): Aib
YXEGTFTSDYSIYXDRXAQXNFVNWLLAQRPSSGAPPPS

SEQ ID NO: 48 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA
  MOD_RES (20)..(20): Aib
YXEGTFISDYSIALDRXHQXNFVNWLLAQRPSSGAPPPS

SEQ ID NO: 49 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 50 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G2E3-C15DA
  MOD_RES (17)..(17): Aib
  MOD_RES (20)..(20): Aib
YXEGTFISDYSIAXDRXAQXNFVNWLLAQRPSSGAPPPS

SEQ ID NO: 51 - protein, 39 aa, Artificial Sequence; identical to SEQ ID NO: 50
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEG-C15DA
  MOD_RES (17)..(17): Aib
  MOD_RES (20)..(20): Aib

SEQ ID NO: 52 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G2E3-C15DA
  MOD_RES (17)..(17): Aib
  MOD_RES (20)..(20): Aib

SEQ ID NO: 53 - protein, 39 aa, Artificial Sequence; identical to SEQ ID NO: 33
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence OEGgEgEgE-C15DA

SEQ ID NO: 54 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G2E3-C15DA
  MOD_RES (20)..(20): Aib
YXEGTFISDYSIALDRXAQXNFVNWLLAQSPSSGAPPPS

SEQ ID NO: 55 - protein, 39 aa, Artificial Sequence; identical to SEQ ID NO: 54
  source: /note=“Description of Artificial Sequence Synthetic polypeptide”
  MOD_RES (2)..(2): Aib
  MOD_RES (17)..(17): Lys, wherein the side chain of Lys is connected to the sequence OEGgEgEgE-C15DA
  MOD_RES (20)..(20): Aib

SEQ ID NO: 56 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G2E3-C15DA
  MOD_RES (20)..(20): Aib

SEQ ID NO: 57 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G4E2-C15DA
  MOD_RES (17)..(17): Aib
  MOD_RES (20)..(20): Aib

SEQ ID NO: 58 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C15DA
  MOD_RES (17)..(17): Aib
  MOD_RES (20)..(20): Aib

SEQ ID NO: 59 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gEgE-C15DA
  MOD_RES (17)..(17): Aib
  MOD_RES (20)..(20): Aib

SEQ ID NO: 60 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G3gEgE-C15DA
  MOD_RES (17)..(17): Aib
  MOD_RES (20)..(20): Aib
YXEGTFISDYSIXXDRXAQXNFVNWLLAQR

SEQ ID NO: 61 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5gE-C15DA

SEQ ID NO: 62 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5E-C15DA

SEQ ID NO: 63 - protein, 39 aa, Artificial Sequence; identical to SEQ ID NO: 48
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 64 - protein, 39 aa, Artificial Sequence; identical to SEQ ID NO: 48
  source: /note=“Description of Artificial Sequence Synthetic polypeptide”
  MOD_RES (2)..(2): Aib
  MOD_RES (17)..(17): Lys, wherein the side chain of Lys is connected to the sequence G5gEgE-C15DA
  MOD_RES (20)..(20): Aib

SEQ ID NO: 65 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence OEGgEgE-C15DA
  MOD_RES (20)..(20): Aib
YXEGTFISDYSIALDRXHQXNFVNWLLAQSPSSGAPPPS

SEQ ID NO: 66 - protein, 39 aa, Artificial Sequence; identical to SEQ ID NO: 65
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 67 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence OEGgEgE-C15DA
  MOD_RES (20)..(20): Aib
YXEGTFISDYSIALDRXHQXDFVNWLLAQRPSSGAPPPS

SEQ ID NO: 68 - protein, 39 aa, Artificial Sequence; identical to SEQ ID NO: 67
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 69 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib
YXEGTFISDYSIALDRXHQXDFVNWLLAQSPSSGAPPPS

SEQ ID NO: 70 - protein, 39 aa, Artificial Sequence; identical to SEQ ID NO: 69
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 71 - protein, 39 aa, Artificial Sequence; identical to SEQ ID NO: 33
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence OEGgEgE-C15DA

SEQ ID NO: 72 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G5gE-C15DA
  MOD_RES (20)..(20): Aib

SEQ ID NO: 73 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C15DA
  MOD_RES (20)..(20): Aib

SEQ ID NO: 74 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G5gE-C15DA
  MOD_RES (20)..(20): Aib
YXEGTFISDYSIALDRXXQXDFVNWLLAQRPSSGAPPPS

SEQ ID NO: 75 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G5gE-C15DA
  MOD_RES (20)..(20): Aib
YXEGTFISDYSIALDRXXQXDFVNWLLAQSPSSGAPPPS

SEQ ID NO: 76 - protein, 39 aa, Artificial Sequence; identical to SEQ ID NO: 74
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C15DA
  MOD_RES (20)..(20): Aib

SEQ ID NO: 77 - protein, 39 aa, Artificial Sequence; identical to SEQ ID NO: 75
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C15DA
  MOD_RES (20)..(20): Aib

SEQ ID NO: 78 - protein, 39 aa, Artificial Sequence; identical to SEQ ID NO: 50
  source: /note=“Description of Artificial Sequence Synthetic polypeptide”
  MOD_RES (2)..(2): Aib
  MOD_RES (14)..(14): Lys, wherein the side chain of Lys is connected to the sequence G4gEgE-C15DA
  MOD_RES (17)..(17): Aib
  MOD_RES (20)..(20): Aib

SEQ ID NO: 79 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G4gE-C15DA
  MOD_RES (17)..(17): Aib
  MOD_RES (20)..(20): Aib

SEQ ID NO: 80 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C15DA
  MOD_RES (17)..(17): Aib
  MOD_RES (20)..(20): Aib
YXEGTFISDYSIAXDRXAQXDFVNWLLAQSPSSGAPPPS

SEQ ID NO: 81 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C15DA
  MOD_RES (17)..(17): Aib
  MOD_RES (20)..(20): Aib
YXEGTFISDYSIAXDRXAQXDFVNWLLAQRPSSGAPPPS

SEQ ID NO: 82 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5gEgE-C15DA
  MOD_RES (17)..(17): Aib
  MOD_RES (20)..(20): Aib

SEQ ID NO: 83 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C15DA
  MOD_RES (17)..(17): Aib
  MOD_RES (20)..(20): Aib

SEQ ID NO: 84 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQRPSSGAPPPS

SEQ ID NO: 85 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C15DA
YXEGTFISDYSIALDRXHQXNFVNWLLAXS

SEQ ID NO: 86 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQS

SEQ ID NO: 87 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence OEGgEgE-C15DA

SEQ ID NO: 88 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDRXAQXNFVNWLLAXRPSSGAPPPS

SEQ ID NO: 89 - protein, 39 aa, Artificial Sequence; identical to SEQ ID NO: 88
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C15DA

SEQ ID NO: 90 - protein, 30 aa, Artificial Sequence; identical to SEQ ID NO: 42
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence G5gEgE-C15DA

SEQ ID NO: 91 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence G5gEgE-C15DA
YXEGTFISDYSIALDRXAQXDFVNWLLAQX

SEQ ID NO: 92 - protein, 30 aa, Artificial Sequence; identical to SEQ ID NO: 42
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15DA

SEQ ID NO: 93 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA
YXEGTFISDYSIALDRXAQXXFVNWLLAQSPSSGAPPPS

SEQ ID NO: 94 - protein, 39 aa, Artificial Sequence; identical to SEQ ID NO: 93
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 95 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence OEGgEgE-C15DA
  MOD_RES (17)..(17): Aib
  MOD_RES (20)..(20): Aib

SEQ ID NO: 96 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence GGEEE-C15DA
  MOD_RES (17)..(17): Aib
  MOD_RES (20)..(20): Aib

SEQ ID NO: 97 - protein, 35 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQRGGGGS

SEQ ID NO: 98 - protein, 35 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C15DA
  MOD_RES (17)..(17): Aib
  MOD_RES (20)..(20): Aib
YXEGTFISDYSIAXDRXAQXDFVNWLLAQRGGGGS

SEQ ID NO: 99 - protein, 34 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDRXAQXDFVNWLLAXSPSSG

SEQ ID NO: 100 - protein, 39 aa, Artificial Sequence; identical to SEQ ID NO: 50
  source: /note=“Description of Artificial Sequence Synthetic polypeptide”
  MOD_RES (2)..(2): Aib
  MOD_RES (14)..(14): Lys, wherein the side chain of Lys is connected to the sequence GGEEE-C15DA
  MOD_RES (17)..(17): Aib
  MOD_RES (20)..(20): Aib

SEQ ID NO: 101 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgE-C15DA
YXEGTFISDYSIXLDRXAQXDFVNWLLAQXPSSGAPPPS

SEQ ID NO: 102 - protein, 39 aa, Artificial Sequence; identical to SEQ ID NO: 33
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15DA

SEQ ID NO: 103 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA
YXEGTFISDYSIXLDRXAQXXFVNWLLAQSPSSGAPPPS

SEQ ID NO: 104 - protein, 39 aa, Artificial Sequence; identical to SEQ ID NO: 103
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 105 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA
  MOD_RES (20)..(20): Aib
YXEGTFISDYSIALDRXAQXDFVNWLLAQRPSSGAPPPS

SEQ ID NO: 106 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence GGEEE-C15DA
  MOD_RES (20)..(20): Aib

SEQ ID NO: 107 - protein, 39 aa, Artificial Sequence; identical to SEQ ID NO: 75
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence GGPAPAP-C15DA
  MOD_RES (20)..(20): Aib

SEQ ID NO: 108 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence GGPAPAP-C15DA
YXEGTFISDYSIALDRXAQXDFVNWLLAXSPSSGAPPPS

SEQ ID NO: 109 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIXLDRXAQXXFVNWLLAQR

SEQ ID NO: 110 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDRXAQXDFVNWLLAXRPSSGAPPPS

SEQ ID NO: 111 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C15DA
  MOD_RES (17)..(17): Aib
  MOD_RES (20)..(20): Aib
YXEGTFISDYSIXXDRXAQXDFVNWLLAQRGGGGS

SEQ ID NO: 112 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIXLDRXXQXNFVNWLLAQSPSSGAPPPS

SEQ ID NO: 113 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence OEGgEgEgE-C15DA
YXEGTFISDYSIXLDRXAQXXFVNWLLAQK

SEQ ID NO: 114 - protein, 30 aa, Artificial Sequence; identical to SEQ ID NO: 113
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence GG-EEE-C15DA

SEQ ID NO: 115 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIXLDRXAQXXFVNWLLAQRGGGGS

SEQ ID NO: 116 - protein, 30 aa, Artificial Sequence; identical to SEQ ID NO: 60
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence OEGgEgEgE-C15DA
  MOD_RES (17)..(17): Aib
  MOD_RES (20)..(20): Aib

SEQ ID NO: 117 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgE-C15DA
  MOD_RES (17)..(17): Aib
  MOD_RES (20)..(20): Aib
YXEGTFISDYSIXXDRXAQXDFVNWLLAQRPSSGAPPPS

SEQ ID NO: 118 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIXLDRXXQXDFVNWLLAQR

SEQ ID NO: 119 - protein, 30 aa, Artificial Sequence; identical to SEQ ID NO: 118
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgE-C15DA
  MOD_RES (20)..(20): Aib

SEQ ID NO: 120 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C16DA
  MOD_RES (20)..(20): Aib

SEQ ID NO: 121 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C16DA
  MOD_RES (17)..(17): Aib
  MOD_RES (20)..(20): Aib

SEQ ID NO: 122 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16DA

SEQ ID NO: 123 - protein, 39 aa, Artificial Sequence; identical to SEQ ID NO: 21
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C16DA
  MOD_RES (20)..(20): Aib

SEQ ID NO: 124 - protein, 39 aa, Artificial Sequence; identical to SEQ ID NO: 22
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C16DA

SEQ ID NO: 125 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIXXDRXAQXNFVNWLLAQRPSSKKKKKK

SEQ ID NO: 126 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C16DA
  MOD_RES (17)..(17): Aib
  MOD_RES (20)..(20): Aib

SEQ ID NO: 127 - protein, 39 aa, Artificial Sequence; identical to SEQ ID NO: 81
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C16DA
  MOD_RES (17)..(17): Aib
  MOD_RES (20)..(20): Aib

SEQ ID NO: 128 - protein, 39 aa, Artificial Sequence; identical to SEQ ID NO: 54
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA
  MOD_RES (20)..(20): Aib

SEQ ID NO: 129 - protein, 39 aa, Artificial Sequence; identical to SEQ ID NO: 117
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgE-C16DA
  MOD_RES (17)..(17): Aib
  MOD_RES (20)..(20): Aib

SEQ ID NO: 130 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgE-C16DA
  MOD_RES (17)..(17): Aib
  MOD_RES (20)..(20): Aib
YXEGTFISDYSIXXDRXAQXDFVNWLLAQSPSSGAPPPS

SEQ ID NO: 131 - protein, 39 aa, Artificial Sequence; identical to SEQ ID NO: 84
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16DA

SEQ ID NO: 132 - protein, 39 aa, Artificial Sequence; identical to SEQ ID NO: 54
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 133 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib
YXEGTFISDYSIALDRXAQXDFVNWLLAQSPSSGAPPPS

SEQ ID NO: 134 - protein, 35 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXNFVNWLLAQSGGGGS

SEQ ID NO: 135 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib
YXEGTFISDYSIXXDRXAQXNFVNWLLAQRGGGGS

SEQ ID NO: 136 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C15DA
  MOD_RES (20)..(20): Aib
YXEGTFISDYSIXLDRXAQXNFVNWLLAQRGGGGS

SEQ ID NO: 137 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15DA
YXEGTFISDYSIXLDRXAQXNFVNWLLAQXGGGGS

SEQ ID NO: 138 - protein, 39 aa, Artificial Sequence; identical to SEQ ID NO: 105
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 139 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G2E3-C15DA
  MOD_RES (17)..(17): Aib
  MOD_RES (20)..(20): Aib

SEQ ID NO: 140 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib
YXEGTFISDYSIXXDRXAQXNFVNWLLAQSPSSGAPPPS

SEQ ID NO: 141 - protein, 39 aa, Artificial Sequence; identical to SEQ ID NO: 130
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 142 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIXXDRXAQXDFVNWLLAQSG

SEQ ID NO: 143 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIXXDRXAQXDFVNWLLAQRG

SEQ ID NO: 144 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16DA

SEQ ID NO: 145 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C16DA
  MOD_RES (20)..(20): Aib

SEQ ID NO: 146 - protein, 39 aa, Artificial Sequence; identical to SEQ ID NO: 21
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C16DA
  MOD_RES (20)..(20): Aib

SEQ ID NO: 147 - protein, 39 aa, Artificial Sequence; identical to SEQ ID NO: 81
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 148 - protein, 39 aa, Artificial Sequence; identical to SEQ ID NO: 50
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 149 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 150 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQSG

SEQ ID NO: 151 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 152 - protein, 39 aa, Artificial Sequence; identical to SEQ ID NO: 80
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 153 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDRXAQXDFVNWLLAQSPSSGAPPPG

SEQ ID NO: 154 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDSXAQXDFVNWLLAQRPSSGAPPPS

SEQ ID NO: 155 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDSXAQXNFVNWLLAQSPSSGAPPPS

SEQ ID NO: 156 - protein, 39 aa, Artificial Sequence; identical to SEQ ID NO: 80
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 157 - protein, 39 aa, Artificial Sequence; identical to SEQ ID NO: 130
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 158 - protein, 37 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDRXAQXDFVNWLLAQSPSSGAPG

SEQ ID NO: 159 - protein, 34 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDRXAQXDFVNWLLAQSPSSG

SEQ ID NO: 160 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDRXAQXDFVNWLLAQSPG

SEQ ID NO: 161 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDRXAQXNFVNWLLAQSG

SEQ ID NO: 162 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXNFVNWLLAQSG

SEQ ID NO: 163 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXDFVNWLLAQRG

SEQ ID NO: 164 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXDFVNWLLAQSG

SEQ ID NO: 165 - protein, 39 aa, Artificial Sequence; identical to SEQ ID NO: 54
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib

SEQ ID NO: 166 - protein, 39 aa, Artificial Sequence; identical to SEQ ID NO: 105
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib

SEQ ID NO: 167 - protein, 39 aa, Artificial Sequence; identical to SEQ ID NO: 133
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib

SEQ ID NO: 168 - protein, 39 aa, Artificial Sequence; identical to SEQ ID NO: 133
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib

SEQ ID NO: 169 - protein, 31 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib
YXEGTFISDYSIALDRXAQXDFVNWLLAQSG

SEQ ID NO: 170 - protein, 31 aa, Artificial Sequence; identical to SEQ ID NO: 163
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C16DA
  MOD_RES (20)..(20): Aib

SEQ ID NO: 171 - protein, 31 aa, Artificial Sequence; identical to SEQ ID NO: 164
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C16DA
  MOD_RES (20)..(20): Aib

SEQ ID NO: 172 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXNFVNWLLAQRG

SEQ ID NO: 173 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C16DA
  MOD_RES (17)..(17): Aib
  MOD_RES (20)..(20): Aib
YXEGTFISDYSIAXDRXAQXDFVNWLLAQSG

SEQ ID NO: 174 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C16DA
  MOD_RES (17)..(17): Aib
  MOD_RES (20)..(20): Aib
YXEGTFISDYSIAXDRXAQXDFVNWLLAQRG

SEQ ID NO: 175 - protein, 31 aa, Artificial Sequence; identical to SEQ ID NO: 169
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C16DA
  MOD_RES (20)..(20): Aib

SEQ ID NO: 176 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIXLDRXAQXXFVNWLLAQKG

SEQ ID NO: 177 - protein, 31 aa, Artificial Sequence; identical to SEQ ID NO: 143
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C16DA
  MOD_RES (17)..(17): Aib
  MOD_RES (20)..(20): Aib

SEQ ID NO: 178 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDSXAQXDFVNWLLAQKG

SEQ ID NO: 179 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G5-C15DA
  MOD_RES (20)..(20): Aib
YXEGTFISDYSIXLDSXAQXDFVNWLLAQKG

SEQ ID NO: 180 - protein, 31 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib
YXEGTFISDYSIALDSXAQXDFVNWLLAQKG

SEQ ID NO: 181 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G4gE-C15DA
  MOD_RES (17)..(17): Aib
  MOD_RES (20)..(20): Aib
YXEGTFISDYSIAXDRXAQXDFVNWLLAQKG

SEQ ID NO: 182 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
  MOD_RES (30)..(30): Lys(Ac)
YXEGTFISDYSIAXDRXAQXDFVNWLLAQXG

SEQ ID NO: 183 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIXXDRXAQXDFVNWLLAQSPG

SEQ ID NO: 184 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIXLDRXAQXXFVNWLLAQSPG

SEQ ID NO: 185 - protein, 32 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQSPG

SEQ ID NO: 186 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C16DA
  MOD_RES (20)..(20): Aib
YXEGTFISDYSIXLDRXXQXDFVNWLLAQSPG

SEQ ID NO: 187 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G5-C15DA
  MOD_RES (20)..(20): Aib
YXEGTFISDYSIXLDRXAQXDFVNWLLAQSPG

SEQ ID NO: 188 - protein, 39 aa, Artificial Sequence; identical to SEQ ID NO: 140
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 189 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 190 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 191 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (26)..(26): Lys, wherein the side chain of Lys is connected to the sequence gE-C16
YXEGTFISDYSIALDRXHQXNFVNWXLAQR

SEQ ID NO: 192 - protein, 40 aa, Artificial Sequence
  source: /note=“Description of Artificial Sequence Synthetic polypeptide”
  MOD_RES (2)..(2): Aib
  MOD_RES (20)..(20): Aib
  MOD_RES (40)..(40): Lys, wherein the side chain of Lys is connected to C16DA
YXEGTFTSDYSIYLDKQAAXEFVNWLLAGGPSSGAPPPSX

SEQ ID NO: 193 - protein, 30 aa, Artificial Sequence
  source: /note=“Description of Artificial Sequence Synthetic polypeptide”
  MOD_RES (14)..(14): Lys, wherein the side chain of Lys is connected to the sequence 2OEGgE-C16DA
YGEGTFISDYSIAXDRIHQQNFVNWLLAQK

SEQ ID NO: 194 - protein, 30 aa, Artificial Sequence; identical to SEQ ID NO: 193
  source: /note=“Description of Artificial Sequence Synthetic polypeptide”
  MOD_RES (14)..(14): Lys, wherein the side chain of Lys is connected to the sequence PEG10gE-C16DA

SEQ ID NO: 195 - protein, 30 aa, Artificial Sequence; identical to SEQ ID NO: 193
  source: /note=“Description of Artificial Sequence Synthetic polypeptide”
  MOD_RES (14)..(14): Lys, wherein the side chain of Lys is connected to the sequence PEG8gE-C16DA

SEQ ID NO: 196 - protein, 30 aa, Artificial Sequence; identical to SEQ ID NO: 193
  source: /note=“Description of Artificial Sequence Synthetic polypeptide”
  MOD_RES (14)..(14): Lys, wherein the side chain of Lys is connected to the sequence PEG6gE-C16DA

SEQ ID NO: 197 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
  MOD_RES (20)..(20): Aib
YXEGTFISDYSIAXDRIAQXNFVNWLLAQK

SEQ ID NO: 198 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence gEgEgE-C15DA
  MOD_RES (17)..(17): Aib
  MOD_RES (20)..(20): Aib

SEQ ID NO: 199 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 200 - protein, 31 aa, Artificial Sequence; identical to SEQ ID NO: 143
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 201 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C16DA
  MOD_RES (20)..(20): Aib

SEQ ID NO: 202 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgEgE-C15DA

SEQ ID NO: 203 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXHQXXFVNWLLAQKG

SEQ ID NO: 204 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16 (Pal)
YXEGTFISDYSIXLDRXAQXXFVNWLLAQRG

SEQ ID NO: 205 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16 (Pal)

SEQ ID NO: 206 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 207 - protein, 31 aa, Artificial Sequence
  source: /note=“Description of Artificial Sequence Synthetic polypeptide”
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YGEGTFISDYSIALDRXAQXXFVNWLLAQKG

SEQ ID NO: 208 - protein, 31 aa, Artificial Sequence; identical to SEQ ID NO: 204
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 209 - protein, 31 aa, Artificial Sequence; identical to SEQ ID NO: 204
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16DA

SEQ ID NO: 210 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQKPSSGAPPPS

SEQ ID NO: 211 - protein, 30 aa, Artificial Sequence; identical to SEQ ID NO: 25
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 212 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVNWLLAQRG

SEQ ID NO: 213 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence OEGgEgE-C15DA

SEQ ID NO: 214 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA

SEQ ID NO: 215 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 3OEGgEgE-C15DA

SEQ ID NO: 216 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5gEgE-C15DA

SEQ ID NO: 217 - protein, 31 aa, Artificial Sequence; identical to SEQ ID NO: 26
  source: /note=“Description of Artificial Sequence Synthetic polypeptide”
  MOD_RES (2)..(2): d-Ala
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 218 - protein, 31 aa, Artificial Sequence
  source: /note=“Description of Artificial Sequence Synthetic polypeptide”
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YAEGTFISDYSIALDRXAQXXFVNWLLAQKG

SEQ ID NO: 219 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEG-C15DA

SEQ ID NO: 220 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDKXAQXXFVNWLLAQKG

SEQ ID NO: 221 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIALDRXXQXDFVNWLLAQKG

SEQ ID NO: 222 - protein, 31 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgEgE-C15DA
  MOD_RES (20)..(20): Aib
YXEGTFISDYSIALDRXAQXDFVNWLLAQKG

SEQ ID NO: 223 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C15DA
YXEGTFISDYSIALDRXAQXDFVNWLLAXKG

SEQ ID NO: 224 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15DA
YXEGTFISDYSIALDRXAQXDFVNWLLAQXG

SEQ ID NO: 225 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXAQXXFVEWLLAQKG

SEQ ID NO: 226 - protein, 31 aa, Artificial Sequence; identical to SEQ ID NO: 222
  source: /note=“Description of Artificial Sequence Synthetic polypeptide”
  MOD_RES (2)..(2): Aib
  MOD_RES (17)..(17): Aib
  MOD_RES (20)..(20): Lys, wherein the side chain of Lys is connected to the sequence 2OEGgEgE-C15DA

SEQ ID NO: 227 - protein, 30 aa, Artificial Sequence
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C16DA
YXEGTFISDYSIALDRIHQQNFVNWLLAQX

SEQ ID NO: 228 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
YXEGTFISDYSIAXDRIHQQNFVNWLLAQK

SEQ ID NO: 229 - protein, 30 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C16DA
YXEGTFISDYSIALDRXHQQNFVNWLLAQK

SEQ ID NO: 230 - protein, 30 aa, Artificial Sequence; identical to SEQ ID NO: 227
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C16DA

SEQ ID NO: 231 - protein, 30 aa, Artificial Sequence; identical to SEQ ID NO: 227
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C15DA

SEQ ID NO: 232 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C16DA
  MOD_RES (17)..(17): Aib
  MOD_RES (20)..(20): Aib
YXEGTFISDYSIAXDRXAQXNFVNWLLAQR

SEQ ID NO: 233 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C16DA
YXEGTFISDYSIAXDRIAQQNFVNWLLAQR

SEQ ID NO: 234 - protein, 30 aa, Artificial Sequence; identical to SEQ ID NO: 232
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
  MOD_RES (17)..(17): Aib
  MOD_RES (20)..(20): Aib

SEQ ID NO: 235 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
  MOD_RES (17)..(17): Aib
YXEGTFISDYSIAXDRXAQQNFVNWLLAQR

SEQ ID NO: 236 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
  MOD_RES (20)..(20): Aib
YXEGTFISDYSIAXDRIAQXNFVNWLLAQR

SEQ ID NO: 237 - protein, 30 aa, Artificial Sequence; identical to SEQ ID NO: 233
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA

SEQ ID NO: 238 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C16DA
YXEGTFISDYSIAXDRIAQQNFVNWLLAQK

SEQ ID NO: 239 - protein, 30 aa, Artificial Sequence; identical to SEQ ID NO: 2
  source: /note=“Description of Artificial Sequence Synthetic polypeptide”
  MOD_RES (2)..(2): Aib
  MOD_RES (14)..(14): Lys, wherein the side chain of Lys is connected to the sequence 4OEGgE-C16DA
  MOD_RES (17)..(17): Aib

SEQ ID NO: 240 - protein, 30 aa, Artificial Sequence; identical to SEQ ID NO: 228
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C15DA

SEQ ID NO: 241 - protein, 30 aa, Artificial Sequence; identical to SEQ ID NO: 229
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA

SEQ ID NO: 242 - protein, 30 aa, Artificial Sequence
  source: /note=“Description of Artificial Sequence Synthetic polypeptide”
  MOD_RES (14)..(14): Lys, wherein the side chain of Lys is connected to the sequence 2OEGgE-C16DA
  MOD_RES (20)..(20): Aib
YAEGTFISDYSIAXDRIHQXNFVNWLLAQK

SEQ ID NO: 243 - protein, 30 aa, Artificial Sequence; identical to SEQ ID NO: 17
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C15DA
  MOD_RES (17)..(17): Aib
  MOD_RES (20)..(20): Aib

SEQ ID NO: 244 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C15DA
  MOD_RES (17)..(17): Aib
  MOD_RES (20)..(20): Aib

SEQ ID NO: 245 - protein, 30 aa, Artificial Sequence
  source: /note=“Description of Artificial Sequence Synthetic polypeptide”
  MOD_RES (14)..(14): Lys, wherein the side chain of Lys is connected to the sequence 2OEGgE-C15DA
  MOD_RES (17)..(17): Aib
  MOD_RES (20)..(20): Aib
YGEGTFISDYSIAXDRXHQXNFVNWLLAQK

SEQ ID NO: 246 - protein, 30 aa, Artificial Sequence
  source: /note=“Description of Artificial Sequence Synthetic polypeptide”
  MOD_RES (14)..(14): Lys, wherein the side chain of Lys is connected to the sequence 2OEGgE-C15DA
  MOD_RES (17)..(17): Aib
  MOD_RES (20)..(20): Aib
YSEGTFISDYSIAXDRXHQXNFVNWLLAQK

SEQ ID NO: 247 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIXLDRXAQXXFVNWLLAQRPSSGAPPPS

SEQ ID NO: 248 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C15DA
  MOD_RES (17)..(17): Aib
  MOD_RES (20)..(20): Aib
YXEGTFISDYSIAXDRXAQXNFVNWLLAQKG

SEQ ID NO: 249 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence OEGgEgEgE-C15DA
  MOD_RES (20)..(20): Aib

SEQ ID NO: 250 - protein, 30 aa, Artificial Sequence; identical to SEQ ID NO: 17
  source: /note=“Description of Artificial Sequence Synthetic polypeptide”
  MOD_RES (2)..(2): Aib
  MOD_RES (14)..(14): Lys, wherein the side chain of Lys is connected to the sequence G2E3-C15DA
  MOD_RES (17)..(17): Aib
  MOD_RES (20)..(20): Aib

SEQ ID NO: 251 - protein, 30 aa, Artificial Sequence; identical to SEQ ID NO: 17
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 252 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5-C15DA
YXEGTFISDYSIALDRXHQXXFVNWLLAQS

SEQ ID NO: 253 - protein, 30 aa, Artificial Sequence; identical to SEQ ID NO: 25
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence GGPAPAP-C15DA

SEQ ID NO: 254 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDKXAQXDFVNWLLAXK

SEQ ID NO: 255 - protein, 30 aa, Artificial Sequence; identical to SEQ ID NO: 36
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence OEGgEgE-C15DA

SEQ ID NO: 256 - protein, 30 aa, Artificial Sequence; identical to SEQ ID NO: 43
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence GGPAPAP-C15DA
  MOD_RES (20)..(20): Aib

SEQ ID NO: 257 - protein, 30 aa, Artificial Sequence; identical to SEQ ID NO: 12
  source: /note=“Description of Artificial Sequence Synthetic polypeptide”
  MOD_RES (2)..(2): Aib
  MOD_RES (14)..(14): Lys, wherein the side chain of Lys is connected to the sequence GGPAPAP-C15DA
  MOD_RES (17)..(17): Aib
  MOD_RES (20)..(20): Aib

SEQ ID NO: 258 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G3gEgE-C15DA
  MOD_RES (17)..(17): Aib
  MOD_RES (20)..(20): Aib

SEQ ID NO: 259 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G3gEgE-C15DA
  MOD_RES (17)..(17): Aib
  MOD_RES (20)..(20): Aib

SEQ ID NO: 260 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C15DA
  MOD_RES (17)..(17): Aib
  MOD_RES (20)..(20): Aib
YXEGTFISDYSIXXDRXAQXNFVNWLLAQS

SEQ ID NO: 261 - protein, 30 aa, Artificial Sequence; identical to SEQ ID NO: 60
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gEgE-C15DA
  MOD_RES (17)..(17): Aib
  MOD_RES (20)..(20): Aib

SEQ ID NO: 262 - protein, 30 aa, Artificial Sequence; identical to SEQ ID NO: 60
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G3E2-C15DA
  MOD_RES (17)..(17): Aib
  MOD_RES (20)..(20): Aib

SEQ ID NO: 263 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G3E2-C15DA
  MOD_RES (17)..(17): Aib
  MOD_RES (20)..(20): Aib
YXEGTFISLYSIXXDRXAQXNFVNWLLAQR

SEQ ID NO: 264 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5gEgE-C15DA

SEQ ID NO: 265 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISDYSIALDRXHQXXFVNWLLAQR

SEQ ID NO: 266 - protein, 30 aa, Artificial Sequence; identical to SEQ ID NO: 252
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 267 - protein, 30 aa, Artificial Sequence; identical to SEQ ID NO: 86
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 268 - protein, 30 aa, Artificial Sequence; identical to SEQ ID NO: 86
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 269 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA
YXEGTFISLYSIALDRXAQXXFVNWLLAQS

SEQ ID NO: 270 - protein, 39 aa, Artificial Sequence; identical to SEQ ID NO: 48
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib

SEQ ID NO: 271 - protein, 39 aa, Artificial Sequence; identical to SEQ ID NO: 33
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence G5gEgE-C15DA

SEQ ID NO: 272 - protein, 39 aa, Artificial Sequence; identical to SEQ ID NO: 33
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence G5gE-C15DA

SEQ ID NO: 273 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G5gE-C15DA
  MOD_RES (20)..(20): Aib
YXEGTFISDYSIALDRXXQXNFVNWLLAQRPSSGAPPPS

SEQ ID NO: 274 - protein, 39 aa, Artificial Sequence; identical to SEQ ID NO: 273
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C15DA
  MOD_RES (20)..(20): Aib

SEQ ID NO: 275 - protein, 39 aa, Artificial Sequence; identical to SEQ ID NO: 80
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C15DA
  MOD_RES (17)..(17): Aib
  MOD_RES (20)..(20): Aib

SEQ ID NO: 276 - protein, 39 aa, Artificial Sequence; identical to SEQ ID NO: 50
  source: /note=“Description of Artificial Sequence Synthetic polypeptide”
  MOD_RES (2)..(2): Aib
  MOD_RES (14)..(14): Lys, wherein the side chain of Lys is connected to the sequence G5gEgE-C15DA
  MOD_RES (17)..(17): Aib
  MOD_RES (20)..(20): Aib

SEQ ID NO: 277 - protein, 39 aa, Artificial Sequence; identical to SEQ ID NO: 48
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C15DA
  MOD_RES (20)..(20): Aib

SEQ ID NO: 278 - protein, 39 aa, Artificial Sequence; identical to SEQ ID NO: 84
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA

SEQ ID NO: 279 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C15DA
YXEGTFISDYSIALDRXHQXNFVNWLLAXR

SEQ ID NO: 280 - protein, 31 aa, Artificial Sequence; identical to SEQ ID NO: 212
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5gE-C15DA

SEQ ID NO: 281 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgE-C15DA
YXEGTFISDYSIALDRXAQXNFVNWLLAXSPSSGAPPPS

SEQ ID NO: 282 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence G5gE-C15DA
  MOD_RES (20)..(20): Aib
YXEGTFISDYSIALDRXXQXNFVNWLLAQR

SEQ ID NO: 283 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA

SEQ ID NO: 284 - protein, 30 aa, Artificial Sequence; identical to SEQ ID NO: 86
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence G5gE-C15DA

SEQ ID NO: 285 - protein, 30 aa, Artificial Sequence; identical to SEQ ID NO: 86
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgE-C15DA

SEQ ID NO: 286 - protein, 39 aa, Artificial Sequence; identical to SEQ ID NO: 34
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C15DA
  MOD_RES (20)..(20): Aib

SEQ ID NO: 287 - protein, 39 aa, Artificial Sequence; identical to SEQ ID NO: 21
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgE-C15DA
  MOD_RES (20)..(20): Aib

SEQ ID NO: 288 - protein, 39 aa, Artificial Sequence; identical to SEQ ID NO: 50
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C15DA
  MOD_RES (17)..(17): Aib
  MOD_RES (20)..(20): Aib

SEQ ID NO: 289 - protein, 39 aa, Artificial Sequence; identical to SEQ ID NO: 20
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C15DA
  MOD_RES (17)..(17): Aib
  MOD_RES (20)..(20): Aib

SEQ ID NO: 290 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence OEGgEgEgE-C15DA
  MOD_RES (17)..(17): Aib
  MOD_RES (20)..(20): Aib
YXEGTFISDYSIXXDRXAQXNFVNWLLAQK

SEQ ID NO: 291 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C15DA
  MOD_RES (20)..(20): Aib
YXEGTFISDYSIXLDRXXQXNFVNWLLAQR

SEQ ID NO: 292 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G5-C15DA
  MOD_RES (20)..(20): Aib
YXEGTFISDYSIXLDRXAQXNFVNWLLAQRPSSKKKKKK

SEQ ID NO: 293 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEG-C15DA
YXEGTFISDYSIXLDRXAQXXFVNWLLAQRPSSKKKKKK

SEQ ID NO: 294 - protein, 39 aa, Artificial Sequence; identical to SEQ ID NO: 34
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C16DA
  MOD_RES (20)..(20): Aib

SEQ ID NO: 295 - protein, 35 aa, Artificial Sequence; identical to SEQ ID NO: 97
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15DA

SEQ ID NO: 296 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C15DA
  MOD_RES (20)..(20): Aib
YXEGTFISDYSIXLDRXAQXNFVNWLLAQSPSSGAPPPS

SEQ ID NO: 297 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 298 - protein, 39 aa, Artificial Sequence; identical to SEQ ID NO: 81
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib

SEQ ID NO: 299 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDSXAQXNFVNWLLAQRPSSGAPPPS

SEQ ID NO: 300 - protein, 31 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib
YXEGTFISDYSIALDRXAQXDFVNWLLAQRG

SEQ ID NO: 301 - protein, 39 aa, Artificial Sequence; identical to SEQ ID NO: 105
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C15DA
  MOD_RES (20)..(20): Aib

SEQ ID NO: 302 - protein, 31 aa, Artificial Sequence; identical to SEQ ID NO: 300
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C16DA
  MOD_RES (20)..(20): Aib

SEQ ID NO: 303 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C15DA
  MOD_RES (17)..(17): Aib
  MOD_RES (20)..(20): Aib
YXEGTFISDYSIAXDKXAQXDFVNWLLAQKG

SEQ ID NO: 304 - protein, 40 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence gE-C16
YXEGTFISDYSIAMDKXHQQDFVNWLLAQKGKKNDWKHNI

SEQ ID NO: 305 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys(Ac)

SEQ ID NO: 306 - protein, 31 aa, Artificial Sequence; identical to SEQ ID NO: 204
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys(Ac)

SEQ ID NO: 307 - protein, 40 aa, Artificial Sequence
  source: /note=“Description of Artificial Sequence Synthetic polypeptide”
  VARIANT (2)..(2): /replace=“Aib” or “D-Ala” or “Gly” or “Pro”
  VARIANT (9)..(9): /replace=“Leu”
  VARIANT (13)..(13): /replace=“Aib”
  VARIANT (14)..(14): /replace=“Aib” or “Lys”
  VARIANT (16)..(16): /replace=“Ser” or “Lys”
  VARIANT (17)..(17): /replace=“Aib” or “Ile”
  VARIANT (18)..(18): /replace=“His” or “Lys”
  VARIANT (20)..(20): /replace=“Aib” or “Lys” or “Ala”
  VARIANT (21)..(21): /replace=“Asn” or “Lys”
  VARIANT (24)..(24): /replace=“Glu”
  VARIANT (26)..(26): /replace=“Lys”
  VARIANT (28)..(28): /replace=“Lys” or “Aib”
  VARIANT (30)..(30): /replace=“Gly” or “Ser” or “Lys”
  VARIANT (31)..(31): /replace=“Pro” or “ ”
  VARIANT (32)..(32): /replace=“Gly” or “ ”
  VARIANT (33)..(33): /replace=“Gly” or “ ”
  VARIANT (34)..(34): /replace=“Lys” or “Asn” or “ ”
  VARIANT (35)..(35): /replace=“Asp” or “Ser” or “Lys” or “ ”
  VARIANT (36)..(36): /replace=“Trp” or “Lys” or “ ”
  VARIANT (37)..(37): /replace=“Lys” or “Gly” or “ ”
  VARIANT (38)..(38): /replace=“His” or “Lys” or “ ”
  VARIANT (39)..(39): /replace=“Asn” or “Gly” or “Lys” or “ ”
  VARIANT (40)..(40): /replace=“Lys” or “ ”
  SITE (1)..(40): /note=“Variant residues given in the sequence have no preference with respect to those in the annotations for variant positions”
YAEGTFISDYSIALDRQAQQDFVNWLLAQRGSSGAPPPSI

SEQ ID NO: 308 - protein, 40 aa, Artificial Sequence
  source: /note=“Description of Artificial Sequence Synthetic polypeptide”
  VARIANT (2)..(2): /replace=“Aib” or “Ala” or “D-Ala” or “Gly”
  VARIANT (13)..(13): /replace=“Aib” or “Ala”
  VARIANT (14)..(14): /replace=“Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (16)..(16): /replace=“Ser” or “Lys”
  VARIANT (17)..(17): /replace=“Aib” or “Gln” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (18)..(18): /replace=“His” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (19)..(19): /replace=“Ala”
  VARIANT (20)..(20): /replace=“Aib” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (21)..(21): /replace=“Glu” or “Asp” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (24)..(24): /replace=“Glu”
  VARIANT (26)..(26): /replace=“Lys, wherein the side chain of Lys is connected to The sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (29)..(29): /replace=“Aib” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (30)..(30): /replace=“Gly” or “Lys” or “Ser” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  VARIANT (31)..(31): /replace=“Pro” or “ ”
  VARIANT (32)..(32): /replace=“Lys” or “Pro” or “Gly” or “ ”
  VARIANT (33)..(33): /replace=“Lys” or “Gly” or “ ”
  VARIANT (34)..(34): /replace=“Lys” or “Asn” or “ ”
  VARIANT (35)..(35): /replace=“Asp” or “Ser” or “Lys” or “ ”
  VARIANT (36)..(36): /replace=“Trp” or “Lys” or “ ”
  VARIANT (37)..(37): /replace=“Lys” or “Gly” or “ ”
  VARIANT (38)..(38): /replace=“His” or “Lys” or “ ”
  VARIANT (39)..(39): /replace=“Asn” or “Lys” or “Gly” or “ ”
  VARIANT (40)..(40): /replace=“ ” or “Lys, wherein the side chain of Lys is connected to the sequence gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE”
  SITE (1)..(40): /note=“Variant residues given in the sequence have no preference with respect to those in the annotations for variant positions”
YSEGTFISDYSIYLDRIAQQNFVNWLLAQRGSSGAPPPSI

SEQ ID NO: 309 - protein, 5 aa, Artificial Sequence
  source: /note=“Description of Artificial Sequence Synthetic peptide”
GGGGG

SEQ ID NO: 310 - protein, 5 aa, Artificial Sequence
  source: /note=“Description of Artificial Sequence Synthetic peptide”
GGEEE

SEQ ID NO: 311 - protein, 5 aa, Artificial Sequence
  source: /note=“Description of Artificial Sequence Synthetic peptide”
  MOD_RES (4)..(4): gamma-Glu
  MOD_RES (5)..(5): gamma-Glu
GGGXX

SEQ ID NO: 312 - protein, 7 aa, Artificial Sequence
  source: /note=“Description of Artificial Sequence Synthetic peptide”
GGPAPAP

SEQ ID NO: 313 - protein, 5 aa, Artificial Sequence
  source: /note=“Description of Artificial Sequence Synthetic peptide”
  MOD_RES (5)..(5): gamma-Glu
GGGGX

SEQ ID NO: 314 - protein, 6 aa, Artificial Sequence
  source: /note=“Description of Artificial Sequence Synthetic peptide”
  MOD_RES (6)..(6): gamma-Glu
GGGGGX

SEQ ID NO: 315 - protein, 7 aa, Artificial Sequence
  source: /note=“Description of Artificial Sequence Synthetic peptide”
  MOD_RES (6)..(6): gamma-Glu
  MOD_RES (7)..(7): gamma-Glu
GGGGGXX

SEQ ID NO: 316 - protein, 39 aa, Artificial Sequence
  source: /note=“Description of Artificial Sequence Synthetic polypeptide”
  VARIANT (2)..(2): /replace=“Aib” or “Ser”
  VARIANT (6)..(6): /replace=“Leu”
  VARIANT (7)..(7): /replace=“Thr”
  VARIANT (13)..(13): /replace=“Aib” or “Tyr”
  VARIANT (14)..(14): /replace=“Lys” or “Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (14)..(14): /replace=”CONT. FROM ABOVE or OEGgEgEgE“
  SITE (14)..(14): /note=”Each sequence connected to the side chain of Lys may be linked to one of the following lipids C14-C18 monoacid or C14-C18 diacid“
  VARIANT (16)..(16): /replace=”Arg“ or ”Ser“
  VARIANT (17)..(17): /replace=”Aib“ or ”Lys“ or ”Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (17)..(17): /replace=“CONT. FROM ABOVE or OEGgEgEgE”
  SITE (17)..(17): /note=“Each sequence connected to the side chain of Lys may be linked to one of the following lipids C14-C18 monoacid or C14-C18 diacid”
  VARIANT (18)..(18): /replace=“His” or “Lys” or “Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (18)..(18): /replace=”CONT. FROM ABOVE or OEGgEgEgE“
  SITE (18)..(18): /note=”Each sequence connected to the side chain of Lys may be linked to one of the following lipids C14-C18 monoacid or C14-C18 diacid“
  VARIANT (20)..(20): /replace=”Lys“ or ”Aib“ or ”Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (20)..(20): /replace=“CONT. FROM ABOVE or OEGgEgEgE”
  SITE (20)..(20): /note=“Each sequence connected to the side chain of Lys may be linked to one of the following lipids C14-C18 monoacid or C14-C18 diacid”
  VARIANT (21)..(21): /replace=“Lys” or “Asn” or “Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (21)..(21): /replace=”CONT. FROM ABOVE or OEGgEgEgE“
  SITE (21)..(21): /note=”Each sequence connected to the side chain of Lys may be linked to one of the following lipids C14-C18 monoacid or C14-C18 diacid“
  VARIANT (28)..(28): /replace=”Aib“ or ”Lys“ or ”Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (28)..(28): /replace=“CONT. FROM ABOVE or OEGgEgEgE”
  SITE (28)..(28): /note=“Each sequence connected to the side chain of Lys may be linked to one of the following lipids C14-C18 monoacid or C14-C18 diacid”
  VARIANT (29)..(29): /replace=“Lys” or “Aib” or “Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE, GGPAPAP, OEGgEgE,
  VARIANT (29)..(29): /replace=”CONT. FROM ABOVE or OEGgEgEgE“
  SITE (29)..(29): /note=”Each sequence connected to the side chain of Lys may be linked to one of the following lipids C14-C18 monoacid or C14-C18 diacid“
  VARIANT (30)..(30): /replace=”Ser“ or ”Arg“ or ”Lys(Ac)“ or ”Lys, wherein the side chain of Lys is connected to the sequence 1OEGgE, 2OEG, 2OEGgE, 2OEGgEgE, 2OEGgEgEgE, 3OEGgE, 3OEGgEgE, G2E3, G3gEgE, G4E2, G4gE, G4gEgE, GGGGG, G5E, G5gE, G5gEgE, gE, gEgEgE, GGEEE,
  VARIANT (30)..(30): /replace=“CONT. FROM ABOVE GGPAPAP, OEGgEgE, or OEGgEgEgE”
  SITE (30)..(30): /note=“Each sequence connected to the side chain of Lys may be linked to one of the following lipids C14-C18 monoacid or C14-C18 diacid”
  VARIANT (31)..(31): /replace=“Gly” or “ ”
  VARIANT (32)..(32): /replace=“Gly” or “ ”
  VARIANT (33)..(33): /replace=“Gly” or “ ”
  VARIANT (34)..(34): /replace=“Lys” or “ ”
  VARIANT (35)..(35): /replace=“Ser” or “Lys” or “ ”
  VARIANT (36)..(36): /replace=“Lys” or “ ”
  VARIANT (37)..(37): /replace=“Lys” or “Gly” or “ ”
  VARIANT (38)..(38): /replace=“Lys” or “ ”
  VARIANT (39)..(39): /replace=“Gly” or “Lys” or “ ”
  SITE (1)..(39): /note=“Variant residues given in the sequence have no preference with respect to those in the annotations for variant positions”
YGEGTFISDYSIALDKIAQQDFVNWLLAQKPSSGAPPPS

SEQ ID NO: 317 - protein, 6 aa, Artificial Sequence
  source: /note=“Description of Artificial Sequence Synthetic peptide”
GGGGEE

SEQ ID NO: 318 - protein, 6 aa, Artificial Sequence
  source: /note=“Description of Artificial Sequence Synthetic peptide”
  MOD_RES (5)..(5): gamma-Glu
  MOD_RES (6)..(6): gamma-Glu
GGGGXX

SEQ ID NO: 319 - protein, 6 aa, Artificial Sequence
  source: /note=“Description of Artificial Sequence Synthetic peptide”
GGGGGE

SEQ ID NO: 320 - protein, 32 aa, Artificial Sequence
  source: /note=“Description of Artificial Sequence Synthetic polypeptide”
  MOD_RES (2)..(2): Aib
  VARIANT (13)..(13): /replace=“Aib”
  MOD_RES (17)..(17): Aib
  VARIANT (18)..(18): /replace=“Lys” or “Lys, wherein the side chain of Lys is connected to the sequence 2OEGgEgE or GGGGG”
  SITE (18)..(18): /note=“Each sequence connected to the side chain of Lys may be linked to one of the following lipids C15 diacid or C16 diacid”
  MOD_RES (20)..(20): Aib
  VARIANT (21)..(21): /replace=“Asp” or “Lys, wherein the side chain of Lys is connected to the sequence 2OEGgEgE or GGGGG”
  SITE (21)..(21): /note=“Each sequence connected to the side chain of Lys may be linked to one of the following lipids C15 diacid or C16 diacid”
  VARIANT (30)..(30): /replace=“Ser”
  VARIANT (31)..(31): /replace=“Pro”
  VARIANT (32)..(32): /replace=“ ”
  SITE (1)..(32): /note=“Variant residues given in the sequence have no preference with respect to those in the annotations for variant positions”
YXEGTFISDYSIALDRXAQXKFVNWLLAQKGG

SEQ ID NO: 321 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15 diacid

SEQ ID NO: 322 - protein, 30 aa, Artificial Sequence; identical to SEQ ID NO: 15
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15 diacid

SEQ ID NO: 323 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15 diacid
  MOD_RES (20)..(20): Aib

SEQ ID NO: 324 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15 diacid

SEQ ID NO: 325 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C15 diacid

SEQ ID NO: 326 - protein, 31 aa, Artificial Sequence; identical to SEQ ID NO: 143
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence GGGGG-C15 diacid
  MOD_RES (17)..(17): Aib
  MOD_RES (20)..(20): Aib

SEQ ID NO: 327 - protein, 39 aa, Artificial Sequence; identical to SEQ ID NO: 22
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15 diacid

SEQ ID NO: 328 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence GGGGG-C15 diacid
  MOD_RES (17)..(17): Aib
  MOD_RES (20)..(20): Aib

SEQ ID NO: 329 - protein, 39 aa, Artificial Sequence; identical to SEQ ID NO: 21
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C15 diacid
  MOD_RES (20)..(20): Aib

SEQ ID NO: 330 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16 diacid

SEQ ID NO: 331 - protein, 31 aa, Artificial Sequence; identical to SEQ ID NO: 26
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C16 diacid

SEQ ID NO: 332 - protein, 32 aa, Artificial Sequence; identical to SEQ ID NO: 186
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C16 diacid
  MOD_RES (20)..(20): Aib

SEQ ID NO: 333 - protein, 32 aa, Artificial Sequence; identical to SEQ ID NO: 186
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C16 diacid
  MOD_RES (20)..(20): Aib

SEQ ID NO: 334 - protein, 39 aa, Artificial Sequence; identical to SEQ ID NO: 140
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence GGGGG-C15 diacid
  MOD_RES (17)..(17): Aib
  MOD_RES (20)..(20): Aib

SEQ ID NO: 335 - protein, 40 aa, Artificial Sequence
  source: /note=“Description of Artificial Sequence Synthetic polypeptide”
  VARIANT (2)..(2): /replace=“Aib” or “D-Ala” or “Ser” or “Gly”
  VARIANT (13)..(13): /replace=“Aib” or “Ala”
  VARIANT (14)..(14): /replace=“Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (16)..(16): /replace=“Ser” or “Lys”
  VARIANT (17)..(17): /replace=“Aib” or “Gln” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (18)..(18): /replace=“His” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (20)..(20): /replace=“Aib” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (21)..(21): /replace=“Asn” or “Glu” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (24)..(24): /replace=“Glu”
  VARIANT (26)..(26): /replace=“Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (28)..(28): /replace=“Aib” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (29)..(29): /replace=“Aib” or “Gly” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (30)..(30): /replace=“Lys” or “Ser” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (31)..(31): /replace=“Pro” or “ ”
  VARIANT (32)..(32): /replace=“Gly” or “ ”
  VARIANT (33)..(33): /replace=“Gly” or “ ”
  VARIANT (34)..(34): /replace=“Lys” or “ ”
  VARIANT (35)..(35): /replace=“Lys” or “Ser” or “ ”
  VARIANT (36)..(36): /replace=“Lys” or “ ”
  VARIANT (37)..(37): /replace=“Lys” or “Gly” or “ ”
  VARIANT (38)..(38): /replace=“Lys” or “ ”
  VARIANT (39)..(39): /replace=“Lys” or “Gly” or “ ”
  MOD_RES (40)..(40): Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE
  SITE (40)..(40): /note=“May or may not be present”
  SITE (1)..(40): /note=“Variant residues given in the sequence have no preference with respect to those in the annotations for variant positions”
YAEGTFISDYSIYLDRIAQQDFVNWLLAQRGSSGAPPPSX

SEQ ID NO: 336 - protein, 8 aa, Artificial Sequence
  source: /note=“Description of Artificial Sequence Synthetic peptide”
  SITE (1)..(8): /note=“This sequence may encompass 2-8 residues”
GGGGGGGG

SEQ ID NO: 337 - protein, 10 aa, Artificial Sequence
  source: /note=“Description of Artificial Sequence Synthetic peptide”
  SITE (1)..(10): /note=“This sequence may encompass 2-10 residues”
GGGGGGGGGG

SEQ ID NO: 338 - protein, 6 aa, Artificial Sequence
  source: /note=“Description of Artificial Sequence Synthetic peptide”
  SITE (1)..(6): /note=“This sequence may encompass 2-6 residues”
GGGGGG

SEQ ID NO: 339 - protein, 6 aa, Artificial Sequence
  source: /note=“Description of Artificial Sequence Synthetic peptide”
  MOD_RES (6)..(6): gamma-Glu
GGGGGX

SEQ ID NO: 340 - protein, 7 aa, Artificial Sequence
  source: /note=“Description of Artificial Sequence Synthetic peptide”
  MOD_RES (7)..(7): gamma-Glu
GGGGGGX

SEQ ID NO: 341 - protein, 6 aa, Artificial Sequence
  source: /note=“Description of Artificial Sequence Synthetic peptide”
  SITE (1)..(6): /note=“This sequence may encompass 5-6 residues”
GGGGGG

SEQ ID NO: 342 - protein, 6 aa, Artificial Sequence
  source: /note=“Description of Artificial Sequence Synthetic peptide”
GGGGGG

SEQ ID NO: 343 - protein, 9 aa, Artificial Sequence
  source: /note=“Description of Artificial Sequence Synthetic peptide”
  SITE (1)..(9): /note=“This sequence may encompass 1-9 residues”
GGGGGGGGG

SEQ ID NO: 344 - protein, 5 aa, Artificial Sequence
  source: /note=“Description of Artificial Sequence Synthetic peptide”
  MOD_RES (1)..(1): gamma-Glu
  MOD_RES (2)..(2): gamma-Glu
  MOD_RES (3)..(3): gamma-Glu
  MOD_RES (4)..(4): gamma-Glu
  MOD_RES (5)..(5): gamma-Glu
  SITE (1)..(5): /note=“This sequence may encompass 1-5 residues”
XXXXX

SEQ ID NO: 345 - protein, 10 aa, Artificial Sequence
  source: /note=“Description of Artificial Sequence Synthetic peptide”
  SITE (1)..(10): /note=“This sequence may encompass 1-10 residues”
GGGGGGGGGG

SEQ ID NO: 346 - protein, 4 aa, Artificial Sequence
  source: /note=“Description of Artificial Sequence Synthetic peptide”
GGGG

SEQ ID NO: 347 - protein, 9 aa, Artificial Sequence
  source: /note=“Description of Artificial Sequence Synthetic peptide”
GGGGGGGGG

SEQ ID NO: 348 - protein, 6 aa, Artificial Sequence
  source: /note=“Description of Artificial Sequence Synthetic peptide”
  SITE (1)..(6): /note=“This sequence may encompass 3-6 residues”
GGGGGG

SEQ ID NO: 349 - protein, 4 aa, Artificial Sequence
  source: /note=“Description of Artificial Sequence Synthetic peptide”
  MOD_RES (1)..(1): gamma-Glu
XGGG

SEQ ID NO: 350 - protein, 4 aa, Artificial Sequence
  source: /note=“Description of Artificial Sequence Synthetic peptide”
  MOD_RES (1)..(1): epsilon-Lys
XGGG

SEQ ID NO: 351 - protein, 39 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C15 diacid
  MOD_RES (20)..(20): Aib

SEQ ID NO: 352 - protein, 31 aa, Artificial Sequence; identical to SEQ ID NO: 143
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence GGGGG-C15 diacid
  MOD_RES (17)..(17): Aib
  MOD_RES (20)..(20): Aib

SEQ ID NO: 353 - protein, 39 aa, Artificial Sequence; identical to SEQ ID NO: 22
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C15 diacid

SEQ ID NO: 354 - protein, 39 aa, Artificial Sequence; identical to SEQ ID NO: 19
  source: /note=“Description of Artificial Sequence Synthetic polypeptide”
  MOD_RES (2)..(2): Aib
  MOD_RES (14)..(14): Lys, wherein the side chain of Lys is connected to the sequence GGGGG-C15 diacid
  MOD_RES (17)..(17): Aib
  MOD_RES (20)..(20): Aib

SEQ ID NO: 355 - protein, 39 aa, Artificial Sequence; identical to SEQ ID NO: 21
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C15 diacid
  MOD_RES (20)..(20): Aib